(19) United States
(12) Patent Application Publication
(10) Pub. No.: US 2006/0141529 A1
(43) Pub. Date: Jun. 29, 2006

(54) Compositions, kits and assays containing reagents directed to cortactin and an ARG/ABL protein kinase

(76) Inventors: Anthony John Koleske, Cheshire, CT (US); Scott Nile Boyle, Hamden, CT (US); Greg Michaud, Clinton, CT (US); Barry I. Schweitzer, Cheshire, CT (US); Paul Predki, Hamden, CT (US)

Correspondence Address: BIOTECHNOLOGY LAW GROUP;C/O PORTFOLIOIP, PO BOX 52050, MINNEAPOLIS, MN, 55402, US

(21) Appl. No.: 11/191,644

(22) Filed: Jul. 27, 2005

Related U.S. Application Data
(60) Provisional application No. 60/591,827, filed on Jul. 27, 2004.

(51) Int. Cl. G01N 33/53

(52) U.S. Cl. 435/7.1

(57) ABSTRACT

It has been determined Cortactin interacts with an ARG/ABL protein kinase. Thus, provided herein are compositions, kits and assays containing reagents directed to Cortactin and an ARG/ABL protein kinase.

### RELATED PATENT APPLICATION

This application claims the benefit of U.S. Provisional Patent Application No. 60/591,827, filed 27 Jul. 2004, entitled “Method for providing protein microarrays,” naming Paul F. Predki and Barry Schweitzer as inventors, and designated by attorney docket no. 10959-022-888. This provisional patent application is incorporated herein by reference in its entirety.

### FIELD OF THE INVENTION

The invention generally relates to a protein tyrosine kinase in a family including ARG and ABL and an interacting protein referred to as Cortactin.

### BACKGROUND

Protein tyrosine kinases (PTKs) include a region that catalyzes the transfer of a phosphoryl moiety from adenosine triphosphate to one or more tyrosine amino acids in a protein substrate. PTKs often include other regions, such as SRC homology 2 (SH2) and SRC homology 3 (SH3 domains), which can interact with cellular binding partners. Dysregulation of several PTKs have been implicated in cancers and other diseases. About 90 PTKs encoded by the human genome form a large family, of which approximately 58 members are receptor PTKs, distributed into 20 subfamilies, and about 32 members are non-receptor PTKs, distributed into 10 subfamilies. One of the non-receptor PTK subfamilies is referred to as the ABL subfamily and includes two members, ABL and ARG, which sometimes are referred to as ABL-1 and ABL-2, respectively. It has been postulated that ABL and ARG influence cytoskeletal regulatory molecules important for synapse assembly and remodeling, stimulate catalase activity and catalase degradation in oxidative stress responses, and are involved in the signaling of apoptosis.

The ABL-1 gene is about 225 kilobases, is expressed as a 6 kilobase or 7 kilobase alternatively-spliced mRNA transcript, and each transcript encodes a 145 kiloDalton protein isoform. It is believed one protein isoform is localized to the nucleus, and the other isoform, having an N-terminal glycine capable of being myristoylated, is localized to the plasma membrane. Each ABL isoform includes a SH3 domain, a SH2 domain and a kinase catalytic domain. Catalytic activity of ABL is negatively regulated by its SH3 domain, and deletion of the SH3 domain transforms ABL-1 into an oncogene. It has been reported that the tyrosine kinase activity of ABL also is regulated by a specific interaction with retinoblastoma protein (RB1) and CRK binding partners.

ABL-1 has been implicated in cell differentiation, cell division, cell adhesion, and stress responses. A portion of the ABL genomic nucleotide sequence is susceptible to chromosomal translocation. In chronic myeloid leukemia (CML) the ABL gene is translocated from chromosome 9 to the center of the BCR gene on chromosome 22, yielding a chimeric BCR-ABL RNA translated into a protein of molecular weight 210 kiloDaltons. In acute lymphocytic leukemia (ALL), ABL is translocated into the 5-prime region of the BCR gene, yielding a BCR-ABL fusion in which the first exon of BCR is linked to the second ABL exon and encoding a 190-kD protein kinase.

The ABL-2 gene also is expressed as two alternatively-spliced mRNA variants bearing different 5-prime termini, each approximately 12 kilobases in length. As for ABL, one of the ARG isoforms is capable of being myristoylated. ARG protein isoforms share high sequence identity with the ABL isoforms, especially in the tyrosine kinase domain and the SH2 and SH3 domains. A portion of the ARG genomic nucleotide sequence is susceptible to chromosomal translocation. In certain myeloid leukemias, ETV6/TEL is fused with ABL-2, resulting in a chimeric protein consisting of the helix-loop-helix oligomerization domain of ETV6 and the SH2, SH3, and protein tyrosine kinase domains of ABL2. The reciprocal transcript ABL2-ETV6 also has been identified.

Cortactin has been identified as a substrate of the SRC protein tyrosine kinase. The name Cortactin reflects biological characteristics of the protein, which binds to F-actin and localizes to the cell cortex, including membrane ruffles and lamellipodia. These features, and the presence of an SH3 domain and proline-rich regions in the Cortactin protein, suggested Cortactin might link signaling events to the actin cytoskeleton. It has been reported that Cortactin can directly influence actin polymerization and endocytosis, and phosphorylation of Cortactin stimulated by SRC modulates its activity in vivo. It also has been reported Cortactin phosphorylation and subcellular localization are affected by receptor PTKs. The Cortactin gene was identified as EMS1, a putative oncogene encoding one of the transcripts amplified in certain human carcinomas (Schuuring, 1993). Directed overexpression of EMS1/cortactin was reported to increase the motility of and invasion of fibroblasts and the metastatic potential of breast cancer cells. Cortactin is also enriched in ‘invadopodia’ from invasive tumor cells, cellular protrusions associated with degradation of extracellular matrix. These findings indicate Cortactin may play a role in promoting cell motility of cancer cells and normal cells in response to growth factor stimulation.

### SUMMARY

It has been discovered that Cortactin interacts with ARG and ABL. Accordingly, provided herein are compositions, kits and assays useful for identifying molecules that modulate an interaction between Cortactin and an ARG/ABL PTK, and/or useful for monitoring or identifying a biological function or effect of the interaction between Cortactin and an ARG/ABL PTK.

Thus, provided herein is a composition comprising a Cortactin reagent and an ARG/ABL reagent. The term “Cortactin reagent” as used herein generally refers to a reagent that specifically interacts with a Cortactin protein, fragment or nucleic acid, or comprises a Cortactin protein, fragment or nucleic acid. For example, a “Cortactin reagent” includes, but is not limited to, an isolated Cortactin protein or fragment; a nucleic acid comprising a nucleotide sequence that encodes a Cortactin protein or fragment; a molecule that specifically binds to a Cortactin protein or fragment; a nucleic acid or other molecule that specifically hybridizes to a Cortactin-encoding nucleotide sequence; a cell that over-expresses or under-expresses a Cortactin protein or fragment; and others described in greater detail hereafter. Similarly, The term “ARG/ABL reagent” as used herein generally refers to a reagent that specifically interacts with an ARG and/or ABL protein, fragment or nucleic acid, or comprises an ARG or ABL protein, fragment or nucleic acid. Thus, an “ARG/ABL reagent” includes, but is not limited to, an isolated ARG/ABL protein or fragment; a nucleic acid comprising a nucleotide sequence that encodes an ARG/ABL protein or fragment; a molecule that specifically binds to an ARG/ABL protein or fragment; a nucleic acid or other molecule that specifically hybridizes to an ARG/ABL-encoding nucleotide sequence; a cell that over-expresses or under-expresses an ARG/ABL protein or fragment; and others described in greater detail hereafter. Any combination of a Cortactin reagent and an ARG/ABL reagent can be prepared, provided or utilized. For example, a nucleic acid encoding a Cortactin protein or fragment linked to a fluorescent protein may be combined with a cell that over-expresses or under-expresses an ARG protein or fragment; an isolated Cortactin protein or fragment may be combined with an isolated ARG protein or fragment; and a nucleotide sequence encoding a Cortactin protein or fragment may be combined with another nucleotide sequence that encodes an ARG protein or fragment in a nucleic acid or a cell.

In certain embodiments, a Cortactin reagent is an isolated Cortactin protein, and sometimes an isolated Cortactin protein comprises the amino acid sequence of SEQ ID NO: 24 or SEQ ID NO: 25. A Cortactin reagent sometimes is an isolated Cortactin protein fragment, sometimes is a Cortactin protein fragment that includes one or more of a SH3 domain and/or a HS1 domain. A Cortactin protein fragment sometimes includes one or more regions delineated by the following approximate amino acid termini in SEQ ID NO: 24: amino acids 496 to 548, 83 to 119, 120 to 156, 157 to 193, 194 to 230, 231 to 267 and 268 to 304. The amino acid termini for these regions may vary by one to fifteen amino acids. In some embodiments, the Cortactin reagent is an isolated nucleic acid comprising a nucleotide sequence that encodes a Cortactin protein or fragment, and sometimes the isolated nucleic acid comprises a nucleotide sequence of SEQ ID NO: 21, 22, 23, 26 or 28.

In some embodiments, a Cortactin reagent is a Cortactin protein or fragment in association with a detectable label (e.g., a fluorescent label, luminescent label, light scattering label, antibody epitope label). A detectable label sometimes is covalently linked to the Cortactin protein or fragment, and at times the detectable label is non-covalently linked to the Cortactin protein or fragment (e.g., via a binding pair interaction such as between biotin and either avidin or streptavidin). A detectable label sometimes is a luminescent protein or fluorescent protein (e.g., a green fluorescent protein (GFP) or color variant thereof (e.g., yellow, blue or red fluorescent protein)). When the detectable label is a fluorescent molecule, the fluorescent molecule sometimes interacts with another fluorescent molecule in the composition in a fluorescence-resonance energy transfer (FRET) interaction (e.g., a Cortactin protein or fragment is linked to a fluorescent donor molecule and an ARG protein or fragment is linked to a fluorescent acceptor molecule that excites at an emission wavelength of the donor).

In certain embodiments, a Cortactin reagent is a molecule that specifically binds to a Cortactin protein or fragment. The molecule sometimes is an antibody that specifically binds to Cortactin, and sometimes the antibody is in association with a detectable label. The molecule in some embodiments is an isolated Cortactin binding partner or portion thereof, and sometimes the molecule is a Cortactin binding partner or portion thereof in association with a detectable label. In some embodiments, a Cortactin protein or fragment is phosphorylated and a binding molecule specifically binds to a site on Cortactin comprising one or more phosphoryl moieties.

In some embodiments, a Cortactin reagent is a modulator of a biological function of a Cortactin protein or fragment. A modulator may be an inhibitor or activator of a biological function. A “biological function” of a Cortactin protein or fragment sometimes is binding to an ARG/ABL protein or fragment, sometimes is phosphorylation by an ARG/ABL protein or fragment, and at times is binding of Cortactin to a binding partner other than an ARG/ABL protein or fragment, such as actin or a microtubule component, for example. In certain embodiments, a Cortactin reagent is an inhibitor of a nucleic acid that encodes a Cortactin protein or fragment. An inhibitor of a nucleic acid decreases the amount of nucleic acid product (e.g., mRNA) or amino acid product (e.g., protein) encoded by the target nucleic acid. A nucleic acid inhibitor sometimes is a siRNA, RNAi, antisense or ribozyme nucleic acid.

In certain embodiments, a Cortactin reagent is a cell that over-expresses a Cortactin protein or fragment and/or a nucleic acid encoding a Cortactin protein or fragment. In some embodiments, a cell that under-expresses a Cortactin protein, fragment or nucleic acid product contains no nucleic acid that can encode such a product (e.g., the cell is from a knock-out mouse) and no detectable amount of the product is produced. A cell that under-expresses a Cortactin protein, fragment or nucleic acid sometimes is in contact with a nucleic acid inhibitor that blocks or reduces the amount of the product produced by the cell in the absence of the inhibitor. An over-expressing or under-expressing cell may be within an organism (in vivo) or from an organism (ex vivo or in vitro). Organisms include, but are not limited to, mice, rats, hamsters, rabbits, cats, dogs, monkeys, apes and humans. A cell may be genetically modified before, during or after it is excised for in vitro or ex vivo applications. A modified cell product, such as a fixed cell (e.g., cell fixed to glass slide) or lysate preparation of a cell described herein, for example, may be utilized as a reagent.

In some embodiments, the ARG/ABL reagent is an ARG reagent; the ARG/ABL reagent is an isolated ARG protein; the isolated ARG protein comprises the amino acid sequence of SEQ ID NO: 4 or SEQ ID NO: 5; the ARG/ABL reagent is an isolated ARG protein fragment; the ARG protein fragment includes one or more of a SH3 domain, SH2 domain, protein tyrosine kinase catalytic domain, actin-binding domain and/or microtubule binding domain; the ARG protein fragment includes one or more regions delineated by the following approximate amino acid termini in SEQ ID NO: 4: amino acids 75 to 128, 136 to 227 and 244 to 506. In certain embodiments, the ARG/ABL reagent is an ABL reagent; the ARG/ABL reagent is an isolated ABL protein; the isolated ABL protein comprises the amino acid sequence of SEQ ID NO: 9 or SEQ ID NO: 10; the ARG/ABL reagent is an isolated ABL protein fragment; the ABL protein fragment includes one or more of a SH3 domain, SH2 domain, protein tyrosine kinase catalytic domain, actin-binding domain and/or microtubule binding domain; the ABL protein fragment includes one or more regions delineated by the following approximate amino acid termini in SEQ ID NO: 10: amino acids 84 to 137, 145 to 236 and 253 to 515. The amino acid termini for these regions may vary by one to fifteen amino acids. In some embodiments, the ARG/ABL reagent is an isolated nucleic acid comprising a nucleotide sequence that encodes an ARG/ABL protein or fragment, and sometimes the isolated nucleic acid comprises a nucleotide sequence of SEQ ID NO: 1, 2, 3, 6, 7, 8, 13, 15, 17 or 19.

In some embodiments, an ARG/ABL reagent is an ARG/ABL protein or fragment in association with a detectable label (e.g., a fluorescent label, luminescent label, light scattering label, antibody epitope label). A detectable label sometimes is covalently linked to the ARG/ABL protein or fragment, and at times the detectable label is non-covalently linked to the ARG/ABL protein or fragment (e.g., via a binding pair interaction such as between biotin and either avidin or streptavidin). A detectable label sometimes is a luminescent protein or fluorescent protein (e.g., a green fluorescent protein (GFP) or color variant thereof (e.g., yellow, blue or red fluorescent protein)). When the detectable label is a fluorescent molecule, the fluorescent molecule sometimes interacts with another fluorescent molecule in the composition in a fluorescence-resonance energy transfer (FRET) interaction (e.g., an ARG/ABL protein or fragment is linked to a fluorescent donor molecule and a Cortactin protein or fragment is linked to a fluorescent acceptor molecule that excites at an emission wavelength of the donor).

In certain embodiments, an ARG/ABL reagent is a molecule that specifically binds to an ARG/ABL protein or fragment. The molecule sometimes is an antibody that specifically binds to ARG/ABL, and sometimes the antibody is in association with a detectable label. The molecule in some embodiments is an isolated ARG/ABL binding partner or portion thereof, and sometimes the molecule is an ARG/ABL binding partner or portion thereof in association with a detectable label. In some embodiments, an ARG/ABL protein or fragment is phosphorylated and a binding molecule specifically binds to a site on an ARG/ABL comprising one or more phosphoryl moieties.

In some embodiments, an ARG/ABL reagent is a modulator of a biological function of an ARG/ABL protein or fragment. A modulator may be an inhibitor or activator of a biological function or biological activity. A “biological function” of an ARG/ABL protein or fragment sometimes is binding to an ARG/ABL protein or fragment, sometimes is phosphorylation by an ARG/ABL protein or fragment, and at times is binding of ARG/ABL to a binding partner other than a Cortactin protein or fragment, such as actin or a microtubule member, for example. In certain embodiments, an ARG/ABL reagent is an inhibitor of a nucleic acid that encodes an ARG/ABL protein or fragment.

In certain embodiments, an ARG/ABL reagent is a cell that over-expresses an ARG/ABL protein or fragment and/or a nucleic acid encoding an ARG/ABL protein or fragment. Over-expressing cells may be stably transfected or transiently transfected with a nucleic acid that encodes the ARG/ABL protein or fragment or nucleic acid. In some embodiments, an ARG/ABL reagent is a cell that under-expresses an ARG/ABL protein or fragment or nucleic acid product. In some embodiments, a cell that under-expresses an ARG/ABL protein, fragment or nucleic acid product contains no nucleic acid that can encode such a product (e.g., the cell is from a knock-out mouse) and no detectable amount of the product is produced. A cell that under-expresses an ARG/ABL protein, fragment or nucleic acid sometimes is in contact with a nucleic acid inhibitor that blocks or reduces the amount of the product produced by the cell in the absence of the inhibitor. An over-expressing or under-expressing cell may be within an organism (in vivo) or from an organism (ex vivo or in vitro).

In some embodiments, a composition comprises a Cortactin protein or fragment and an ARG and/or ABL protein or fragment. One or more or all of the proteins or fragments often are isolated or purified. In some embodiments, the proteins and/or fragments are in a complex, and sometimes the complex is isolated or purified. In certain embodiments, provided are isolated crystals comprising a Cortactin protein or fragment an ARG and/or ABL protein or fragment. In such compositions, complexes and crystals, the Cortactin protein or fragment sometimes is in association with an ARG and/or ABL protein or fragment, sometimes is bound to the ARG and/or ABL protein or fragment, sometimes is directly bound to the ARG and/or ABL protein or fragment, and sometimes is indirectly bound to the ARG and/or ABL protein or fragment.

A composition may include a solid support, and in some embodiments the solid support is in association with or capable of being associated with an ARG/ABL reagent and/or a Cortactin reagent. Other components can be included, such as a detection reagent or apparatus (e.g., a microscope).

Also provided herein are kits comprising one or more Cortactin reagents and one or more ARG/ABL reagents. Any of the reagents described herein may be included in a kit. A kit comprises one or more of the components in any number of separate containers, packets, tubes, vials, microtiter plates and the like, or the components may be combined in various combinations in such containers. A kit sometimes includes instructions for using included components, and sometimes components not included in the kit, in one or more methods, such as a method described hereafter.

Also provided are methods for identifying a molecule that modulates an interaction between an ARG/ABL protein or fragment and a Cortactin protein or fragment, comprising: contacting the ARG/ABL protein or fragment and the Cortactin protein or fragment with a test molecule, wherein the ARG/ABL protein or fragment and the Cortactin protein or fragment interact in the absence of the test molecule; and detecting the presence, absence or amount of an interaction between the ARG/ABL protein or fragment and the Cortactin protein or fragment; whereby the test molecule is identified as a molecule that modulates the interaction when the detected interaction differs from the interaction in the absence of test molecule. The term “interaction” as used herein generally includes covalent modification and/or non-covalent association (e.g., binding) of one protein or fragment with the other protein or fragment. In some embodiments a phosphorylation event is detected, in which a Cortactin protein or fragment is phosphorylated by an ARG/ABL protein or fragment. Phosphorylation may be detected by any suitable method (e.g., detection of a phosphotyrosine antibody or radioactive phosphoryl moiety on a protein or peptide (e.g., from a gamma ³²P or ³³P ATP substrate or derivative thereof)). In embodiments where phosphorylation of a Cortactin protein or fragment is detected, the Cortactin protein or fragment typically will include a tyrosine that can be phosphorylated by an ARG/ABL having catalytic activity (although control assays can include a Cortactin protein or fragment not containing the tyrosine (e.g., a site-specific substitution or deletion of the tyrosine)). An association event is detected in some embodiments, which can be detected by any suitable method (e.g., FRET, immuno-precipitation, solid phase capture, gel electrophoresis co-migration).

In the methods described above, each protein or fragment and test molecule may be added sequentially or contemporaneously, or in a combination thereof, in any order and in any sequence. Any ARG/ABL protein or fragment and any Cortactin protein or fragment may be utilized, and can be selected from those described herein. The ARG/ABL proteins or fragments and/or Cortactin protein or fragments often are isolated proteins or fragments. Any test molecule may be utilized, such as an organic molecule, protein, antibody or antibody fragment, or siRNA, RNAi, ribozyme or antisense nucleic acid. Other molecules or reagents may be added to the system, such as one or more solid supports in heterogeneous assay formats, one or more detection reagents and one or more binding partners of a protein or fragment in the system (e.g., microtubule component, actin). The assay may be conducted in a cell-free system or in a system that includes intact cells.

Also included herein are methods for identifying a molecule that modulates a biological activity in a system containing an ARG/ABL reagent and a Cortactin reagent, comprising: contacting the ARG/ABL reagent, the Cortactin reagent and a test molecule in the system and detecting the presence, absence or amount of the biological activity, whereby the test molecule is identified as a molecule that modulates the biological activity when the detected biological activity differs from the biological activity in the absence of test molecule. The term “biological activity” or “biological function” as used herein generally includes activities and functions of ARG, ABL and Cortactin described above and others. A biological activity can include, for example, localization of a Cortactin protein or fragment or ARG/ABL protein or fragment to a cellular location; phosphorylation of a Cortactin protein or fragment or ARG/ABL protein or fragment; interaction of a Cortactin protein or fragment with an ARG/ABL or an interaction of either with another binding partner (e.g., actin, microtubule component, Crk, CrkL, Nck, Grb2, PSTPIP1, 3BP1, 3BP2, AAP1, SHP1, p85, Mena, CAP, HPK1, Cbl, RFX1, p73, Rin1, PKCδ, ATM, MEKK-1, hTERT, RAFT1, Pag, Rad51, Cables, Scar/Wave, NR2D, EphB2 p130Cas, APP, TrkA, c-Jun, CD19, ALP); a change in cellular level, cell localization or phosphorylation state of a protein other than ABL, ARG or Cortactin affected by an increased or reduced interaction between an ARG/ABL protein or fragment and a Cortactin protein or fragment (e.g., Arp2/3 complex protein, transcription factor, nuclear protein or cytosolic protein); and a change in cell proliferation and/or a metastatic event in response to an increased or decreased interaction between an ARG/ABL protein or fragment and a Cortactin protein or fragment (e.g., a change in local invasion, passive transport, lodgement, proliferation or other metastatic process carried-out by a cancer cell). As with other assays described herein, any test molecule may be utilized, such as an organic molecule, protein, antibody or antibody fragment, or siRNA, RNAi, ribozyme or antisense nucleic acid. Other molecules or reagents may be added to the system, such as one or more solid supports in heterogeneous assay formats, one or more detection reagents and one or more binding partners of a protein or fragment in the system (e.g., microtubule component, actin). The assay may be conducted in a cell-free system or in a system that includes intact cells. Each reagent and test molecule may be added sequentially or contemporaneously, or in a combination thereof, in any order and in any sequence. Any ARG/ABL reagent and any Cortactin reagent may be utilized, and can be selected from those described herein.

Also provided herein are methods for identifying a condition that modulates a biological activity in a system containing an ARG/ABL reagent and a Cortactin reagent, comprising: exposing the ARG/ABL reagent and/or the Cortactin reagent to a test condition in the system; and detecting the presence, absence or amount of the biological activity, whereby the test condition is identified as a condition that modulates the biological activity when the detected biological activity differs from the biological activity in the absence of the test condition. Any test condition may be utilized, such as contacting a system with a growth factor, mitogenic factor, chemotherapeutic or other anti-cancer agent, a cell transformation condition (e.g., radiation). Other molecules or reagents may be added to the system, such as one or more test molecules, one or more solid supports in heterogeneous assay formats, one or more detection reagents and one or more binding partners of a protein or fragment in the system (e.g., microtubule component, actin). The assay may be conducted in a cell-free system or in a system that includes intact cells. The system may be contacted with each reagent and test condition sequentially or contemporaneously, or in a combination thereof, in any order and in any sequence. Any ARG/ABL reagent and any Cortactin reagent may be utilized, and can be selected from those described herein.

These and other embodiments are described in greater detail in the description which follows.

### BRIEF DESCRIPTION OF NUCLEOTIDE AND AMINO ACID SEQUENCES

SEQ ID NOs: 1-5 are human ABL-2 nucleotide sequences and encoded ARG amino acid sequences. SEQ ID NO: 1 is a human ABL-2 genomic nucleotide sequence (AL139132.16 GI:16972764). SEQ ID NO: 2 is a human ARG isoform 1 mRNA nucleotide sequence (NM_{—}005158.2 GI:6382059). SEQ ID NO: 3 is a human ARG isoform 2 mRNA nucleotide sequence (NM_{—}007314.1 GI:6382061). SEQ ID NO: 4 is a human ARG isoform 1 amino acid sequence (NP_{—}005149.2 GI:6382060). SEQ ID NO: 5 is a human ARG isoform 2 amino acid sequence (NP_{—}009298.1 GI:6382062).

SEQ ID NOs: 6-10 are human ABL-1 nucleotide sequences and encoded ABL amino acid sequences. SEQ ID NO: 6 is a human ABL-1 genomic nucleotide sequence (U07563.1 GI:514264). SEQ ID NO: 7 is a human ABL-1 isoform 1 mRNA nucleotide sequence (NM_{—}005157.3 GI:62362413). SEQ ID NO: 8 is a human ABL-1 isoform 2 mRNA nucleotide sequence (NM_{—}007313.2 GI:62362411). SEQ ID NO: 9 is a human ABL isoform 1 amino acid sequence (NP_{—}005148.2 GI:62362414). SEQ ID NO: 10 is a human ABL isoform 2 amino acid sequence (NP_{—}009297.2 GI:62362412).

SEQ ID NO: 11 is a mouse ABL amino acid sequence and SEQ ID NO: 12 is a mouse ARG amino acid sequence.

SEQ ID NOs: 13-16 are nucleotide sequences and amino acids sequences of BCR-ABL fusions. SEQ ID NO: 13 is a human BCR-ABL nucleotide sequence (p210; 2706 nucleotides or 2781 nucleotides of BCR fused to ABL first common exon; 2706 nucleotide fusion). SEQ ID NO: 14 is a human BCR-ABL fusion amino acid sequence (2706 nucleotide fusion). SEQ ID NO: 15 is a human BCR-ABL nucleotide sequence (2781 nucleotide fusion). SEQ ID NO: 16 is a human BCR-ABL amino acid sequence (2781 nucleotide fusion).

SEQ ID NOs: 17-20 are nucleotide sequences and amino acid sequences of TEL-ARG fusions. SEQ ID NO: 17 is a human TEL-ARG fusion nucleotide sequence (TEL 1033 fused to ARG 362). SEQ ID NO: 18 is a human TEL-ARG amino acid sequence (TEL 1033 fused to ARG 362). SEQ ID NO: 19 is a human TEL-ARG nucleotide sequence (TEL 1033 fused to 425 ARG). SEQ ID NO: 20 is a human TEL-ARG amino acid sequence (TEL 1033 fused to 425 ARG).

SEQ ID NOs: 21-29 are Cortactin nucleotide sequences and amino acid sequences. SEQ ID NO: 21 is a human Cortactin genomic nucleotide sequence (AB036705.1 GI:40645043). SEQ ID NO: 22 is a human Cortactin isoform 1 mRNA (NM_{—}005231.2 GI:20357551). SEQ ID NO: 23 is a human Cortactin isoform 2 mRNA (NM_{—}138565.1 GI:20357555). SEQ ID NO: 24 is a human Cortactin isoform 1 amino acid sequence (NP_{—}005222.2 GI:20357552). SEQ ID NO: 25 is a human Cortactin isoform 2 amino acid sequence (NP_{—}612632.1 GI:20357556). SEQ ID NO: 26 is a nucleotide sequence generated for an alternative human Cortactin isoform in which the fifth cortactin repeat (exon 10) is not included (e.g., van Rossum, et al. J. Biol. Chem. 278 (46): 45672-45679 (2003)), and SEQ ID NO: 27 is the encoded amino acid sequence generated. SEQ ID NO: 28 is a murine Cortactin nucleotide sequence, and SEQ ID NO: 29 is the encoded murine Cortactin amino acid sequence.

### DETAILED DESCRIPTION

ARG/ABL PTKs and Cortactin have been associated with certain cancers and metastasis. Accordingly, the compositions, kits and assays provided herein are expected as being useful for developing anti-cancer therapeutics by identifying molecules that modulate an interaction between Cortactin and an ARG/ABL PTK, and identifying biological effects of interactions between Cortactin and an ARG/ABL PTK.

The term “about” as used herein refers to a value sometimes within 10% of the underlying parameter (i.e., plus or minus 10%), a value sometimes within 5% of the underlying parameter (i.e., plus or minus 5%), a value sometimes within 2.5% of the underlying parameter (i.e., plus or minus 2.5%), or a value sometimes within 1% of the underlying parameter (i.e., plus or minus 1%), and sometimes refers to the parameter with no variation. Thus, a distance of “about 20 nucleotides in length” includes a distance of 19 or 21 nucleotides in length (i.e., within a 5% variation) or a distance of 20 nucleotides in length (i.e., no variation) in some embodiments. As used herein, the article “a” or “an” can refer to one or more of the elements it precedes (e.g., a nucleic acid comprising “a” promoter sequence may comprise one promoter sequence or multiple promoter sequences).

ARG/ABL and Cortactin Nucleic Acid, Protein and Fragment Reagents

An ARG/ABL nucleic acid often includes a nucleotide sequence that can encode an ARG and/or ABL protein having an amino acid sequence of SEQ ID NO: 4, 5, 9, 10, 11 or 12, a substantially identical variant thereof or a fragment of the forgoing. An ARG/ABL nucleic acid also may include a nucleotide sequence that can encode an ARG or ABL fusion protein having an amino acid sequence of SEQ ID NO: 14, 16, 18 or 20, a substantially identical variant thereof or a fragment of the forgoing. An ARG/ABL nucleic acid may include in some embodiments a nucleotide sequence from SEQ ID NO: 1, 2, 3, 6, 7, 8, 13, 15, 17 or 19. A Cortactin nucleic acid often includes a nucleotide sequence that can encode a Cortactin protein having an amino acid sequence of SEQ ID NO: 24, 25, 27 or 29, a substantially identical amino acid variant thereof, or a fragment of the foregoing. A Cortactin nucleic acid may include in some embodiments a nucleotide sequence from SEQ ID NO: 21, 22, 23, 26 or 28. The term “ARG/ABL protein or fragment” generally includes a native ARG and/or ABL isoform or fragment thereof, an ARG and/or ABL fusion (e.g., fusions between BCR and ABL or fusions between TEL and ARG) and a substantially identical variant or fragment thereof (e.g., a variant amino acid sequence 95%, 96%, 97%, 98% or 99% identical to an amino acid sequence in SEQ ID NO: 4, 5, 9, 10, 11 or 12, or having one, two, three, four, five, six, seven, eight or nine amino acid substitutions to such a sequence). An ARG/ABL protein or fragment includes in some embodiments an amino acid sequence of SEQ ID NO: 4, 5, 9, 10, 11, 12, 14, 16, 18 or 20, or is a subsequence of one of the foregoing. The term “Cortactin protein or fragment” generally includes a native Cortactin isoform or fragment thereof and a substantially identical variant or fragment thereof (e.g., a variant amino acid sequence 95%, 96%, 97%, 98% or 99% identical to the amino acid sequence in SEQ ID NO: 24, 25, 27 or 29, or having one, two, three, four, five, six, seven, eight or nine amino acid substitutions to such a sequence). A Cortactin protein or fragment includes in some embodiments an amino acid sequence of SEQ ID NO: 24, 45, 27 or 29, or is a subsequence of one of the foregoing. An ARG/ABL nucleic acid or Cortactin nucleic acid also can encode a complementary nucleic acid capable of encoding one of the foregoing amino acid sequences (e.g., an ARG/ABL nucleic acid may include a genomic nucleotide sequence or precursor RNA sequence). A Cortactin protein or fragment or nucleic acid and an ARG or ABL protein or fragment or nucleic acid can include a nucleotide sequence or amino acid sequence from any species. The sequence sometimes is a mammalian sequence (e.g., mouse, rat, hamster, ungulate, monkey) and often is a human sequence.

The term “substantially identical variant” as used herein refers to a nucleotide or amino acid sequence sharing sequence identity to a nucleotide sequence or amino acid sequence of ARG, ABL or Cortactin. Included are nucleotide sequences or amino acid sequences 55% or more, 60% or more, 65% or more, 70% or more, 75% or more, 80% or more, 85% or more, 90% or more, 95% or more (each sometimes within a 1%, 2%, 3% or 4% variability) identical to a ARG, ABL or Cortactin nucleotide sequence or encoded amino acid sequence, or has one to ten nucleotide or amino acid substitutions. One test for determining whether two nucleotide sequences or amino acids sequences are substantially identical is to determine the percent of identical nucleotide sequences or amino acid sequences shared.

Calculations of sequence identity can be performed as follows. Sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is sometimes 30% or more, 40% or more, 50% or more, often 60% or more, and more often 70% or more, 80% or more, 90% or more, or 100% of the length of the reference sequence. The nucleotides or amino acids at corresponding nucleotide or polypeptide positions, respectively, are then compared among the two sequences. When a position in the first sequence is occupied by the same nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences. Comparison of sequences and determination of percent identity between two sequences can be accomplished using a mathematical algorithm. Percent identity between two amino acid or nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444-453 (1970) algorithm which has been incorporated into the GAP program in the GCG software package (available at the http address www.gcg.com), using either a Blossum 62 matrix or a PAM250 matrix, and a gap weight of 16, 14, 12, 10, 8, 6, or 4 and a length weight of 1, 2, 3, 4, 5, or 6. Percent identity between two nucleotide sequences can be determined using the GAP program in the GCG software package (available at http address www.gcg.com), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Another manner for determining whether two nucleic acids are substantially identical is to assess whether a polynucleotide homologous to one nucleic acid will hybridize to the other nucleic acid under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C.

An example of a substantially identical nucleotide sequence to a nucleotide sequence of SEQ ID NO: 1, 2, 3, 6, 7, 8, 13, 15, 17, 19, 21, 22, 23, 26 or 28 is one that has a different nucleotide sequence but still encodes the same amino acid sequence encoded by the nucleotide sequence of 1, 2, 3, 6, 7, 8, 13, 15, 17, 19, 21, 22, 23, 26 or 28. Another example is a nucleotide sequence that encodes a protein having an amino acid sequence 70% or more identical to, sometimes 75% or more, 80% or more, or 85% or more identical to, and often 90% to 99% identical to an amino acid sequence encoded by a nucleotide sequence in SEQ ID NO: 1, 2, 3, 6, 7, 8, 13, 15, 17, 19, 21, 22, 23, 26 or 28.

Nucleotide sequences in SEQ ID NO: 1, 2, 3, 6, 7, 8, 13, 15, 17, 19, 21, 22, 23, 26 or 28 and encoded amino acid sequences can be used as “query sequences” to perform a search against public databases to identify other family members or related sequences, for example. Such searches can be performed using the NBLAST and XBLAST programs (version 2.0) of Altschul et al., J. Mol. Biol. 215: 403-10 (1990). BLAST nucleotide searches can be performed with the NBLAST program, score=100, wordlength=12 to obtain nucleotide sequences homologous to nucleotide sequences in SEQ ID NO: 1, 2, 3, 6, 7, 8, 13, 15, 17, 19, 21, 22, 23 or 26. BLAST polypeptide searches can be performed with the XBLAST program, score=50, wordlength=3 to obtain amino acid sequences homologous to those encoded by SEQ ID NO: 1, 2, 3, 6, 7, 8, 13, 15, 17, 19, 21, 22, 23, 26 or 28. To obtain gapped alignments for comparison purposes, Gapped BLAST can be utilized as described in Altschul et al., Nucleic Acids Res. 25(17): 3389-3402 (1997). When utilizing BLAST and Gapped BLAST programs, default parameters of the respective programs (e.g., XBLAST and NBLAST) can be used (see the http address www.ncbi.nlm.nih.gov). Thus, a protein having a substantially identical amino acid sequence to an amino acid sequence encoded by a nucleotide sequence of SEQ ID NO: 1, 2, 3, 6, 7, 8, 13, 15, 17, 19, 21, 22, 23, 26 or 28 and shares one or more or all of the same biological functions with a protein having an amino acid sequence of SEQ ID NO: 4, 5, 9, 10, 11, 12, 14, 16, 18, 20, 24, 25, 27 or 29.

Substantially identical nucleotide sequences may include altered codons for enhancing expression of an amino acid sequence in a particular expression system. One or more codons may be altered, and sometimes 10% or more or 20% or more of the codons are altered for optimized expression in an expression system that may include bacteria (e.g., E. coli.), yeast (e.g., S. cervesiae), human (e.g., 293 cells), insect, or rodent (e.g., hamster) cells.

An ABL or ARG protein variant or fragment thereof can include one or more amino acid substitutions, deletions or insertions. Any amino acid may be substituted by a conservative or non-conservative substitution. For example, an ABL protein or fragment may be modified at the following positions: positions 315, 255, 351, 311 and 253. In some embodiments, position 315 may be substituted with isoleucine, position 255 may be substituted with lysine or valine, position 351 may be substituted with a threonine, position 311 may be substituted with a leucine and position 253 may be substituted with a histidine. In some embodiments, one or more tyrosine, threonine or serine amino acids capable of being phosphorylated in an ARG or ABL protein or fragment are substituted or deleted. Such phosphorylateable amino acids may be substituted by an amino acid such as phenylalanine, alanine, valine or cysteine. Corresponding positions in an ARG protein or fragment, which can be determined by a sequence alignment, may be substituted. ARG and ABL proteins or fragments containing an N-terminal region can be produced with or without an N-terminal methionine amino acid (e.g., an inhibitor of an exoprotease that cleaves the N-terminal methionine can be included when protein is being synthesized), and a protein that includes an N-terminal glycine can include an N-terminal fatty acid (e.g., myristoyl moiety).

An ARG protein fragment can include one or more functional regions. Functional ARG protein regions are described hereafter with respect to amino acid positions in isoform 1 (SEQ ID NO: 4). Isoform 1 includes amino acids encoded by exon 1A but not exon 1B, and isoform 2 includes amino acids encoded by exon 1B but not exon 1A. Corresponding regions in isoform 2, ARG fusions or in ABL proteins or fusions can be determined by sequence alignment. The ARG protein comprises a SRC homology 3 domain (SH3 domain), which spans a region from about amino acid 75 to about amino acid 128 and is expected to binds to proline-rich and binds to region in a binding partner comprising one or more proline and hydrophobic amino acids. Also included is a SRC homology 2 domain (SH2 domain), which spans a region from about amino acid 136 to about amino acid 227, and binds to a region in a binding partner comprising one or more phosphotyrosine amino acids and hydrophobic amino acids. The ARG protein also comprises a catalytic protein tyrosine kinase domain, which spans a region from about amino acid 244 to about amino acid 506. Also included are two F-actin binding domains spanning from amino acids 688-930 and 1034-1182 and a microtubule binding domains from amino acid 924-1090 There are also 3 conserved Pro-X-X-Pro motifs at residues 573-576, 622-625, and 664-667, which can serve as binding site for SH3 domain-containng proteins. Positional boundaries of the aforementioned ARG regions can vary by about one to fifteen amino acids. A functional region also may include one or more amino acids that can be phosphorylated in ARG, such as a tyrosine, threonine or serine, and each phosphorylateable amino acid may be flanked on each side by one to fifty amino acids from a native ARG protein. Where an ARG protein fragment includes one or more functional regions, the region may be flanked on each side by a native amino acid sequence in an ARG protein.

An ABL protein fragment may include one or more functional regions. Functional ABL protein regions are described hereafter with respect to amino acid positions in isoform 2 (SEQ ID NO: 10). Isoform 1 includes amino acids encoded by exon 1A but not exon 1B and is localized in the nucleus. Isoform 2 includes amino acids encoded by exon 1B but not exon 1A, and includes an N-terminal glycine that may be myristoylated and may localize the isoform to the plasma membrane. Corresponding regions in isoform 1 can be determined by sequence alignment. The ABL protein comprises a SRC homology 3 domain (SH3 domain), which spans a region from about amino acid 84 to about amino acid 137. Also included is a SRC homology 2 domain (SH2 domain), which spans a region from about amino acid 145 to about amino acid 236. The ABL protein also comprises a catalytic protein tyrosine kinase domain, which spans a region from about amino acid 253 to about amino acid 515. Also included are a DNA binding region from amino acid 687-951, a G-actin-binding domain from amino acids 977-1046 and an F-actin binding domain from 1015-1142. ABL also contains 3 Pro-X-X-Pro motifs at residues 552-555, 595-598, and 638-641 that can bind to SH3-domain containing proteins. ABL also has three nuclear localization motifs from 624-628. 729-733, and 782-785 and a nuclear export sequence 1102-1106. Positional boundaries of the aforementioned ABL regions can vary by about one to fifteen amino acids. A functional region also may include one or more amino acids that can be phosphorylated in ABL, such as a tyrosine, threonine or serine, and each phosphorylateable amino acid may be flanked on each side by one to fifty amino acids from a native ABL protein. Where an ABL protein fragment includes one or more functional regions, the region may be flanked on each side by a native amino acid sequence in an ABL protein.

A Cortactin protein variant or fragment thereof can include one or more amino acid substitutions, deletions or insertions. Any amino acid may be substituted by a conservative or non-conservative substitution. In some embodiments, one or more tyrosine, threonine or serine amino acids capable of being phosphorylated in an ARG or ABL protein or fragment are substituted or deleted. Such phosphorylateable amino acids may be substituted by an amino acid such as phenylalanine, alanine, valine or cysteine. For example, positions in the following positions in a Cortactin protein or peptide may be deleted or substituted: 384, 409, 416, 427, 433 and/or 449. These positions are in SEQ ID NO: 25 and any phosphorylateable amino acid in SEQ ID NO: 24 may be deleted or substituted.

A Cortactin protein fragment can include one or more functional regions. Functional Cortactin protein regions are described hereafter with respect to amino acid positions in isoform 1 (SEQ ID NO: 24). Corresponding regions in isoform 2 can be determined by sequence alignment. The Cortactin protein comprises a SRC homology 3 domain (SH3 domain), which spans a region from about amino acid 496 to about amino acid 548. Also included are HS1 domains repeated at six locations in the protein spanning amino acid positions 83 to 119, 120 to 156, 157 to 193, 194 to 230, 231 to 267 and 268 to 304. Positional boundaries of the terminal domains can vary by about one to fifteen amino acids. Positional boundaries of the aforementioned Cortactin regions can vary by about one to fifteen amino acids. A functional region also may include one or more amino acids that can be phosphorylated in Cortactin, such as a tyrosine, threonine or serine, and each phosphorylateable amino acid may be flanked on each side by one to fifty amino acids from a native Cortactin protein. Where a Cortactin protein fragment includes one or more functional regions, the region may be flanked on each side by a native amino acid sequence in a Cortactin protein.

In certain embodiments, an ARG or ABL fragment is 10 or more, 15 or more, 20 or more, 25 or more, 50 or more, 100 or more, 200 or more, 300 or more, 400 or more, 500 or more, 600 or more, 700 or more, 800 or more, 900 or more, 1000 or more, 1100 or more amino acids from an ARG or ABL protein or substantially identical variant thereof, and can be 1200 or more, 1300 or more, 1400 or more, 1500 or more, 1600 or more, 1700 or more, 1800 or more, 1900 or more, or 2000 or more amino acids from an ARG or ABL protein fusion or substantially identical variant thereof. An ARG or ABL fragment can include a region that binds to a Cortactin protein or fragment directly or indirectly, binds to actin or a microtubule component directly or indirectly and/or binds to a binding partner directly or indirectly. In some embodiments, a Cortactin fragment is 10 or more, 15 or more, 20 or more, 25 or more, 50 or more, 100 or more, 200 or more, 300 or more, 400 or more or 500 or more amino acids from a Cortactin protein or substantially identical variant thereof, and can include a region that binds to an ARG and/or ABL protein or fragment directly or indirectly, binds to actin or a microtubule component directly or indirectly and/or binds to a binding partner directly or indirectly.

An ARG, ABL or Cortactin protein or fragment may contain one or more unnatural amino acids. Unnatural amino acids include but are not limited to D-isomer amino acids, ornithine, diaminobutyric acid, norleucine, pyrylalanine, thienylalanine, naphthylalanine and phenylglycine, alpha and alpha-disubstituted amino acids, N-alkyl amino acids, lactic acid, halide derivatives of natural amino acids such as trifluorotyrosine, p-Cl-phenylalanine, p-Br-phenylalanine, p-I-phenylalanine, L-allyl-glycine, beta-alanine, L-alpha-amino butyric acid, L-gamma-amino butyric acid, L-alpha-amino isobutyric acid, L-epsilon-amino caproic acid, 7-amino heptanoic acid, L-methionine sulfone, L-norleucine, L-norvaline, p-nitro-L-phenylalanine, L-hydroxyproline, L-thioproline, methyl derivatives of phenylalanine (Phe) such as 4-methyl-Phe, pentamethyl-Phe, L-Phe (4-amino), L-Tyr (methyl), L-Phe (4-isopropyl), L-Tic (1,2,3,4-tetrahydroisoquinoline-3-carboxyl acid), L-diaminopropionic acid, L-Phe (4-benzyl), 2,4-diaminobutyric acid, 4-aminobutyric acid (gamma-Abu), 2-amino butyric acid (alpha-Abu), 6-amino hexanoic acid (epsilon-Ahx), 2-amino isobutyric acid (Aib), 3-amino propionic acid, ornithine, norleucine, norvaline, hydroxyproline, sarcosine, citrulline, homocitrulline, cysteic acid, t-butylglycine, t-butylalanine, an amino acid derivitized with a heavy atom or heavy isotope (e.g., Au, deuterium, ¹⁵N; useful for synthesizing protein applicable to X-ray crystallographic structural analysis or nuclear magnetic resonance analysis), phenylglycine, cyclohexylalanine, fluoroamino acids, designer amino acids such as beta-methyl amino acids, Ca-methyl amino acids, Na-methyl amino acids, naphthyl alanine, and the like.

ARG/ABL nucleic acids and Cortactin nucleic acids also are referred to herein as “nucleic acid reagents.” A nucleic acid reagent can be from any source or composition, such as DNA, cDNA, RNA or mRNA, for example, and can be in any form (e.g., linear, circular, supercoiled, single-stranded, double-stranded, and the like). A nucleic acid reagent sometimes is a plasmid, phage, autonomously replicating sequence (ARS), centromere, artificial chromosome or other nucleic acid able to replicate or be replicated in vitro or in a host cell. Such nucleic acid reagents are selected for their ability to guide production of the desired protein or nucleic acid molecule. When desired, the nucleic acid reagent can be altered as known in the art such that codons encode for a different amino acid than is normal, including unconventional or unnatural amino acids (including detectably labeled amino acids).

A nucleic acid reagent can comprise certain elements often selected according to the intended use of the nucleic acid. Any of the following elements can be included in or excluded from a nucleic acid reagent. A nucleic acid reagent, for example, may include one or more or all of the following nucleotide elements: one or more promoter elements, one or more 5′ untranslated regions (5′UTRs), one or more regions into which a target nucleotide sequence may be inserted (an “insertion element”), one or more target nucleotide sequences, one or more 3′ untranslated regions (3′UTRs), and a selection element. A nucleic acid reagent is provided with one or more of such elements and other elements may be inserted into the nucleic acid before the template is contacted with an in vitro transcription and/or translation system. In some embodiments, a provided nucleic acid reagent comprises a promoter, 5′UTR, optional 3′UTR and insertion element(s) by which a target nucleotide sequence is inserted (i.e., cloned) into the template. In certain embodiments, a provided nucleic acid reagent comprises a promoter, insertion element(s) and optional 3′UTR, and a 5′ UTR/target nucleotide sequence is inserted with an optional 3′UTR. The elements can be arranged in any order suitable for in vitro transcription and/or translation, and in some embodiments a nucleic acid reagent comprises the following elements in the 5′ to 3′ direction: (1) promoter element, 5′UTR, and insertion element(s); (2) promoter element, 5′UTR, and target nucleotide sequence; (3) promoter element, 5′UTR, insertion element(s) and 3′UTR; and (4) promoter element, 5′UTR, target nucleotide sequence and 3′UTR.

A promoter element typically is required for DNA synthesis and/or RNA synthesis. A promoter often interacts with a RNA polymerase. A polymerase is an enzyme that catalyses synthesis of nucleic acids using a preexisting nucleic acid reagent. When the template is a DNA template, an RNA molecule is transcribed before protein is synthesized. Enzymes having polymerase activity suitable for use in the present methods include any polymerase that is active in the chosen system with the chosen template to synthesize protein. The cell-free extract often includes a suitable polymerase, such as RNA polymerase II, SP6 RNA polymerase, T3 RNA polymerase, T7 RNA polymerase, RNA polymerase III and phage derived RNA polymerases. These and other polymerases are known and nucleic acid sequences with which they interact are known. Such sequences are readily accessed by the artisan, such as by searching one or more public or private databases, for example, and the sequences are readily adapted to nucleic acid reagents described herein.

A 5′ UTR may comprise one or more elements endogenous to the nucleotide sequence from which it originates, and sometimes includes one or more exogenous elements. A 5′ UTR can originate from any suitable nucleic acid, such as genomic DNA, plasmid DNA, RNA or mRNA, for example, from any suitable organism (e.g., virus, bacterium, yeast, fungi, plant, insect or mammal). The artisan may select appropriate elements for the 5′ UTR based upon the transcription and/or translation system being utilized. A 5′ UTR sometimes comprises one or more of the following elements known to the artisan: translational enhancer sequence, transcription initiation site, transcription factor binding site, translation regulation site, translation initiation site, translation factor binding site, ribosome binding site, replicon, enhancer element, internal ribosome entry site (IRES), and silencer element.

A 5′UTR in the nucleic acid reagent can comprise a translational enhancer nucleotide sequence. A translational enhancer nucleotide sequence often is located between the promoter and the target nucleotide sequence in a nucleic acid reagent. A translational enhancer sequence often binds to a ribosome, sometimes is an 18S rRNA-binding ribonucleotide sequence (i.e., a 40S ribosome binding sequence) and sometimes is an internal ribosome entry sequence (IRES). An IRES generally forms an RNA scaffold with precisely placed RNA tertiary structures that contact a 40S ribosomal subunit via a number of specific intermolecular interactions. Examples of ribosomal enhancer sequences are known and can be identified by the artisan (e.g., Mignone et al., Nucleic Acids Research 33: D141-D146 (2005); Paulous et al., Nucleic Acids Research 31: 722-733 (2003); Akbergenov et al., Nucleic Acids Research 32: 239-247 (2004); Mignone et al., Genome Biology 3(3): reviews0004.1-0001.10 (2002); Gallie, Nucleic Acids Research 30: 3401-3411 (2002); Shaloiko et al., http address www.interscience.wiley.com, DOI: 10.1002/bit.20267; and Gallie et al., Nucleic Acids Research 15: 3257-3273 (1987)). A translational enhancer sequence sometimes is a eukaryotic sequence, such as a Kozak consensus sequence or other sequence (e.g., hydroid polyp sequence, GenBank accession no. U07128). A translational enhancer sequence sometimes is a prokaryotic sequence, such as a Shine-Dalgarno consensus sequence. In certain embodiments, the translational enhancer sequence is a viral nucleotide sequence. A translational enhancer sequence sometimes is from a 5′UTR of a plant virus, such as Tobacco Mosaic Virus (TMV), Alfalfa Mosaic Virus (AMV); Tobacco Etch Virus (ETV); Potato Virus Y (PVY); Turnip Mosaic (poty) Virus and Pea Seed Borne Mosaic Virus, for example. In certain embodiments, an omega sequence about 67 bases in length from TMV is included in the nucleic acid reagent as a translational enhancer sequence (e.g., devoid of guanosine nucleotides and includes a 25 nucleotide long poly (CAA) central region). In some embodiments, a translational enhancer sequence comprises one or more ARC-1 or ARC-1 like sequence, such as one of the following nucleotide sequences GCCGGCGGAG, CUCAUAAGGU, GACUUUGAUU, CGGAACCCAA, AUACUCCCCC and CCUUGCGACC, or a substantially identical sequence thereof. In certain embodiments, a translational enhancer sequence comprises an IRES sequence, such as one or more of EMBL nucleotide sequences J04513, X87949, M95825, M12783, AF025841, AF013263, AF006822, M17169, M13440, M22427, D14838 and M17446, or a substantially identical nucleotide sequence thereof. An IRES sequence may be a type I IRES (e.g., from enterovirus (e.g., poliovirus), rhinovirus (e.g., human rhinovirus)), a type II IRES (e.g., from cardiovirus (e.g., encephalomyocraditis virus), aphthovirus (e.g., foot-and-mouth disease virus)), a type III IRES (e.g., from Hepatitis A virus) or other picornavirus sequence (e.g., Paulos et al. supra, and Jackson et al., RNA 1: 985-1000 (1995)).

A 3′ UTR may comprise one or more elements endogenous to the nucleotide sequence from which it originates and sometimes includes one or more exogenous elements. A 3′ UTR may originate from any suitable nucleic acid, such as genomic DNA, plasmid DNA, RNA or mRNA, for example, from any suitable organism (e.g., a virus, bacterium, yeast, fungi, plant, insect or mammal). The artisan can select appropriate elements for the 3′ UTR based upon the transcription and/or translation system being utilized. A 3′ UTR sometimes comprises one or more of the following elements known to the artisan: transcription regulation site, transcription initiation site, transcription termination site, transcription factor binding site, translation regulation site, translation termination site, translation initiation site, translation factor binding site, ribosome binding site, replicon, enhancer element, silencer element and polyadenosine tail. A 3′ UTR often includes a polyadenosine tail and sometimes does not, and if a polyadenosine tail is present, one or more adenosine moieties may be added or deleted from it (e.g., about 5, about 10, about 15, about 20, about 25, about 30, about 35, about 40, about 45 or about 50 adenosine moieties may be added or subtracted).

A “target nucleotide sequence” as used herein encodes a nucleic acid, peptide, polypeptide or protein of interest, and may be a ribonucleotide sequence or a deoxyribonucleotide sequence. The term “nucleic acid” as used herein is generic to polydeoxyribonucleotides (containing 2′-deoxy-D-ribose or modified forms thereof), to polyribonucleotides (containing D-ribose or modified forms thereof), and to any other type of polynucleotide which is an N-glycoside of a purine or pyrimidine bases, or modified purine or pyrimidine bases. A target nucleic acid sometimes is an untranslated ribonucleic acid and sometimes is a translated ribonucleic acid. An untranslated ribonucleic acid may include, but is not limited to, a small interfering ribonucleic acid (siRNA), a short hairpin ribonucleic acid (shRNA), other ribonucleic acid capable of RNA interference (RNAi), an antisense ribonucleic acid, or a ribozyme. A translatable target nucleotide sequence (e.g., a target ribonucleotide sequence) sometimes encodes a peptide, polypeptide or protein, which are sometimes referred to herein as “target peptides,” “target polypeptides” or “target proteins.” Any peptides, polypeptides or proteins may be encoded by a target nucleotide sequence and may be selected by a person of ordinary skill in the art. Representative proteins include antibodies, enzymes, serum proteins (e.g., albumin), hormones (e.g., growth hormone, erythropoietin, insulin, etc.), cytokines, etc., and include both naturally occurring and exogenously expressed polypeptides. The term “protein” as used herein refers to a molecule having a sequence of amino acids linked by peptide bonds. This term includes fusion proteins, oligopeptides, polypeptides, cyclic peptides, polypeptides and polypeptide derivatives. A protein or polypeptide sometimes is of intracellular origin (e.g., located in the nucleus, cytosol, or interstitial space of host cells in vivo) and sometimes is a cell membrane protein in vivo.

A translatable nucleotide sequence generally is located between a start codon (AUG in ribonucleic acids and ATG in deoxyribonucleic acids) and a stop codon (e.g., UAA (ochre), UAG (amber) or UGA (opal) in ribonucleic acids and TAA, TAG or TGA in deoxyribonucleic acids), and sometimes is referred to herein as an “open reading frame” (ORF). A nucleic acid reagent sometimes comprises one or more ORFs. An ORF may be from any suitable source, sometimes from genomic DNA, mRNA, reverse transcribed RNA or complementary DNA (cDNA) or a nucleic acid library comprising one or more of the foregoing, and is from any organism species, such as human, insect, nematode, bovine, equine, canine, feline, rat or mouse, for example. An ARG, ABL or Cortactin protein or fragment encoding nucleotide sequences often is utilized as an ORF herein.

A nucleic acid reagent sometimes comprises a nucleotide sequence adjacent to an ORF that is translated in conjunction with the ORF and encodes an amino acid tag. The tag-encoding nucleotide sequence is located 3′ and/or 5′ of an ORF in the nucleic acid reagent, thereby encoding a tag at the C-terminus or N-terminus of the protein or peptide encoded by the ORF. Any tag that does not abrogate in vitro transcription and/or translation may be utilized and may be appropriately selected by the artisan. A tag sometimes specifically binds a molecule or moiety of a solid phase or a detectable label, for example, thereby having utility for isolating, purifying and/or detecting a protein or peptide encoded by the ORF. In some embodiments, a tag comprises one or more of the following elements: FLAG (e.g., DYKDDDDKG), V5 (e.g., GKPIPNPLLGLDST), c-MYC (e.g., EQKLISEEDL), HSV (e.g., QPELAPEDPED), influenza hemaglutinin, HA (e.g., YPYDVPDYA), VSV-G (e.g., YTDIEMNRLGK), bacterial glutathione-S-transferase, maltose binding protein, a streptavidin- or avidin-binding tag (e.g., pcDNA™6 BioEase™ Gateway® Biotinylation System (Invitrogen)), thioredoxin, β-galactosidase, VSV-glycoprotein, a fluorescent protein (e.g., green fluorescent protein or one of its many color variants (e.g., yellow, red, blue)), a polylysine or polyarginine sequence, a polyhistidine sequence (e.g., His₆) or other sequence that chelates a metal (e.g., cobalt, zinc, copper), and/or a cysteine-rich sequence that binds to an arsenic-containing molecule. In certain embodiments, a cysteine-rich tag comprises the amino acid sequence CC-Xₙ-CC, wherein X is any amino acid and n is 1 to 3, and the cysteine-rich sequence sometimes is CCPGCC. In certain embodiments, the tag comprises a cysteine-rich element and a polyhistidine element (e.g., CCPGCC and His₆).

A tag often conveniently binds to a binding partner. For example, some tags bind to an antibody (e.g., FLAG) and sometimes specifically bind to a small molecule. For example, a polyhistidine tag specifically chelates a bivalent metal, such as copper, zinc and cobalt; a polylysine or polyarginine tag specifically binds to a zinc finger; a glutathione S-transferase tag binds to glutathione; and a cysteine-rich tag specifically binds to an arsenic-containing molecule. Arsenic-containing molecules include LUMIO™ agents (Invitrogen, California), such as FlAsH™ (EDT₂[4′,5′-bis(1,3,2-dithioarsolan-2-yl)fluorescein-(1,2-ethanedithiol)₂]) and ReAsH reagents (e.g., U.S. Pat. No. 5,932,474 to Tsien et al., entitled “Target Sequences for Synthetic Molecules;” U.S. Pat. No. 6,054,271 to Tsien et al., entitled “Methods of Using Synthetic Molecules and Target Sequences;” U.S. Pat. Nos. 6,451,569 and 6,008,378; published U.S. Patent Application 2003/0083373, and published PCT Patent Application WO 99/21013, all to Tsien et al. and all entitled “Synthetic Molecules that Specifically React with Target Sequences”). Such antibodies and small molecules sometimes are linked to a solid phase for convenient isolation of the target protein or target peptide, as described in greater detail hereafter.

A tag sometimes comprises a sequence that localizes a translated protein or peptide to a component in a system, which is referred to as a “signal sequence” or “localization signal sequence” herein. A signal sequence often is incorporated at the N-terminus of a target protein or target peptide, and sometimes is incorporated at the C-terminus. Examples of signal sequences are known to the artisan, are readily incorporated into a nucleic acid reagent, and often are selected according to the cells from which a cell-free extract is prepared. A signal sequence in some embodiments localizes a translated protein or peptide to a cell membrane. Examples of signal sequences include, but are not limited to, a nucleus targeting signal (e.g., steroid receptor sequence and N-terminal sequence of SV40 virus large T antigen); mitochondia targeting signal (e.g., amino acid sequence that forms an amphipathic helix); peroxisome targeting signal (e.g., C-terminal sequence in YFG from S. cerevisiae); and a secretion signal (e.g., N-terminal sequences from invertase, mating factor alpha, PHO5 and SUC2 in S. cerevisiae; multiple N-terminal sequences of B. subtilis proteins (e.g., Tjalsma et al., Microbiol. Molec. Biol. Rev. 64: 515-547 (2000)); alpha amylase signal sequence (e.g., U.S. Pat. No. 6,288,302); pectate lyase signal sequence (e.g., U.S. Pat. No. 5,846,818); precollagen signal sequence (e.g., U.S. Pat. No. 5,712,114); OmpA signal sequence (e.g., U.S. Pat. No. 5,470,719); lam beta signal sequence (e.g., U.S. Pat. No. 5,389,529); B. brevis signal sequence (e.g., U.S. Pat. No. 5,232,841); and P. pastoris signal sequence (e.g., U.S. Pat. No. 5,268,273)).

A tag sometimes is directly adjacent to the amino acid sequence encoded by an ORF (i.e., there is no intervening sequence) and sometimes a tag is substantially adjacent to a the ORF encoded amino acid sequence (e.g., an intervening sequence is present). An intervening sequence sometimes includes a recognition site for a protease, which is useful for cleaving a tag from a target protein or peptide. In some embodiments, the intervening sequence is cleaved by Factor Xa (e.g., recognition site I(E/D)GR), thrombin (e.g., recognition site LVPRGS), enterokinase (e.g., recognition site DDDDK), TEV protease (e.g., recognition site ENLYFQG) or PreScission™ protease (e.g., recognition site LEVLFQGP), for example.

An intervening sequence sometimes is referred to herein as a “linker sequence,” and may be of any suitable length selected by the artisan. A linker sequence sometimes is about 1 to about 20 amino acids in length, and sometimes about 5 to about 10 amino acids in length. The artisan may select the linker length to substantially preserve target protein or peptide function (e.g., a tag may reduce target protein or peptide function unless separated by a linker), to enhance disassociation of a tag from a target protein or peptide when a protease cleavage site is present (e.g., cleavage may be enhanced when a linker is present), and to enhance interaction of a tag/target protein product with a solid phase. A linker can be of any suitable amino acid content, and often comprises a higher proportion of amino acids having relatively short side chains (e.g., glycine, alanine, serine and threonine).

A nucleic acid reagent sometimes includes a stop codon between a tag element and an insertion element or ORF, which can be useful for translating an ORF with or without the tag. Mutant tRNA molecules that recognize stop codons (described above) suppress translation termination and thereby are designated “suppressor tRNAs.” Suppressor tRNAs can result in the insertion of amino acids and continuation of translation past stop codons (e.g., U.S. Patent Application No. 60/587,583, filed Jul. 14, 2004, entitled “Production of Fusion Proteins by Cell-Free Protein Synthesis,”; Eggertsson, et al., (1988) Microbiological Review 52(3):354-374, and Engleerg-Kukla, et al. (1996) in Escherichia coli and Salmonella Cellular and Molecular Biology, Chapter 60, pps 909-921, Neidhardt, et al. eds., ASM Press, Washington, D.C.). A number of suppressor tRNAs are known, including but not limited to, supE, supP, supD, supF and supZ suppressors, which suppress the termination of translation of the amber stop codon; supB, glT, supL, supN, supC and supM suppressors, which suppress the function of the ochre stop codon and glyT, trpT and Su-9 suppressors, which suppress the function of the opal stop codon. In general, suppressor tRNAs contain one or more mutations in the anti-codon loop of the tRNA that allows the tRNA to base pair with a codon that ordinarily functions as a stop codon. The mutant tRNA is charged with its cognate amino acid residue and the cognate amino acid residue is inserted into the translating polypeptide when the stop codon is encountered. Mutations that enhance the efficiency of termination suppressors (i.e., increase stop codon read-through) have been identified. These include, but are not limited to, mutations in the uar gene (also known as the prfA gene), mutations in the ups gene, mutations in the sueA, sueB and sueC genes, mutations in the rpsD (ramA) and rpsE (spcA) genes and mutations in the rplL gene.

Thus, a nucleic acid reagent comprising a stop codon located between an ORF and a tag can yield a translated ORF alone when no suppressor tRNA is present in the translation system, and can yield a translated ORF-tag fusion when a suppressor tRNA is present in the system. In some embodiments, the stop codon is located 3′ of an insertion element or ORF and 5′ of a tag, and the stop codon sometimes is an amber codon. Suppressor tRNA sometimes are within a cell-free extract (e.g., the cell-free extract is prepared from cells that produce the suppressor tRNA), sometimes are added to the cell-free extract as isolated molecules, and sometimes are added to a cell-free extract as part of another extract. A provided suppressor tRNA sometimes is loaded with one of the twenty naturally occurring amino acids or an unnatural amino acid (described herein). Suppressor tRNA can be generated in cells transfected with a nucleic acid encoding the tRNA (e.g., a replication incompetent adenovirus containing the human tRNA-Ser suppressor gene can be transfected into cells). Vectors for synthesizing suppressor tRNA and for translating ORFs with or without a tag are available to the artisan (e.g., Tag-On-Demand™ kit (Invitrogen Corporation, California); Tag-On-Demand™ Suppressor Supernatant Instruction Manual, Version B, 6 Jun. 2003, at http address www.invitrogen.com/content/sfs/manuals/tagondemand_supernatant_man.pdf; Tag-On-Demand™ Gateway® Vector Instruction Manual, Version B, 20 Jun., 2003 at http address www.invitrogen.com/content/sfs/manuals/tagondemand_vectors_man.pdf; and Capone et al., Amber, ochre and opal suppressor tRNA genes derived from a human serine tRNA gene. EMBO J. 4:213, 1985).

Any convenient cloning strategy known to the artisan may be utilized to incorporate an element, such as an ORF, into a nucleic acid reagent. Known methods can be utilized to insert an element into the template independent of an insertion element, such as (1) cleaving the template at one or more existing restriction enzyme sites and ligating an element of interest and (2) adding restriction enzyme sites to the template by hybridizing oligonucleotide primers that include one or more suitable restriction enzyme sites and amplifying by polymerase chain reaction (described in greater detail herein). Other cloning strategies take advantage of one or more insertion sites present or inserted into the nucleic acid reagent, such as an oligonucleotide primer hybridization site for PCR, for example, and others described hereafter.

In some embodiments, the nucleic acid reagent includes one or more recombinase insertion sites. A recombinase insertion site is a recognition sequence on a nucleic acid molecule that participates in an integration/recombination reaction by recombination proteins. For example, the recombination site for Cre recombinase is loxP, which is a 34 base pair sequence comprised of two 13 base pair inverted repeats (serving as the recombinase binding sites) flanking an 8 base pair core sequence (e.g., FIG. 1 of Sauer, B., Curr. Opin. Biotech. 5:521-527 (1994)). Other examples of recombination sites include attB, attP, attL, and attR sequences, and mutants, fragments, variants and derivatives thereof, which are recognized by the recombination protein λ Int and by the auxiliary proteins integration host factor (IHF), FIS and excisionase (Xis) (e.g., U.S. Pat. Nos. 5,888,732; 6,143,557; 6,171,861; 6,270,969; 6,277,608; and 6,720,140; U.S. patent application Ser. No. 09/517,466, filed Mar. 2, 2000, and Ser. No. 09/732,914, filed Aug. 14, 2003, and in U.S. patent publication no. 2002-0007051-A1; Landy, Curr. Opin. Biotech. 3:699-707 (1993)). Examples of recombinase cloning nucleic acids are in Gateway® systems (Invitrogen, California), which include at least one recombination site for cloning a desired nucleic acid molecules in vivo or in vitro. In some embodiments, the system utilizes vectors that contain at least two different site-specific recombination sites, often based on the bacteriophage lambda system (e.g., att1 and att2), and are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the Gateway® system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

In certain embodiments, the nucleic acid reagent includes one or more topoisomerase insertion sites. A topoisomerase insertion site is a defined nucleotide sequence recognized and bound by a site-specific topoisomerase. For example, the nucleotide sequence 5′-(C/T)CCTT-3′ is a topoisomerase recognition site bound specifically by most poxvirus topoisomerases, including vaccinia virus DNA topoisomerase I. After binding to the recognition sequence, the topoisomerase cleaves the strand at the 3′-most thymidine of the recognition site to produce a nucleotide sequence comprising 5′-(C/T)CCTT-PO₄-TOPO, a complex of the topoisomerase covalently bound to the 3′ phosphate via a tyrosine in the topoisomerase (e.g., Shuman, J. Biol. Chem. 266:11372-11379, 1991; Sekiguchi and Shuman, Nucl. Acids Res. 22:5360-5365, 1994; U.S. Pat. No. 5,766,891; PCT/US95/16099; and PCT/US98/12372). In comparison, the nucleotide sequence 5′-GCAACTT-3′ is a topoisomerase recognition site for type IA E. coli topoisomerase III. An element to be inserted often is combined with topoisomerase-reacted template and thereby incorporated into the nucleic acid reagent (e.g., http address www.invitrogen.com/downloads/F-13512_Topo_Flyer.pdf; http address at www.invitrogen.com/content/sfs/brochures/710_{—}021849%20_B_TOPOCloning_bro.pdf; TOPO TA Cloning® Kit and Zero Blunt® TOPO® Cloning Kit product information).

A nucleic acid reagent sometimes contains one or more origin of replication (ORI) elements. In some embodiments, a template comprises two or more ORIs, where one functions efficiently in one organism (e.g., a bacterium) and another functions efficiently in another organism (e.g., a eukaryote). In some embodiments, an ORI may function efficiently in insect cells and another ORI may function efficiently in mammalian cells. A nucleic acid reagent also sometimes includes one or more transcription regulation sites.

A nucleic acid reagent often includes one or more selection elements. Selection elements often are utilized using known processes to determine whether a nucleic acid reagent is included in a cell. In some embodiments, a nucleic acid reagent includes two or more selection elements, where one functions efficiently in one organisms and another functions efficiently in another organism. Examples of selection elements include, but are not limited to, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic (e.g., Diphtheria toxin) or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like).

Certain nucleotide sequences sometimes are added to, modified or removed from one or more of the nucleic acid reagent elements, such as the promoter, 5′UTR, target sequence, or 3′UTR elements, to enhance or potentially enhance transcription and/or translation before or after such elements are incorporated in a nucleic acid reagent. In some embodiments, one or more of the following sequences may be modified or removed if they are present in a 5′UTR: a sequence that forms a stable secondary structure (e.g., quadruplex structure or stem loop stem structure (e.g., EMBL sequences X12949, AF274954, AF139980, AF152961, S95936, U194144, AF116649 or substantially identical sequences that form such stem loop stem structures)); a translation initiation codon upstream of the target nucleotide sequence start codon; a stop codon upstream of the target nucleotide sequence translation initiation codon; an ORF upstream of the target nucleotide sequence translation initiation codon; an iron responsive element (IRE) or like sequence; and a 5′ terminal oligopyrimidine tract (TOP, e.g., consisting of 5-15 pyrimidines adjacent to the cap). A translational enhancer sequence and/or an internal ribosome entry site (IRES) sometimes is inserted into a 5′UTR (e.g., EMBL nucleotide sequences J04513, X87949, M95825, M12783, AF025841, AF013263, AF006822, M17169, M13440, M22427, D14838 and M17446 and substantially identical nucleotide sequences). An AU-rich element (ARE, e.g., AUUUA repeats) and/or splicing junction that follows a non-sense codon sometimes is removed from or modified in a 3′UTR. A polyadenosine tail sometimes is inserted into a 3′UTR if none is present, sometimes is removed if it is present, and adenosine moieties sometimes are added to or removed from a polyadenosine tail present in a 3′UTR. Thus, some embodiments are directed to a process comprising: determining whether any nucleotide sequences that reduce or potentially reduce translation efficiency are present in the elements, and removing or modifying one or more of such sequences if they are identified. Certain embodiments are directed to a process comprising: determining whether any nucleotide sequences that increase or potentially increase translation efficiency are not present in the elements, and incorporating such sequences into the nucleic acid reagent.

An ORF sometimes is mutated or modified (for example, by point mutation, deletion mutation, insertion mutation, and the like) to alter, enhance or increase, reduce, substantially reduce or eliminate the activity of the encoded protein or peptide. The protein or peptide encoded by a modified ORF sometimes is produced in a lower amount or may not be produced at detectable levels, and in other embodiments, the product or protein encoded by the modified ORF is produced at a higher level (e.g., codons sometimes are modified so they are compatible with tRNA in cells used to prepare a cell-free extract). To determine the relative activity, the activity from the product of the mutated ORF (or cell containing it) can be compared to the activity of the product or protein encoded by the unmodified ORF (or cell containing it).

A stop codon at the end of an ORF sometimes is modified to another stop codon, such as an amber stop codon described above. In some embodiments, a stop codon is introduced within an ORF, sometimes by insertion or mutation of an existing codon. An ORF comprising a modified terminal stop codon and/or internal stop codon often is translated in a system comprising a suppressor tRNA that recognizes the stop codon. An ORF comprising a stop codon sometimes is translated in a system comprising a suppressor tRNA that incorporates an unnatural amino acid during translation of the target protein or target peptide. Methods for incorporating unnatural amino acids into a target protein or peptide are known, which include, for example, processes utilizing a heterologous tRNA/synthetase pair, where the tRNA recognizes an amber stop codon and is loaded with an unnatural amino acid (e.g., http address www.iupac.org/news/prize/2003/wang.pdf). Examples of unnatural amino acids are described above.

A nucleic acid reagent is of any form useful for in vitro or in vivo transcription and/or translation. A nucleic acid sometimes is a plasmid, such as a supercoiled plasmid, sometimes is a linear nucleic acid (e.g., a linear nucleic acid produced by PCR or by restriction digest), sometimes is single-stranded and sometimes is double-stranded. A nucleic acid reagent for in vitro transcription and/or translation can be prepared by any suitable process. A nucleic acid reagent sometimes is prepared by an amplification process, such as a polymerase chain reaction (PCR) process or transcription-mediated amplification process (TMA). In TMA, two enzymes are used in an isothermal reaction to produce amplification products detected by light emission (see, e.g., Biochemistry 1996 Jun. 25; 35(25):8429-38 and http address www.devicelink.com/ivdt/archive/00/11/007.html). Standard PCR processes are known (e.g., U.S. Pat. Nos. 4,683,202; 4,683,195; 4,965,188; and 5,656,493), and generally are performed in cycles. Each cycle includes heat denaturation, in which hybrid nucleic acids dissociate; cooling, in which primer oligonucleotides hybridize; and extension of the oligonucleotides by a polymerase (i.e., Taq polymerase). An example of a PCR cyclical process is treating the sample at 95° C. for 5 minutes; repeating forty-five cycles of 95° C. for 1 minute, 59° C. for 1 minute, 10 seconds, and 72° C. for 1 minute 30 seconds; and then treating the sample at 72° C. for 5 minutes. Multiple cycles frequently are performed using a commercially available thermal cycler. PCR amplification products sometimes are stored for a time at a lower temperature (e.g., at 4° C.) and sometimes are frozen (e.g., at −20° C.) before analysis.

In some embodiments, a nucleic acid reagent, protein reagent, protein fragment reagent or other reagent described herein is isolated or purified. The term “isolated” as used herein refers to material removed from its original environment (e.g., the natural environment if it is naturally occurring, or a host cell if expressed exogenously), and thus is altered “by the hand of man” from its original environment. The term “purified” as used herein with reference to molecules does not refer to absolute purity. Rather, “purified” refers to a substance in a composition that contains fewer substance species in the same class (e.g., nucleic acid or protein species) other than the substance of interest in comparison to the sample from which it originated. “Purified,” if a nucleic acid or protein for example, refers to a substance in a composition that contains fewer nucleic acid species or protein species other than the nucleic acid or protein of interest in comparison to the sample from which it originated. Sometimes, a protein or nucleic acid is “substantially pure,” indicating that the protein or nucleic acid represents at least 50% of protein or nucleic acid on a mass basis of the composition. Often, a substantially pure protein or nucleic acid is at least 75% on a mass basis of the composition, and sometimes at least 95% on a mass basis of the composition.

Specific Interaction Reagents

An ARG/ABL reagent or Cortactin reagent sometimes is a molecule that specifically interacts with (e.g., binds to) an ARG, ABL or Cortactin protein or fragment or nucleic acid. The latter class of reagents sometimes are referred to herein as “specific interaction reagents” or “specific binding reagents.” A specific binding reagent sometimes is in association with detectable label described in greater detail hereafter. Examples of specific binding reagents that bind to ARG, ABL and/or Cortactin proteins or fragments include antibodies and antibody fragments; ARG, ABL or Cortactin binding partners and chemical compounds. Examples of specific binding reagents that bind to ARG, ABL and/or Cortactin nucleic acids include organic molecules and antisense, ribozyme and siRNA nucleic acids.

A variety of antibodies and antibody fragments are available to and can be generated by the artisan for use as a specific binding reagent. An antibody or antibody fragment selected by the artisan sometimes binds to an ARG and/or ABL protein or fragment, Cortactin protein or fragment and/or a Cortactin/ABL, Cortactin/ARG or Cortactin/ABL/ARG complex. Such an antibody or antibody fragment can bind to the complex without significantly disrupting binding between the ARG, ABL and Cortactin molecules in some embodiments, or alternatively can bind and disrupt interactions between ARG, ABL and/or Cortactin in other embodiments.

Antibodies sometimes are IgG, IgM, IgA, IgE, or an isotype thereof (e.g., IgG1, IgG2a, IgG2b or IgG3), sometimes are polyclonal or monoclonal, and sometimes are chimeric, humanized or bispecific versions of such antibodies. Polyclonal and monoclonal antibodies that bind specific antigens are commercially available, and methods for generating such antibodies are known. In general, polyclonal antibodies are produced by injecting an isolated antigen (e.g., ARG, ABL or Cortactin protein or fragment) into a suitable animal (e.g., a goat or rabbit); collecting blood and/or other tissues from the animal containing antibodies specific for the antigen and purifying the antibody. Methods for generating monoclonal antibodies, in general, include injecting an animal with an isolated antigen (e.g., often a mouse or a rat); isolating splenocytes from the animal; fusing the splenocytes with myeloma cells to form hybridomas; isolating the hybridomas and selecting hybridomas that produce monoclonal antibodies which specifically bind the antigen (e.g., Kohler & Milstein, Nature 256:495 497 (1975) and StGroth & Scheidegger, J Immunol Methods 5:1 21 (1980)).

Methods for generating chimeric and humanized antibodies also are known (see, e.g., U.S. Pat. No. 5,530,101 (Queen, et al.), U.S. Pat. No. 5,707,622 (Fung, et al.) and U.S. Pat. Nos. 5,994,524 and 6,245,894 (Matsushima, et al.)), which generally involve transplanting an antibody variable region from one species (e.g., mouse) into an antibody constant domain of another species (e.g., human). Antigen-binding regions of antibodies (e.g., Fab regions) include a light chain and a heavy chain, and the variable region is composed of regions from the light chain and the heavy chain. Given that the variable region of an antibody is formed from six complementarity-determining regions (CDRs) in the heavy and light chain variable regions, one or more CDRs from one antibody can be substituted (i.e., grafted) with a CDR of another antibody to generate chimeric antibodies. Also, humanized antibodies are generated by introducing amino acid substitutions that render the resulting antibody less immunogenic when administered to humans.

A specific binding reagent sometimes is an antibody fragment, such as a Fab, Fab′, F(ab)′2, Dab, Fv or single-chain Fv (ScFv) fragment, and methods for generating antibody fragments are known (see, e.g., U.S. Pat. Nos. 6,099,842 and 5,990,296 and PCT/GB00/04317). In some embodiments, a binding partner in one or more hybrids is a single-chain antibody fragment, which sometimes are constructed by joining a heavy chain variable region with a light chain variable region by a polypeptide linker (e.g., the linker is attached at the C-terminus or N-terminus of each chain) by recombinant molecular biology processes. Such fragments often exhibit specificities and affinities for an antigen similar to the original monoclonal antibodies. Bifunctional antibodies sometimes are constructed by engineering two different binding specificities into a single antibody chain and sometimes are constructed by joining two Fab′ regions together, where each Fab′ region is from a different antibody (e.g., U.S. Pat. No. 6,342,221). Antibody fragments often comprise engineered regions such as CDR-grafted or humanized fragments. In certain embodiments the binding partner is an intact immunoglobulin, and in other embodiments the binding partner is a Fab monomer or a Fab dimer.

The artisan may select and prepare a binding partner of ARG, ABL or Cortactin as a specific binding reagent. Multiple binding partners of ARG and ABL are known, including CRK (NP_{—}058431.2), PDE4D4 (AAC00042.1), RB (NP_{—}000312.1), Robo1 (NP_{—}002932.1), 14-3-3 delta and eta (NP_{—}003396.1), BCR(NP_{—}004318.2), p85 (NP_{—}852664.1), PLC-gamma1 (NP_{—}002651.2), GAP (NP_{—}002881.1), SRC (NP_{—}005408.1), WAVE-1 (NP_{—}003922.1), hNAP1 BP (BAB55675.1) and p47phox (NP_{—}000256.1). An ABL protein or fragment also may be utilized as a specific binding reagent as ABL can interact with itself in trans via its SH2 domain. Also an ARG protein or fragment can be utilized as a specific binding reagent as ABL and ARG interact with one another. Cortactin binding partners include AMAP1 (NP_{—}060952.2) and Paxillin (NP_{—}002850.1). The name of each of the foregoing binding partners is followed by an identifier in a public database accessed at http address www.ncbi.nih.gov/entrez/query.fcgi?db=gene that provides further information for each binding partner, the latter of which is incorporated herein by reference. The artisan may utilize a fragment of a binding partner that binds to an ARG, ABL and/or Cortactin protein or fragment as a specific binding reagent in specific embodiments. The artisan also may optimize a binding reagent for a specific use or identify new binding reagents using a variety of procedures. For example, binding partners may be identified by lysing cells and analyzing cell lysates by electrophoretic techniques. Alternatively, a two-hybrid assay or three-hybrid assay can be utilized (e.g., U.S. Pat. No. 5,283,317; Zervos et al., Cell 72:223-232 (1993); Madura et al., J. Biol. Chem. 268: 12046-12054 (1993); Bartel et al., Biotechniques 14: 920-924 (1993); Iwabuchi et al., Oncogene 8: 1693-1696 (1993); and Brent WO94/10300). A two-hybrid system is based on the modular nature of most transcription factors, which consist of separable DNA-binding and activation domains. The assay often utilizes two different DNA constructs. In one construct, an ARG, ABL or Cortactin nucleic acid (sometimes referred to as the “bait”) is fused to a gene encoding the DNA binding domain of a known transcription factor (e.g., GAL-4). In another construct, a DNA sequence from a library of DNA sequences that encodes a potential binding partner (sometimes referred to as the “prey”) is fused to a gene that encodes an activation domain of the known transcription factor. Sometimes, an ARG, ABL or Cortactin nucleic acid is to the activation domain. If the “bait” and the “prey” molecules interact in vivo, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to identify the potential binding partner.

The artisan of ordinary skill can select a specific binding reagent from a large complement of chemical compounds. Examples of inhibitors that can inhibit ARG or ABL protein kinases are 4-[(4-methyl-1-piperazinyl)methyl]-N-[4-methyl-3-[[4-(3-pyridinyl)-2-yrimidinyl]amino]-phenyl]benzamide methanesulfonate (also referred to as Imatinib Mesylate), 4-amino-N-(2,5-dihydroxybenzyl)methyl benzoate (also referred to as Tyrphostin AG957) and 4-amino-5-(4-methylphenyl)-7-(t-butyl)pyrazolo[3,4-d]pyrimidine (also referred to as PP1). Other protein kinase inhibitors are known (e.g., http address www.proteinkinase.de/html/protein_kinase_inhibitors.html), and ATP-competitive molecules may be utilized (e.g., AMP-PNP; adenosine; staurosporine; staurosporine aglycone; certain isoquinolines (e.g., isoquinoline-IQP, -IQS and -IQB); balanol; bis-indole maleimide; di-anilinophthaliomide; N-(2-chloro-6-methylphenyl)-2-(6-(4-(2-hydroxyethyl)piperazin-1-yl)-2-methylpyrimidin-4-ylamino)thiazole-5-carboxamide (e.g., Shah et al., Science 305: 399-401 (2004)); N-(3-nitro-6-methylphenyl)-4-(3′-pyridyl)-2-pyrimidineamine or methyl 4-(2,5-dihydroxybenzylamino)benzoate (e.g., Wang et al., PNAS 102: 3208-3212 (2005)); certain quinazolines and others (e.g., compounds described at http address www.chemsoc.org/exemplarchem/entries/jagfin/jagfin/contents/therap.htm; http address www.chemsoc.org/exemplarchem/entries/jagfin/jagfin/contents/inhco.htm; and in Table 1 of Druker & Lydon, J. Clin. Invest. 105(1):3-7 (2000)). Compounds can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive (see, e.g., Zuckermann et al., J. Med. Chem. 37: 2678-85 (1994)); spatially addressable parallel solid phase or solution phase libraries; synthetic library methods requiring deconvolution; “one-bead one-compound” library methods; and synthetic library methods using affinity chromatography selection. Biological library and peptoid library approaches are typically limited to peptide libraries, while the other approaches are applicable to peptide, non-peptide oligomer or small molecule libraries of compounds (Lam, Anticancer Drug Des. 12: 145, (1997)). Examples of methods for synthesizing molecular libraries are described, for example, in DeWitt et al., Proc. Natl. Acad. Sci. U.S.A. 90: 6909 (1993); Erb et al., Proc. Natl. Acad. Sci. USA 91: 11422 (1994); Zuckermann et al., J. Med. Chem. 37: 2678 (1994); Cho et al., Science 261: 1303 (1993); Carrell et al., Angew. Chem. Int. Ed. Engl. 33: 2059 (1994); Carell et al., Angew. Chem. Int. Ed. Engl. 33: 2061 (1994); and in Gallop et al., J. Med. Chem. 37: 1233 (1994). Libraries of compounds may be presented in solution (e.g., Houghten, Biotechniques 13: 412-421 (1992)), or on beads (Lam, Nature 354: 82-84 (1991)), chips (Fodor, Nature 364: 555-556 (1993)), bacteria or spores (Ladner, U.S. Pat. No. 5,223,409), plasmids (Cull et al., Proc. Natl. Acad. Sci. USA 89: 1865-1869 (1992)) or on phage (Scott and Smith, Science 249: 386-390 (1990); Devlin, Science 249: 404-406 (1990); Cwirla et al., Proc. Natl. Acad. Sci. 87: 6378-6382 (1990); Felici, J. Mol. Biol. 222: 301-310 (1991); Ladner supra.).

A compound sometimes is a small molecule. Small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The artisan can select and prepare a nucleic acid specific binding reagent for use. Nucleic acids may comprise or consist of analog or derivative nucleic acids, such as polyamide nucleic acids (PNA) and others exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; 5,614,622; 5,739,314; 5,955,599; 5,962,674; 6,117,992; WIPO publications WO 00/56746, WO 00/75372 and WO 01/14398, and related publications. An antisense nucleic acid sometimes is designed, prepared and/or utilized by the artisan to inhibit an ARG, ABL or Cortactin nucleic acid. An “antisense” nucleic acid refers to a nucleotide sequence complementary to a “sense” nucleic acid encoding an ARG, ABL or Cortactin protein or fragment (e.g., complementary to the coding strand of a double-stranded cDNA molecule or complementary to an mRNA sequence). The antisense nucleic acid can be complementary to an entire coding strand, or to a portion thereof or a substantially identical sequence thereof. In another embodiment, the antisense nucleic acid molecule is antisense to a “noncoding region” of the coding strand of a nucleotide sequence.

An antisense nucleic acid can be complementary to the entire coding region of an mRNA encoded by an ARG, ABL or Cortactin nucleotide sequence, and often the antisense nucleic acid is an oligonucleotide antisense to only a portion of a coding or noncoding region of the mRNA. For example, the antisense oligonucleotide can be complementary to the region surrounding the translation start site of the mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid can be constructed using chemical synthesis or enzymic ligation reactions using standard procedures. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used). Antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

When utilized in animals, antisense nucleic acids typically are administered to a subject (e.g., by direct injection at a tissue site) or generated in situ such that they hybridize with or bind to cellular mRNA and/or genomic DNA encoding a polypeptide and thereby inhibit expression of the polypeptide, for example, by inhibiting transcription and/or translation. Alternatively, antisense nucleic acid molecules can be modified to target selected cells and then are administered systemically. For systemic administration, antisense molecules can be modified such that they specifically bind to receptors or antigens expressed on a selected cell surface, for example, by linking antisense nucleic acid molecules to peptides or antibodies which bind to cell surface receptors or antigens. Antisense nucleic acid molecules can also be delivered to cells using the vectors described herein. Sufficient intracellular concentrations of antisense molecules are achieved by incorporating a strong promoter, such as a pol II or pol III promoter, in the vector construct.

Antisense nucleic acid molecules sometimes are alpha-anomeric nucleic acid molecules. An alpha-anomeric nucleic acid molecule forms specific double-stranded hybrids with complementary RNA in which, contrary to the usual beta-units, the strands run parallel to each other (Gaultier et al., Nucleic Acids. Res. 15: 6625-6641 (1987)). Antisense nucleic acid molecules also can comprise a 2′-o-methylribonucleotide (Inoue et al., Nucleic Acids Res. 15: 6131-6148 (1987)) or a chimeric RNA-DNA analogue (Inoue et al., FEBS Lett. 215: 327-330 (1987)). Antisense nucleic acids sometimes are composed of DNA or PNA or any other nucleic acid derivatives described previously.

An antisense nucleic acid is a ribozyme in some embodiments. A ribozyme having specificity for an ARG, ABL or Cortactin nucleotide sequence can include one or more sequences complementary to such a nucleotide sequence, and a sequence having a known catalytic region responsible for mRNA cleavage (e.g., U.S. Pat. No. 5,093,246 or Haselhoff and Gerlach, Nature 334: 585-591 (1988)). For example, a derivative of a Tetrahymena L-19 IVS RNA is sometimes utilized in which the nucleotide sequence of the active site is complementary to the nucleotide sequence to be cleaved in a mRNA (e.g., Cech et al. U.S. Pat. No. 4,987,071; and Cech et al. U.S. Pat. No. 5,116,742). ARG, ABL and Cortactin mRNA sequences also may be utilized to select a catalytic RNA having a specific ribonuclease activity from a pool of RNA molecules (e.g., Bartel & Szostak, Science 261: 1411-1418 (1993)).

Specific binding reagents sometimes are nucleic acids that can form triple helix structures with an ARG, ABL or Cortactin nucleic acid. ARG, ABL or Cortactin expression can be inhibited by targeting nucleotide sequences complementary to the regulatory region of a nucleotide sequence referenced herein or a substantially identical sequence (e.g., promoter and/or enhancers) to form triple helical structures that prevent transcription of a gene in target cells (see e.g., Helene, Anticancer Drug Des. 6(6): 569-84 (1991); Helene et al., Ann. N.Y. Acad. Sci. 660: 27-36 (1992); and Maher, Bioassays 14(12): 807-15 (1992). Triple helix formation can be enhanced by generating a “switchback” nucleic acid molecule. Switchback molecules are synthesized in an alternating 5′-3′,3′-5′ manner, such that they base pair with first one strand of a duplex and then the other, eliminating the necessity for a sizeable stretch of purines or pyrimidines being present on one strand of a duplex.

An artisan may select an interfering RNA (RNAi) or siRNA specific binding reagent for use. The nucleic acid selected sometimes is the RNAi or siRNA or a nucleic acid that encodes such products. The term “RNAi” as used herein refers to double-stranded RNA (dsRNA) which mediates degradation of specific mRNAs, and can also be used to lower or eliminate gene expression. The term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule directed against a gene. For example, a siRNA is capable of inhibiting or down regulating gene expression or viral replication, for example by mediating RNA interference “RNAi” or gene silencing in a sequence-specific manner; see for example Zamore et al., 2000, Cell, 101, 25-33; Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831). There is no particular limitation in the length of siRNA as long as it does not show toxicity. Examples of modified RNAi and siRNA include STEALTH™ forms (Invitrogen Corp., Carlsbad, Calif.), forms described in U.S. Patent Publication No. 2004/0014956 (application Ser. No. 10/357,529) and U.S. patent application Ser. No. 11/049,636, filed Feb. 2, 2005), and other forms described hereafter.

A siNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siNA can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double stranded structure, for example wherein the double stranded region is about 19 base pairs); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the siNA is assembled from a single oligonucleotide, where the self-complementary sense and antisense regions of the siNA are linked by means of a nucleic acid based or non-nucleic acid-based linker(s). The siNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The siNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (for example, where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate. In certain embodiments, the siNA molecule of the invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic interactions, and/or stacking interactions. In certain embodiments, the siNA molecules of the invention comprise nucleotide sequence that is complementary to nucleotide sequence of a target gene. In another embodiment, the siNA molecule of the invention interacts with nucleotide sequence of a target gene in a manner that causes inhibition of expression of the target gene.

The double-stranded RNA portions of siRNAs in which two RNA strands pair are not limited to the completely paired forms, and may contain non-pairing portions due to mismatch (the corresponding nucleotides are not complementary), bulge (lacking in the corresponding complementary nucleotide on one strand), and the like. Non-pairing portions can be contained to the extent that they do not interfere with siRNA formation. The “bulge” used herein preferably comprise 1 to 2 non-pairing nucleotides, and the double-stranded RNA region of siRNAs in which two RNA strands pair up contains preferably 1 to 7, more preferably 1 to 5 bulges. In addition, the “mismatch” used herein is contained in the double-stranded RNA region of siRNAs in which two RNA strands pair up, preferably 1 to 7, more preferably 1 to 5, in number. In a preferable mismatch, one of the nucleotides is guanine, and the other is uracil. Such a mismatch is due to a mutation from C to T, G to A, or mixtures thereof in DNA coding for sense RNA, but not particularly limited to them. Furthermore, in the present invention, the double-stranded RNA region of siRNAs in which two RNA strands pair up may contain both bulge and mismatched, which sum up to, preferably 1 to 7, more preferably 1 to 5 in number. The terminal structure of siRNA may be either blunt or cohesive (overhanging) as long as siRNA enables to silence the target gene expression due to its RNAi effect.

As used herein, siRNA molecules need not be limited to those molecules containing only RNA, but further encompasses chemically-modified nucleotides and non-nucleotides. In addition, as used herein, the term RNAi is meant to be equivalent to other terms used to describe sequence specific RNA interference, such as post transcriptional gene silencing, translational inhibition, or epigenetics. For example, siRNA molecules of the invention can be used to epigenetically silence genes at both the post-transcriptional level or the pre-transcriptional level. In a non-limiting example, epigenetic regulation of gene expression by siRNA molecules of the invention can result from siRNA mediated modification of chromatin structure to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

RNAi may be designed by those methods known to those of ordinary skill in the art. In one example, siRNA may be designed by classifying RNAi sequences, for example 1000 sequences, based on functionality, with a functional group being classified as having greater than 85% knockdown activity and a non-functional group with less than 85% knockdown activity. The distribution of base composition was calculated for entire the entire RNAi target sequence for both the functional group and the non-functional group. The ratio of base distribution of functional and non-functional group may then be used to build a score matrix for each position of RNAi sequence. For a given target sequence, the base for each position is scored, and then the log ratio of the multiplication of all the positions is taken as a final score. Using this score system, a very strong correlation may be found of the functional knockdown activity and the log ratio score. Once the target sequence is selected, it may be filtered through both fast NCBI blast and slow Smith Waterman algorithm search against the Unigene database to identify the gene-specific RNAi or siRNA. Sequences with at least one mismatch in the last 12 bases may be selected.

Nucleic acid reagents include those which are engineered, for example, to produce dsRNAs. Examples of such nucleic acid molecules include those with a sequence that, when transcribed, folds back upon itself to generate a hairpin molecule containing a double-stranded portion. One strand of the double-stranded portion may correspond to all or a portion of the sense strand of the mRNA transcribed from the gene to be silenced while the other strand of the double-stranded portion may correspond to all or a portion of the antisense strand. Other methods of producing dsRNAs may be used, for example, nucleic acid molecules may be engineered to have a first sequence that, when transcribed, corresponds to all or a portion of the sense strand of the mRNA transcribed from the gene to be silenced and a second sequence that, when transcribed, corresponds to all or portion of an antisense strand (i.e., the reverse complement) of the mRNA transcribed from the gene to be silenced.

Nucleic acid molecules which mediate RNAi may also be produced ex vivo, for example, by oligonucleotide synthesis. Oligonucleotide synthesis may be used for example, to design dsRNA molecules, as well as other nucleic acid molecules (e.g., other nucleic acid molecules which mediate RNAi) with one or more chemical modification (e.g., chemical modifications not commonly found in nucleic acid molecules such as the inclusion of 2′-O-methyl, 2′-O-ethyl, 2′-O-propyl, 2′-fluoro, etc. groups).

In some embodiments, a dsRNA to be used to silence a gene may have one or more (e.g., one, two, three, four, five, six, etc.) regions of sequence homology or identity to a gene to be silenced. Regions of homology or identity may be from about 20 bp (base pairs) to about 5 kbp (kilo base pairs) in length, 20 bp to about 4 kbp in length, 20 bp to about 3 kbp in length, 20 bp to about 2.5 kbp in length, from about 20 bp to about 2 kbp in length, 20 bp to about 1.5 kbp in length, from about 20 bp to about 1 kbp in length, 20 bp to about 750 bp in length, from about 20 bp to about 500 bp in length, 20 bp to about 400 bp in length, 20 bp to about 300 bp in length, 20 bp to about 250 bp in length, from about 20 bp to about 200 bp in length, from about 20 bp to about 150 bp in length, from about 20 bp to about 100 bp in length, from about 20 bp to about 90 bp in length, from about 20 bp to about 80 bp in length, from about 20 bp to about 70 bp in length, from about 20 bp to about 60 bp in length, from about 20 bp to about 50 bp in length, from about 20 bp to about 40 bp in length, from about 20 bp to about 30 bp in length, from about 20 bp to about 25 bp in length, from about 15 bp to about 25 bp in length, from about 17 bp to about 25 bp in length, from about 19 bp to about 25 bp in length, from about 19 bp to about 23 bp in length, or from about 19 bp to about 21 bp in length.

A hairpin containing molecule having a double-stranded region may be used as RNAi. The length of the double stranded region may be from about 20 bp (base pairs) to about 2.5 kbp (kilo base pairs) in length, from about 20 bp to about 2 kbp in length, 20 bp to about 1.5 kbp in length, from about 20 bp to about 1 kbp in length, 20 bp to about 750 bp in length, from about 20 bp to about 500 bp in length, 20 bp to about 400 bp in length, 20 bp to about 300 bp in length, 20 bp to about 250 bp in length, from about 20 bp to about 200 bp in length, from about 20 bp to about 150 bp in length, from about 20 bp to about 100 bp in length, 20 bp to about 90 bp in length, 20 bp to about 80 bp in length, 20 bp to about 70 bp in length, 20 bp to about 60 bp in length, 20 bp to about 50 bp in length, 20 bp to about 40 bp in length, 20 bp to about 30 bp in length, or from about 20 bp to about 25 bp in length. The non-base-paired portion of the hairpin (i.e., loop) can be of any length that permits the two regions of homology that make up the double-stranded portion of the hairpin to fold back upon one another.

Any suitable promoter may be used to control the production of RNA from the nucleic acid reagent, such as a promoter described above. Promoters may be those recognized by any polymerase enzyme. For example, promoters may be promoters for RNA polymerase II or RNA polymerase III (e.g., a U6 promoter, an H1 promoter, etc.). Other suitable promoters include, but are not limited to, T7 promoter, cytomegalovirus (CMV) promoter, mouse mammary tumor virus (MMTV) promoter, metalothionine, RSV (Rous sarcoma virus) long terminal repeat, SV40 promoter, human growth hormone (hGH) promoter. Other suitable promoters are known to those skilled in the art and are within the scope of the present invention.

Double-stranded RNAs used in the practice of the invention may vary greatly in size. Further the size of the dsRNAs used will often depend on the cell type contacted with the dsRNA. As an example, animal cells such as those of C. elegans and Drosophila melanogaster do not generally undergo apoptosis when contacted with dsRNAs greater than about 30 nucleotides in length (i.e., 30 nucleotides of double stranded region) while mammalian cells typically do undergo apoptosis when exposed to such dsRNAs. Thus, the design of the particular experiment will often determine the size of dsRNAs employed.

In many instances, the double stranded region of dsRNAs contained within or encoded by nucleic acid molecules used in the practice of the invention will be within the following ranges: from about 20 to about 30 nucleotides, from about 20 to about 40 nucleotides, from about 20 to about 50 nucleotides, from about 20 to about 100 nucleotides, from about 22 to about 30 nucleotides, from about 22 to about 40 nucleotides, from about 20 to about 28 nucleotides, from about 22 to about 28 nucleotides, from about 25 to about 30 nucleotides, from about 25 to about 28 nucleotides, from about 30 to about 100 nucleotides, from about 30 to about 200 nucleotides, from about 30 to about 1,000 nucleotides, from about 30 to about 2,000 nucleotides, from about 50 to about 100 nucleotides, from about 50 to about 1,000 nucleotides, or from about 50 to about 2,000 nucleotides. The ranges above refer to the number of nucleotides present in double stranded regions. Thus, these ranges do not reflect the total length of the dsRNAs themselves. As an example, a blunt ended dsRNA formed from a single transcript of 50 nucleotides in total length with a 6 nucleotide loop, will have a double stranded region of 23 nucleotides.

As suggested above, dsRNAs used in the practice of the invention may be blunt ended, may have one blunt end, or may have overhangs on both ends. Further, when one or more overhang is present, the overhang(s) may be on the 3′ and/or 5′ strands at one or both ends. Additionally, these overhangs may independently be of any length (e.g., one, two, three, four, five, etc. nucleotides). As an example, STEALTH™ RNAi is blunt at both ends.

Also included are sets of RNAi and those which generate RNAi. Such sets include those which either (1) are designed to produce or (2) contain more than one dsRNA directed against the same target gene. As an example, the invention includes sets of STEALTH™ RNAi wherein more than one STEALTH™ RNAi shares sequence homology or identity to different regions of the same target gene.

RNAi and siRNA reagents, as well as methods and tools for designing RNAi and siRNA reagents, are available to the artisan (e.g., https address rnaidesigner.invitrogen.com/rnaiexpress/). Examples of Cortactin RNAi molecules include, for example, nucleic acids having the nucleotide sequences:

GGTGTGGAACAAGACCGAATGGATA;
GATAAGTCAGCTGTCGGCCACGAAT;
GGGAGAATGTCTTTCAAGAGCATCA;
GGCCACGAATATCAGTCGAAACTTT;
TCACAGAGAGATTACTCCAAAGGTT;
GGCAAATACGGTATCGACAAGGACA;
TCTTTCAAGAGCATCAGACCCTTAA;
TCAACATACACAAGCTGAGGGAGAA;
GGTATCGACAAGGACAAAGTGGATA;
and
GCCGTTGGCTTTGAGTATCAAGGCA.

Examples of ABL-2 (ARG) RNAi molecules include, for example, nucleic acids having the following nucleotide sequences:

GCCACTGAGAGTGACCCTAATCTCT;
CCACTGAGAGTGACCCTAATCTCTT;
GGTGAGTGGAGTGAAGTTCGCTCTA;
GGCGTCTGGAAGAAATACAGCCTTA;
GGAAATCAAGCATCCTAATCTGGTA;
GCTGCTCTACATGGCCACTCAGATT;
TCTTGCCTACAATACCTTCTCAATT;
TCAATTAAATCTGACGTCTGGGCTT;
CCCGGCTACCTATACTTCCTTCCAA;
GGACACTGAAGAAACAGGTGGAGAA;
GCCACTGAGAGTGACCCTAATCTCT;
GGTGAGTGGAGTGAAGTTCGCTCTA;
GGCGTCTGGAAGAAATACAGCCTTA;
GGAAATCAAGCATCCTAATCTGGTA;
GCTGCTCTACATGGCCACTCAGATT;
TCTTGCCTACAATACCTTCTCAATT;
TCAATTAAATCTGACGTCTGGGCTT;
CCCGGCTACCTATACTTCCTTCCAA;
GGACACTGAAGAAACAGGTGGAGAA;
and
GGGTAACTTCTCATCTGTTGCTTCT.

Examples of ABL-1 (ABL) RNAi molecules include, for example, nucleic acids having the following nucleotide sequences:

GGAATGGTGTGAAGCCCAAACCAAA;
GCCGAGTTGGTTCATCATCATTCAA;
GGAGGTGGAAGAGTTCTTGAAAGAA;
ACCACTTGGTGAAGGTAGCTGATTT;
GCCTACAACAAGTTCTCCATCAAGT;
TCCAGTATCTCAGACGAAGTGGAAA;
GGAAGACAGTTTGACTCGTCCACAT;
CCCTCTCATATCAACCCGAGTGTCT;
ACGTTCTGCGTGAGCTATGTGGATT;
GAGGCCATCAACAAACTGGAGAATA;
GGAGATAACACTCTAAGCATAACTA;
GGAATGGTGTGAAGCCCAAACCAAA;
GCCGAGTTGGTTCATCATCATTCAA;
ACCACTTGGTGAAGGTAGCTGATTT;
GCCTACAACAAGTTCTCCATCAAGT;
TCCAGTATCTCAGACGAAGTGGAAA;
GGAAGACAGTTTGACTCGTCCACAT;
CCCTCTCATATCAACCCGAGTGTCT;
ACGTTCTGCGTGAGCTATGTGGATT;
and
GAGGCCATCAACAAACTGGAGAATA.

Such nucleic acids can be provided individually in or in combination with one or more others and can be tested and utilized in methods described herein. The nucleic acids can be synthesized in a chemical form suitable for administration to cells, such as STEALTH™ RNAi.

ARG/ABL Reagents and Cortactin Reagents in Association with a Detectable Label

An ARG/ABL reagent or Cortactin reagent sometimes is in association with detectable label. The detectable label can be covalently linked to the reagent, and sometimes is in association with the reagent in a non-covalent linkage. Non-covalent linkages can be effected by a binding pair, wherein one binding pair member is in association with the reagent and the other binding pair member is in association with the detectable label. Any suitable binding pair can be utilized to effect a non-covalent linkage, including, but not limited to, antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, nucleic acid/complementary nucleic acid (e.g., DNA, RNA, PNA). Covalent linkages also can be effected by a binding pair, such as a chemical reactive group/complementary chemical reactive group (e.g., sulfhydryl/maleimide, sulfhydryl/haloacetyl derivative, amine/isotriocyanate, amine/succinimidyl ester, and amine/sulfonyl halides). Methods for attaching such binding pairs to reagents and effecting binding are known to the artisan.

Any detectable label suitable for detection of an interaction or biological activity in a system can be appropriately selected and utilized by the artisan. Examples of detectable labels are fluorescent labels such as fluorescein, rhodamine, and others (e.g., Anantha, et al., Biochemistry (1998) 37:2709 2714; and Qu & Chaires, Methods Enzymol. (2000) 321:353 369); radioactive isotopes (e.g., ¹²⁵I, ¹³¹I, ³⁵S, ³¹P, ³²P, ¹⁴C, ³H, ⁷Be, ²⁸Mg, ⁵⁷Co, ⁶⁵Zn, ⁶⁷Cu, ⁶⁸Ge, ⁸²Sr, ⁸³Rb, ⁹⁵Tc, ⁹⁶Tc, ¹⁰³Pd, ¹⁰⁹Cd, and ¹²⁷Xe); light scattering labels (e.g., U.S. Pat. No. 6,214,560, and commercially available from Genicon Sciences Corporation, CA); chemiluminescent labels and enzyme substrates (e.g., dioxetanes and acridinium esters), enzymic or protein labels (e.g., green fluorescence protein (GFP) or color variant thereof, luciferase, peroxidase); other chromogenic labels or dyes (e.g., cyanine), and labels described previously. Use of reagents in association with a detectable label are described in greater detail hereafter.

Cell Reagents

An ARG/ABL reagent or Cortactin reagent sometimes is a cell that over-expresses or under-expresses an ARG/ABL protein or fragment, a Cortactin protein or fragment or a nucleic acid encoding the foregoing. A cell reagent may over-express or under-express one of an ARG, ABL or Cortactin protein, fragment or nucleic acid product, or may over-express or under-express a combination or all of an ARG, ABL or Cortactin protein, fragment or nucleic acid product. A cell reagent can be processed in a variety of manners. For example, an artisan may prepare a lysate from a cell reagent and optionally isolate or purify components of the cell, may transfect the cell with a nucleic acid reagent, may fix a cell reagent to a slide for analysis (e.g., microscopic analysis) and can immobilize a cell to a solid phase.

A cell that “over-expresses” an ARG, ABL and/or Cortactin protein or fragment or nucleic acid product produces at least two, three, four or five times or more of the product as compared to a native cell from an organism that has not been genetically modified and/or exhibits no apparent symptom of a cell-proliferative disorder. Over-expressing cells may be stably transfected or transiently transfected with a nucleic acid that encodes the ARG, ABL and/or Cortactin protein or fragment or nucleic acid (e.g., a nucleic acid reagent described above). A cell that “under-expresses” an ARG, ABL and/or Cortactin protein or fragment or nucleic acid product produces at least five times less of the product as compared to a native cell from an organism that has not been genetically modified and/or exhibits no apparent symptom of a cell-proliferative disorder. In some embodiments, a cell that under-expresses an ARG, ABL and/or Cortactin protein, fragment or nucleic acid product contains no nucleic acid that can encode such a product (e.g., the cell is from a knock-out mouse) and no detectable amount of the product is produced. Methods for generating knock-out animals and using cells extracted therefrom are known (e.g., Miller et al., J. Cell. Biol. 165: 407-419 (2004)). A cell that under-expresses an ARG, ABL and/or Cortactin protein, fragment or nucleic acid, for example, sometimes is in contact with a nucleic acid inhibitor that blocks or reduces the amount of the product produced by the cell in the absence of the inhibitor. An over-expressing or under-expressing cell may be within an organism (in vivo) or from an organism (ex vivo or in vitro).

The artisan may select any cell that over-expresses or under-expresses an ARG, ABL and/or Cortactin protein or fragment or nucleic acid. Cells include, but are not limited to, bacterial cells (e.g., Escherichia spp. cells (e.g., Expressway™ HTP Cell-Free E. coli Expression Kit, Invitrogen, California) such as DH10B, Stb12, DH5-alpha, DB3, DB3.1 for example), DB4, DB5, JDP682 and ccdA-over (e.g., U.S. application Ser. No. 09/518,188), Bacillus spp. cells (e.g., B. subtilis and B. megaterium cells), Streptomyces spp. cells, Erwinia spp. cells, Klebsiella spp. cells, Serratia spp. cells (particularly S. marcessans cells), Pseudomonas spp. cells (particularly P. aeruginosa cells), and Salmonella spp. cells (particularly S. typhimurium and S. typhi cells); photosynthetic bacteria (e.g., green non-sulfur bacteria (e.g., Choroflexus spp. (e.g., C. aurantiacus), Chloronema spp. (e.g., C. gigateum)), green sulfur bacteria (e.g., Chlorobium spp. (e.g., C. limicola), Pelodictyon spp. (e.g., P. luteolum), purple sulfur bacteria (e.g., Chromatium spp. (e.g., C. okenii)), and purple non-sulfur bacteria (e.g., Rhodospirillum spp. (e.g., R. rubrum), Rhodobacter spp. (e.g., R. sphaeroides, R. capsulatus), Rhodomicrobium spp. (e.g., R. vanellii)); yeast cells (e.g., Saccharomyces cerevisiae cells and Pichia pastoris cells); insect cells (e.g., Drosophila (e.g., Drosophila melanogaster), Spodoptera (e.g., Spodoptera frugiperda Sf9 and Sf21 cells) and Trichoplusa (e.g., High-Five cells); nematode cells (e.g., C. elegans cells); avian cells; amphibian cells (e.g., Xenopus laevis cells); reptilian cells; and mammalian cells (e.g., NIH3T3, 293, CHO, COS, VERO, C127, BHK, Per-C6, Bowes melanoma and HeLa cells). These and other suitable cells are available commercially, for example, from Invitrogen Corporation, (Carlsbad, Calif.), American Type Culture Collection (Manassas, Va.), and Agricultural Research Culture Collection (NRRL; Peoria, Ill.).

Detection of Interactions and Biological Activities

Reagents may be contacted in any convenient format and system by the artisan. As used herein, the term “system” refers to an environment that receives the assay components, including but not limited to microtiter plates (e.g., 96-well or 384-well plates), silicon chips having molecules immobilized thereon and optionally oriented in an array (see, e.g., U.S. Pat. No. 6,261,776 and Fodor, Nature 364: 555-556 (1993)), microfluidic devices (see, e.g., U.S. Pat. Nos. 6,440,722; 6,429,025; 6,379,974; and 6,316,781) and cell culture vessels. The system can include attendant equipment, such as signal detectors, robotic platforms, pipette dispensers and microscopes. A system sometimes is cell free, sometimes includes one or more cells, sometimes includes or is a cell sample from an animal (e.g., a biopsy, organ, appendage), and sometimes is a non-human animal. Cells may be extracted from any appropriate subject, such as a mouse, rat, hamster, rabbit, guinea pig, ungulate (e.g., equine, bovine, porcine), monkey, ape or human subject, for example.

The artisan can select test molecules and test conditions based upon the system utilized and the interaction and/or biological activity parameters monitored. Any type of test molecule can be utilized, including any reagent described herein, and can be selected from chemical compounds, antibodies and antibody fragments, binding partners and fragments, and nucleic acid molecules, for example. Specific embodiments of each class of such molecules are described above. One or more test molecules may be added to a system in assays for identifying molecules that modulate an interaction or biological activity. Test molecules and other components can be added to the system in any order. The system can be exposed to any appropriate test condition, including a condition that induces DNA damage (e.g., ionizing radiation) or induces mitogenic stimulation, and/or exposing the sample to an infective agent (e.g., a virus, bacterium, or fungus). A sample exposed to a particular condition or test molecule often is compared to a sample not exposed to the condition or test molecule so that any changes in interactions or biological activities can be observed and/or quantified.

One or more system components and/or reagents may be immobilized to a solid support. The attachment between a component and the solid support may be covalent or non-covalent (see, e.g., U.S. Pat. No. 6,022,688 for non-covalent attachments). The term “solid support” or “solid phase” as used herein refers to a wide variety of materials including solids, semi-solids, gels, films, membranes, meshes, felts, composites, particles, and the like typically used by those of skill in the art to sequester molecules. The solid phase can be non-porous or porous. Suitable solid phases include those developed and/or used as solid phases in solid phase binding assays. See, e.g., chapter 9 of Immunoassay, E. P. Diamandis and T. K. Christopoulos eds., Academic Press: New York, 1996, hereby incorporated by reference. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. See, e.g., Leon et al., Bioorg. Med. Chem. Lett. 8: 2997 (1998); Kessler et al., Agnew. Chem. Int. Ed. 40: 165 (2001); Smith et al., J. Comb. Med. 1: 326 (1999); Orain et al., Tetrahedron Lett. 42: 515 (2001); Papanikos et al., J. Am. Chem. Soc. 123: 2176 (2001); Gottschling et al., Bioorg. And Medicinal Chem. Lett. 11: 2997 (2001). For example, ARG, ABL or Cortactin proteins or fragments sometimes are purified by a polyhistidine tag-chelating resin (e.g., ProBond™ purification system (Invitrogen, California)) and/or a cysteine-rich tag purification resin (e.g., Lumio® agent (Invitrogen, California) linked to a solid phase). Provided also are arrays comprising one or more, two or more, three or more, etc., of ARG, ABL or Cortactin proteins or fragments immobilized at discrete sites on a solid support in an ordered array. Such arrays sometimes are high-density arrays, such as arrays in which each spot comprises at least 100 protein molecules per square centimeter. Solid supports include but are not limited to a glass slide, a microchip, a microtiter plate, a chromatography support, a nanotube, and the like. Types of solid supports, linker molecules for covalent and non-covalent attachments to solid supports, and methods for immobilizing nucleic acids, proteins and other molecules to solid supports are known (e.g., U.S. Pat. Nos. 6,261,776; 5,900,481; 6,133,436; and 6,022,688; and WIPO publication WO 01/18234).

Assay systems sometimes are heterogeneous or homogeneous. In heterogeneous assays, one or more reagents and/or assay components are immobilized on a solid phase, and complexes are detected on the solid phase at the end of the reaction. In homogeneous assays, the entire reaction is carried out in a liquid phase. In either approach, the order of addition of reactants can be varied to obtain different information about the molecules being tested. For example, test compounds that agonize target molecule/binding partner interactions can be identified by conducting the reaction in the presence of the test molecule in a competition format. Alternatively, test molecules that agonize preformed complexes, e.g., molecules with higher binding constants that displace one of the components from the complex, can be tested by adding a test compound to the reaction mixture after complexes have been formed. In embodiments described herein, a complex often comprises an ARG and/or ABL reagent and a Cortactin reagent, and sometimes comprises an ARG reagent, an ABL reagent or a Cortactin reagent.

In a heterogeneous assay embodiment, one or more reagents or assay components are anchored to a solid surface (e.g., a microtiter plate), and a non-anchored component or reagent often is labeled, either directly or indirectly. The anchored molecule can be immobilized by non-covalent or covalent attachments. Alternatively, an immobilized antibody specific for the molecule to be anchored can be used to anchor the molecule to the solid surface. A partner of the immobilized species is exposed to the coated surface with or without a test molecule. After the reaction is complete, unreacted components are removed (e.g., by washing) such that a significant portion of any complexes formed remain immobilized on the solid surface. Where the non-immobilized species is pre-labeled, the detection of label immobilized on the surface is indicative of complex formation. Where the non-immobilized species is not pre-labeled, an indirect label can be used to detect complexes anchored to the surface (e.g., by using a labeled antibody specific for the initially non-immobilized species). Depending upon the order of addition of reaction components, test compounds that inhibit complex formation or disrupt preformed complexes can be detected.

In certain embodiments, a protein or peptide test molecule or reagent is linked to a phage via a phage coat protein. Molecules capable of interacting with the protein or peptide linked to the phage are immobilized to a solid phase, and phages displaying proteins or peptides that interact with the immobilized components adhere to the solid support. Nucleic acids from the adhered phages then are isolated and sequenced to determine the sequence of the protein or peptide that interacted with the components immobilized on the solid phase. Methods for displaying a wide variety of peptides or proteins as fusions with bacteriophage coat proteins are well known (Scott and Smith, Science 249: 386-390 (1990); Devlin, Science 249: 404-406 (1990); Cwirla et al., Proc. Natl. Acad. Sci. 87: 6378-6382 (1990); Felici, J. Mol. Biol. 222: 301-310 (1991)). Methods are also available for linking the test polypeptide to the N-terminus or the C-terminus of the phage coat protein. The original phage display system was disclosed, for example, in U.S. Pat. Nos. 5,096,815 and 5,198,346. This system used the filamentous phage M13, which required that the cloned protein be generated in E. coli and required translocation of the cloned protein across the E. coli inner membrane. Lytic bacteriophage vectors, such as lambda, T4 and T7 are more practical since they are independent of E. coli secretion. T7 is commercially available and described in U.S. Pat. Nos. 5,223,409; 5,403,484; 5,571,698; and 5,766,905.

In some embodiments, the reaction can be conducted in a liquid phase in the presence or absence of test molecule, where the reaction products are separated from unreacted components, and the complexes are detected (e.g., using an immobilized antibody specific for one of the binding components to anchor any complexes formed in solution, and a labeled antibody specific for the other partner to detect anchored complexes). Again, depending upon the order of addition of reactants to the liquid phase, test compounds that inhibit complex or that disrupt preformed complexes can be identified.

In some homogeneous assay embodiments, a preformed complex comprising a reagent and/or other component is prepared. One or both of the target molecule or binding partner is labeled, and the signal generated by the label(s) is quenched upon complex formation (e.g., U.S. Pat. No. 4,109,496 that utilizes this approach for immunoassays). Addition of a test molecule that competes with and displaces one of the species from the preformed complex can result in the generation of a signal above background. In this way, test substances that disrupt target molecule/binding partner complexes can be identified.

In an embodiment for identifying test molecules that antagonize or agonize formation of a complex comprising a reagent and/or other assay component, a reaction mixture containing components of the complex is prepared under conditions and for a time sufficient to allow complex formation. The reaction mixture often is provided in the presence or absence of the test molecule. The test molecule can be included initially in the reaction mixture, or can be added at a time subsequent to the addition of the target molecule and its binding partner. Control reaction mixtures are incubated without the test molecule or with a placebo. Formation of any complex is detected. Decreased formation of a complex in the reaction mixture containing test molecule as compared to in a control reaction mixture indicates that the molecule antagonizes target molecule/binding partner complex formation. Alternatively, increased formation of a complex in the reaction mixture containing test molecule as compared to in a control reaction mixture indicates that the molecule agonizes target molecule/binding partner complex formation. In certain embodiments, complex formation of target molecule/binding partner can be compared to complex formation of mutant target molecule/binding partner (e.g., amino acid modifications in a protein or fragment reagent). Such a comparison can be useful in cases where it is desirable to identify test molecules that modulate interactions of mutant but not non-mutated target gene products.

In some embodiments, the artisan detects an interaction between an ARG, ABL and/or Cortactin reagent, sometimes an interaction between one or more of such reagents with one or more other molecules, and sometimes a modulatory effect of a test molecule on such an interaction. As used herein, the term “interaction” typically refers to reversible binding of particular system components to one another, and such interactions can be quantified. Often, binding affinity is quantified by plotting signal intensity as a function of a range of concentrations or amounts of a reagent, reactant or other system component. Quantified interactions can be expressed in terms of a concentration or amount of a reagent required for emission of a signal that is 50% of the maximum signal (IC₅₀). Also, quantified interactions can be expressed as a dissociation constant (K_{d} or Kᵢ) using kinetic methods known in the art.

A variety of signals can be detected to identify the presence, absence or amount of an interaction. A fluorescence signal is typically monitored in the assays by exciting a fluorophore at a specific excitation wavelength and then detecting fluorescence emitted by the fluorophore at a different emission wavelength. Many nucleic acid interacting fluorophores and their attendant excitation and emission wavelengths are known (e.g., those described above). Standard methods for detecting fluorescent signals also are known, such as by using a fluorescence detector. Background fluorescence may be reduced in the system with the addition of photon reducing agents (see, e.g., U.S. Pat. No. 6,221,612), which can enhance the signal to noise ratio.

Another signal that can be detected is a change in refractive index at a solid optical surface, where the change is caused by the binding or release of a refractive index enhancing molecule near or at the optical surface. These methods for determining refractive index changes of an optical surface are based upon surface plasmon resonance (SPR). SPR is observed as a dip in light intensity reflected at a specific angle from the interface between an optically transparent material (e.g., glass) and a thin metal film (e.g., silver or gold). SPR depends upon the refractive index of the medium (e.g., a sample solution) close to the metal surface. A change of refractive index at the metal surface, such as by the adsorption or binding of material near the surface, will cause a corresponding shift in the angle at which SPR occurs. SPR signals and uses thereof are further exemplified in U.S. Pat. Nos. 5,641,640; 5,955,729; 6,127,183; 6,143,574; and 6,207,381, and WIPO publication WO 90/05295 and apparatuses for measuring SPR signals are commercially available (Biacore, Inc., Piscataway, N.J.). In certain embodiments, an ARG, ABL and/or Cortactin reagent can be linked via a linker to a chip having an optically transparent material and a thin metal film, and interactions between and/or with the reagents can be detected by changes in refractive index.

Other signals representative of structure may also be detected, such as NMR spectral shifts (see, e.g., Arthanari & Bolton, Anti-Cancer Drug Design 14: 317-326 (1999)), mass spectrometric signals and fluorescence resonance energy transfer (FRET) signals (e.g., Lakowicz et al., U.S. Pat. No. 5,631,169; Stavrianopoulos et al. U.S. Pat. No. 4,868,103). In FRET approaches, a fluorophore label on a first, “donor” molecule is selected such that its emitted fluorescent energy will be absorbed by a fluorescent label on a second, “acceptor” molecule, which in turn is able to fluoresce due to the absorbed energy. Alternately, the “donor” polypeptide molecule may simply utilize the natural fluorescent energy of tryptophan residues. Labels are chosen that emit different wavelengths of light, such that the “acceptor” molecule label may be differentiated from that of the “donor”. Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. A FRET binding event can be conveniently measured using standard fluorometric detection means well known (e.g., using a fluorimeter). Molecules useful for FRET are known (e.g., fluorescein and terbium). FRET can be utilized to detect interactions in vitro or in vivo.

Interaction assays sometimes are performed in a heterogeneous format where interactions are detected using a solid phase and a detectable label in association with an ARG, ABL and/or Cortactin protein, fragment or nucleic acid is separated from unassociated label. An example of such a format is an immunoprecipitation assay. Interaction assays sometimes are performed in a format in which a detectable label in association with an ARG, ABL or Cortactin protein, fragment or nucleic acid is separated from unassociated labels using other types of separation processes. Such assays may or may not include use of a solid phase. Multiple separation processes are available, such as gel electrophoresis, sedimentation (e.g., gradient sedimentation) and flow cytometry processes, for example. Flow cytometry processes include, for example, such as flow microfluorimetry (FMF) and fluorescence activated cell sorting (FACS); U.S. Pat. No. 6,090,919 (Cormack, et al.); U.S. Pat. No. 6,461,813 (Lorens); and U.S. Pat. No. 6,455,263 (Payan)). In some embodiments, cells also may be washed of unassociated detectable label, and detectable label associated with cellular components may be visualized (e.g., by microscopy).

The presence, absence or amount of a particular biological activity of an ARG, ABL and/or Cortactin protein, fragment, nucleic acid or other reagent also can be assessed in a system. The term “biological activity” or “biological function” generally includes modification of an ARG, ABL and/or Cortactin protein, fragment or nucleic acid or a modification effected by such a molecule. Any modification can be monitored, such as addition or removal of a phosphoryl, alkyl (e.g., methyl), fatty acid (e.g., myristoyl or palmitoyl), isoprenyl, glycosyl (e.g., polysaccharide), acetyl or peptidyl (e.g., ubiquitin). Multiple glycosidic linkages are known to the artisan, including but not limited to N-glycosidic linkages (e.g., GlcNAc-β-Asn, Glc-β-Asn, Rha-Asn and Glc-β-Arg linkages); O-glycosidic linkages (e.g., linkages to Ser, Thr, Tyr, Hyp [hydroxyproline], and Hyl [hydroxylysine]; GalNAc-Ser/Thr, GalNAc-β-Ser/Thr, Gal-Ser/Thr, Man-Ser/Thr, Fuc-Ser/Thr, Glc-β-Ser, Pse-Ser/Thr, DiActrideoxyhexose-Ser/Thr, FucNAc-β-Ser/Thr, Xyl-β-Ser, Glc-Thr, GlcNAc-Thr, Gal-β-Hyl, Gal-Hyp, Gal-β-Hyp, Ara-Hyp Ara-β-Hyp, GlcNAc-Hyp, Glc-Tyr and Glc-β-Tyr linkages); C-mannosyl linkages (e.g., mannosyl linkage to C-2 of the Trp through a C—C bond); phosphoglycosyl linkages (e.g., attachment of sugar (e.g., GlcNAc, Man, Xyl, and Fuc) to protein via a phosphodiester bond; GlcNAc-1-P-Ser, Man-1-P-Ser, Xyl-1-P-Ser, Fuc-β-1-P-Ser linkages); and glypiated linkages (e.g., Man is linked to phosphoethanolamine, which in turn is attached to the terminal carboxyl group of a protein). Extent of glycosylation can be assessed by the artisan using known methods (e.g., Spiro, Glycobiology 12: 43R-56R (2002)). Kinetic parameters descriptive of modification characteristics in the system can be assessed, including for example, assessing Kₘ, k_{cat}, kₒₙ, k_{off} parameters.

Addition and/or removal of a phosphate moiety from an ARG, ABL or Cortactin protein or fragment or a binding partner thereof can be detected in a variety of systems selected by the artisan. In some embodiments, the gamma phosphoryl moiety of adenosine triphosphate (ATP), which is transferred to a protein substrate by protein kinases, or a derivative thereof is detectably labeled. In such embodiments, the detectably labeled gamma phosphoryl moiety transferred to a substrate is detected. In some embodiments, an ATP having a ³²P or ³³P gamma phosphoryl moiety is utilized in an assay. In certain embodiments, The gamma phosphate of ATP can be detectably labeled by any method known to the skilled artisan. In certain embodiments, the gamma moiety includes a ³⁵S atom.

In certain embodiments, the substrate is immobilized to a solid phase (e.g., a substrate array) and phosphorylation activity is monitored. A reaction buffer may be utilized in such a system that includes components conducive to phosphorylation reactions. These conditions include, for example, pH, salt concentration, concentration of Mg²⁺, and detergent concentration. After incubation in the reaction buffer, the microarray is washed to remove any labeled ATP and the product is quantified via the detectably labeled phosphate that has been transferred during the kinase reaction from ATP to the substrate. Signal intensity is proportional to the amount of labeled phosphate on the substrate and corresponds to phosphorylation activity. In some embodiments, a substrate is labeled with a detectable phosphoryl moiety and dephosphorylation of the substrate is detected.

Without being bound by theory, some kinases and phosphatases act on a substrate only in a particular molecular context. Such a molecular context may, e.g., consist of certain scaffold proteins. In certain embodiments, such scaffold proteins are provided with the reaction buffer. In some embodiments, the scaffold proteins are also immobilized on the surface of a solid support.

In certain embodiments, a kinase reaction can be visualized and optionally quantified using antibodies that bind specifically to phosphorylated proteins or peptides. Such antibodies include, but are not limited to antibodies that bind to phospho-serine, antibodies that bind to phosphor-threonine or antibodies that bind to phospho-tyrosine. The antibody sometimes is specific for the phosphoryl amino acid regardless of the amino acid sequence surrounding the phosphoryl amino acid, and in some embodiments, the antibody specifically binds to an epitope comprising the phosphoryl amino acid and one or more surrounding amino acids. The antibody that binds to the phosphorylated protein or peptide may include a detectable label or can be associated with a detectable label during the assay. In some embodiments, a secondary antibody is used to detect the antibody bound to the phosphorylated protein or peptide. The amount of phosphorylated substrate can be detected, and such assays are useful for detecting phosphorylation and/or dephosphorylation activity. In some assay embodiments, phosphorylation is detected by fluorescence polarization after contacting a sample with a peptide substrate linked to a fluorophore and an antibody that specifically binds to the phosphorylated peptide (e.g., PolarScreen™ kinase assay; http address www.invitrogen.com/content.cfm?pageid=10568).

In certain assay embodiments, phosphorylation is detected by FRET. In an embodiment a sample is contacted with a peptide substrate linked to two fluorophores capable of FRET (e.g., one fluorophore at the N-terminus and one at the C-terminus) and a protease that specifically cleaves the peptide substrate differentially based upon its phosphorylation state (e.g., Z′-LYTE™ protein kinase and phosphatase assays (http address www.invitrogen.com/content.cfm?pageid=9866)). In some embodiments, a sample is contacted with (1) a peptide substrate containing a first fluorophore and (2) a detection molecule linked to a second fluorophore capable of FRET with the first fluorophore linked to the peptide (e.g., LanthaScreen™ TR-FRET Assay (http address www.invitrogen.com/content.cfm?pageid=10513)). In the latter embodiments, the detection molecule sometimes is an antibody that specifically binds to phosphorylated peptide and not specifically to non-phosphorylated peptide (e.g., terbium-labeled phospho-tyrosine specific antibody). The detection molecule sometimes is a molecule that is part of a binding pair (e.g., biotin), the peptide is linked to the other binding pair member (e.g., streptavidin or avidin) and the assay system is contacted with a protease that differentially cleaves phosphorylated and non-phosphorylated peptide. These assays can be utilized in homogenous or heterogeneous formats.

In certain embodiments, phosphorylation can be detected using a molecule that binds to phosphate and is linked to a detectable label. A dye can be utilized as a detectable label, such as a dye comprising a metal-chelating moiety. In a specific embodiment, a phosphorylated protein or peptide is detected using a metal-chelating dye. Metal-chelating dyes include, without limitation, BAPTA, IDA, DTPA, phenanthrolines and derivatives thereof (e.g., U.S. Pat. Nos. 4,603,209; 4,849,362; 5,049,673; 5,453,517; 5,459,276; 5,516,911; 5,501,980; and 5,773,227). In specific embodiments, a dye in Pro-Q Diamond stain (Molecular Probes, Oregon) is utilized (e.g., gel or microarray stain).

Other phosphorylation detection systems that may be utilized include commercially available kits such as the PhosphoELISA (Biosource International) and fluorescence-based assays. Suitable fluorescence-based assay systems utilize reagents with novel metal binding amino acid residues exhibiting chelation-enhanced fluorescence (CHEF) upon binding to Mg²⁺ (e.g., US 2005/0080242A2 and US 2005/0080243A1).

A biological activity also can include localization of an ARG, ABL and/or Cortactin protein, fragment, nucleic acid or binding partner thereof, to a cellular location. Any cellular location may be detected, such as the nucleus, nucleolus, cytoplasm, mitochondria, Golgi components, plasma membrane, membrane ruffles and actin and/or microtubule structures (e.g., lamellipodial protrusions, retractions, and phase-dense ruffles; Wang et al., PNAS 98: 14865-14870 (2001) and Miller et al. J. Cell Biol. 407-419 (2004)), for example. The amount of a particular protein or fragment localized to a cellular region, and the amount of type of structure formed at a region (e.g., the amount of membrane ruffles) can be determined, for example. Multiple methods are available to the artisan for observing cellular localization. The artisan may utilize microscopy to analyze cellular localization, such as immunofluorescence microscopy, time-lapse microscopy and kymography, for example. A representative procedure for immunofluorescence microscopy is as follows. Cells are plated on glass coverslips coated with 10 μg/ml fibronectin (Sigma-Aldrich) and blocked with 1% BSA (GIBCO BRL) 48-72 h after infection and are allowed to attach for 30 min. Cells are rinsed before fixation with PHEM buffer (60 mM Pipes, 25 mM Hepes, 10 mM EDTA, 2 mM MgCl2, pH 6.9) that is prewarmed to 37° C. Cells are fixed with 4% PFA (prewarmed to 37° C.) for 20 min at RT and then permeabilized with 0.5% or 1% Triton X-100 for 10 min. Cells are stained with anti-tubulin antibodies (clone DM 1A; Sigma-Aldrich), Alexa 594-labeled secondary antibodies (Molecular Probes), and Alexa 350-phalloidin (Molecular Probes). Cells are imaged on a microscope (model TE2000-S; Nikon) at 40× or 100×. Stock solutions of nocodazole (Sigma-Aldrich) or latrunculin A (Sigma-Aldrich) are dissolved in DMSO. Nocodazole or latrunculin A is diluted in growth media at the concentrations indicated in the figure legends and added to cells for 30 min at 37° C. before fixation. Control cells are treated with DMSO alone.

A representative procedure for time-lapse microscopy and kymography is as follows. For time-lapse microscopy, cells are adapted to microscopy media (growth media with 10 mM Hepes [GIBCO BRL]) for about 16 h and plated on fibronectin-coated, BSA-blocked glass coverslips. Cells are imaged between 30 min and 2 h after plating using a microscope (model TE2000-S; Nikon) driven by Openlab software (Improvision). Cells are maintained at 37° C. during imaging with an in-line flow heater and a heated chamber (Warner Instruments). 40× phase contrast and YFP movies are about 10 min long with frames taken every 10-12 seconds. For kymography, phase-contrast time-lapse sequences are obtained as described above. Each cell is overlaid with a template containing eight equally spaced radiating lines with the center located on the cell nucleus. Kymographs are made along each of the eight lines at the intersecting point along the cell periphery using ImageJ software (NIH). Kymographs are analyzed for frequency of lamellipodial protrusions, retractions, and phase-dense ruffles as described by Hinz and colleagues (Hinz et al., 1999).

The artisan also may employ sedimentation procedures to identify cell localization of particular reagents. A representative co-sedimentation assay procedure is as follows. Phosphocellulose-purified tubulin is prepared from frozen chick brains following the protocol described in Hyman et al. (1991). Tubulin is polymerized at a final concentration of 18 μM at 37° C. for 30 min. The polymerization buffer contained 100 mM Pipes, pH 6.8, 1 mM MgSO₄, 1 mM EGTA, 1 mM GTP, and 15 nM paclitaxel. For binding assays, 0.25 μM ARG or ARG mutant protein is mixed with increasing concentrations of MTs (0-8 μM) at 25° C. for 15 min in a binding buffer containing 20 mM Pipes, pH 6.8, 100 mM KCL, 1 mM DTT, 1 mM GTP, and 15 nM paclitaxel. Mixtures are pelleted by centrifugation at 120,000 g for 30 min at 20° C. As a control, ARG or ARG mutant protein is subjected to centrifugation alone, in the absence of MTs. The pellet (P) and supernatant (S) fractions are re-covered and separated by SDS-PAGE and stained with Coomassie blue. Protein bands are quantified by densitometry. Binding affinity is determined from the concentration of ARG bound to MTs for each concentration of MTs in the assays. Data are analyzed using KaleidaGraph software (Synergy Soft-ware) and fitted to the equation y=((r+K_{d}+x)−square root(r+K_{d}+x)²−4(rx))/2, where r=[ARG]total, x=[MT]ₜₒₜₐₗ, y=[ARG]_{bound}, and K_{d} is the dissociation constant of the ARG-MT complex.

The term biological activity also includes cross-linking of particular cell components that contribute to cell structure and/or cell movement. For example, actin and microtubule (MT) cross-linking can be monitored by the artisan. Representative cross-linking procedures are provided hereafter. A representative fluorescence assay for monitoring F-actin-MT cross-linking is founded upon the phenomena that rhodamine-labeled MTs are stabilized by paclitaxel, and F-actin is stabilized by a 1:4 mix of phalloidin/Alexa 488-phalloidin. Purified ARG or ARG mutant proteins (0.5 μM), for example, can be mixed with 1 μM MTs at 25° C. for 10 min. After F-actin addition (1 μM), the mixture is incubated for an additional 15 min. The mixture is then diluted fivefold (or twofold for ARG557-930) and visualized by fluorescence microscopy at 63× magnification. In a representative co-sedimentation assay for F-actin-MT cross-linking, MTs are polymerized as described above and F-actin is prepared using known methodology. 1 μM ARG is incubated with 1 μM MTs in binding buffer at 25° C. for 10 min. After addition of 1 μM F-actin, the reaction is incubated for an additional 15 min at 25° C. The mixture is pelleted by centrifugation at 5,000 g for 10 min at 20° C. to pellet F-actin bundles and associated proteins. The pellet (P) and supernatant (S) fractions are recovered, separated by SDS-PAGE, and visualized using Coomassie blue staining.

A biological activity also includes metastasis, which can be monitored in response to an increased or decreased interaction between an ARG and/or ABL protein or fragment and a Cortactin protein or fragment. A metastatic event can include, for example, a local invasion, passive transport, lodgement and/or proliferation. Metastatic events may be observed in vitro or in vivo. Cell proliferation and invasion may be monitored in vitro or in vivo (e.g., tumor growth), for example. A test molecule may be administered to an animal or in an in vitro system to determine whether proliferation or invasion is modulated.

A representative in vitro assay for cell proliferation is as follows. An aliquot of cells are plated in Boyden chambers with 8 μm pore membranes that are coated with growth-factor reduced matrigel (Becton Dickinson). In addition to growth factors, matrigel contains basement membrane components such as collagens, laminin, and proteoglycans, providing physiological components for cells such as breast cancer cells. One day after contact with a test molecule, cells are trypsinized and resuspended in media without serum and plated on top of a matrigel-coated membrane, which is suspended over media containing 5% serum. Cells are allowed to grow for 6 days, and then are fixed in 2% glutaraldehyde and stained with 0.2% crystal violet and the number, orientation and morphology of the cells are observed.

A representative invasion assay is as follows. The metastatic potential of tumor cells can be assessed in vitro using Boyden chambers. One or more cell lines having no or little metastatic activity and one or more cell lines known as having metastatic potential (e.g., breast cancer cell line MDA-MB-231) are tested. Cells are replated 5 days after contact with a test molecule on matrigel-coated Boyden chambers suspended on media containing 10% serum. Cells are stained with crystal violet 20 hrs later and photographed. Cells that remain on top of the membrane are scrubbed off and the cells that had invaded through the matrigel and grew on the bottom of the membrane are photographed. Cell numbers, orientation and morphology in the bottom of the membrane are assessed.

A biological activity also includes a change in cellular level, cellular localization or a phosphorylation state of a protein or cell component other than ARG, ABL or Cortactin affected by an increased or reduced interaction between an ARG/ABL protein or fragment and a Cortactin protein or fragment (e.g., transcription factor, nuclear protein, cytosolic protein, response element). In some embodiments, the biological activity is localization, phosphorylation state or levels of a protein in a Arp2/3 complex, the latter of which nucleates actin filaments. Response elements include, but are not limited to, nuclear factor activated T-cells response element (NFAT), interferon stimulated response element (ISRE), GAS, Smad binding element (SBE), activator protein 1 (AP-1) response element, c-fox promoter (SIE, SRE, CRE), cAMP response element (CRE), serum response element (SRE), 1E2F DNA binding element, hyoxia responsive elements (HRE), lymphoid enhancing factor (LEF)/T cell factor (TCF) DNA binding element, nuclear factor of NFkappaB cells response element, SIE and Gli. An effect of a molecule that interacts with an ARG, ABL or Coractin molecule on these and other response elements can be detected to determine pathway members associated with an interaction between ABL/ARG and Cortactin (e.g., CellSensor™ vectors combine lentivirus with beta-lactamase to enable signal transduction pathway analysis using GeneBLAzer® technology (http address www.invitrogen.com/content.cfm?pageid=10523) Invitrogen; Carlsbad, Calif.).

In addition to the reagents and components described above, the artisan may add other useful components to the system. In some embodiments, one or more detergents are added to a system, for example. Detergents include, but are not limited to, a detergent described above, anionic detergents such as sodium n-dodecyl sulfate (SDS); dihydroxy or trihydroxy bile acids (and their salts), such as cholic acid (sodium cholate), deoxycholic acid (sodium deoxycholate), taurodeoxycholic acid (sodium taurodeoxycholate), taurocholic acid (sodium taurocholate), glycodeoxycholic acid (sodium glycodeoxycholate), glycocholic acid (sodium glycocholate); cationic detergents such as cetyl trimethylammonium bromide (CTAB); non-ionic detergents such as the polyoxyethylenes NP-40, TRITON® X-100, TRITON® X-114, C₁₂E₈, C₁₂E₉, GENAPOL® X-080, GENAPOL® X-100, LUBROL® PX, BRIJ® 35, TWEEN® 20, and TWEEN® 20; alkyl glycosides such as dodecyl-β-D-maltoside (“dodecyl maltoside”), n-nonyl-β-D-glucopyranoside, n-octyl-β-D-glucopyranoside (“octyl glucoside”), n-heptyl-β-D-glucopyranoside, and n-hexyl-β-D-glucopyranoside; alkylamine oxides such as lauryl dimethylamine oxide (LDAO); and zwitterionic detergents, such as CHAPS, CHAPSO, n-dodecyl-N,N-dimethylglycine, and ZWITTERGENTS® 3-08, 3-10, 3-12, 3-14, and 3-16.

Molecules Effecting an Interaction or Affecting a Biological Activity

In embodiments where test molecules are screened for an effect on a biological activity or an interaction with ARG, ABL or Cortactin or a complex thereof, test molecules identified as having an effect or an interaction can be analyzed and compared to one another (e.g., ranked). Molecules identified as having an interaction or affecting a biological activity in the methods described above are referred to hereafter as “candidate molecules.” Provided herein are candidate molecules identified by screening methods described herein, information descriptive of such candidate molecules, and methods of using candidate molecules (e.g., for therapeutic treatment of a condition).

Accordingly, provided is structural information descriptive of a candidate molecule identified by a method described herein. In certain embodiments, information descriptive of molecular structure (e.g., chemical formula or sequence information) sometimes is stored and/or renditioned as an image or as three-dimensional coordinates. The information often is stored and/or renditioned in computer readable form and sometimes is stored and organized in a database. In certain embodiments, the information may be transferred from one location to another using a physical medium (e.g., paper) or a computer readable medium (e.g., optical and/or magnetic storage or transmission medium, floppy disk, hard disk, random access memory, computer processing unit, facsimile signal, satellite signal, transmission over an internet or transmission over the world-wide web).

Also provided are methods for using a candidate molecule identified by a method described herein. Such uses include preparation of a formulation, preparation of a medicament and use as a therapeutic, for example. In some embodiments, provided is a method for treating a disorder, comprising administering a molecule identified by a method described herein to a subject in an amount effective to treat the disorder, whereby administration of the molecule treats the disorder. The terms “treating,” “treatment” and “therapeutic effect” as used herein refer to ameliorating, alleviating, lessening, and removing symptoms of a disease or condition. In some embodiments involving a nucleic acid candidate molecule, such as in gene therapies, antisense thereapies, and siRNA or RNAi therapies, the nucleic acid may integrate with a host genome or not integrate. Any suitable formulation of a candidate molecule can be prepared for administration. Any suitable route of administration may be used, including but not limited to oral, parenteral, intravenous, intramuscular, topical and subcutaneous routes.

In cases where a candidate molecule is sufficiently basic or acidic to form stable nontoxic acid or base salts, administration of the candidate molecule as a salt may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example by reacting a sufficiently basic candidate molecule such as an amine with a suitable acid affording a physiologically acceptable anion. Alkali metal (e.g., sodium, potassium or lithium) or alkaline earth metal (e.g., calcium) salts of carboxylic acids also are made.

In some embodiments, a candidate molecule is administered systemically (e.g., orally) in combination with a pharmaceutically acceptable vehicle such as an inert diluent or an assimilable edible carrier. A candidate molecule may be enclosed in hard or soft shell gelatin capsules, compressed into tablets, or incorporated directly with the food of the patient's diet. For oral therapeutic administration, the active candidate molecule may be combined with one or more excipients and used in the form of ingestible tablets, buccal tablets, troches, capsules, elixirs, suspensions, syrups, wafers, and the like. Such compositions and preparations should contain at least 0.1% of active candidate molecule. The percentage of the compositions and preparations may be varied and may conveniently be between about 2 to about 60% of the weight of a given unit dosage form. The amount of active candidate molecule in such therapeutically useful compositions is such that an effective dosage level will be obtained.

Tablets, troches, pills, capsules, and the like also may contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active candidate molecule, sucrose or fructose as a sweetening agent, methyl and propylparabens as preservatives, a dye and flavoring such as cherry or orange flavor. Any material used in preparing any unit dosage form is pharmaceutically acceptable and substantially non-toxic in the amounts employed. In addition, the active candidate molecule may be incorporated into sustained-release preparations and devices.

The active candidate molecule also may be administered intravenously or intraperitoneally by infusion or injection. Solutions of the active candidate molecule or its salts may be prepared in a buffered solution, often phosphate buffered saline, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms. The candidate molecule is sometimes prepared as a polymatrix-containing formulation for such administration (e.g., a liposome or microsome). Liposomes are described for example in U.S. Pat. No. 5,703,055 (Feigner, et al.) and Gregoriadis, Liposome Technology vols. I to III (2nd ed. 1993).

Pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active candidate molecule in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present candidate molecules may be applied in liquid form. Candidate molecules often are administered as compositions or formulations, in combination with a dermatologically acceptable carrier, which may be a solid or a liquid. Examples of useful dermatological compositions used to deliver candidate molecules to the skin are known (see, e.g., Jacquet, et al. (U.S. Pat. No. 4,608,392), Geria (U.S. Pat. No. 4,992,478), Smith, et al. (U.S. Pat. No. 4,559,157) and Wortzman (U.S. Pat. No. 4,820,508).

Candidate molecules may be formulated with a solid carrier, which include finely divided solids such as talc, clay, microcrystalline cellulose, silica, alumina and the like. Useful liquid carriers include water, alcohols or glycols or water-alcohol/glycol blends, in which the present candidate molecules can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Generally, the concentration of the candidate molecule in a liquid composition often is from about 0.1 wt % to about 25 wt %, sometimes from about 0.5 wt % to about 10 wt %. The concentration in a semi-solid or solid composition such as a gel or a powder often is about 0.1 wt % to about 5 wt %, sometimes about 0.5 wt % to about 2.5 wt %. A candidate molecule composition may be prepared as a unit dosage form, which is prepared according to conventional techniques known in the pharmaceutical industry. In general terms, such techniques include bringing a candidate molecule into association with pharmaceutical carrier(s) and/or excipient(s) in liquid form or finely divided solid form, or both, and then shaping the product if required. The candidate molecule composition may be formulated into any dosage form, such as tablets, capsules, gel capsules, liquid syrups, soft gels, suppositories, and enemas. The compositions also may be formulated as suspensions in aqueous, non-aqueous, or mixed media. Aqueous suspensions may further contain substances which increase viscosity, including for example, sodium carboxymethylcellulose, sorbitol, and/or dextran. The suspension may also contain one or more stabilizers.

The amount of the candidate molecule, or an active salt or derivative thereof, required for use in treatment will vary not only with the particular salt selected but also with the route of administration, the nature of the condition being treated and the age and condition of the patient and will be ultimately at the discretion of the attendant physician or clinician. Candidate molecules generally are used in amounts effective to achieve the intended purpose of reducing the number of targeted cells; detectably eradicating targeted cells; treating, ameliorating, alleviating, lessening, and removing symptoms of a disease or condition; and preventing or lessening the probability of the disease or condition or reoccurrence of the disease or condition. A therapeutically effective amount sometimes is determined in part by analyzing samples from a subject, cells maintained in vitro and experimental animals. For example, a dose can be formulated and tested in assays and experimental animals to determine an IC50 value for killing cells. Such information can be used to more accurately determine useful doses.

A useful candidate molecule dosage often is determined by assessing its in vitro activity in a cell or tissue system and/or in vivo activity in an animal system. For example, methods for extrapolating an effective dosage in mice and other animals to humans are known to the art (see, e.g., U.S. Pat. No. 4,938,949). Such systems can be used for determining the LD50 (the dose lethal to 50% of the population) and the ED50 (the dose therapeutically effective in 50% of the population) of a candidate molecule. The dose ratio between a toxic and therapeutic effect is the therapeutic index and it can be expressed as the ratio ED50/LD50. The candidate molecule dosage often lies within a range of circulating concentrations for which the ED50 is associated with little or no toxicity. The dosage may vary within this range depending upon the dosage form employed and the route of administration utilized. For any candidate molecules used in the methods described herein, the therapeutically effective dose can be estimated initially from cell culture assays. A dose sometimes is formulated to achieve a circulating plasma concentration range covering the IC50 (i.e., the concentration of the test candidate molecule which achieves a half-maximal inhibition of symptoms) as determined in in vitro assays, as such information often is used to more accurately determine useful doses in humans. Levels in plasma may be measured, for example, by high performance liquid chromatography.

Another example of effective dose determination for a subject is the ability to directly assay levels of “free” and “bound” candidate molecule in the serum of the test subject. Such assays may utilize antibody mimics and/or “biosensors” generated by molecular imprinting techniques. The candidate molecule is used as a template, or “imprinting molecule”, to spatially organize polymerizable monomers prior to their polymerization with catalytic reagents. Subsequent removal of the imprinted molecule leaves a polymer matrix which contains a repeated “negative image” of the candidate molecule and is able to selectively rebind the molecule under biological assay conditions (see, e.g., Ansell, et al., Current Opinion in Biotechnology 7: 89-94 (1996) and in Shea, Trends in Polymer Science 2: 166-173 (1994)). Such “imprinted” affinity matrixes are amenable to ligand-binding assays, whereby the immobilized monoclonal antibody component is replaced by an appropriately imprinted matrix (see, e.g., Vlatakis, et al., Nature 361: 645-647 (1993)). Through the use of isotope-labeling, “free” concentration of candidate molecule can be readily monitored and used in calculations of IC50. Such “imprinted” affinity matrixes can also be designed to include fluorescent groups whose photon-emitting properties measurably change upon local and selective binding of candidate molecule. These changes can be readily assayed in real time using appropriate fiber optic devices, in turn allowing the dose in a test subject to be quickly optimized based on its individual IC50. An example of such a “biosensor” is discussed in Kriz, et al., Analytical Chemistry 67: 2142-2144 (1995).

Exemplary doses include milligram or microgram amounts of the candidate molecule per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid described herein, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific candidate molecule employed, the age, body weight, general health, gender, and diet of the subject, the time of administration, the route of administration, the rate of excretion, any drug combination, and the degree of expression or activity to be modulated.

In some embodiments, a candidate molecule is utilized to treat a cell proliferative condition. In such treatments, the terms “treating,” “treatment” and “therapeutic effect” can refer to reducing or stopping a cell proliferation rate (e.g., slowing or halting tumor growth), reducing the number of proliferating cancer cells (e.g., removing part or all of a tumor) and alleviating, completely or in part, a cell proliferation condition. Cell proliferative conditions include, but are not limited to, cancers of the colorectum, breast, lung, liver, pancreas, lymph node, colon, prostate, brain, head and neck, skin, liver, kidney, and heart. Examples of cancers include hematopoietic neoplastic disorders, which are diseases involving hyperplastic/neoplastic cells of hematopoietic origin (e.g., arising from myeloid, lymphoid or erythroid lineages, or precursor cells thereof). The diseases can arise from poorly differentiated acute leukemias, e.g., erythroblastic leukemia and acute megakaryoblastic leukemia. Additional myeloid disorders include, but are not limited to, acute promyeloid leukemia (APML), acute myelogenous leukemia (AML) and chronic myelogenous leukemia (CML) (reviewed in Vaickus, Crit. Rev. in Oncol./Hemotol. 11:267-297 (1991)); lymphoid malignancies include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease. Candidate molecules also can be utilized to target cancer related processes and conditions, such as increased angiogenesis, by inhibiting angiogenesis in a subject. Candidate molecules also me be utilized to inhibit metastasis of cancerous cells.

Kits

Kits comprise one or more containers, which contain one or more of the compositions and/or components described herein. A kit comprises one or more of the components in any number of separate containers, packets, tubes, vials, microtiter plates and the like, or the components may be combined in various combinations in such containers. A kit often comprises an ARG/ABL reagent and a Cortactin reagent. A kit in some embodiments includes one reagent described herein and provides instructions that direct the user to another ARG, ABL or Cortactin reagent not included in the kit.

A kit can include reagents described herein in any combination. A kit may comprise two, three, four, five or more reagents described herein. For example, a kit can include (1) an ARG- or ABL-specific binding reagent (e.g., an antibody) and a Cortactin-specific binding reagent (e.g., an antibody); (2) a nucleic acid that encodes an ARG or ABL protein or fragment linked to a detectable label (e.g., GFP or color variant thereof) and a Cortactin-specific binding reagent (e.g., an antibody); (3) a nucleic acid useful for encoding an ARG or ABL protein or fragment linked to a detectable label (e.g., GFP or color variant thereof), ARG or ABL ORF nucleic acid, and a Cortactin-specific binding reagent (e.g., an antibody); (4) ARG- or ABL-directed RNAi or siRNA and Cortactin-directed RNAi or siRNA; (5) ARG- or ABL-directed RNAi or siRNA and Cortactin-specific antibody; (6) ARG- or ABL-directed RNAi or siRNA and nucleic acid that encodes Cortactin protein or fragment in association with a detectable label (e.g., GFP or a color variant thereof), (6) ARG- or ABL-directed RNAi or siRNA and Cortactin ORF; (7) Cortactin ORF and ABL or ARG ORF; and other combinations of reagents described herein.

A kit sometimes is utilized in conjunction with a method described herein, and sometimes includes instructions for performing one or more methods described herein and/or a description of one or more compositions or reagents described herein. Instructions and/or descriptions may be in printed form and may be included in a kit insert. A kit also may include a written description of an internet location that provides such instructions or descriptions.

### EXAMPLES

The examples set forth below illustrate but do not limit the invention.

### Example 1

### Identification of ARG/Cortactin Interaction

Screening with a ProtoArray™ microarray identified multiple protein kinase substrates. Each ProtoArray™ microarray contains thousands of S. cerevisiae or H. sapiens proteins spotted in high density on glass slides. These slides can be probed to identify protein interactions with DNA, proteins, lipids, sugars, small molecules, and enzymes. A study demonstrating that these arrays can be used to reveal substrates of protein kinases was carried out on the Yeast ProtoArray™ microarray, which contains over 4000 unique yeast proteins spotted in duplicate. The experimental outline is simple. A solution comprising a kinase and radioactive ATP was incubated on a Yeast ProtoArray™ microarray, and then the slide was washed and exposed to a phosphoimager. The experiment identified 41 proteins specifically phosphorylated by the exogenous kinase.

The protein tyrosine kinase ARG was utilized to test this microarray platform for identifying specific protein substrates. This kinase, along with its closely related homolog ABL, is known to be involved in the etiology of chronic myeloid leukemia (CML) and is a target for the anti-cancer agent Gleevec®. Human ProtoArray™ microarrays were manufactured with 1500 different quality-controlled recombinant human proteins produced in high-throughput insect cell expression and parallel purification systems (Invitrogen, California). A known ABL/ARG substrate, CRK, was printed in regular intervals on the array as a positive control. The Human ProtoArray™ microarray was incubated with radiolabeled ATP alone; proteins that showed a signal on this array were kinases present on the array that autophosphorylated. Another array was incubated with ARG in the presence of radiolabeled ATP. This kinase phosphorylated the control substrate CRK in every subarray; in addition, nine other proteins that did not yield signal with ATP alone were observed to be phosphorylated in the presence of ARG. Adding an ARG/ABL kinase-specific inhibitor specifically decreased phosphorylation of CRK and the nine other microarray identified substrates, confirming that these proteins were phosphorylated by ARG kinase.

Verification of specific phosphorylation by a human kinase. ARG kinase is known to specifically phosphorylate tyrosine residues on certain proteins. To verify that ARG maintained this specificity for tyrosine residues in array-based experiments, Human ProtoArray™ microarrays were treated sequentially with ARG kinase followed by a phosphotyrosine phosphatase. All proteins phosphorylated by ARG kinase on the array are dephosphorylated by the phosphotyrosine phosphatase, confirming that ARG kinase substrates on the array are appropriately phosphorylated on tyrosine residues. Signals from proteins that autophosphorylate (i.e., that show signal in the absence of exogenous kinase) were not affected by phosphotyrosine phosphatase treatment, indicating that these were kinases that autophosphorylate serine/threonine residues.

Substrate phosphorylation is kinase-specific. The results with ARG kinase on Human ProtoArray™ microarrays clearly demonstrated this kinase is highly selective in the protein substrates that it phosphorylates. In order for this application of the ProtoArray™ technology to be useful to a wide range of kinase biologists, the ability to distinguish phosphorylation patterns of different kinases must be established. Consequently, ProtoArray™ microarrays printed with 2500 different human proteins were incubated with 33P-ATP and either ARG or PKC kinase or with ³³P-ATP alone. Phosphorylation signals specific to each kinase were clearly observed. The majority of signals present in both experiments were due to autophosphorylation by some of the ˜400 kinases printed on the array. Analysis of the whole array revealed dozens of proteins that were specific to one of the kinases.

ProtoArray™ data is used to generate a new kinase pathway. In addition to biochemical validation, it is also desirable to see concordance of ProtoArray™ results with published data. A search of the literature and publicly available databases revealed that one of the proteins proven to be a substrate for ARG on a Human ProtoArray™ microarray, Shp1, had indeed been annotated as a substrate for this kinase. Using a protein-protein interaction assay on a Human ProtoArray™ microarray, it was demonstrated that ARG kinase forms a stable interaction with Shp1. Shp1 is a phosphotyrosine phosphatase localized at the plasma membrane; our data, as well as the published data, are therefore consistent with co-localization and co-regulation of Shp1 phosphatase and ARG kinase. Other published reports indicate that following activation by SRC, ARG and ABL kinases translocate into the nucleus, although the functional consequences of this translocation have not been clarified. ProtoArray™ results, however, showed these kinases phosphorylated several transcription factors that may have roles in cell cycle function. An RNA polymerase was also phosphorylated, providing another line of evidence that these kinases regulate RNA transcription and gene expression. Equally intriguing is the finding that a membrane-associated receptor present on the array was phosphorylated by ARG kinase. Interaction of this receptor with a membrane-associated kinase has been shown by others to result in the activation of two kinases that have been implicated in oncogenesis. This finding represents a new and potentially therapeutically relevant link between the ARG/ABL kinases and cancer.

One of the ARG substrates identified in the microarray analysis described above, Cortactin, was assessed for in vitro and in vivo interaction with ARG and ABL as described in Example 2.

### Example 2

### In Vitro and In Vivo Confirmation of Cortactin Interaction with ARG and ABL

The interaction determined in Example 1 between Cortactin and ARG was confirmed in vitro and in vivo. An interaction between Cortactin and ABL also was determined.

Production of Recombinant Proteins

Wild type forms of ABL and ARG were constructed from mouse cDNAs using PCR. Proteins were produced and purified as described previously (e.g., Tanis et al., Two distinct phosphorylation pathways have additive effects on abl family kinase activation, Mol Cell Biol. 23(11):3884-96 (2003); Hernandez et al., Adhesion-Dependent Regulation of p190RhoGAP in the Developing Brain by the Abl-Related Gene Tyrosine Kinase, Curr Biol. 14(8): 691-6 (2004)). Cortactin protein was purified from baculovirally infected insect cells. Cells lysed in lysis buffer (containing 50 mM Hepes pH 7.25, 150 mM NaCl, 5 mM EDTA, 1% Triton, 5% glycerol, 0.5 mM DTT, 10 μg/ml Pepstatin A, 10 μg/ml Chymostatin, 10 μg/ml Leupeptin, 50 μg/ml Aprotonin, 1 mM Benzamidine, and 1 mM PMSF) rotated end-over-end for 30 minutes at 4° C. Lysates were cleared by centrifugation in Ti 70.1 rotor (40,000 RPM or 100,000×g) for 1 hour. Supernatant from centrifugation was added to glutathione-agarose (Sigma-Aldrich G4510), which had been previously washed with lysis buffer, and were rotated for 1-2 hours at 4° C. Glutathione-agarose was added to column (Pierce) and washed with 10 column volumes of lysis buffer. Cortactin was eluted with elution buffer (50 mM Hepes pH 7.25, 5% glycerol, 200 mM NaCl, 0.01% Triton, 20 mM glutathione, 10 μg/ml Pepstatin A, 10 μg/ml Chymostatin, 10 μg/ml Leupeptin, 50 μg/ml Aprotonin, 1 mM Benzamidine, and 1 mM PMSF). Protein concentration was obtained from Bradford assay and purity was assayed by gel electrophoresis.

Steady-State In Vitro Kinase Assays

Kinase assays contained 25 mM HEPES pH 7.25, 5% glycerol, 100 mM NaCl, 5 mM MgCl₂, 5 mM MnCl₂, 1 mM NaVO₄, 20 ng/μl BSA, 10 nM kinase, and substrate in concentrations as needed. After 5 minute preincubation at 30° C., the reaction was initiated by adding 5 μM cold ATP, and 0.75 μCi ATP. The reaction was quenched by the addition of ice-cold Laemmli SDS buffer after 5 minutes. The proteins were resolved by SDS PAGE. The gels were dried, exposed for autoradiography, and quantitated using Molecular Dynamics PhosphorImaging System and ImageQuant software. Michealis constant (K_{M}) and k_{cat} were calculated using KaleidaGraph software and scintillation counting, respectively.

Cell Culture

Wild Type, abl−/−, arg−/−, ablf/farg−/− cell lines have been described and were maintained as described previously (e.g., Miller et al., The Abl-related gene (Arg) requires its F-actin-microtubule cross-linking activity to regulate lamellipodial dynamics during fibroblast adhesion, J Cell Biol. 165(3): 407-19 (2004)).

PDGF-Stimulation and Immunoprecipitation

Wild-type (WT), abl−/−, arg−/−, or ablf/farg−/− cells were plated at 1E6 cells/10 cm dish and serum starved for 36-48 hours in DMEM containing 0.1% Fetal Bovine Serum (Gibco 26140-079), 100 units/ml Penicillin-Streptomycin (Gibco 15140-122), 2 mM L-Glutamine (Gibco 25030-081). Recombinant PDGF-BB (R&D systems 220-BB) was added to serum-starved cells at 5 ng/ml. At indicated time points, media was aspirated, and cells were washed with ice-cold PBS. PBS was aspirated, and cells were lysed in modified RIPA buffer (50 mM Tris pH 7.2, 150 mM NaCl, 1% NP-40, 0.5% Deoxycholate, 0.1% SDS, and 1 mM EDTA, 2 mM NaF, 1 mM Na3VO4, 10 μg/ml Pepstatin A, 10 μg/ml Chymostatin, 10 μg/ml Leupeptin, 50 μg/ml Aprotonin, 1 mM Benzamidine, and 1 mM PMSF). Cell lysates were rotated end-over-end at 4° C. for 30 minutes and then spun at 14,000 RPM. Supernatant was pre-cleared with Protein A/G-agarose (Oncogene IP10) for 45 minutes at 4° C. and then spun at 8,000 RPM to pellet beads. Pre-cleared supernatant was added to fresh tubes containing 2 μg anti-cortactin antibody, clone 4F11 (Upstate 05-180). Tubes rotated end-over-end for 1-2 hours at 4° C. Protein A/G-agarose was added for 1 additional hour, at which time tubes were spun at 8,000 RPM. Immunoprecipitants were washed three times in modified RIPA buffer and then subjected to SDS-PAGE and immunoblot analysis. Phosphotyrosine was detected by cocktail of anti-phosphotyrosine antibodies (anti-PY antibodies) (Upstate 4G10, Santa Cruz PY20, Santa Cruz PY99) and donkey-anti-mouse (DAM) HRP-conjugate (Jackson). Cortactin was detected by Santa Cruz (H-191) and donkey-anti-rabbit-HRP conjugate and chemilluminesce.

Cortactin Isoform 2 is an In Vitro Substrate of ABL and ARG Kinases

Steady-state kinase assays with Cortactin isoform 2 and ABL or ARG demonstrate that ABL and ARG can phosphorylate Cortactin in vitro with a K_{M} of 110 nM and 60 nM for ABL and ARG, respectively. These K_{M} constants are lower than for other known substrates of ABL/ARG (Tanis et al. and Hernandez et al., supra) and suggest a favorable interaction between each of the kinases and Cortactin. The k_{cat} for ABL and ARG is 0.05 min−1.

ABL and ARG also are contacted with Cortactin variants in which one or more tyrosine amino acids are substituted with non-phosphorylateable amino acids such as phenylalanine or alanine. The presence, absence and/or amount of phosphorylation for each Cortactin variant substrate is determined, and phosphorylation sites on Cortactin are assessed. Cortactin variants include, for example, a tyrosine to phenylalanine substitution at each of the following positions in SEQ ID NO: 25: 384, 409, 416, 427, 433 and 449 (e.g., one substitution in each Cortactin variant).

Cortactin is Phosphorylated in PDGF-Activated Cells in ABL/ARG Dependent Manner

Growth factor stimulation (PDGF) activates ABL kinase activity and increases phosphorylation on Cortactin (e.g., Plattner et al., c-Abl is activated by growth factors and Src family kinases and has a role in the cellular response to PDGF, Genes Dev. 13(18): 2400-11 (1999); Weed & Parsons, Cortactin: coupling membrane dynamics to cortical actin assembly, Oncogene. 20(44): 6418-34 (2001)). It was determined using the methods described in this Example that PDGF stimulates phosphorylation of Cortactin 3-fold in wild type cells but not in abl−/−, arg−/−, and abl−/−arg−/−. This evidence suggests ABL family kinases and Cortactin interact in vivo in an established system.

The entirety of each patent, patent application, publication and document referenced herein hereby is incorporated by reference, including all tables, drawings, and figures. All patents and publications are herein incorporated by reference to the same extent as if each was specifically and individually indicated to be incorporated by reference. Citation of the above patents, patent applications, publications and documents is not an admission that any of the foregoing is pertinent prior art, nor does it constitute any admission as to the contents or date of these publications or documents. All patents and publications mentioned herein are indicative of the skill levels of those of ordinary skill in the art to which the invention pertains.

Modifications may be made to the foregoing without departing from the scope, spirit and basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. One skilled in the art readily appreciates that the present invention is well adapted to carry out the objects and obtain the ends and advantages mentioned, as well as those inherent therein. The examples provided herein are representative of specific embodiments, are exemplary, and are not intended as limitations on the scope of the invention.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

What is claimed is:

1. A method for identifying a molecule that modulates an interaction between an ARG/ABL protein or fragment and a Cortactin protein or fragment, comprising:
contacting the ARG/ABL protein or fragment and the Cortactin protein or fragment with a test molecule, wherein the ARG/ABL protein or fragment and the Cortactin protein or fragment interact in the absence of the test molecule; and
detecting the presence, absence or amount of an interaction between the ARG/ABL protein or fragment and the Cortactin protein or fragment,
whereby a difference in the interaction in the presence of the test molecule versus the absence of test molecule identifies the test molecule as a molecule that modulates the interaction.

2. The method of claim 1, wherein the interaction is phosphorylation of the Cortactin protein or fragment.

3. The method of claim 1, wherein the interaction is binding of the ARG/ABL protein or fragment to the Cortactin protein or fragment.

4. A method for identifying a molecule that modulates a biological activity in a system containing an ARG/ABL reagent and a Cortactin reagent, comprising:
contacting the ARG/ABL reagent, the Cortactin reagent and a test molecule in the system; and
detecting the presence, absence or amount of the biological activity,
whereby a difference in the biological activity in the presence of the test molecule versus the absence of test molecule identifies the test molecule as a molecule that modulates the biological activity.

5. The method of claim 4, wherein the biological activity is cellular localization of a Cortactin protein or fragment.

6. The method of claim 4, wherein the biological activity is phosphorylation of Cortactin.

7. The method of claim 4, wherein the biological activity is synthesis of a transcription factor.

8. The method of claim 4, wherein the biological activity is interaction of an ARG/ABL protein or fragment with a binding partner.

9. The method of claim 4, wherein the biological activity is interaction of a Cortactin protein or fragment with a binding partner.

10. The method of claim 4, wherein the ARG/ABL reagent and/or Cortactin reagent is an isolated protein or fragment.

11. The method of claim 10, wherein the protein or fragment is in association with a detectable label.

12. The method of claim 10, wherein the detectable label is covalently linked to the protein or fragment.

13. The method of claim 10, wherein the detectable label is a fluorescent protein.

14. The method of claim 10, wherein the detectable label is a fluorescent molecule.

15. The method of claim 14, wherein the fluorescent molecule interacts with another fluorescent molecule in the system in a fluorescence-resonance energy transfer (FRET) interaction.

16. The method of claim 4, wherein the ARG/ABL reagent and/or Cortactin reagent is a molecule that specifically binds to an ARG/ABL protein or fragment or a Cortactin protein or fragment, respectively.

17. The method of claim 16, wherein the molecule is an antibody.

18. The method of claim 17, wherein the antibody is in association with a detectable label.

19. The method of claim 4, wherein the ARG/ABL reagent and/or Cortactin reagent is an isolated binding partner of ARG/ABL or Cortactin, respectively, or portion thereof.

20. The method of claim 19, wherein the binding partner or portion thereof is in association with a detectable label.

21. The method of claim 4, wherein the ARG/ABL reagent and/or Cortactin reagent is an isolated nucleic acid comprising a nucleotide sequence that encodes an ARG/ABL protein or fragment or a Cortactin protein or fragment, respectively.

22. The method of claim 4, wherein the ARG/ABL reagent and/or Cortactin reagent is an inhibitor of a biological function of an ARG/ABL protein or fragment or a Cortactin protein or fragment, respectively.

23. The method of claim 4, wherein the ARG/ABL reagent and/or Cortactin reagent is an inhibitor of a nucleic acid that encodes an ARG/ABL protein or fragment or a Cortactin protein or fragment, respectively.

24. The method of claim 23, wherein the inhibitor is a siRNA, RNAi, antisense or ribozyme nucleic acid.

25. The method of claim 4, wherein the ARG/ABL reagent and/or Cortactin reagent is a cell that over-expresses an ARG/ABL protein or fragment or a Cortactin protein or fragment, respectively.

26. The method of claim 4, wherein the ARG/ABL reagent and/or Cortactin reagent is a cell that under-expresses an ARG/ABL protein or fragment or a Cortactin protein or fragment, respectively.

27. A kit comprising a Cortactin reagent and an ARG/ABL reagent.

28. The kit of claim 27, wherein Cortactin reagent and/or ARG/ABL reagent is a Cortactin protein or fragment or an ARG/ABL protein or fragment, respectively.

29. A composition comprising a Cortactin reagent and an ARG/ABL reagent.

30. The composition of claim 29, wherein Cortactin reagent and/or ARG/ABL reagent is a Cortactin protein or fragment or an ARG/ABL protein or fragment, respectively.


SEQUENCE LISTING (29 sequences)

SEQ ID NO: 1 - DNA, 157866 bp, Homo sapiens
ataaaccaatccaaccaagtttttacctggacagcatttcttcatgttgaccttgaagatgtaacaagagtctttgttaaggtccctgctacagtccagaatctccttgagtataaacctcactgatcctccagtctggtgatgactgtattattaaaggaagtcagaccatttcctctgctaaaggcttacaaattacatatttagtaatcttttcagaagattttgccaaggtttggttgtcaattttggaatctacctctttttccatttttaaatttaaactttaaaaaatggttgaattgttttttgaaagttgcttcaatgtctgccggtctcctggcacctgctgctttctccaggatacctcaaagaggaccaagcagagtttgggtatctttgcagcctctctccatgctctgggatgtatctgaaccaattcaaaggtgcaggacacgctcttcctgtgtaacaggatgatttctcaaagtattaatgtgagtgtgttctcactgaaatgtcctggtcctaagattggtgaaagcaactgggtccctatgtgttccttcccatggggaagttttgcacaaagaggccaaaagcagaagtacctacttgattatctattcctggtgttggttgaaagagtatgataaatatttttagtggcaattggaagagtaagacagaactccctgaggcaaaaaaaaaaaaaaaaaaaaaagagagagagagagaaaacatcaatcagagtgggagggagtgctaaggaagtggtgagagctgggttgatggctgcatggtgccctggggatgggcataggcagcagagtgccccatcccagcaataattttcacaccccacacatggcattgagacagaaccttcaagggaccactctgggtttggacaggacaaagtggctctgtcatctactaagatccacagccataatggcctggaactgtgtgaccttgggcaagttcctgacctctctgggccttggttcctcatctgtgaaatgaggatggtagcattccttgtctcaggattattgttaggattaaatcagttgatacatataaatacttagaaccatgcctagcatgtagtgttatgttgttgatgatgatgattgttttaaggtgcagcccccgaattctggagaaaggagagagccttggtaataatttaaattgggtttcctgccaagctggtaaggaagttcaaggaagactccaagctggcacttggagtcagtttaagtcaacttgaagaaaatagagaaatgtggctgggagcagtggctcacgtctgtaatcacagcactttgggaggctgaggcaggtggatcacttgaggccaggagttcaagaccagcctggccaacatgatgaaacctcgtctctactaaaaatacaaaaattggctgggtgtggtggtgcatgcctgtagtccctgctactcaggaggctgaggcacaagaattgcttgaacccgagaagcagaggttgcagtaggccaagattgtaccactgcacactagcctgggtgaaatgcctcaaaaaaaaaaaaaaagaaaagaaaagaaatagagaaatgtgacctttcttgcacacttaaatttatgaaccaatattctcaacttctccactatctcactcctgtatcaggttacaatgtcctgttaaaattatgttttatatttcagggaacagagagcacctgagtgggctctgtcatctactaagatttcccaccttcccaaagcagccggtctaccctttcttattcatcatattaatcttcttccaaacaaaactgtgatagaaaggcttcctgaagatgtctgtgtcctaatctccagaatccatgaatgaatgtgttgtgttacatagcaaagaggatgtagatagaattaaggaatgcagagagaattaaagttgctaatcagctgcccttaaaatagggatggtggcttgggttattttggtggatcctatcacaagggtccttataagtgggagagggggccaggcatggtggctcacacctgtaatcccagcactttgggaggccaaggcaggcagatcacttgaggtaaggagttcgagaccagcctggccaacatggtgaaaccccatctctactaaaaatacaaaaactagccaggcgtggtggtgtgtacctgtaatcccagctacttgggagactgaggcacgagaatcttttgagcctgggaagcgaaggctgcagtgagccaagatcatgctactgtcctccaccctgggcaacagagcaagacactatctcaaaaagaaagaaagaaaatcaggagagggaaacagaagagggaaagcccaagagatggcagagtggaaagacatggcccaacatttctggctgtgagaaagcgaggaaggggccataagccaaggtacatgggcaacctctagaaagctgggaaattcaagggaacagattatcccttagagcctccagaaggaagggatgcagccctggtgacagcttgattttcagtccagtgagacccatgtcagccttctgacctcagagctgtaagattatacatctgtgatgttttaagcctctaagtctgtggtaatgtgttttcacagcaataggaatgaatagacaaaaccaccaatatagaaacaataatcaaacaaaaacacttgtttttgcaaagctcagctttagcttttctgaccctgttttcgaaagttcttatctttctcctgagctcgtcttttcttctctgcccctggtcccagaactgatgcaggttcttttgcactaagagctctctaagaagtgctgtgtttatttaaacatgtccctctctttcatggaagtcattgctggtatccgctgggcactcccttttctcctcacatttcttgtctttgtctctggagtcacgccttcctttcctctggactttctgaagtcctgtcctaaggcttggtgcgtgggactgactagttaaaatctagtgccaagtgctcagaacatcaagacacaacaaagtcactcaagatccgttgggcagaatctcctgaaattgctttttttttaagatctaaaagtcaagtatcagtaattctataaaagttcaacataagaaaagagaagacaggcaaataactacaggtgtgaaaaatatactacagaaacagaaacaagaacaggtgtcatggtggagggggacaaatacataagaaaatagtaatgagaaatgctatggagaaaatagtggctcacacctgtagtcccagcacttagggagaccaaggcaggagggtcacttgagcccatgagtttgagaccagcctgggcaacatggcaaaaccctgtctccacaaaaacatacaaaactagccaggcatggtggtgcttacttgtagtcctagctacttgggaggctgaggtaggtggattgtttgagcccaggaggtcaaggctgcagtgaaccatggttgtaccactgcactccagcgtgggtgaaagagcaagactctgtctcttaaaacaacaacaaaaaaccctcagccctgcaatggggcacacctgggagcccctttggattgggtggtcagggaagggccctgggaggcaatgacctgctgtgttacgctcaggaggttggccaggaccccgtggaagactcagggagccagcaggggacgctaggcaggggaggacagggcccagtccctctgtggctcactcactctggagtgggtgtggggatgtgctggcagggccagtccaggagcctagttaaaggggggtcctgtggtagcccatgcatgggatgatggtggcctacagtgaaaccatgatggtgggctggggaagagtggaagtacttaggatggggaagaagcaggacttttggtgattgagtgtgttggggcaggagcaggggaggaagagggaggtgaaaggggtgactctggttcactattaggggactggatggttgtgctactctgcaacacaaggaactctgggaaagggggagattgggtggccagagacgtgcggagatgggcatggaggagctcacaagcagcagttctggaaatgaatgctgagcatggcagctggacgacaaggtggcccaatggcccgtgcctacagtctgcacacccttgggtagtctcctgcccttgaatctgggctatgctctctggttttcatggacgctaagagggcacagtcacctcctcacaagatctgccatcagcactttctgattttttgtgcttggtcaagattaagtctagagtagctgcccccagaaggagccatttgaaaagattattgaggattttccaacactctgcttccagaagaattagataccccacctcctctggtttaatccagccttttggggttggctccacaggtgatttacataggaaatacgtgggactcctccaaggtagaagtttttatggttgcgcctggcagccttcactgaccctcatccacatagacaagtggccacaggtacactccactcagtcaccccacactccagaggccttgggtagactttcatgtgttcacagacagatgtagcttttcacggtcttcacattcacacgctcaagtacaaggtcagtccctgccgatgcaagttacaaggaagcacaacctgtgctagggcagccagtgcatcccttccagctggccaggagccctcagcaggcctgaaagctgggaggagaggtgcccagctaggtggaaacagaagggaaggtgcaggactgtgggtgatcctggcagctggcttgctcctggaaggccaggtgggacatctgcaaggtaagggcctggaccctgggcagaaattgtaagtctctggtcttcaaggcctttaggagatgagaatgttgaagcctccgtcttaccaactaaacctctcggtaccaagagggcctggcctgcaaggtagaaaccaaaaccccctctaacacacacacatgcacacacacacctctaaacattcaagtgtacagatgctagcactgtactagcattgtttctgcaagcctgagagaagctgagctctgtttccattctcctctcacatctgcctggtgggaatgctctaagagggggttcccagactttgttgcacattaagatcaccagggaaggaagctttcacaaatcttgatgttcaggtcactgctcatgccaatgaaatcgaatatttgggtgtaagacccagacatcagtattaactggtgactcctatgtgcagccaacttcaagaaccaaagatctttgttggttttttttttttttttgagacagagcctcactctatcactcagactggagtgcagtggcacaatcttggctcactgcaacatccgcctcccaggttcaagtgattctcctgcctcagcctcccaagtagctgggactacaggtgtgtgcaatcatgcccagctgatttttttgtatttttagtagagacagggttttatcatgttggccagctggtctcaaactcctgacctcaagtgatctgccctcctcagcctcccaaagtgctgggattacaggcgtgagccaccatgcccagcccaaagacttttattcatcatcacacacaggcctgtggtggagatttttaaaaattgaggtgaaattcatataaacttactcattttaaagtatacaatttagtggcatttcgtacattcacactgttgtgtaaccatcacttccatctagttccaaaacattttcatcaccctaaggagaaaccctgtacacattaagcaagcattcccctctccccctctcccctcagtccctgccaaccacaaatctgcttctgtttctatggatttacctattctgtgtatttcatataagtagaactagacaatatttgacattttgtttctaacttctttgcttggcataagttcatccatgttgtagcatgaatcagtacttccttcttatggctgaataatattaccctgtatgactaggacacactgtatttatccatttaaccgctgatagacatttggattgttttcacctttgggctactgcgaataatgctgctttgaatattcatgtacaagtctttgtttgaatctctatttttgattctttggggtattctgggagcagaattgctgggttatatggtaatttgatgctacttttttttttttctagacagggtctgtctgtagcccaggctggagtgcagtggcaagatcatggcccactgcagccttggcctcctgggctcaggtgatcctcctccctcagtctcccaagtagctgggaccacaggcatgtgccaccacacctggctaattttttaaaaaaaattttgtagagatggtttttccctatgttgcccagtctggtctcaaacccctgacctcaagtgatccgcctgcctcagcctcccagagtgctgggattacaggcgtgagccacccacttctggctgatgcttatcttttaatgaactgccaaactgtttcccacacatgctgcaccattgcacattcccaccagcaatgtacaaatgttccaatttgtccacatccttgttaacactgtctgtttttcatttttttattattatagccatccttgtaagtgtaaagtggtatcttattgtggttttaattttcatctccctaatgaggaataatatcgagcattttttcttgtacttgttaactatttgtatatcttctttggagaagagtctattcaagacccagatactttatgcctcctaatcctcatagcaccactgagaggaaaggattgttcacatttttctagatgaagaaactgaggctgagagaatgccccaactccaattgatgacagaaagcaagatctgccgatgcttgaaattcataccatacacacgccccaaatattccagtgtgcagatgcttgtgccaaatgagaaatacagtcaaaaagctacagggggctgggtgcagtggctcatgcctataattccagcactttgggaggccgagatgggtggatcacatgaggtcaggagttcgagaccagcctggccaacatggtgaaaccctgtttctactaaaagtacaaaaattagccaggcatggtgtcgggcacctgtaatcccagctacgtgggagactgaggcaggagaatcacttgaacccaggaggcagaggttgcagtgagccaagatcattccattgcattccagcctaggtgacaagagcaaaaattctgtctcaaaaaaaaaaaaaaaaaaagctacagagtgtggaggaggaggggtaagcacaaaaaaactggagcccctgaggagctcttcaggtagagagggagtggggttccatatgggtttggagtgacagagtgagagagagagatacagtagagagaccaagtgtgagtgccaggaaccagcaaggcatattgaaggagatgcatttttttgtcaggcctgtttctatagcagagaaagacacagttggcatgttcagcggggggcccagggtggaggtcactcaggttttatgctctggactcctttgtgccctgagaatttcccaggcttggccacaatcagctccagctgctgattctgggatggactcagccacagcctcacccaggtcacacaaatggaagtggtaagtcgatgctccgcacaggcagctgctacagctgcaggaagcctggaggtgtgagtcgatatttctgttagactctcagtttcatcagaaatcagtgccagaaaatggctttgcagctgtgtgtgtgtgtgtgtgtgtgtgtgtgtgtgtgtgtgtgtgtgtctatgggctccaacaccctttagaagcctatctctgtactttgaactctaagaggtgcaaagtgagattgaggggtaaggagggatgagggggaaggagttctgatgatctgaggctggaggagaaataaggaccagagccgccctcaggccttggagagcagccttcacctctggaaatgatgccgaaatatttcagcctctctctcctggggtttcctgtttcctatttggtctctggtttccaggcaacacatgaatacccccaaactcctgaagtgtatgggtctccaaatgtccttcttcctctaaagctcacatctgtaatgagtttcccatctgctcagtcgcccagggcagcccatccattctgctgtctccagagacaaaagcaatcgaagatgcttgtggtttgaggtgtgtgtgtgtttctaccctccccccaaccctgccactttgtaggtttctccagtttaatgagtcttcctgcaaagtcggtttggctgaattgcattattagggtggggagcaggcaggagatttagggtttgctgatttgatgcagtaaagaactgagtctggcaggagcccagcaatttagaagaaaagtgggggaataagccctaaacttgacccactggtgagctattggctggctttctctctctctcaggagagagtggaggggctgtcttcaggaactctggtgggcttcaggggacttgaaacttccttagattcatgataaactggattcatgattcactcccaccagattcagagaacctggtggcagtgaagtataaaattgaagggggcaaaattatggggaattgcactaatttttgtgtttagtataattaatacatagcaattgcattaatatttgcataataatagtacttcaaggcttagaacaacagtccattgtcactcttatccagctctgagctccagtctcaagggcaacctatttttagttttaactattggtaaaaatttaccttggagaggggtgtattagtctgttttcacgctgctgataaggacatacctgagactgggcaatttacaaaaaaaaaaaagaggtttaatgcactcacaattccacgtggctagggaggcctcacaatcatggcagaaggtaaaagacacttcttacatggcggtggcaagagagagaatgagagccaagtgaaagcagtttcccctcataaaaccatcagatctcatgagacttattcactaccctgagaacaatataggagaaatggcccccaggattcagttatcttccaccggctccctcccacaacttgtgggaattatgggagtataattcaaaacgagatttgggtggggacacagagccgaaccatatcaaggggcaaagcaaggtgactgaatagaatctcccagtaattgtccctctgcaggaacatcaaattgaacaactatccacacaaaaaagcaccttctgaagagctacagatgcactggagagggtaggaaggacaatctcacattgcctaaaccacccctaccccaaccccaggcaggacggtaggagacagaaactgcctgcttggggaacagaaagggaagtgagtgtgaaactttgaattagaacttagtgccagccccaccacaatggaacaaagctccatgcagagctcctgcagcctgaaattccaggctggggcccacagagggagaatttatacctagcccagggcagaggggaatctaccgcccaggcaggaggagctagagtccctgtccacttgatcaccaggaaactaaagtggcatcaggacccaaataaacttcagtggctggcaagcaggctttagaaaccatggtccttggatgagacccagtaatgtgctggtctgagaggccattggctgaaaagtgacccagtattcagaggtaaagccagctggacttcctgggtcgagtggggacttggagaacttttctgtccagctaaaggattgtaaacacaccaatcagcgctctgtgtctagctaaaggattgtaaatgcaccaatcagcactctgtgtctagctaaaggattgtaaatgcaccaatcagcactctgtctagctaaaggattgtaaacacaccaatcagcactctgtagaaatgcaccaatcagcactctgcgtctagctaaaggattgtaaatgtaccaatcagcactctgtaaaaacacaccaatcagcactctgtataatggaccaatcagcaggacgtgagcggggccaaataagggaataaaagctggccaccagagccagcagtggcaacacactcaggtccccttccacactgtggaagctttgttctttcactcttcacaataaatcctgcagcatcactctttgggtctgcactacctttatgagctgtaacactcaccacgagggtctgcagcttcattcctgaagtcagcgagaccacaaacccaccaggaggaaaaaacaactctggacgcaccacctttaagagctgtaacactcaccgcaagggtctgcggcttcactcctgaagtcagtgagaccacgaacccaccgcaaggaataaactccagacacatctgaacatctgaaggaaaaaactccagacacaccatctttaagaactgtaacactcactgcgagggtccacggcttcattcttgaagtcagcgagaccaagaacccacaagaagggataaattccagacacagtatgacatcagtggtgtctgccacaaaagtacccatgtcacccctccctcagctccaggcagtgcagttcagagagactccctctacttgggaagaaagaagagtacaggagactttgcctgtgaatctagtgtcagttttgccacagtaaaacaaaacacagggctgaacccccacctggacatacttctagacttgtcctgggacagaagggaatccatcaccatggcaggaaaaacccaagtcccagcctgcttcaccaccagctgaccaaagtgaccttggcctttgaataaatatcagcagtattaaggcagtagcagctgcaggcattgagctagccctggtactgcaccggtcagggagactataggctttggtatgacctagcatagtgccagctgcagaggccatggatatgtccatgtcagccctcccccaccagggaactatctctgattttccagaactacatcagggttggcgatcttggagtctgcaagactcatagtgaaacttggcttagggccctctctactgcaaaatggctgcactgaccacaggtttagtgaactcaacaggtggtcccttttgaattattcgaaggctccctgaagaacagatacaaacaaggccagacagtgaagactggaataagtatctacttcatcaatgcccagatattgatgaatgtatgcaagcatcagaaacattcagaaaatatggtctcactaaatagactaaataaggcaccagtaatgaccctggagagatggagatgtgtgacctctcagccaaggaattcaaaatagctgttttgaggaagttcagtcaacttaaacacacacacacacacacacacacacacacacacacacacacacacacacacaaaattcagaaatttatcagagaaatttaacaaaaagactgaaaatttgaaaataaatagaaatcctggacctgaaaaatataatacgatagatcaagtaaaaaatgcatttgacatcttcaacagaagaattgatcaaacagaagaaagaatcaggctatttgaaaatgcacagaggagaaaaaggaaagacaaacacgagaattaaaaaatagcttcaaaagagcaaatgtaagagtcgttggccttaaagagggagtagagacagaaaaaggggtagaaagtttattcaaagaaataatatcatggagtggggagaagaaaaaaaataatatcagaaaactttccaaacctaaagaaagttatgaatatccagttacgagatggtcaaagagcaccaagcagattgaacccaaataagactcaagccatataaaaaatcagactctcaaagttaaacaagaaaaagaggatcctaaaatggcaagaaaaaagaaacaaacaacatataaagaagctccaggccaggtacagtggctcacacctgtaatcccaacactttgggaggctgaggtcggcagatcacctgaggtcaggagttcaagactagcctggccatcatggcaaaactgcatccctactaaacaatacaaaaattagctaggcatggtggctggtgcctgtaatcctagctactcaggaggctgaggaagggagaattgcttgaacctgggagttggaggttgcagtgagcagagatcacaccattgcactccagcctgggcaacagagtgagactctgtcaaaaaaaaaaaaaaaagctccaatacatctgtctgacagtggacttcccagtggaaatcttagaggccaggaaagagtgggatgccatattcaaagtgatgaagaaaacaaaaattacaaactgagaatactgtatccagtaaagctatctttcaaacacaaaggagagataaagactttgtcaggcaaacaaaagctgaaggaatttatcaccaccagacctgtcttacaagatatgtgaaaaaaagagttcttccatctgaaataaaaggatgctaatgtgcaaaaagaaagcatttgaagatataaaactcactggtaaaactatgtacatagccaggctcagtgcctcaagcctgtaatcccagcactttgggaggctgaggtgggtggatcacttgaggtcaggagtttaaggccagcctggccaacatagtgaaaccccatctctactagaattacaaaaattagccaggtatggtggcgggcacctgtaatcccagcttctagggaggctgaggcgcaagaatcactcgaacccaggaggcagaggttgcagtgagccgagatcgtgccactgcactccagcctgggtgacagagcaagactctttctaaaaaaaaataagtgtgtacatagacaaattcagaataatatagtaattgtagtacgtaagctacttgtgtctttagtatgaacactaaaagacacacctatcaaaattaataactacaatttgttaagagagaaacaatataaaaagatttaaattgacaacaaataatcaaaatgcagagggggatggaggtaagtatctttttcctctgtctctttttttgcaatcaaaattaagatgtcatcttaataatttattatagctaaagataagcctttaaaataatttactacagctaaagctaagtctcatggtaactacaaagcaaaaacccgtaatagatacactgaaaaaacaaggaattaaaatatactaccaaagaaaatcgcttaaaccacaaagaaaaacaataagaaaggaagagaggagttataaagaaccaggaaacaggtaacaaaatggtagtagcaaatccttacctgtcaataacaacattgaaggcaaatggacttaattatccaattagaagacacagagtgactgcataggttttaaaaaaaaaaaaaaaagtcccaactatatgttgcctacaggaaactcccttcacctgaaaagccacacataggttgaaagcaaagggatggaaaaaggtattctacataaatggaaaccaaaaacaagcaggaatagccatacttttatcagataaaacaagcttcaagtcaaaagctctaaaaggaaacaaagagagagaagatcattatacaatgataaaaggatcaattcagcaagaggaagtaacaactgtaaatacatatgtacccaacactgaagcacccagatatataaaataatattagagctcaagtgagagatagaccccaatataatgataattggtaacattaacactccactttcagcactggataacatctagacagaaaataaataaagaagcatcaggcttaatctgcactatagaccaagtggacctaacagaaagttacagaaaatttcatcgaatagctacagaatacacattcttttcatcagcacctggaacattctccagtatataccatatgttaaaccacaaaacaagcctcaacaaatttttcaaggtcaaaatcatatcaagtatcttcttaagccacagtagaataaaactagaaatcactaacaagaacaactttggaaaccatgcaaatacatgaaaattaaacaacatgctcctaaacaactactgagtcaaagaagaaattaaaaaggaaatagaaacatttcttgaaacaaatgaaaaatggaaatacaacatactaaaacttatgggatatagcaaaaacagtactcagagggaaatttatagcaataaacacctacatccaaaaaagtagaaaactttcaaataacgaacctaatgatgcacctcgaggaactagaaaagcaagaataaaccaaccccaaagttagtagaagaaaggaaataataaaaatcagaacagaactaaatgaaatagacactaaaaaattacaaagaatcaaaaaaccaaaaagttggttatttgaaaatataaacaaaatcaataaattactagctaaactaaccaagaaaaaaagaagtcccaaataaataaaattagaaatgataaaggagacattacaactgatatcacagaaatacaaacgatcggccaggtgctgtggctcatgcctgtaatcccagcactttgggaggccgaggcaggtggctcacttgaggtcaggagtttgtgaccagcccagccaacatggtgaatccctgtctctactaaaaatacaacaaattggctgagtgtggtggcaggcgcctgtaatcccagctacttgggaggctgaggcatgagaatcacttgaacccaggagggagaagttgcagtgagtcaagatcctaccactgcactctagtctaggtgacagagcaaaactctgtctcaaaaagaaaaagaaaaaagtacaaaagatctttagagactattatgaaaaattatatgctaacaaattagaaaacctagaggacatggataaattcctagacacatataacctaccaaaattgaaccaggaagaaatagacctgaaaagaccaataacaagtaatgagattgaatcagtaataaaaagtttcccagcaaaaaaacccaggaccagatagctttattgcaaaattctaccaatttttattaaatttttttagaaacatgatctcactctgtcactcagtctggagtacagtcgtgtgatcatagctcactgcagcctcaaactcaggctcaagtgattctcccacatcagcctcctgagtcactaggactataggtgcataccataatgcccagctaattatttttttgtagacacagggtcttgttatgttgcccaggctggtctccagctcctggcctcaagcaaccctcccacctcaacctcccaaaatgctgggactacagatctgagccactgcactcagcctacaaatcatttgaaaaagatttaacaccaattcttctcaagttattccaaaaagtgaagaggagggaattcttcctaactcatgctatgagaccagcattgccctgataccaaaaccagacaagactacaacaacaaaagaaaacttcaggccaccattctttatgaacataaattcaaactttttaacaaattactgccaaatcaagttcaacagcacatcaaaaagataatacacatgatgaagtgggatttatcccaaggatgcaaggatgtttcaacatacacaaagcaataaatgtgatgtatcacattaacagaatgaaggataaaaaccatataatcatctcaatagacacagaaatagcatgtaataaaatttaacattccttcatgataaaattctcaacagattaggcacagaaggaacacacttcaacataataaaggttatatatgacaaacccacaactaacatcatactaaatagggaaaggttgaaaccccttcctctaaaaactggaacaagacaaggatgtccattttcaccatccctatttaacatagtattgggagtcttagccagagcaactaggaagaaaaagaaaggacatccaaatttggaaagagaaaatcaaattgtcccttattgcagatgacataaccttatatatagaaaagcctagactccaccaaaaactgttacgactaataaacaaattcagtaaagttgcaggatacaaaataaacatataaaaatcagtagtgtttctatacagcaataataaatgaactgaaaaagcaagaaatgaaccccatttacaatagctaaaaaaaaaaaaggaataaacttaaccaaggatgcaaaagaccttcacaatgaaaactagagaacactgataaaagaaactgaagaagacacaaacaaatgaaaatacatcccatgctcatggattggaaaaattaatattatcaaaatgactaaagcaatttacaggttcaatgtaatctctatcaacataccagacattcttcatgggaatagaaaaacaatcctaaaatacatatggaaccacaaaagacctcaaatagctaaagcaatatatatagaaaaaaaaatagcaggaggcatcatactacctgacttcaaaatacacgacaaagctatagaggtatagtaaccaaaacagcatagtattggtataaaaacagacacatagaacaatggaacagaatagagaatccagaaatccacatatttaaagccaactaatttttgacaaaggcaccaagaacatacattgggaaaaggacaccttcttcaataaatggtgctgggaaatattgaagaaggacaccttcttcaaaaaacggtgctgggaactatatgcagaagaatgaaactagacccttattctcaccatatacaaaaatcaacccaaaatggattaaagactgaaaagtaagacccaaaactataaaaacgctagaagaaaacataaggaaaatgtgcttcagcccattggtctaagcaaagattttatgtctaaggcttcaaaagcacagctaaagaaaatagataaatgagactatattaaactaaaaaccttctccccagcaaaggaaacaatcaagagagtgaagagacaacctacagaatgggagaaaatatgtgcaagctactcatttcgcaagggataacatctagaatataccaggaactcaacactaaaaaaaaaaaaaaaccactaaaaagtggagaaagaatctagaattatagacattcctcaaaagaaaatatacaaatgaccaaagaatatgtattgaatagcaggaggcatcatactactgatgaatactattgaatatgaaaaaaatgctcaatatcgaccaggtgcagtggctcacacctgtaatcccaccactttgggaggccaaggggggcagatcacctgaggtcaggagttcgtgaccagcctgaccaacatggtgagaccccatctctactaaaaatacaaaaattagccagacatggcggtgcacacctgtaatcctagctactcgggaggctgaggtgggaggattgcttgaacctgggaggcagaggttgcagtgagcagagatcatgcctctgcctaggtgacagagtgggactccgcctcaaaaacacactctatcactgatcatcaaggaaatgcaaaccacaattagatatcatctcaccccagttagaatgctacaatatggtttggatatttgtcccctccaaatctcatgttgaaatgtgatccccagtgttggagtagggcctggtggcaggtgtttggatcatgggggaatatgcctcatgaatggcttagtgtcatccccatggtgatgagtgagttcttgctctgagttcatgcaagatctagttgtttaaaagtgtgtggtacctcctccctctctcacttgctcctgctctcaccttgtgatacaggtgttttgtcttcaccttccaccatgattttaagcttcctgaggccctcaccaggagcagatgccagcgccatgcttcctgtacagcctgcagatctgtaagccaattaaacctcttttctttataaattacccagctagcaatacaaattactggcattatagcaatgcaaaatggcctaataccagctcttatcaaaagggcaaaaattaacaaatgctgatgagaatgcagagaaaagggagctcatacactgttgatgggaatatgaggtagggcagccattacagaacacagtatggaggtttctgaaaaaactataaacagaaccaccttatgattcattcagcggtcccgctactgggtatttatccaaaggaaaggaaatcagtatgtcaaagcggtacctgcacccctatgtttattacagcgctattcacaatagtgatgatatgaaaccgtcctaaatgtccatcggtggatgaacagataaagaaaatgtggcttatatacacaatgaaatactatttggccataaaaagtaaaatcctgtcatttttagcaacaaggatggaactggaggacattctgttaagtgaaataagccaggaacagaaaaacaagtatcacatgttctcactcatatgtgagagctaagaacactgatctcatggaggtagagagtagaatgacaattaccagagactgggaagggtggggagataaagagatgttggctaatgggtgcaaacatacagttacatagaaaaactaagttctagtgtttaatagcacactggggtgaatacagttaacaataatttattgtaaatttcaaaacaactaaaagaaaatatttgaaatattcccaacacaaggaaatgacatatgtttgaggtggtagatattccaattaccttgacttgattgttcattacacctcgtattcgtgtatcaaaataggacgtgtgccccagaaatttgtatgattattatatatctttttttttttttttttttttttttgagatggagtctcgctctgtcacccaggctggagtgcagtggcatgatcttggctcactgcaagctccgcctcctgggttcatgccattctcctgcctcagcctcctaagtagctgggactacaggcacccgccaacacgcgtggctatttttttgtattttttagtagagatggggtttcactgtgttagccaggatggtctcgatctcctgacctcaagatccgcctgcctcggcctcccaaagtgctgggattataggcgtgagccaccgcgcccggcctctattttcaaaatttaccttcatatttaaaggctctgatctatttttcattgcagactttttttattgacttccaccataaaagataaggatttctctctctctcttttttttttttttgagatggagtctctctctttcacccaggctggggtgcaatggcacaatcatggctcactacaacctcaacttcctgggttcaagcaatcctcctatctcagcctcccaaatagctgggaccacaggcacatgcccccatgcctggctaatttttaaatttttggtagagatggggctgccttatgttgcccaggctggactcaaactcctgagctcaagtgatcctcctgcctcagcctcccaaagtgctggattacaggggtgagccaccacacccagttgggatttatctcttacaacaacctcccctctctcccacacacatagacacacttccactcttccatctcacagtaagttgtatggcaatatataaaaatcaagattcagtatttgtattattgctaagtaaacactgttcctagctgagtcttgtgatgtcagattacattttctttcctatacagagtcttcacgtctaccctacccccacattcagaattgccttttttttccattgttaaacaatagatgttggcaaagatgcagagaaggcccagcacggtggctcacgcctgtaatcccagcactttgggaggccaagatggctggatcaccaggtcgggagatcaagaccatcctggctaacacggtgaaaccctgtctctactaaaaatacaaaaaaattaactgggcgtggtggcgggcacctgtagtcccagctactcaggaggctgaggcaggagaatggcctgaacctgggaggcggagtttgcagcgagtcgagatcacaccactgcactccagcctgggtgacagagcgagactccatctcaaaaaaaaaaaagatgcagagaaaagggaacacttatacactgttggtagggaagtaaatcagtacaacctccctggaaaatagtatgaagatttctcaaagaactaaaaatggaactaccccctttgatccaccaatcccatgactgggtatctttcccaaaggaaaataaatcattttatccaaaagacacctgcactatgtttattgcagcactatttctaatagcaaaatcatggaatcaacctaagtgttcatcaacagatgactggattctaaaatgtgatatagatacaccatggaatactactcagctataaaaagaatgaaaccatgtcttttgcagcaacatggatggacctggaggccgttatcctaagtgaaataactcagaaacagaaagtcaaaaaccacatgttctcacttatacgtgggagctaaacaatgtgtacacatggccatacaaagtgaatcatagacactggagactccaaaaagtgggagggtgaggaggcgggtgagggatgaaatattacctattgggtacaacatacactatctgagtgatgagtacactaaagcccagactataccatcaggcaatatatccatgtaacacaactgcacttgtacccctaaatctataaggacaaacaatacaatcaataattttgaaagacaacaaataattgccttttttgtttgctttcttttctatgtacccatcactcatttttctcagaactgccccctcacccctccgtgtaattagatggccaggtaatgggccacttttttcctctgagatatccctcccccagccacagcctcctgctcttatctggctctgagagctcagagctgccatctcagaacttccctttgatcttattctggagattttcatccctctcctgagctgcttttctccccatctttctctttctttgttcacccctgcattttggtggagctcctgttccagtagtttcctgggaaaagggttggggggttggaaatcgttttaaagtcttacattctgaaaaggcctttagtctgtctttataagatttcaagtttaggccaggctcagtagctcatgcctgtaatcccaacactttgggaggctgaggcaggagaatcacttgaggccaggagtttgagaccagcctaaacaacatagtgagaccctgtctcaataataataataataataataataataataataataataataataataataatgagatttccaagtttggctggggattgaattggagaaagacatgtcctgtaattttgaaggtcttctacctgccaatgctgcaggtgagaaacctcatcccattcccattactgatcttaatatgtgacttcttttctctacaccaccggctactgggattctctctttattcttgatactctgacaattcaagattcggtgccttggtgagggtctgttttcatcgttcttgttgggtattctgtgggtccttcgaactgttgatttatggcctcagtgctgaaaatgttttttatagtagtttattgataacctctcctcagttttctctggtctttcccactgaaacgtcttcagtatgatatttgacctttcttattttccatctctttttgttcgacttctttggagagttccatgaattttttttttaacttattattattatactttaagttttagggtacatgtgcacaatgtgcaggttagttacatatgtatacatgtgccatgcttgtgcgctgcacccactaactcatcatctagcattaggtatatctcccagtgctatccctcccccctccccccaccccacaacagtccccagagtgtgatgttccccttcctgtgtccacatgttctcattgttcaattcccacctatgagtgagaatatgcggtgtttggttttttgttcttgcgatagtttactgagaatgatgatttctaatttcatccacgtccctacaaaggacatgaactcatcattttttatggctgcatagtattccatggtgtatatgtgccacattttcttaatccagtctatcattgttggacatttgggttggttccaagtctttgctattgtgaataatgctgcaataaacatacgtgtgcatgtgtctttatagcagcatgatttatagtcctttgggtatatacccagtaatgggatggctgggtcaaatggtatttctagttctagatccctgaggaatagccacactgacttccacaatggttgaactggtttacagtcccaccaacagtgtaaaagtgttcctatttgtccacatcctctccaggacctgttgtctcctgactttttaatgattgccattctaactggtgtgagatggtgtctcattgtggttttgatttgcatttctctgatggccagtgatgatgagcattttttcttgtgttttttggctgcataaatgtcttcttttgagagaagtgtctgttcatgtccttcgcccacttgttgatggggttgtttgtttttttcttgtaaatttgtttgagttcattgtagattctggatattagcactttgtcagatgagtaggttgcgaaaattttctcccattttgtaggttgcctgttcactctgatggtagtttcttttgctgtgcagaagctctttagtttaattagatcccatttgtcaattttggcttttgttgctattccttttggtgttttagacgtgaagtccttgcccatgcctatgtcctgaatggtaatgcctaggttttcttctagggtttttatggttttaggtctaacgtttaagtctttaatgcatcttgaattgatttttgtataaggtgtaaggaagggatccagtttcagctttctacatatggctagccagttttcccagcaccatttattaaatagggaatcctttccccattgcttgattttctcaggtttgtcaaagatcagatagttgtagatatgcggcgttatttctgagggctctgttctgttccattgatctatatctctgttttggtaccagtaccatgctgttttggttactgtagtcttgtagtatagtttgaagtcaggtagtgtgatgcctccagctttgttcttttggcttaggattgacttggcaatgcgggctcttttttggttccatatgaactttaaagtacttttttccaattctgtggagaaagtcattggtagcttgatggggatggcattgaatctgtaaattaccttgggcagtatggccattttcaggatattgattcttcctacccatgagcatggaatgttcttccatttgtttgtatcctcttttatttcgttgagcagtggtttgtagttctccttgaagaggtccttcacatcccttgtaagttggattcctaggtattttattctttttgaagcaattgtgaatgggagttcactcatgatttggctctctgtttgtctgttgttggtgtataagaatgcttgtgatttttgtacattgattttgtatcctgagactttgctgaagttgcttatcagcttaaggagattttgggctgagacgatggggttttctagatatacaatcatgtcgtctgcaaacagggacaatttgacttcctcttttcctaattgaataccctttatttccttctcctgcctaattgcctggccagaacttccaacactatgttgaataggagtggtgagagagggcatccctgtcttgtgccagttttcaaagggaatgcttccagtttttgcccattcagtatggtattggctgtgggtttgtcgtagatagctcttattattttgagatatgtcccatcagtacctaatttattgagagtttttagcatgaagcgttgttgaattttgtcaaaggccttttctgcatctattgagataatcatgtggtttttgtctttggttctgtttgtatgctggattacatttattgatttgcgtatattgaaccagccttgcgtcccagggatgaagcccacttgatcatggtggataagctttttgatgtgctgctgaattcagtttgccagtattttattgaggatttttgcatccatgttcatcaaggatattggtctaaaattctcttttttggttgtgtctctgcctggctttggtatcaggatgatgctggcctcataaaatgagttagggaggattccctctttttctattgattggaatagtttcagaaggaacggtaccatttcctccttgtacctctggtagaattcggctgtgtatccatctggtcctggactccttttggttggtaagctattgattattgccacaatttcaggtcctgttattggtctattcagagattcaacttcttcctggtttagttcttgggagagtgtatgtgtcaaggaatttatccatttcttctagattttctagtttatttgcatagaggtgtttgtagtattctctgatggtagtttgtatttctgtggaatcggtggtgatatcccctttatcattttttattgcatctatttgattcttctctttttctttattagtcttgctagcggtctatcaattttgttgatcctttcaaaaaaccagctcctggattcattaattttttgaagggttttttgtgtctctatttccttcagttctgctctgattttagttatttcttgccttctgctagcttttgaatgtgtttgctctggcttttctagttcttttaattgtgatattagggtgtcaattttagatctttcctgctttctcttgtgggcatgtagtgctataaatttccctctacatactgctttgaatgcgtcccagagattctggtatgttgtgtctttgttctcgttggtttcaaagaacatctttatttctgccttcatttcgttatgtacccagtagtcattcaggagcaggttgttcagtttccatgtagttgagcggttttgagtgagattcttaatcctgagttctagtttgattgcactgtggtctgagagatagtttgttataatttctgttcttttacatttgctgaggagagctttacttccaactatgtggtcaattttggaataggtgtggtgtggtgctgaagaaaatgtatattctgttgatttggggtggagagttctgtagatgcctattagatccgcttggtacagagctgagttcaattcctgggtatccttgttgactttctgtctcgttgatctgtctaatgttgacagtggggtgttaaagtctcccattattaatgtgtgggagtctaagtctctttgtaggtcactcaggacttgctttatgaatctgggtgctcctgtattgggtgcatatatatttaggatagttagctcttcttgttgaattgatccctttagcattatgtaatggccttgtttgtctcttttgatctttgttggtttaaagtctgttttatcagagactaggattgcaacccctgcctttttttgttttacatttgcttggtagatcttcctccatcctttcattttgagcctgtgtgtgtctctgcacgtgagatgggtttcctgaatacagcacactgatgggtcttgactctttatccaatttgccagtctgtgtcttttaattggagcatttagtccatttacatttaaagttaatattgttatgtgtgaatttgatcctgtcattatgatgttagctggtgattttgctcgttagttgctgcagtttcttcatagccttgatggtctttacattttggcatgattttgcagcatctggtaccggttgttcctttccatgtttagcgcttccttcaggagctcttttagggcaggcctggtggtgacaaaatctctcagcatttgcttgtctgtaaaggattttatttctccttcacttatgaagcttagtttggctagatattaaattctgggttaaaaattcttttctttaagaatgttgaatattggcccccactctcttctggcttgtagagtttctgccgagagatccgctgttagtctgatgggcttccctttgagggtaacccgacctctctctctggctgcccttaacattttttccatcatttcaactttggtgaatctgacaattatgtgtcttggagttgctcttcttgaggagtatctttgtggtgttctctgtatttcctgaatctaaatgttggcctgccttgctagattagggaagttctcctggataatatcctgcagagtgttttccaacttggttccattctccccgtcactttcaggtacaccaatcagatgtagatttggtcttttcacatagtcccatatttcttggaggatttgttcgtttctttttattcttttttctctaaacactccttctcacttcatttcattcatttcatcttccatcgctgataccctttcttccagttgatcgcatcggctcctgaggcttctgcattcttcatgtagttctcaagccttggttttcagctccatcagctcctttaagcacttctctatattggttattctagttatacattcttctaaatttttttcaaagttttcaacttctttgcctttggtttgaatttcctcctgtagctcagagtaatttgatcatctgaagccttcttctctcaactcgtcaaagtcattctccgtccagctttgttccattgctggtgaggaactgcattcctttggaggaggagaggcgctctgctttttagagtttccagttttctgctctgttttttccccatctttgtggttttatctacttttggtctttgatgatagtgatgtacagatgggtttttggtgtggatgtcctttctgtttgttagttttccttctaaaagacaggaccctcagctgcaggtctgttggagtacccagccatgtgaggtgtcagtgtgcccctgctagggggtgcctcccagttaggctgctcgggggtcaggggtcagggacccacttgaggaggcagtctgcccattctcagatctccagctgcgtgctgggagaaccactgctctcttcaaagctgtcagacagggacatttaagtctgcagagattactgctgtctttttgtttgtctgtgtcctgcccccagaggtggagcctacagaggcaggcaggcctccttgagctgtggtgggctccaaccagttcgagcttcccagctgctttgtttacctaagcaagcctgggcaatggcgggcacccctccgccagcctcgctgccaacttgcagtttgatctcagactgctgtgctagcaatcagcgagactccgtgggcataggaccctccgagccatgtgcgggatataatctcctggtgcgccgttttttaagcccatcggaaaagtgcagtctttgggtgggagtgacccgattttccaggtgccatctgtcacccctttctttgactaggaaagagaactccctgaccccttgcacttcctgagtgaggcaatgcctcgccctgcttcggcttgtgcacagtgcgtgcacccactgacctgcacccactgtctggcactccctagtgagatgaacctggtacctcagatggaaatgcagaaatcacccatcttctgtgtcgctcatgctcaagttccatgaattttatattccaactctctcactaagtttattgtgactatcatatttttcatttccaagagctacttcttgttctctgggtgttcctatttaaaaaaaaaaaaaaaaagcatcctttttatttgatggatcttatctttcagaggatgatgacaactgttttaaagtttcttactgtaccttacatcatctttgtttcctgaactcctttgcagttctgtttatttgtgcatttgctttgggccctctcttgaacattggaggctgtctggtatggataaaaaaaatacataagccaaacaaaacaaaacaaaaactacacataaataaacctccctggctgggcctggtggctcatgcctataatcccagcactttgggaggctgaggcgggaggatcacttgaagtcagttcaagaccagcctgggcaacatagcaagagcctgtgtcttctaaaaataaaaaaattagctgggcatgacgatgcatacctgtagttccaggtattcaagaggtagacatgggagaatcacttgagctcaggagttcaaggctgcagtgagctatgatcacaccactgcactctagcctgggtaacagagtgagaccctgtctcaaaaaaaaaaaaaagtataatataaatacatgctcctctttaagaaaaaaaatagaggttttcttttcagtaaagtgctttgctgggtaaattcattagtgtagaattttaaattccaaatcagactgcattttgctatgcattgtattttctgttctttggatcacctgacacccaaagaatagtaagaaacttcttttgaattaaaatttgtttttaatataagaaacacattgtaaactcactggtaaaagtgagtactccttgtacatctccatcagagctcttaagtgcattgtcagtgggcagtaatattttgaaaggaatctttttctgagcagtaggtctcaacagtttgcttaaaatattcagtacaccatgctgtaaacagatgggctcttattattctttgtttttttttgtttgtttatttgtttttgagacggagtcttactcttgtcacccaggctggagtgcaatggcgcagtctcggctcactgcaacctccacctcccaggctcaagcgattctcctgcctcagcctccagagagcagctgggattacaggtacccaccaccacgcccggctaatttttgtatttttagtagagacagggttttgccatgttggccagtctagtctcaaactcctgaacttaggtgatccacctgccttggcctcccaaagtgctgaaattacaggtgtgagccactgcacccagcctgaacactcctattcttgaactagtcaccaaatttgtgaaattttgtttaaataattacaataatcataaatctacagggaaataaaagattaatgattcaaagtgatgtgaacctttcataaaaaacatggttttcaaaactcagcgcatggccctctcacataagaataattctacatattctcctactgctgcagcagaggggtggttgttttaaataaagtgatttctgaacattttccctaaaatctgaacacagaaagtttgttgttttcttaccaatggctttctcctcctctggtgaccccaggctatctgggtggttgagtggggctgctctaagcatggggacaaggcacaagggctgttgggcttctctacagagtggccaggccgagacctacctatttcgttgggagaatccagttttagtagctataggtcttttctcctgggctggccagtttttccagagaatcctctgatctcccactggaaagtagcagctgggctgctcagaactgggtgaaagagggtccctagggactcaggacacaggacgggaatttcactctatttccttgatttcaatgcatgcttttctcctccctctgctgggcttagttccctggaatccaggtctgtctgtcctgtttctccagagaatgaacttcctgtgtcccacaaggggtgaggggaggagaggtgggctctgagtgtacgttatacagacttcacgccttcctgccctctcctctgccttctgagggagatggcacctgcctccgcttccttgcttctcacaggccccttctacttactccattttgtcttcgaaagggttgttggcatcacttatctttgttcttagaagttgacatgttttttcttttttgagacaaggtctcattctgttgcccaggctggagtgcaatggtgtgatcatagcacactgcagcctcaaactcctgggctcaagcaatcttcctgcctcgacctcctgagtaactaggactacaggtgctcaccatgatgcccagctaatttttaaattttttatagagacagagtcttgctgtgttgcccaggccagtctcaaactcctggagtcaagtgatcctcctgcctcagcctcccaaagtgctgggattgcaggggtgacccaccatgcctgtcctgcaaatgttattaacagcatcttaaaatggttgatcttttccagaaggttttcagtttactttgtccagatccatcagaggaattactatctgggactatgaccttacaaaaggccttactatggccttgcaaaagctatgggcttataaaatgtattttttaaataatcagacttaaaagttgaaattactccttgatctataggctgcagagtatgaaagcaacattactccttgtacatctccatcagagctcttaagtgcattgtcagtgggcagtaatattttgaaaggaatctttttctgagcagtaggtctcaacagtgggcttaaaatattcagtacaccatgctgtaaacagatgggctccaactattccatttatagagcacaaacagagtagatttagcaaccctcaaaggcactaggattttctttttctttttctttttctttattattttttttttttgagatggagtctcactctgtcacccaggctggagtgcagtggtgcaatcttggctcactgaaagctctgcctcctgggttcacaccattcttctgcctcagcctcccaagtagctgagactacaggcgcccaccaccacgcctagctaatttttttgtatttttagtagagatggggtttcaccgtgttagccaggatggtcttgatctcctgacctcatgatccacccaccttggcctcccaaagtgctgggattacaggcgtgagccactgcgcccagccagcactaggattttcagaaaggcaaatgagccttggcttcaactttaagtcatcaggggcattagccctaaagaagagagtcagcctgtcctttgaagctttgaagccaggcattgacttatctagctataaaagtcccagatggcatctttttctagaaacaggctatttaacctacattgcaaatgtttttggtgtagccactttcatcagtgatcttagctggatcttctctggataacatgctgcaacttctaaatcagcacctgctgcttcaccttgaatttttatgtgatggagatggatttacctcatgaacaaatctctgctagcttccaagtactgttctgcagcttcctcacctctctcagccttcatagaattgaagacagttagagtcttgctctgcgttaggctttgggttaagggaatgttgtggctagtttgatcttctgtgcagaccagtcaaactttctccatatcggcaatagggctgttttgctttttaatcatttgtatggtcactggaatagcacttttaatttctgttaagaactgttcctttgcatttacaacttggctacctgtctgacacaagaggcctaagtttcagccgatctcagcttttgacctgccttcctcactatgcttaatcattcctagcttttgatttaaagtaagagatgtatgactcttccttccactcatacactttgaggtcattgtaaggttattaactggcctaatttcagtattgctgtgtcttggggaataggcccaaggagggaggaggaagggacagagagagagagatggggaaatgactggtgggtggagcagtcagaacacatacaacatttatcaattaagttcaccatcttgtatggatgcagtttgtggagcttcaaaactattatactaaagccaggcactgtgatgcatgcctatagtcccagctacttgagaagctgaggcaggaggattgcttgagctcaggagttcaaggtcagcctgggcaatataacaaggctgtctctaaaaaaacaaacaagcaaaactaacaacaaaaaatcacactgataacatcaaaaataactgatcacagatcaccataacagataaaacaataataaaaaagttagaaatattgcaagaattaccaaaatgtcacacagagacacaaagtgagcacatggcaccaatagatttgctcaatgcaggtttaccacaaaccctcaatttgaaaccaatgcgatatttctgcaaagtgccataatacaaggtatgcctgtattgaataatgcaatgttcaaatcttctagcaaagtgaacagaactacacagaatcatgctttgaaattgccaatttaggaaattatatttgttttcaaaacttgttatttcttggaactccactcataggatttgttaaaagtgtgtataaaaaatacataaacctattaatattattttatttgcaacaataactcatagaaccaagtagagccacaaacatgccctcataatattgcaacagctatacacaagcgagattatgtgatgaccacgtctctcctatgcattcctgtgcccattcccaacaatgtccaccttaaccttcaatttggagttgtcagcattttcctgctccaaattttgacttgaagtcaatggagtccttcaaatcggaaatttccctatgttgattaagcttcagtgctcatagaactatcatacttttgcctgttttatcaaattcatttttctcaggttttgggatttgaaaaaaatatatgatttcccaaaaaaaaatgcccagaagaaatccctgcatgaaaacactaggccatgatctttgcctgattgctaaaggaagaatcttgttaaacgaaataatatcataaatgagaacgtgcatgggggtagtgaaagcaatgggttaggccctggttgctgcctgttagaagagccctggccctcacacacccacagcccaggcacgatgtgatgtaaccctttattatttcccaggacatctcagatctgttttcctctgaaatattaggactcacatcagccctttggtcacagttaacagtgcatcatttacatctatgtgtgactttgtcactctgtggcacccaagtagaaagaagaaggtaaaggcacattagaagaatccattctattccctttctgctgtcctagctgtcaaatcagcagaaagcttagacagaccgtatgtttactggaagggagggtagagccccagagaggcttcggactgagacctgttctgccttctgtctggaaagggaagaaattcctccaccagaagcaacaagagttgggaagcagagatcaaagaaagcaggcaatttctgcaggaagggggaagggaaaaagggagggaagagtgatgggtcccaggcgggggcggatttagcatgaaactaatgaagcttcagcctcagcacccctcgcttgcaaggacccttccaagttcctgggtgaggcccctagaaattggtttaaaatttgcaaaatttggaaaagtgagatatattaacaacaatcaagactacagccatgtgtggtagcacttgcctgtagtgccacctactcagaaggctaaggctgggggatcacctgaggccacgagttcaagatcagcctggacaacctagtgagccttccgtctctaaaaaaataaaaataaaaaattaagactacaggctgggcatggtggctcacacctgtaagtaatcccagcactttgggaggccgaggcgggcggatcacctgaggtcaggagttcgagaccagcctggccaacatggtgaaacgctgtctctactaaaactacaaaaattagctgggggtggtggtgggcacctgtaatcccagctacttgggaggctgaggcaggagaattgcttgaacatggaaggcagaggttgcaatgagctgagatcaagccactgtactcaagcctggatgacagagcaagactcgtctcaaaaaaaaaaaaaaaaaaattaagaattaagactacagtcttttccctccctgacatccctcagtcacacttcctttcttgttgggtggtatttggagtggctgtgggcatttttaatctctatctaaggagaaattgagttgagagtatgttcacttatgtgatttgcagtcactttcaagtatagtttctagcagcccctccctcagtcaaggaatggcttctaggggcatctccattgtctactgtattgacttaataccaaaggtcctatccaaatataaatgtgactttcaatacctggcaccggaagtaggtgggaagtggaacagaaagaatgttgaaaacatatggagtcagaagctagtttgtagaaaattctttcagctatggtaaatgtaaaaggcagggttcgcttttcactgatgtctagtcaacattttcttcttccaggatattcccccttgctgtgactatttttttttttcttttttgagatggagtctccctctgctgccaggctggagtgcagtgatgcaatctcggctcactgcaacctctgcctcttgagttcaagcagttatactgcctcagcctcctgagtagctaggactacaagcgcccgccaccatgcccagctaatttttgtatttttagtagagacggagtttcaccatgttggccaggatggtcttgatctcttgacctcgtgatctgccctccttggcctcccaaaatgctgggattacaggcgtgagccaccgtgcccggcctttttttctttctttctttcttcctttttttttttttctgagatggagtttcgctgttgttgcccaggctggagtgcaacggtgcaatctcagctcaatgcaacctctgcctcctgggttcaagcgattctcctgcctcagcctcctgagtagctgggattacaggcatgcactaccacgcctggataattttttgtatttttagtagagacggggtttttccatgttggtcaggctggtcttgaactcctgacctcaggtgatccacccgcctcggcctcccaaagtgctgggattacaggtgtgagccaccatgcctgacctgtcgtgactattcttaatactgctgcgaatacaattataaacgcactgtacatttatttatttgttgggattgaaaaaatagaatgtatgaaattcttgagttaaccataaacctatagcaatattacagaaatttttgatcaactatgctggaaaaaacactaaattatctttctattatttctatagaaaatggcactacaaaattgttggcatttgaagcggtcatcaaagagtaggcagacagaagcacatggaaaaatattatagaagtgtatcagttaataaaaacgtgaattttctggattttgtgatgtttgtggtatttgtcagcctttaaaaatttataacttgatgtcatctattttctcattctaaatatttacttttatattcaactttgtatttttttattttgtgttatttttcttgaagagatctccaaattatataagcttcagacttcacaaaacttgatctgtgcctggactgtggtgtggggcccagggatttataccaacagccattcttggccctcagcagcctctagggtccctagcccattctccaaagtccgcatcccagccctcatctcaggccctgagagccatcaacaacataaggctgaggaactctttgcttctctggggagcaggagggtgggtgagccagtgagacatgcatttgctggattagacctctggggacttttcccccagcaaagtcctgcctggcagggtggttggccattgtgtcagtacccaccaccctcctgtgaagggtgagcccctggcccttgtggcctgggctgaggtctcaaccaccagcagcagtacttgaggctgagtctgctgtagactttaaaaacaggactggtggccagacacagtggcttatgcctataatctcagcattttgggaggccaaggcaggagaattgcttgagcccaggagttcaagaccagcctgggcaacatagggaaacctcgcctctagaaaaactttttaaaagccaggcatggggccaggcgcggtggctcactcctgtgatcccagcactttgggaggccgaggtgggaggatcacttgaggtcaggagtttgtgaccagtctggccaccatggtgaagccccatatctactaaaaatacaaaaattagcaggacgtggtggcacatgcctgtaatcccagatacccgagaggctgaggcaagagaatcgcttgaacctgggaggcggagtttgcagtgagccaagaaggtgccactgcacttcagcctgggcgacagagcaagaccctatctcaaaaaataatttttaaaaacccaggcatggtggcgcatgcctacagtcccagctactcgggaggctgaggcctgcttgagcctgggaagccgaggctgcagtgagctgtgatcatcccactgcactccagcctgggtgacagagcaataccccatctccaaaacaaaaacaaaaacaaaaacaaaaaacaggaccagcaggaatagtctgctgtggattgggacccctgaatgttccagagggatttaagtgtgcggaattcagacgtcttccctgttgtggctggccttcccgcctccccagccgtctgcccgaatgagtgcttactaatgagccagagagctctgagccggcctgtgtagggcatccatgcagggctccaggggcccctctcagtgaaacaacatgactgtgaaaacaatctgctgctcttcttcttcaccctgaccttgggtcattctggcgtaaaagattactttgctcccagcctcggggaagtttttgtacaattagctacaactcggcagaaagaccctgcagagagagccctggggtcaccaactcaagcatgaaaggacactgaccccgtgtggtcatgtttggaggtgcagcacggggagagaggaaaggccccggcacggtcctttggccaaacgacccctgcaggccctccctgaccgaccttgctctgtccctgctcacttcacaagaccctggggagaggacctggccctatcagggcctgctgactcgttctcccctggcgaaatgtccccaccaggtgctctcaactctggggtatgacttcacaggccatttgagttcagatgggtaaggcgtgtaggaaatgagctcaaagggtaggatggattttgatggggtaagaagagagtgtttctcactcacccaaagcactgagcacagtgtcccaaacataatgtggagtaaacacgcgcatgcacacgtgcacttcaggtgcaggagacagcctaagcaggggcaggcaggcaggcaggtatggaatgtgcatggagggctggagagtggagctgcttttctgcggagatttcaggttaggtggagcactgagctgaattttctagaagctggattcctaagccccagttctgggctctttctctgaccacacagctgtgtagaaggaaaggggcctgagttctgccatctgggctaggagtcggtatagagccagccagttgtagcatttctgattccatctgaattcctaacacatcaaaatcagattccattctggacattcgtttttttgttgacgctgtgattgaggtctcataaaaagaaggtgccatgcctaggacaaatgaggaaggagaggctcagaaaagttaaatagctgtgtcttaagtcacagccactcagtgtgtgaaaagccaagctggggtttgaacatgataaaaatacaccatggccgctgggctccaaagtgcaacctgcagcttctaaaggaactaaaacctaacttgtttccttctcccccagctcaatccactctccccgctgtagcagagccatcgcctcaaggcaaattgcatcccactgctcaaaactgagcaactcttctctggtctcaggataaaaatggtatgtgtgtcttggcttcacagacctccctaacctcaccccttgctctcgccacatcactcgccattcttccctcctgtgacatggaacttcagccttggttccatgccttggcctcccaaaatgctgagattataggcagttccctaaacagccagcctctcagatgtggaggaccttgatttgtaaacaaatcaccaggggattttgataaaatacagattccaattcaccaagcctggtttgggggtccaggttctgaagctctaagaagctcccaggtagtatgaatcttccaggtccacagaccacaggtgagtaaagaggtgctaaacacaccatgttcccctcacctttgaccgccaccaaacttcctttgatctgaaacttttctccttcctccccaaggccccctcctgtatctttcacccgggtggtaaggctacttcctctccaggcctcagcttaggtgctgctgcctcccggaagtactcttaacccctttgagaatgattaggtcatgtcttatctgttagagatgcaaataatcccagcactttgggaggccaaggcaggcaaatcagctgaggtcaggagtttgaatccagcctgggcaacatggcaaaaccccatctctactaaaaatacaaaaattagccaggcatggtgatgcacacctgtaatcccaactacttgggaggctaaggcacaagaatcgcttaaacttgagaggcagaggttgcagtgagcagagatcgcatcactgtactccagcctgagtgacagggcgacactctgtctcaaaaaaaaaaaaaaaagaaagaaagaaagaaagaaatgggtgaaacgggccaggcacagtggttcacacctgtaatcccagcaccttgggaggccaaggatcacttgagaccaggagtttgagaccagcttgggcaacatagtgagaccctgtctctacaaaaaatacaaaaattagctgggtgtagtggtgcgtgcctgtagtcccggctactctggaggctgaggtgagaggatcaccagagctggggagatcaaggctacagtgagctgtgattgctccactgtactacagactgggtgacacagtgagaccctctcagaaaaaaaaaaggaaaaagaaatggatgaaatgataatgataggtgggatttgtcttaaaatactcaagaaaaaatatgggaacaggtacatgaaacaaaaatggcaaaatgtcgatcattttgattgttgttgaagctgggtgatgaatatgtgagagctcattgtactatcctctcaatatttgtgtttgtttggaagttttcaaaataaaaagctttaaaaaaatttttaatagacatagggtcttgctgtgttgcccaagttgctcatgagctcctgggctcaggcagtcctcccaccttggcctctcaaagtactgggattacaggtgtgagccaccatacctagccagaataaaaagcttaaaaaaatgaagcctgagataagtgcctctcccatgtacccccttaggcgactgtcctgtctgcccccaccctacagcacgtatcgtactgcctcatgagtgtatagtttttcaagggctgagattgtactgtgttttgttcatttttctacaccagcccctagcacagtggttggcacagagtaaattctcaacatatttttgttgaatgattgaatgaaaatcctgccaggttatgttagttggcagaaaagaagcaaatcattgaaaatgtatgtcgaaatgaaacaacgcatattgttccaccttatgtagagctgtgctgcatctgctcctggaatcctctgtaaaggtcactgtgtcccaaggaagattctgtgtcacatgagagaatccatttaaatcgttaagaagaaagcaaatgtagacactggaactctgccatgtataaggaagaagggtgaatgggtatgtgatccaaaggtgttgaaaacagagagtcattcattcaatcaccaaatattggttcactgtctaagcttcagctttgcctgcaacgctggagactacttctccaaagtagttacttgcatgagattatatgaatggaagcctgtgtatattggccaaatgaatgaggcagaagtgggttttgcggggggtggggtgttgtttgttttgtgcagggataccccaaaagaaaggggagagaacagggcagtgggcgagatggtgagatatctaggccccaccccagtgggaccaaaggggtaactcagagctggaggctaaagagctgactttgtaagacctggggtgtcagtctaatgacagtctgaggcaaccccaggaaccccaggattcgagaatgagcccttcttggctccactgcccatacacatctgctcagacccagtactgctttaatgttctgaggtctctgggtgaccaccagcagcaggcgacagggaaaaaggcctgggttttctggcctgaacaatgggatgaatagggataaacattgacaggcatgagaaataaagtgggaagagcaagctcaaaggagacatagtgtgttcactcaccaaggctgttcaccagagaagcccctttcatgtaagtgagagatacgcttgagaaaaccaaaagagagactcacctgtttcccccaacaagcttccaggtcttgttatcctcagaagcaggaccaggccgggcacggtggctcacacctataatcccagcactttgggaggccgaagcagatgaatcacctgcggtcaggagttcaagaccagcctggccaacatggcaaaacccgatctctactaaaaatacaaaaattagctgggcgtggtggcgggcacctgtaatcccagttatttaggaggctgagacaggagaattgcttgagccccggaggcagaagttgcagtgagctgagattgtgccactgcattccagcctggacaacagagcaagactctgtctcaaaaaaataaataaataaataagaagcaggaccagaggttggagctctaaacagaatcagaaaatcaaggtgggtttcagagtcaggagagccacagcatcaagggaatacagccacatcatggcaagatgaaaatttctgaggctgacagtaagaaaggtgaagtaggctgggtgtggtggctcacacctgtaatcccagcactttgggaggctgaggcacgcagatcacctgaggtcaggaattcaagaccagcctggccaagatggcaaaaccccgtctgtactaaaaatacaaaaattagctgggcgtattggtgcacgccagtaatcccagctactagggaggctgaggaacgagaatctcttgaacctgggaggtggaggttgcagtgagctgagtaagctgagatcgcgccattgcacaccaacctgggtgacagaacgagattctgtctcaaaaaaaaagaaaagaaaagggggtggatggaattctttgatggcacatccctgactcagagtggtaaaactcacataccgatgaccttgtgttcagtcttattcatttctctatgccaggtccctagcacagtgtctaatgcaaaggaggtgccgagtcaatgtttgaaaaatcaatgaatgagatgaatgcacttggaatggtgttgatttttgctttgcctccaagctagaacaaataattgagctcactcgtatccatgttatcattactatccaggctactgtgttaatatagcatttaataactcccttgttctcccaaactttagggaaattgaagcttcagtaatgagggaaatttttgcttgattgttttctgtgggaagtcaaccacagctcacatctggtgccctccatgaagtcttcatcattaacaggattttgtacattttctcctttctgtaaatccagaccttggagaaagagatcctcctcatctttccattaagaggaagtttaattgcatctagactctggttggttagggcttgattttaggtgggcaaagcagacttcagcaaaataggcagagccaagagaggctcctgttgtttgggtcgctgtttttagcagtgaggaaagaccctggggctagaaggatgattggtcactaggtgtgaactacacgttggagatttcatctgcctgggacctagcagatgtccccatgaggtcttctttgccagctaccccatcctgtcttccagagaatgctgcatccagaggtctcttgtcctcttttgctccctggaccttattacttgatcatctagctcccagctatgttccttccactcccatccagggctactgacttccactctataagcagtaacagactggtgcctttcaatcaatagtcattcatttaacaaacatttattgaacccccaatatgtatctagcactgttctaggcattgggaatatagcagtaagtaagataggcaaaattcctaacctcatacagcttatattccaatggggggaatataatatatatatatatatatatatatatatatatatatatatatatatatatatattttttttttttttttttttttttttttttttttttgagttggagtctcactctgtcacccaggctggagtgcagtggtgcaatctcggctcactgcaacctctacctcccggaatcaagagattctcccacctcagcctcccagctagctgggattataggcctttttgtatttttagtagagacagagtttcactatgttggccatgctggtctcaaacttctgaccccaaatgattcactcacctcagcctcccaaagtgctgggattacaggcgtgagccactgcgcccagccaaaaaggaatgctatattcttgctaaacaaatgaacatgcccagaatttctgaaacaggatgcacagagcatccacctctaagagaggatggttcactccaacactcccctctctggccagcaactcgcaacaggcaaagcctgaaccaggggagccactataacaataagcaccgcaacagtggctgactttagtcacgtgccctactccattagccctcagaacagtgtaatgaccactagcattagaccattttgcagaagaggaaacagaggctaagagagatttaataacttggccaggttcacatagctatgctttggcagcgctggaattggaacatggtgaaatcctgtctctactaaaaatacactttgccctgctgctacacttttcagggacagaaaaggaaaactatctcaagagacaatgtgcaacgctgaaatggtgaaggaaaagcttttggggtagtgaaggggagcagatggcctgattcccccccaccctaaatagggtgtgtgtatgtgtgtgagtgtgcacagaaacacagatgcacatacatgaccacacatatgccccgagccttccaaccagaatccacaggcagcaccatctccctctccatagcctttgctgatccttgttcctgaagctagaaagggaggagggcccctagatgagtgtctgaggggcatgtcctcaggacagctctacttagtgacatttgtaagaccagtgctacagttatttcaagcaactatcatttcccttccaggataatctaagatgtgatttttactaatgaaatgaaaactaacactgagcatctattatgtcccagtcactgtgttaggcccatttaaatatgaattatgtattgtctctgactttaaaaggctcaagagagcctgccaactcctgactccaacctatttagccatattgcttgtgcttgtgcttaatttctaaattaaattggaatttcaaggaccctgctgattggcaagtggaattactaaatgatatttaacctaagtgaagttgaacatctcacgacttctggatggcaaggagaagatcaaggagaagtggaccaagaagctggctcttcaaatagccccacccttaccttcaggaaaccttatgtctgctgaactgggtgggagttctggcccctccttccactagttgtcacaactggaatggttaaaagcaatgattcccaccagaatggctaaaatttgaaaactgacaatagcaagaggagcaccaggaactgtcatgcattgctgccaagagtgtaaggtggtacagctactctggacaaatctttggcagtattcactaaacaggacacacatatacattatcagccagcaattccatgcctaggtatataagtaaagtttgtgcacatgtgcattaaaagacatgtataagaaggttcatcatagcacttttggtaacaaccaaaactgaaaacaacccacatgtctagcaagagtgggatggataacaacactgtggtgtatttacacaagggagtactctaagcaagaaggataaatgggctgagtgcagtggctcacacctgtaatctcagcactttgggaggctgaggcgggcagatcatttgaggtcaggagttcgagaccagcctggccaacgtggtgaaacctcgtctttactaaaaatacaaaaaaattagctgggtgtggtggcccatgcctgtaatcccagctactgggggaggctgaagcaggagaatcacttggacctgggagacaggttgcagtgagccgagatgacaccattgcactccagcctgggcaacagagcaagactctgtctcaaaaaaaaagaaggatacttggtctacagacacatgctactatgtgcacagctttcctaaacatcatgttcagtgaaaaagtcagataggagaaaaacacagcggcatgaacccatttacatagccatttatataaagttcaaaaacaggcaaaagtcatctatgttgcttttaaaagccagggtagaagttacctttgagaaagactaagaagatagagattgggagggttaggtcagggtttccgggatgctggtaatgttaacatttcttggcctgggtggctgttacatgggtgtgttcactttgtgatgatttattaatctataaccttatgacttttaagcttttgtgtaaatatattcctcctataaattttctaaaaacaattaggctacatcccaaaagacaagggatagagatataacctaaaaagtcagatatggtcacaggcccaggggcaaggggaggtgaggtttgcttcccaggatccccagtctcccaatatttaccaaagggacttgtgaaaaaggccaggaatgggaaagggggtgtcagggcctttgaggtgggtgggccaactacccagaactgccttggccatactgcatgactctaggcctgtaagcaaggtttctgagaggactggtctcccatcccttgccctcattcacatccccaaaagcaacttttgcccttgggccaatgctaattttctttctgaaatgatcatcagataaggttagatgcttcatatggcctcagttcttctacaaagaaaggtcaatactagtcaggcgcagtggctcacacctgtaatcccagcactttgggaggctgaggcaggaggatcacttgagctcagaagtttgagaccagcctgggcaacatgatgaaaccccatctctactaaaaattcaaaaaattagctgggcgtggtggcacatgcctgtggtcccaggtactcagaggctgaagtaggagggaaaaaaagaaaaagagaaaggtcaacacagataacccaatcaaaaaatacactacaggtttaggccaggcatggtggcttatgcctgtaatcccagcactttgggaggacgaggcaggtagatcaccgggtcagcctggacaacatggtgaaaccccgcctctactaaaaatacaaaaattagctgggcatggtggcaggtgcctgtaatcccagctacttgggaggctgaggcaggagaatcttttgaacccaggaggcggaggttgcagtgagccaagatcgtgccattgcactccagcctgggcaacagggcgagactctgtctcaaaaaaaaaaaaaaaatagactacaggtttaaacaggctcctcaccaaagaggacattcaattggccaagaaacatatacaaagatgctcaacagcattagttatctgagaaatgaaaataaaaattaaaaccgcaatgagaaaccattagacttcccccagaatagccaaaatgaaaaggactgacaataccaagtgttgggatgcagtgcaactctgtatcttatccattgcctgctgggactgtaaattggccaatcacttggtaaactgcttggcagtatccaaacatacccagcagttttactcctgggaatatacccaacagaaatgagagctgtgtccacatgttgaaacatccagaagagaatgttcacagcagctttattcttaatagccaaaaattggattcaacccaaatgtccatcacaggagggtgaataaatatattgaggcatattcatacaatgcaacacaactcagcaatacagaaaagaatgagatgctgcagcacacatcagcctggatgaatttcccataatgtagagttaagagaggccagacgtaagagcacgtactgtgtgatcccatttacatgaagttctaaataggcaacaccaatctatggtggtacaagtcaggacggcaatcctttgcaagattgtgaagtatgaactgggacgaggtgctggaaatgttttgtgtcttgggcggtggttacattgatgtatatacatgttaaagtccatctagttatacacttaagatagtgcacttcagagggcatgtgagttacacttcaataaaaatgtaagtttaaaagaaaggaagaagaaacaaaggaagaaaggaaagaaggaaagaaagagaaggaataagtgaaaggaagactggcaaaattctgccctctcatatgaaaaatggtatagcctttctagaaaataggcattttcttaaaaagttaaaacttcacctactatttgacccaacaatccacttttaggtatttatcctagagaaaggaaaactaatttgacataaaagcctgtatataaatatttatgccagcattgctcatacttgtcaaaaactggccacaacccaaatgtctttcaaactgttaggtctatacaataaaataccacttagtaaagaaaaagaatggatttggggctgggcgtgatggctcacacctgtaatcccagcactttgggaggccaaggtgggtggatcaccagctcaggagatcgagaccatcctggctaacatggtgaaaccccgtctctactaaaaatacaaaaaattagctgggcgtggtggtgggcgcctgtagtcccagctacttgggaggctgaggtgggagaatggcatgaacccaggtggcagagcttgcagtgagccgagatcacaccactgcactccagcctgggtgacagagtgagactccatcaaaaaaaaaaaaagaataaaaaagaatggactactgatatatgcaacaacatgcctgaatctcaaatgcattacactgagtaaaaacccaatctcaaaaggttacatgctatatgattccatttttatgacattctgaaaaagacaaaaatataagaacagaacacagatcagtagttgccagagtttaggaatgggggaaggtctgaatacaaaggggcagagtgagagaaatctttggggtgttagaattgttctgtatcttgcctgaggtgatagttacaaaagtctatatctgtgaaaaatgcatgcagtgtacaccaaaaagttaattttactttatgcaaataaaacatttttcaagtaaaagctggctggacatggtggctgaagcccgtaatcccagcactttgggaggctgaggcaggcggatcacctgaggtcaggaattcgagaacaacctggccaacattgtgaaaccccatctctactaaaaatccaaaaattagccaggcatggtggtggttgcctgtagtcccagctacttgggaggctgaggcaggagaattgcttgaactcgggaggtggaggctgcagtgagccaagatcgtttcactgccttccagcctgggtgacagggtgagactctgtctcacacacacacacaaaaaccaaaaaccaaaaaacaccccaccatttttcaaataaaagcaaaaaaaaaaaaaaaggaaaagaaaaaccatcaaacacacaacccaaaacaaaactctgccccccgtcatgggctctggccatccttcctccaagctcaagcaggaagagcagatggcagaggcctttgaggactaggataggacctggttgagagcagacattcatgagcttaatttcttatcctactgttcatgtggctttggaatgaccatggtcctgtgaaatgccattagctcactgtccattcttgtgtctttgccttttaatccttggacctcaccactcagaaatagctggagctgccccaactccccaaggaagaacttgctctctgacatcaagccaatgaagagtccctttggggcacacaggagaacttatcacccagtagatccttcttacccataacccatggctcctcccagctggtggggccagggccatgagctgttattcatccacatagagggaattatggaacagggccttcaaggtcccccttgatcatacagtgcccgccatgcccccacagggtcatggcccattgcacctcagcctatatgcacatgctggttgcttagctcagaactcctttctctttcccaaggctttgcccctgccctcctggaatgtcaccctttctcccccagatgtggcactatctgttaccttggcttgtagaactttctacacccaagttctactgattctttctcctaaatacccgcaaatgatctcatccttgcctgcttcagaccttcaggctctcttgcccagaccattgctaggcggatagcttctgtgatgccctctctgctctgaccccacacctcttcaattcccctcctcctaaaaaccccttggttccttacctaggcaggcacacaagaacctggccaggcccttgctgcttttcctgatccctactccttcccagacacgtcctcaacactcctataaaacctggccacttggggctaggcacggtggctcatgcctgtaatcccagcactttggaaggccaaggcgggtggatcacctgaggtcaggagttcgagaccatcctggccaacatggtgaaaccccatctctgctaaaaatacaaaaattacctggccatggtggcacatgcctatagtctcagctactcaggaggctgaggcaggagaactgcttgaacctgggaggtggaagttgcagtgagccaagattacaccactgcactccagcctgggtgacagagcaagacactgtctcaaaaaaaaaaaaaaaaagtctggccaccggctcttccactcctccatgtctctgttcatactgatccctctgcctggagtgtccttccctcccctctgtccacctagtaaacccctcattttctttaggccctactccaatccacctctctgtgaagcctttcctgaccacagtgccctccattcatctgccccaggaacattcattcattccacaagtagttatgccccacctatgtgctgggaattaggacagttaaggaccctgctcttggggagtacattcatctgggcaagactgataatccttgctatatcatcctgtataatttcagaatgattaaggtgaagggctgcagaatgataagacactcatggcggccttgctgaggggatgccaagtaagcagatacctgaggagttcctcctaaccctccgtgtcctgagcactctgcattctttttttttttttttttttttttttgagacagggtcttgctctgtcacccaggctggagtgcagtagtgtgatctcggcttattgcaacctccacctcccaggttcaagcgattctcccacttcagcctcccaagcagctgggactacaggtgcgtgccaccgtgccaggttaatttttttaggctggagtgcagtggcgcaatctcagctcactgcaacctctgcctcctgggttcaagcgatcctcctgcctcagccttccaagtagctgggattacaggtgtgcaccaccatgctaggctaatttttgtacttttagtggagacagctgtcaccatgttggccaggatggtctcaaactcctgaactcaggtgatccacctgcctcggcctcccaaaatgctgggattacaagtgtgaggcaccgtgcccggctgtgagcattctgcattctttatggtctctccttgggagggttgtgagacttaacaaataaaaacattgcataggacatacttataccaaaaaattgttcttgaaattcaaatttaatgggaccacctgtgttttatcaggcaaccctacccctaggactcactgccctgtgagttcagagaaggagcaccctcagggcccagcactgtggctggctgcagtaggcactcaataaacactaaaagaatgagccattaagcagatgcatgagcagcacttgctaagttgtactgtattttgcatttgcttgctgactccccacctgcatcgcaagcccctgcttgctgcgcaggcagtgttagatggcatcctctgtacctaatgcagcctgaggagtggtttagggagaatgtatctgcctcttaccatctattagtgcctcccctactaccccaagtaataatcaaatatgaaaaacaattttagaatgtggcaatatatgtaatgacatggaaactgagaaacattttctcaatcatatgtacatgaataaaaacacctttggaaaactttgctatatttataggctgcagtgtataatctatgaaaaaactgcttccatctagtgccctagctacccctttccaaccagacagcctctacgaaacccttcctcttttggaatcgtggagctggcaaaaagagctgaccagtagatgtctgttatacaaatgatggcagaattttactgtgattctgggcctatgtgtcaatcttcccactggactgggatcctgcacaggccagcaaggtgtccttattgtccccaggcccagcatgcagcagcagctcaggaaatggggattgaaaatctatcaagagccacatctcatagttgacgttaaagcttccccgaaggaaggctggttcaaatgcacaccccatgttgccctctggagatcagcaatcccatcaaggcccagcccaaaccacacactggcaagcagcccagaaagggtgtggtccaactgccccacaagtcctgtgaataagcccggtcttcactcccagttcataaaagggccactgtcccagggcagcctcagctcctgttggtttcaactcagaaggagaatgttgttgcttgagcctcttttgagctttaaaaaggacaaggaaaggcactgtacggagtgttttacttttgacttttttttcatgactacaaactgttggatattgaaaaccttgcatttacttgtgaattgccagtctgtgtttgcgtatggaggaaggtaagatttgcagggctggaattattttcattttccttgtaagtttctccagtacttagcttttcattgagcattctatatttatttctgtgtcattatgtcattctttggtgcattttataacctgtgatgctgaaagctgccttccttcacattgtgggctagagtcagatagggcaatacagtcacacagcatccaccagaagttagtgccatggaacacagtcctgaataagcacaccaatttgctacaagaatgatcacagaaagactgtgtataatagtatcaaagaataattcaaagacatagggttaattattagggcaccatttacttgaatacaagggaaaccaaaaatagctttatttcatatatcagagccaaaataagaaataggaaaagaggagaaaggagaaaaatgaaaaactgcagacgtgtgaaagataatgaactttgccttttcttgagtaagctgctgattagcaggcagctagctaagaaggccagagggtgttctgcccttgacatctggatgaatcatcaaagaggataattccagttgcttatggattctccaggagcccttggagtggaactgtgctgtatctgtgacaagttccctctgtgtcacatgtgcctgggtcctgggagaacacctccatctctgttatgcctctctgccctatgacctttatgtaaaaagtctcaatgtaagcagcctaaatgtctaacttaaaaggaaccagctaaacaaacctcagtataacctcagcatggaataccaaagatctttttttttgaaactgagtttcactctgtcgcccaggctggagtacaatggtacaatctcagctcactgcaaactccacctcccaggttcaagcgattttcatgcctcagcctcccgagtagctgggattatgggtgcccaccatcacgcctggctaatttttttatttttagtagaagcggggtttcactatgttggccaggttggtctcaaactcctgacctcaggtgatcctcccgcctcagcctcccaaagtgctaggattgcaggcgtcagccaccatgcccggccccaaagatcttttaaaaatgttgtgatttaagaaaatgttgtagtaaaaatatgtaataatgtagaagttgttcatgatataccattaagttatgagtttgtggggacacactcattccatagcattaaatctgtatcagtcaaggttctctagagaaacagaaccaataggatctatgtatacatatatctctagaaagggacttatgagaaggtattggctcacgtgaatatggaggttgagaagtcccacaatctgctgtctgcaagccgaggacccaggaaagctggtcgtataacctgagagccagagggccgatggtatagattgcagtccagatctgaaggcctgagaagcaggggtagcaacggcaggggaagatccatatcccagctaaagtagtcagagatcacatattcaaccttcttctacatttttgttctgtttataccctgaacagattggatgcagtccacgcacagtggggagggccatctgcttgactcagttcaccaattcaaatgctaatcttttccagaaacaccctaacagatatatctagaaataatatttatctgggcatcccatggcccagtcaagttgacacataaaattaactgttgccataatccagtggggagaaaatggtgcctgaacatgagcagtggttgggtggctggaaggaaggagatggattgagaactctctagcagccacagttggcagaacttggtgctaatcagaggtgggggtgagaaaaatgagtctaagacaactgatgattttggttggatgactggtggtcacattaagtgagaccctttctttctttctggtttttttttttgtttgtttgtttcttttttttttttgagatggagttttgctcttgttgcccaggctggagtgcaatggcacgatcttggctcaccgcaacctctcactcctgggttcaagtgattctcctgcctcagcctcccgagtacctgggattacaggcatgtgccaccatgcccagctaattttggatttttagtagagatggggtttctccatgttggtcaggctggtctcaaactcccgacctcaggtgatttgcccacctcagcctcccaaagtgccgagattacaggtgtgggccactgcgcctggccgagaccctttcttgtgcattgccatggtgaaaataccagcctggtattaccatttctttcaaagcgttgctaacatctctgaaaatcctagcacactcccttcagtggtcccctgtactcccttgcctgagggatctccctgcctacatgccatgcctccttatttcacccatgggccccagaggggtacagcagccatcagtgaggtactggttctttctccctgggcttatagtcccactgtgttgagtaccttgtcttcctctgttcgggtcacctccctgtgccacaaacatctaccttccaggaaaggatcacaaactgtaggactgaagactagatttcaacccatagatgtctatgatttggcctgctctgtgtatgtgtgtgtatacacacacacacacacacacacacacacacacacacacacacttaaatgtaaatgcctttgaaggagggaccaggctctctggtatgtcatgttccctcaacccttctggttgcctcacctttggcctgctttgctcatttcccttcatgcctgacagccacaggcttctgtatgtctaagccccactggaaggaaagatgtagggggtctggtccaccaaggggcagcatctggaccaacagggcactggctttcttccttgtcacatctttctcatccttgctcacagcaggatgagcgcttggagagatggctggcttcctacagaaggttaccaaaagaccaagctatcttcccttgaaaataaaacccaattgcctgtatattagattgaactatataaaattgtccatgttcaactttgccctataaagatgacaatttcacatgattcaacctattaacatggccctggcttagtcgctagtaggacaatggttctcagccttggttgcaaattagaataaactggaggttttaaaaaatacccttgctaggacctcaccacaagactattaaattcaaatttttgcagatgggggcaggcatagagctatttaaaaagctcctctgggctgggcacaatggctcaagcctgtaatcccagcactttgggaggccaaggcaggaggatcgcttgaagtcaggagtttgaggacagcctggccactgtggcaaaaccccatctctactaaaaatacaaaaatcagccaggcatggtggcgcacgcctgtagtcacagctactcgggaggctgaggcaggagaatcgcttgaactcaggaggcagagtttgcagtgagctgagattgcacctctgcactccagcctgggcgacagagggagactttgtctcaaaaagaaaaaaaagctcccctacagccagactgataattgtcctctagcacatccctcggccagccatgagaaaggctcaaagagccttctgctgaactccttaccacattatccatcgatggtagaagcttcctggctttccaagcagggtctcaaagagaagtctgaggaacccagaacttccatgtgactgcaattacttcatgcattgaattgagtcaataaaaaacagcttccatcacaacaaaaaaaaaatgtgtgttaaataccaatatgcctgtggtaccatcctaggcatattacaaaagtattaccttgtgggaattagcaatctggaatttgcaattctctctccacccaatcccttgaaaagccatatgtggcattagagaaattcataacaaactggaaaatgtcaaagatgtggccctggacatttactgaagcgtatatgccacaacaaagtgtacactttggggtagttaaacaaatttctatagccatacatgacagttaagtgatacacagctttgagagccataactcctctgcatttcttactaactctagaaatctaacatatccattaatgtatttttctttttgcttttactgtgcagatttaaagaacaaaataaataggtaaatatattttcagaataaaaaagaaaacatcccgtattacttttgggggtgatgaaaatgttctaaaattagattatgttagtggttgctcaactctgtaaatatactaaaaggcactgagtatattttaagcaagtgagttttgtggtatgtatatctcaataaagcttttcttaaaaataatattgctagagtgaaaaaataaccgaaatattcccaccactttcagaatccacagcccgaatgtctcagcagagaagctgggtgtgctgtgtatgtatgtagcttatgatgctgtccctgggacgtgagttgtttgctcagatggcattgacaggggtgccacgtgatggggtaagtgggcagtccgttgctataagcaagaggcgggctgggagtagctctgcagatctggtggcaaaatgaaagaaaggagttttccctgttaggaaagaaatgcagaagcagctgtccacagacagcccaatgcagagaacaaacctgcctgggaagcagctgaggcagggcacatgctcctggaaggaaacagagagatatgtgtgctcctttactcgctagcctttcccgtcaccctctccccaggctcctggcaccttggaacagcgtgagctttcctagggctcctctcgacttgcttgatgtagaattaatttaattagtttcagagggatccatcatggtggctgtgttgcctaggttaaaagaaactctgtgtgtgtgtgtgcgcgcgcgcgtgtatgcatatatattttgccttcgtgtatgcaaaagcaccttgagacaatatcctccttctaacttgggattgctggactgttattggagtcctagggcttgacaaattggcagagacttggaaattctgaaacagtttaaggaaaatgcttacaatgatgaaggaaccaagaaataagtctctggggctcaattccctgctctgtagtacatgttctcaaaccttcctttcaagcattgcatggctaccatgggccatgtgcagtgttagtgctacagaagacgcagatgtcaatacaacccggggaacggggtcagacagatccccaaagacttcaatacaccccaccgcaagacagttacaaataaagtgctgggtctcagaagccaaggaaagcatgtgcagttgttgggaaggctatgaaaggcttcatgaacaagcagcactgagagctgtataacatttttacaggtgggagtgaggtggatttcctggaggaagaatcaatgtgatcacaaccatgagggcagggaaaggacagcaagtgagccccttaggttggaggataaggaattagcagaaaatggtcctgaaaaggtgactatggcagtaaatgccaagcacaggtgtccctattgaattccgcaggccacggacaagcctctggtgggttccacactggggctaattccaagattggggttccacattggggctaatatccatccttctgctaaggatgcctaccacccttggctggagggggacagccaaattcctgctctggttttaactcagtgagcttcaacacatcaatgtggagaaggtacttcgtatgaggcactctacaagtgggaataaagatgaaatagtgtatgtctttaaaaatccttgcagtttgggaacaaggaagaagggcaaagaggagattagacaacgacatgtagaaccatacatcgtatgagtggtaaaaggagcatcacaagagtcttgacaaagtgctcagacctccttaattggggttcctgccagcttctcaagaatgggaaccaggctttcttcatctctgcatggtctggtagctcagtgcctggcacttggtagacattcattaaatgaatgaatgtatgcatcagtgaatgaatgaataaacaaacaaatgaacaattctcctgtcacgcaggatctgctgaagatggcggctgtaagttcacagagcactgcctaactttggttcatattgttcctctcttctcctacctgtattttgctcacctcaataccagcccacactgttttcctggactgtgattctttcagtgatcacagagtctgtgctgcataatttaagtgtccctattccttgcagctgcttcctgtgtgctagtcatgtaaacgcccttcttaaatgattgcatccctcacaatacttagaatatattgactgttgattggttggaaaataatcccgtacaggcggagggggcagaaactgacatcatggagtgtcaggcacggtgctggtgctatgcatacactcaacaagggcctgggtaatgcaacatggagaagggaaaactggggggcagaacaattttgtcgtctgaaagcctttcacagagaggccctgaacccatagctctccttctctgaggacagaaaaggaggaagtgtgtctgtcctgcagtatgtgggatggatagatggatgcgaaattaagcactgaagtgggttgcttggagaggcaatgactgccccctgccctcacctgaaaatccttaaagacagaagggatcatccgcccaggaagctgaggctgcaggataagctggccttttcctgaacagtgacagttggagcgttcaccctcactgtccttgtcagagttacaggcaagaaaggagaatccttttggataaagtgtcttaaaggaactgttgatttacccttttgctttatggtctcctgatgtagtgagcaaggttcagggagattagctaactgcaggcagactgatcttcctgtgcagagaaataaggtagattctcattatcttccagctcattttcccaagaattatgtattctccggccagaaaacgacctccaaacctccaaaccttttcaactacacgcagcaaaaagctaatacaatgccttctgcatttgactgtacgtctcctgcagtgattaatgctgcctccctaactacattgtccggagcaagtgtcaagcccaattatttctctaggatctcctgatagtaaatgctcagcaaatatacagacgaataacacctaatcatacacaataatcttagagtactttccccatacatttcctaacttaatccttacaacaaccctgtgaagttattaactcccattttccagttaaggaaactgatgctcagcaaagttaactggcacatccagtatcatacagccagtaagaaagaattctgttcagtacgccttactgattaaatgaactatgtggcctgaagaaaaagtgtcagctcagtctttccccatcacgcatcttattccatggagtaacctgcattatatttcagggtcaggcacactaagtttgcgctttagtatatggtgaataaatggatggacacagtggcggctctgtataagtcagctgaaagcaaacaaagggaaggcagaatctttcattcacacgatttatccgcgcccactgggtgcactacggtcgggcattggcgacccaggagcgactgacctgccctcatggggcttacggtccttagcaaaaaacagatgtaaaacaactaatattacagtaaatgaatcataattttgtgagatgctcctgaaagtatgtagcaggacgctccgctctggtccgaatgaagagccatttgcactcagtgtggaaaggctagcctttttctgggcaaaagaagcgggggatacatttagataggcagaagttccgagcagaagtcaaggcaaattaaaaaggcaacaggaggagggaaaatgcttctcacaaacagaactcgacctaggggaccgcaactctccagtcctgctgcttggctgcctacctacctaagaaaagcaggaaagaaaaagagaagacggggtgggggatggaggacaaatacagaaaaagaaaaaagagcgaaggaagtgagaagtgagaggccagtttatgtaagcgggcagggcggcgcggccggcaggagaccggcgccggcgccgagggagttaagcggacgggcccggtatggaagaggttaaggggggcagtccggagcgggtccggtaggggccgcctactgggcgggggcggggccgcgccgagcgagcgctgggattggccgtcgggcaggcgctacgctaggacggtaccaacagccgggggcctcggaggcgggggggtgggtgtgggactgccgctctgcgcggcgagaggtggcctgggaatggccgggccgggggtgggccggagccgctgtggcggcggcggcggctgggggcggtgagcgcggcgtggggctgcccctccccggaggcggcgggggcggccggggccgcgccgcaccgcaccgcgcgggcggccatggagcgagcctagggcccgacaggtgagtggcgcgggggcgggggagggcgcgcgacccccgcccccggcgcggcgggcgggaaaatgcggaccggcacgcgggcggggcacggtacgtagcgcgcgccgggctggggccgcctgggggcggggcgcctccctgaggggccgggcaccgggtgccctgagccgtccccgagtggctgcgggcgggtcccgggcgagggcccgaggcttcctgggagacccgctcgggccgcgtgtccctgtgtgcggtcgcggctgaagggacgcggcctcggccgccacgtccctggcccacctgaggcgcgggcccacctggggccgcctcaggctgccagaggtccttctagtgaccgtctctcgtccccttgcgcggtccccagaggcgattggccccggtgcctctcttgtgttccccggagcgcccgttgaggcagcccagggagcgcgccctcgcggggtctttgctgggcccgcctcgtggccgggaagcgcgagtgacctgcaggccggaggcggaaggcggggcaaccgtgcccggggcccgcggtctcctggggtcctgcgccgggcgctcgcagaagggcacaacttaacgaatgctgtgttagccctcagtgggctgcgcttgagatttcatttccgggtgtgacggccccgcgattaagagcggaccgatacgtttcccttaatgatgaatgtgaatagttgtcgagctgcgtccttgattgtgggttctgataagagattcattctaatctgacttctgttaacctgccagtctccggataagaagagaaggggtagaaggatggattggagggaaagtaggggtaacagggcttggttagtcttaatggaattattagtccttttaaagaccataccatcattgggatggataaggagtttctaatttgggaaaatgagagttgcttgctattgcgacttgggtctagtgccagatatcagcatcttccccacccagccagcttccctctggaggtttgatttctcacttggaagggaagtaggaatgtggggttttttttagcctttagcctttaaactgtaggactttataaatctataatctggtacaaattactattgccatttattagccacgcctatgttttttgcgttgtaaattcttgcaaaaatttcactggcgttgatttctaaaaatctggttagagatctcagtgttcttatgtattacaaacattccttgagtttatgcaggatctttgagctaagcttagagcacttcctcaaatatgattcgttatcacgactgtctgagaaactgaggtacaaatggtaaagatgctaaattatttgcccagactcatagccaatctcagggagatttaaaatcaatgtattctattaataacttttctaaagtttgcaaagatgttggactttggcctctcgaaccgtacaatggaatcagtcagcaggagtccctttcccatttgaaattacttatcaaaggagaatacaaaggcacacacaagtataattcagactctcagcctcactcatgataaagtgcagtgataaatatggatacaggtaaaagatgatcaagacatcttggagttcacagcgtttcttttctgtgtggtgaaaatgtgacattggtatgctctattcgtttctctagaactggtaggacagtagctatgttttggtgaatgaatggaaactagaaaacacatacactgcttttctttttggttcttaagattagagaaaaaacacatcactgcttctatcatctttatttttataatcttttatatcatggaagtattttatgttaaattgataattccttgacttttagttcattcccttgatttccatttgtagattccagccctttcacacagccttaaagtttctgcttcttatctcttctgaattttaaattcctaaatgaaactttctgttccttcagttttagggcagacttcagcttcccacactttgtgtgtctatcatttcattggtgtaaaaccctaaaatcatttcattagtgtaaaaccctaaagccagatacttcagctgttgaattgataactttttaaaatagctaaccacagaggttgtcttttggtgattgccacaatctcataacagaagcaggagggcgttctttgtattctgtaagaatacataaaaagacagttggtcgggcgcggtggcttatgcctgtaatcccagcactttgggaggccgaggcaggcggatcacgaggtcaagagattgagaccatcctggccaacatggtgaaaccccgtctctactaaaaatacaaaaattagctgtgcatggtggtgcacgcctgtagtcccagctactcaggaggctgaggcaggagaatcgcttgaacccaggaggcagaggttgcagtgagctgagattgcaccactgcactccagactgctgacagtgagactccgtctcaaaaaacaaacaaacaaacaacaacaacaacagaaggagactgtcttctgaatctgactcttagtccttctgggactaagtgccaggtggttttgtggattgggatggggaacacagttacaatgaggccctacagtttgttgtaattgttacagtaatctgtgtatctgtgtatttttctctctgcacctgacagtgcctgtatgtagaaggtgcacaataaatattcgtcagttgaatgaataaatggaaaacagtatgagggaaggggaaatattctaatgtatatactaaattatttagttcctctttttgtcaaaagaaaaaaaaagcgtagaaaatgtgtttcggattactgttcaaaatattctgtagaggtgtgttatttgcttctgtgattatatggtgttctaaaccacctggagcctggcagtagcctgtgcaggttccagtcaagtgcagcttcacaaccttgctcctacagtctgttctggctaggctaattgggcagttggattgatagccagtgtttttgcattaatagaagaacttagagctcttggcaatggccatgtcaatagcattgacctgactgcccttgtcccttattttgacggtggaagaggggagttatccctttttaagagtacttctccagcagttccttcttctacacctacatatatcttcctgttttccttcagctgtctctgtaaccccaagtactaatgaattctttaacataggcctgggctgcagaacagaagcttactttgctattgaaaccagcttctgactaacttacaatgtcagcatgactgctcgagctgcctagaagagaaaataatgcaaagatctcacttctgatttggtctttcttaccaaaaacaaaagaagaaatgcctagcttgggatgtaaagaatctacatgacagatgggtgcgtctgtcatcccagtgtgagataagagaaaaatgtaagctgatgcagaagagaccttgagctttggtgtgtgtttaaggtctgtactttctggcaagataacccctttagagaagagggtgctaacttgggcacgttataaaggcaggtcaaggaatatgaaattatgaatgtgtaaagaggacagaaccaagtacagagggaggcagtgaagtctatcattccatgctctcaaatgttaactttctgctttatatacatagcaatgcctcatttacctaatgttggggagtaaggtattatatgtgaaaattttattttagctataccttttgtggaaaaatttttacaatatatgctttggttattattactagcatactggcagtagttgagtgaggctatgagatgatcgaaaaatgtagtttgttgtccactgtgctttttgaattattgaatctacttgagtcgattcagtagtcagtctttgtcttctttcagtggtggaaagctgctcttttgctgctgttagtatccttgctgccctcttttaaatttgcagcttcttatctgtggagtaggagcttcctgacattgaatgtaaaacagctaaattggcttagttatgttctacctgctcttctccaagttttagggtatgaactttgtaaccacttatttttgttgcccagggacgtttttgtttgtttgtttcttatcgttggtgtagatagtgaacgtgtgctctgtataggtgcatttatgttcctgtagttttctgtaaaaaaaaaaacaaaataatatggaagtacttactcgtgtgggttcttgtttcagaaagcactgccaataatacttgaagatctgatttcagccctgtcttgtatcatccttgggcagtgtgttagacaacctcctttcttccttttagcatttccaaattgtctttactcttaatcccagccgatactaggaaccaggttggcaatagcagtggccttgaaaggtggaagcaggatatacagaaggtggcaactttaaattgagtgtatataattggtatacatattcgctatttctgcatcttttatatactgtatacttgaattcattgtaggacttatttccccctgtctctgaaagaggcagtctttaaaccagagagttgccagtttagaagaaattcttataaccctaaaggtctttctcctttgcccttctctccttttattccacaaacacttatcagatgcctcttacaggccctgcacagtgctaagttacttggaagtacaaagcgtgagtaaaatctatcccatcctcaaaaaacacaatatattagaagagacagatgtgccaaaaaaaaaaaaaaatgtgtgtgtgtgtgtttgtgtgtgtgtgagttcatagtataacagagggatcatgcctggagccacctagccataatttcagcacagatctgtttgaggttctcctgcttctgagagtgatctcctgtgtgggctgtcagcttgacgtaccatagaacataaggctaaaaatgcgaggtctattgttgtataaagtttatgtggttttccaagaactgtagtgaattgaacactaaagatggaaagcatatgaaaagctgaaggaaaatgttataatatttgtaatgccgtatttcaacactatacaggaattaccttctataatctagccttaaatggtggttctgtactgacagttgtgctgtgaaatacctggaacttgggaagtgtcattatacctggcttatttaaaggaaaatgcaaattgttactcatttccacgtagtgtcaagattactgcgtgtgttggttttttagaacagaactcgctagcagtatgagctaaagtttttttctttttcatttttcaaggaagagggttgccctaaatcaaggtttaaatgggacaggttgcagagattgattttttttcagttcacttattgaaattattcagttattgcctatttcatttttactttcacatccagcaagaaagactaccttaatgcttcattaatgcctggtaatggttttagttgccttccttcagagggaacttgttagagaatttcatataagtgattctgagtttacagatgtaaataaatgtttaaatggaagtgaaaacagtaaggaaaacctgacacttggaccaatttggattatgtaagaattagattttctacacctgtactcttaagttactcttaaggcttatcttaagggctttagcacatgaaaagctgtcatctttaaaacacagaactaattttccaaactgaaaacagttgtgaaaggcattctattttagcctttttttgtgaagtgggataagttcatgcttcccatctgtttcagttacattgagtgtggtagaattgaactttgtagagaagggaaacaggttggccaaattagtgacagcctcccagtcttctgctttgcatgacactcactctgaagatatctatttgcctagtcttatctacctctcaaatttaggcaagaatttggctcagaaatacaagactactaattactactaaactcttagtaatgcaatgtgtggatttggcccctcactatgtaactgagaagacaacattatggtatattgattccaatgagggttttctttgtcatttttctcctctctggaggtcatctgggtttttgatatggtgataatgactagcaagtgattactgtgggtcatccaggcactaatcttagcacaacacatgtagaggtctcattcttttttttttttttttttttttttttgagacaggatcttgctctgtcacccaggctggaatgcagtggcatgatcttgattcactgcagcctcagcctcccgagtagctggctcattcatttttaacaaccctgtgagttaggtaatagtattcttgtttcatttttttatatgcagaaactgagacccagagaggtcagaagttaaatatctttcccaagacccacattaggtggtaaaggaagcatccaggcctaggcattctgaatccagaggacatgcttttaacccctctgctacagaaagcttatatttaaaataagaaggtgaattaagaagctatttcttaagtggataattagcacagaaaagaagaaaaaatatctatagatactgattaatgctattacttagtattagtttttatttttagttgaagaccctcatgtgaagatgaaaggaagaatagcatgctgttgtggcaggcagcaaagcaggagtgggctggttttcccagggtactcagcatttactgagataataaacatgtaatgctgtttaatattggaggttttgactttctggacatcttgactgtcacacaatactaaaagacttctgaatgctcttaattttacttatcactgccactaaccatcagtagagacatttatttctagggaacggggtgtaaactcataacagttaataataatatgcacagtgccctactgtcactgctagtgaaaaatcaaaagggcagtataccagacagcccacggtaaaagcgtgtgtgtgtctaactttttttgcactaaaagatgtgatttttgctatttatgttttggctgaagtacaaaagttaggctttataaaccggacatgtgtcagtgtgtgaatgtgtgaggttagccttgattgctctgcataagtctaagtagggctctgaagtatgcaagtggattttagtaaatgatccagttgagaatctatttcttcttgtggtacctttaagagactttgttaattatgtatctttctaaatatatacaatgaaaacagaaaaatataaatgacatgtaacttttataatgagtgtattgctcctagaagccattaataataatttacaaatatgacctaacaatttatcaaaccaatttttatttgcacttaatggttaatttagtgattactccctccctgagataattgtttttagtcacgtaatttatttccctatgtcttctcccctggtatttttactatccccaaaaggagttgataggagcagaacagatgtgagaatgttgggttttacaaactacctcaaaataatagtgggttaagacaatgatttgttatttctttctttctttttctttttctttttttcttttgagacagtcttgctctgtcaccaggctggagtgtagtggcgtgatgtcggctcactgcaacctccgcctcctgggttcaagcgattctcctgcctcagcctctcaagtagctgggactacaggcatgtgccaccatgcccagctaattttttgtattttagtagagatggggtttcaccatgttggccaggctggtctcaaactccctaccttgggtgatccgcccatctcacctcccaaagtgctgggattacaggtgtgagccactgcgcccagcctgttatttcttataattatataggtcatctggtctcacgtgttcctgctcaaatggtataggctggggtcacaacagtgactacattctgccagaccacaactgggactggaatgtccttgaaagtgtcatcttcatgtctggcacttcacctggggtgaccaggatagccaagggttggctggacctctctccatatagatcacttggcatttggtagtcttagcttgagcttctttacatggtagctagatcccgtgaaagtggaagctgccaggtctcttaagaccagggcccagaactggcacacagtcatttccacaacattctattgtcaaaacaagttacaaagccagcccagattcaaggggaggaaaaacagactccacctctttctctctctctctctctctttttttttaataatggagacagggtctcactgtgttacccaggctggtctcgaattcctgggctcaggcagtccttctgcctcagcctcccaaagtgttaggattacaggtgtaaaccagcatgtttggccctgactccacctcttgataggataaattgttagtgtctgtctttgcagacagtctacaaaaaattcaaattcaagtcttacctcctatactcaccaaaccttcagagttttagtcaaaccaataattaccaaagtattttgttaaaggatccttgtagttcctactcaggatcaagggcaaagagattgtgtgcataaatataattctataccagttaagaaatcctacccctttgcctcagatctcttttgttgttagtcctctgcatgaagcattcttactaagcatttcctttgtggtgctttgcatttacaaggcatgttacattctacttaagtcataagaacctgatttgaccattaaattagaaacacaagaagtgaaacttatgtttacttttaccataactttcacttttagttgctatgagcagaggttggtatttatttcttctctatgacctcagaggtagtattgtgagcttgtcacttaatcagagggtgattcaaggggacacttctaataaggcttggaatctcattggtggcttgtaggaccctctagtaaggtagtaagcgttaaggccaggtgttctcatgttccttggcagccacatacattgagatttatcacatgatctgtagacccccacaccaacatgcttttctgtaaatgttcctccaccatcagccagatttttttttaagtacaatttccattgtatttttctccagagaatggtctgtcttcagtctttaaggactcagctccttacacgggctttggtgggggtcgtggggcagcacccacaggtctaaatcggggtaggggtgttcggtcctcgtgggcttcatgagattgattcctgactactttgctgtgaattgcacaattcacacagtaatgtagcttcacaaacagcttgggaagcacataggcattgaagacgctgcttcagaaatgtccctgactgctgtggcctccactatatttcgaatgatgaatttcttaatggccttgtccttaggcacgcatcaggcacagttcatgcagcgaatggctgcacgtaactgtggccctttttgacatgacttttgttccttcttttctttgtcatcttggaggcatggaccggagaggagattcttgaaagaagactttacattaagacctcactttttttctgtggaatctatatctcttctcccaaaacaacttcatagtcttacgtttgcccttatacaatatttatcaaatgagtaggatgagtgtgtttgtgtgtgtgtgtgtgtagaatgaaagtgaattagccggctgggcgtggtggctcacacctgtaatcacagcacattggggggctgaggtgggcagatcacttgaggtcaggagttcgagaccagcctgcccaacgtggagaaaccccgtctctactaaaaatacaaaaattagccgggtgtgatggcagacacctgtaatccctgctacttgggaggctgaggcaggacaatcgcttgaatccaggagttggaggttgcagtgagccgagatagcaccactgcactccagcctgggtgacagagcgagactctgtctcaaaataaaaaaagaaagtaagtaaattagcctccgtagcaagccccatatagctgatggagctcaggttgaaaccagggttcagtgggaaaattccttgtaaaggtctactttccagaactccaggctgggagtttatgccagcttttcatgtgaaaactatttacagatatgccatttgtactgccttttttatatttaacacttttaaagttatgttattaccttgagtttgtttgttctttgaggtagtttctgttctgcataacagaaacattaaaataaatgaccgtaatataactggacattgttgcactgtttgaagcatgtatcggaggagctgagggtgtgaaatagagcaggcacattcttctgagaatgttagtgaggtttttcgtgaatagtttgaatatacccagtgaatttgcgtttatttccttagtgtctgaaatggggaatctttggaaaagtccagtttgcacaatatagtatactccttgtcccaaataaaaaaggaagattatatggcatggaaaccctgtctctggtactttctcctctctcacatataaaggagtgaaggggatcattctaaggagccaagattctgaaaggagaattaagaaccatatgcaataaggtattatacaaggccgaatgtagttaagtactggccgaaaggagggagaagttactttaagagtgtaaggagggacgaaaaattgccttagagatggccttggagatgagccttgaaaggaggctcagactttatcaggaggagattggaagaaatgttaagagaaagaggaattttgagggactacagtgtaaggaatgacagaagcaaagagcacctacacatcaagaggaggaccatagcatgttcagttgatccattgtggctaaagctgagaagaggaacaatgggagtgatgtctaacttggaagctatttaggatacagctttaatgccttgccaaggaatctagaatcaaaggtggttgagtgatttgaaaaatgattttggctgcattatctaggctggattggagaacctggaggctcaggcatcagttctttctgtggtccaaaagaaggtgatgagcactgaagttggtggctgtggaaatgaaagacggctgtaaagaacaatgacaaattgcactcttactcattccaccaggatgtggtgactgtaatcacaaacatgaaaacttgtttagtctctttttttttaacctgccctcacttagaatctgcagaaggaaatagtgaaataatagaacccttcagttcccttaatatgaatttatttcttgtttgcttgcttgctttctgtatgtcggtctttttttttttttttttttttgtgagacggagcctcactctgtcgctcaggctggagtgcaatggcacgatctcagctcactgcaacctctccctcccaggttcaagctattgtctcctgcctcagcctcctgagtagctgggattacaggtgcccaccaccacgcccagctaatttttgtatttttcatagagatggggtttcgccgtgttgtccaggctggtcttgaactcctgacctcaggtgatctgcctgcctcggcctcccaaagtgttgggattacaggcgtgaggcactgtccccagccagttcccttaatgtaaatttctaacctagctgagtgagtgaattgtttatggccctgaattgatgaatgtgttcgtctgaggccgcttcaaaaggagaagttttgaaataagtttcaaaacttaaaaggacacaaacttggattgtgggattggaccgtttttcttatctgaattttggaactgttagaacttcttattgcattgatgataaaaacaaaacaaaaaaccacagctctaacctttgtgcaaaatgtttggatgtgatatatttatatagcacgagggggaggattttcttcattctaaatgaatgagcaatacgattaaaagtttatgggttggtcgggacagtggctcatgcctgtaatcccagcactttgggaggctgagtctggcagatcacctgaggtcaggagttcaagaccagcctgaccaacatggagaaaccctgtctctactaaaaatgcaaaagccttagctgggcgtggtggcgcatgcctgtaatcccagctacttgggaggctgaggcaggagaatcgcttgaacccaggaagcagaggttgtggtgagccgagattgctccatggcattccagcctgggcaacacaagcgaaactgcatctcaaaaaaaaaaaaaaagaaaaaagtttatgggttaaccagactttagtcctgaaggatgttctgtagaaagtaaaagcctatgaagctgacccctttgtgccagaattgtattgcatttgtagctagtggcatctggagatgctgaagaagtgattgtcaatacaactagatttccatattgccttttatctctgcagcctgagaagtgtactgttgaacaagcacttgaaaatgttgtgtggtttaggttgcattagaactagcaaaaatctctgaaatcttctctgctagcactgctgttatcagtaatcacagggaactgttttactgagttccacaccaaacttgataccatcacaattacattttagatgcacttgaattgcctttgtttcagctaatagtgttgtgtaccctgggagatgatgatgaaacaaaacaggtggcagggaataattagaatttgaaattaactctcactttctgcttccttcatgaacagaaaaggagtggttaatcactgagaataaacttcattcagctggcccactgcaaagtgttgccctcagaatacattgctctgataagctgcttttgattgtttccagttgttattaagggagcagtatcacgtttaagtgggagtgtcttagcctgaatcatttgctctatttttttgtattggttttattttttggattaatggtttgagttgttaaatgaattggagagtgaagcaacatcctgttatcttcttgtcatctttgtcttacctcagtgctgtgctgtctgcaggcatagagcagagcaagcctgtgcatgtttttaaatggccttatggagtagaaagcaacctctcaggatgcaagtgagacggaattgtatgttcttgacacccttgcaaaaatcttgtatttggagccttttaacttaagaattgcccagagtgtggtcagccccatgctagaaggagcagttctttatgtctgtgacatgaatatccttgttatatgcttgaccattccagacgtagtagtgctgccatagtaccaggctttattagttttctggttatattctgctaatagctagatggtatgggcatatttttttccctttacttaatgtacccagttttgattatccatggtttgcaggtattttaattttgaaaaataattggcgaaggactaagtagaaatatgtgtctggggttgtatttaaatacatgagcagcagatatgacacgtccaggatcgctgaatacttggactcagagacagctattactctcctctgcaccctgtggcattttatttatttatttattatttttttttattgatcattcttgggtgtttcttgcagagggggatttggcagggtcataggacaatagtggagggaaggtcagcagataaacaagtgaacaaaggtctctggttttcctaggcagaggaccctgcggccttccgcagtgtttgtgtccctgggtacttgagattagggagtggtgatgactcttaacgagcatgctgccttcaaacatctgtttaacaaagcacatcttgcaccgcccttaatccatttaaccctgagtggacacagcacatgtttcagagagcacagggatgggggcaaggtcacagatcaacaggatcccaaggcagaagaacttttcttagcacagaacaaaatgaaaagtctcccatgtccacctccttctacacagacacggcaaccatccgactcctcaatcctttccccacccttcccccctttctactccacaaaaccgccactgtcatcatggcccgttcccagtgagccgttgggcacacctcccagacggggtggtggccgggcagaggggctcctctcttcccagcaggggcagccgggcagaggcgcccctcacctcctggacggggtggccggccgggcggggggccaacccccccacttccctcccggactgggcggctggccgggcagaggggctcctcacttcccagtaggggcggccgggcagaggcgcccctcacctcccggacgaggcggctagccgggcggggggctgacccccccacctccctcccggacggggcggctggccgggcggggggctgacccccctacctccctccaggacggggcagctggccgggcagaggggctcctcacttcccagtaggggcggccgggcagaggcgcccctcacctcccggacggggcggctggccgggcgctgatccccccacctccctcccggacggggcggctggcctggcgggggctgaccccccacctccctcccggacggggcggctggcctggcgggggctgacccccacctccctcccggacgggttggctgctgggcggagacgctcctcacttcccagacagggtggctgctgggcggaggggctcctcacttctcagacagggcggttgccaggcagaggctctcctcacttctcagacggggcggccgggcagagacgctcctcacctcccagacggggtcacggccgggcagaggcgctcctcacatcccagacggggccggcgggcagaggcgctccccacatctcagacgatgggcagccgggcagagacgctcctcacttcctagatgggatggcggccgggaagaggcgcctcctcacttccttgatgggatggcggccgggaagaggcgctcctcacttcctagatgggatggcggccgggcagagacgctcctcactttccagactgggcagccaggcagaggggctcctcacatcccagacgatgggcaaccaggcagagacgctcctcacttcccagacggggtggcggccgagcagaggctgcaatctcggccttttgggaggccaaggcaggcggctgggaggtggaggttgtagcgagccgagatcacgccactgcactccagcctgggcaccattgagcactgagtgaacgagactccgtctgcaatcccggcacgtctggaggccgaggctggcggatcactcgcggttaggagctggagaccagcccggccaacacggcaaaaccccgtctccaccaaaaaaatacgaaaaccagtcaggtgtggcaggctgaggcaggagaatcaggcagggaggttgcagtgagccgagatggcagcagtacagtccagcttcggctcggcatcagagggagaccgtggaaagagagggagagggagaccgtggggagacgggagacgggagatgggagacagggagggagagggagagggagacgggagacggggagggagacaggagacgggagacgggccgggagagggagacgggcagggaaaggggagaggggagagggaaaggaccctctggcatttttaggaattgttgtcttaccagaatgagaaccaaatactggtggtttgggatatgcaaaagtcctttatggattttgaaagttaaaaaaatgaaagttaaacaattatgtatagcttgctcttttgtttttgttgtgtttttcctcattgttttggttattaggcatgtgtagccaacctcatggctcctagatggcttgcattccaattaaaatagaaatatttgctttaaaattttcagtgtatgtggttcttgcttattagtgtcacaggatccttggggtgtcactttgccagtgagaaacctctgtggccgtggtgccttctgcctgagtagtgcttgtgcccactgggctcgttctacccactcagccttgcaggccgtgctcagctcgtactactggcctggatcttatacctgccaagggcaagccaggtgcagagcagcaaggggtgtgtggacaagctagcatggggtctggccactgtacacagccacgcacgctggggctgctgtggtggggtaggcagctccaagcataagcacagctgccggttctgtgtgagtctgcagctggaccagagtatcacatgcagcttctgctgcgggtgcccatgtctggacaaggggaacatggtggcatccagaaacttggagacaccaggaacctcagaaccccaaagaggatgtcacagccctggctcggggagaccctaggtctgagctccacaaagggccccagctcttctctctttctgattgcctaccacatggtgagcagggggtgtgtttcagccctgtttgcattacagctctttcagtcctgccatttggtgggttctgagttcttgtcccatgccccacgctgacaactgggggtgagcaaagtggagaggagcttcactgagcaatagaacagctctctggagacccaaagtggatagctctttctgcagccatgttgttctgatgagagtctagctttcagtggagaggagacccacagtgggtagatcccttgtgcacgcaggttgtccggatatctgtttgagtctggctgagtgcagggtttttatgagctcagaagagaggaagtgtgtgctgattggcccatgggtggccatgggtgggcccagaaaaagcaccataagttctcactgtggtttgcggactcttaaccagaaccgacagcccagcccccatgcttcaggccatccctggcttgaaggtggggcttcactggggacccatccctttccgcccaggagcctgcctcctgccaccattcatgtcatccacagtgcccaagctgtttgtaccaagggatgcctgcaggcctacactgagccatcctcagtcctcccttggcctccctcccatgctcattggtgcccaaagtccggagtgggctgagacagcaggggcctggtgtatcagcactgccccaaccacatatatacaggtcacgacatcacctgggctttgccacaattttgctccaccccagagtgggtgcggggagtggggagaggccaggcagcaggagtggcattttggagcctgtgggtgtagctgtggctgggtggctgcagctgtacccaggaacacggggcccctgctccaccatcttggaagagggcagggctcccacctgttcccagctccttccagctccgtggagtgtgcagccctggccatgcctctcccactgcagctggtgtcttcgcagcagctgctccagttgggccactgctgccatcattaggtccttctccagattagttaattttaaattattgaaccttcttgagctcaagaatagaaagcctaaaagggtgagtcatatattagaggcttacaaaagattcaggggtggaaggacagaatttttgatcaaattttgctttgtacttgggcttgcaggaactgtggaaggtgcatcagtgaagaaatggaccaatgtgtataatcatggaatctccttgctaaccatcaccaccagctctccttaatacatgagcaagagtgggtcaggggagaaggaaaagaggtcaacatgaagctaaagcagcgagtcgtgctgttagcaattctccttgtcatttttatcttcaccaaagttttcctgattgacaacttagatacatcagctgccaaccgggaggaccagagggcctttcaccgaatgatgactggcttgcgggtggagctggcacccaagctggaccataccttgcagtctccctgggagattgcagcccagtgggtggttccccgggaagtgtaccctgaagagacaccagagctgggggcagtcatgcatgccatggccaccaagaaaatcattaaagctgatgtgggttataaagggacacagctgaaagccttactgatacttgaaggaggccagaaagttgttttcaaacctaagcggtaagttttgatcttggaagctgcatgtgctagttggttgattcatttaacttgggatttatataagatttattttgtcatcttctcttggaagtctcttcagtaaaataagaggggtagactagatctctaaagtcttttctagcacttaacatgaatctacctttatagtatcatatagaatagtcttcctgccctaaaaatcctctgtgttctacctactcatttttccctctctgccccagaacacctggtaacccactggtctttttactatctccgtagttttgccttttcctgaatgtaatatagttgcaatcacagaatgtaagccctttcagattggcttctttcacttagtaaagtgcatttaaggtttatacatatctttttgtggcatgatagttcatttgtttttatcactgaataatattccattgtctagatgtaccacagcttaattatccattcacttactgaaggacatcttggttgcttccaagtttggggcaattgtgaataaagctgctataaacatccatgtgcaggtttttgtgtggacataaattttcaactcatttggatagataccagaagcataattgctggaccacatggtaagagtacgtttcattttgtaaaaaactgctaaactatcttccaaagtagctgtaccattttgtgtttctaccagcaatgcatgagaattcctgttgctccacatcctcaccagggtttggcatcagtgtttcagatcatagctgttctaatagatctcttatttctttcacctgttttaagtaaagaattgggccattttagttaccttagttaactaaatatgtatgttgttcatgatccctttggacttcctagggatctacagagataatttgtgtgacaattctgtgatacagttaattatttttatttatttatttatttctctacacaagacagcttggagggtatagttaattttctaaaaatgttttctgagtgtatgaatctgaattaggttaacatcatgatggtgatttgtattcttttgccgggagttagttaccaatgaatattctcaaagtttgtaatgtctgaaatcttttgggtactaaacatttttttcattcctcagtagagcttttgaaaaaattataacatgactttagatttctttctggagtgggagaagaaatgtagggaaaagatggacaaaattcaactccctttcttacccaactattagtaaatgaaaggcagtcagagcatgaatgtatagggagcccagaaatggaagggatcactgttggtctggaactgggaaatatatgtcagtgatgtggaaattaggaaagttaaaacatttggtagactttcagagaataaaaatttgactggggctgggcgtgatggctcatgcctgtaatcccagcacttggggaggccgaggtgggtggatcacttgagcctgggtgatgtagtgagactctgtctctataaaaaaataaaaaattagctgggcgtggtggtgtgcacctatagtctcagctacttggaaggctgaggtgggaggatgggaagattgcttgagtgcgagagtttgaggccgcagcaagccatgctcgtgccactgccctccagcctgggtgacagagtgagacctgtctcaaaaaaaaaaaccaaaaaacaaaaaaaaaccgtggttatgtgggcctgggatctaatttctgtaatgcacactatttgccagggattacactaagtacttttgtatctcttaatcctttaaacccttatacgaaggtttaacctttttggacaatttcagttcttatttcctgaataagaaactgaggttcagagagtgtaactaacttgacaaggtcacctcacatcaatcagtggtacagaagtttgaacccatatttgttagagttgtaaacagcttgtattctgccacacacatggaattctctaagtttgtttgcttcccataagtgatgacgtggagcactcatcatcaaaaatcgcatttagctttgaagtttggtctttgacttcttggcctgagagataagcagcataaacagagtaatggaaaaaggcaaactaaagacaaaaataaaatttgtatataaagagatggccgggcacggtggctcacgcctgtaatcccagcactttgggaggccaaggtgggcggatcacttgacgtcaggagtttgagaccagcctggccaacatggcgaaaccccatctctactaaaaatacaaaaaattagccgggcgttttggtatgtgcctgtaatcctagctactcaggagtctgaggcaggagaatcacttgaacctgggaggcgaaggttgcggtgagctgagattgcaccattgcactgcagcctgggcaacaagagcaaaactccatctcaaaaacgtaaaataaaataaataaagagatgagttgtgagcattttgtgtttaaataagaggagatctggctgggtgcggtggctcatgcctgtaatcctagcactttgggaggccgaggcgggcggatcacctgaggtcaggagttcgagaccagcctggctaacatgctgaaaccccgtttctactaaaaatacaaaaaattagctaggcacgtgcctgtaatcccagctactcggaaggctgaggcaggagaatcgcttgaacccggaggcggaggttgtagtgagccgagatcacgccattgcactccagcctgggcaacaggagcgaaactccgtctcaaaaaaaaagagatcctcatcacacctaccacttgtacactggagaattgaaggggagttgggacagcaggaagtgtgttagaagacagatattcccaagagcgtgcagtgttagaccaggtgctttgcaggataggcctgaaatgttcagacaagatataagtggtgggaggagaggccagactgatgagtaggggaggtagtaacagataatgcataaccttagtcacttcttgcttcatcttatatctcttactaccactgcagccttctcattacctccctcctccagaccatcctggaaaccacttccgttcttccccgttcttccccagaagacttattatcactatgttccccagctttaaaacatccagggacttcccattgcccaccagacaaaacccagcctccacagcctaaggtcaagcccctgactgtctggtcctcctctctatactcagccttatcttccttttgctctttgtttctgtcaaactctggccccctgctcccgcatcatctcctaaacagcctgtgtccactcccagcaacagggcactctcttctccaactcagcaaggttttttttggcccctgccataggtgctaactctcttagtcactgcacatactgacctgtgctgctttatatcaggaaatatcacaggctggggaaagtggttgtaagtttaagtgcttgcagaaattgactccagtccctaaacacccagcaaaatgctgttctgacccccagggcttcagaggctatttatttagtcctggtgtaagtgcaaattaatgtccttggtcatactctgatgtccttcagcagaaggataggaatgatttattggcagttactgtatgaattttgctgctctgtcagtgacttaagatggatcactctctggtaagagaagaatctaagtcctttctctgaaagccacttgtggttttaacatttctggagcctgattatccatgtcctgctctcagagatacaaggtgtgtgcagtataggtagatgatatatatgggaatagagtgacacttttatttacacattttgccttacattataagtaaaggtgattttttttttaatgtagctatttccattctagaagtaattgattttcacaggcaaaaacagcagttatgttggaaaaagaacaagaaggcaggggcctagaagcgcgtagaagtgaatgagtaatctcaggaagtactaaaagggaccttggagatcagctagtccaactgtccattttacaggtgagaaaaatgagatccagaattctgattttccatatttcacataaccaaaacatgagctgaggattcaggcctgacttcagtgtagttcttttgattcttattccttttctcttccctccatcttttcctctcctgcccactcccaaagttcctgtattcctctaagcttttgcattttactctgcgcagcctttagatggattggaaagctctctaatgtttcactgaaaggaccatactgcacttcaccacataccttcctgtggctgccctgaagtactaaaaataaagtattcaaggatagcctacatctgtagtagaacagtacctgaatagtgtcagctttttgctgcaggggtatttcaaatttgacttttaatttagaggtagctaatataaaccttttaaaaaattattcctaagtaatcctttgcactaggaacaaacaaacaaaaaattacatttgggtatattgaccatgctaagaagaaactggtgcccaaccagaaggtgatacatggctgtcttctaacaaatgcttcatcattttgccttgaagatatattttgtggatatgatttcactttatattctaaggtcatttaagttgaagttcactgccttttatagtactgttttccacttccctattgctgttttagcatgagagagccaactatccttagtggaacctctcttctccacatcactctcaatttatttctatttcataggtccattgtaaagcctaaaaggtctcctttagattcaggaaatgcaggagaccaatagagaggacataggtgaattaagtacttttttcttcctaattaacagatttaaattctatcttttaaaatggttagtagaaagaaatatgccagtcttcattatcctttgcttatgtcatacatgtgatagaaaatcatcagaatgcagtctgagatgaaagagaccttagaaattacctcctagtccagcccctaatattgtaagtaaggaatctgagacccagagaagagaaaggttttgctcaagcttacccagtcaagtaatgacagccacaccaacctcccactgccagtccaggactccttgtagtacgtcatcataaacttaaaaaaaaagaacaagaatggttattcaaacaacttttttcaaaagcgctgttgccttcccattaacaaagattgagttatatcagactattttcccccaaatttaaatttctcttataagctttgccacagccattctggtaagcaaaagttttttgttttttgagacagagcctcactctgttgcccaggctggagtgcagtggcacagtctcggctcactgcaacctccgcctcccaggttcaagcgattctcctgactcagcctcctgagcagctgggattacaggcattcaccaccacacccaggtaattttggtgttttttagtagagatggggtttcgccatgttggccaggctgttctcgaactaccgacctcaggtgatccacccatcttggcctgccaaagtgctgggattacaggtgtgagccaccgtgcctggccagcaaaagttttaattacacatttttcttcatttcttcaggtcagtctaataaacattttagtagacatttattaagcaccaactatgtgcttaacactgtgctggcaatacaaagacagtccttgccttccagaaaactaccttttatctgtactaaaactacatcaaaagccctttagtggggcaaaaatgacagcttccatgttttattcttagaatgattggtgacgtagatagagaattctagtttccttctctacctcatcttcattccatctaccggcaattcatgctaatccacagttgtttgcagggttgacagagaaggtgactatagcagaatcaggcttgctaatgggtgtattctaccacttgtgctgtcacctgttactggtgtaatccacgtatacatctgtgcttcccattcactttgcaggtatagccgagaccatgtggtggaaggggaaccgtatgctggttatgatagacacaatgcagaggtagcagcctttcacttggacaggtgcgtatgatcacagcagcttatgttcattttgtttgctttcaaaaatctttcttggagaggactcgtggactccttcagaaggatgcaacactaataaataacttgttatcgattttagatagctaattgactagttccattaaacttgtatgagtctcataatttggttggcaccaggaatactgaagtattttacttccatgcttctttaggtcatttcatttctggcctgctctgttattatcatcagtgaagatattcttggtaaataaactttgaacttaaatctctgaattgaactggtcgttaattgggtctagccatcctgttgttttgttatgtatttgttttaagtaagtaatgctgacttatttccatgtgctggaaacatatagcatgaaatatgagttttattattctttatacaaactatgcacagcttcaaaaaccagaataattacaaaaggaaagggccaggcgtagtggctcatgcctgtaatcccaacactttgggaggccaaggtgggcagattgcatgagctcaggagttcgagaccagcctgggcaacatggtgaaaccccgtctctactaaaatacaaaaaattagctgggcgtggtgacaggcacctgtagtcccagcttcttgggaggctgaggcaggagaatcgcttgaacccaggaggcacagcttgcagtgagccgagatcgtgccactgcactccagcctgggcgacagagcgagactctgtctttaaaaaaaaaaaaaaaggaaagaaaatagatgtgaattataagtttgtaagttaactaggaaaattctttttttttctttttcagcaggttactagtcaggaaccttgcagctgaactgtctggtatcccagttactctgctgaccagctctttcataaggccatattggctgactttaaaagaaactgctaacaatatttgttttaaaaaattaatttatgactggatgcagtggctcatacctgtaatcccaacactttaggaggctgaggtgggcagatcacttgagcccaggagttcaagaccagcctggacaacatggagaagccccgtctctactaaaaaaatacaaaaattagctgagtgtggtggcgtgtgcctatagttccaactagtggggaggctgaggagggaggattgcttgagcctgggaggtcgaggctgcagtgagctgtgatcacaccactgcactccagcctgagcaacagagtgggaccctgtctcaataaaataaaataaaataataaataaattttttttctgagacagagtctcactctgtcctccagactggagtgcagtagcctgatcttggcttactgcaacctcgacgtcctgggttcaaacagttatcctgcctcagcctaccgagtagctgggactacaggtacgcaccaccacacccagctaattttttgtatttttagtaaacacggggtttcaccatgttggccaggctggtctggaactcctgactcgaggtgatccgcccacctcagcctcccaaagtgctgggattacagacgtgagtcaccacgcccagcctaaaaatgaatttataaggaaaccagaaaaaaaaaggtgaaatgtttgtctttttctgaattaggattttgtaaacataaaagcaataacttaaaaatcacaaattcaatagtcttgactaaataaaataatgaacttatatagcaagagacaaaatttaaaagcaaacaacaaacggagaaaacacttgaatctaatgtaatctgagtattaatagccttaatatgtgtttatacaaatattaatggtcttaatatgtgtttatacaaacatatgtatgttaaaactctattagaaaatgggttaagagtataaacattgtcaattgctaataaatatgtggggtggggggaggttcatcctcaccagaaatttttaaaaattcagatgagatactgtttttacctgttgaaaatggcaaagcttaaaaaaaagataatactttattgggaggaatacattgagattcatatgctgcttatggcattgttaattgtttcagtctttttgttaagaaccctagcattatatatcaaaattaaggataaatatatcaacattaaggaatgttcatatcctttgaacaaataactgcttttttatacacatatctaagacatttctaggaactaccctagagaaagaatacaaaattcaaagatatatatacaacagtcttagctgtagcattttcaatagtgggaaaactggttgtaacctaaacatctattgatatggaaaagcttagatatgtgtgtacaataccatgaatttttgctgccccttttaagacctagtggtggacagttggataaatccttctcctcacttaatggcacactagaatcaggctttccaagaccttatctgtttgcctcttttcttcgtccccaactcccttcttcctatcctgtttgttggaaaccaacaggcataaagttcctcatttctgtaccagtgttttgtattgcccaacacactaagttttgttgatatatatgtaaagtgatatttcattgttatttcattgttattttgtatttccccattactggcaaagtttatcttttcacagcaggacacctatttgttatcttagagtatttgtgtacttttgatgttaactaactataatgtttcagtttgtgacttgggctttgggagtcttgtatttaaaagtccttccctacctcaaagtctgaaagatgttctcttgccagtcgccctgtcacttttgttaagtgccaccgtagccagcctcaagtgccacagtaacattgcccctcaatgctaaactggctgctgttcttgggcttggcaattttcaatgggaagaaaaaatacttcccaatctaggctgcgtttgtagtcctaggtaccagggaggctgaggtgagaggatcacttgagcccaggagttccaggctgcagggagctatgatcatgccgctgcactctggcctgatcaacagagtgagactcaaaaaccaaccacccaaacaaacaaacaaacaaacaaacaaaacacacacaaaaaacccttcccaaccttttccatttgtggatcttgatacttttctgctttgggttgctgtttcttcattacagtcagatttaaccaccttcccttgcctctgggggtatctctggatttctatgttattttgacaaatctgttgatcttgcctttgtggcttttctaagactgtgtttaggacagaatcagcagccctaaacaatagccatttgtaaaggctttctaatgactggggaaaaggttcatgatacaatgatgagtacaaagcaagatataaaactgcatacaatataatctcgattgtatgtatatacgtaagtgtgagtgtgtgtttatatacccacacttaaagggaaagcatgcttatatgccagaatgtcagcacctgtaattatttctgggttgtggaatcataggtgatttttattttcttcattgtaaattttctgtgtgttccaaattctgcccctccaacttttgttcagttggtgtatattttttagccaggaaatttcatttttttaaaataaatcattgttttcaaccttttagattattagagatatcttcaaatgtctctatatttaatttgctctgtgggcttagccttttagatcagtctgcttttttttttttaaccaacatgctgttttatttaaagatttgaacccatatatgagtttgggtcccattttttttctaatactttcctaaaatctatttttaatttttattatgaaaaaatttctatcatacaaaagtagaaagaatagtataatgagcccatatacccactctgtagatttaaaaagtgttcacattttatcagatttgtcttattctttttgctgaaatgtatattaaaataaattatacacaccttcacattaaattcctaaatatatgtacctctaaaaaattaggtttagctactagctaaaaaccctttacacatttgacatgatgaactgtaatttttaagactgttacttaaaaaacatcccctaagatttttctcttctgttcttcaatcttccaaaccaggattctgggtttccaccgagcccccttggtagttggcagatttgttaatcttcggacagagatcaaacctgtcgccacagagcagctgttgagcaccttcctaactgtaggtaagaagattgtagaggacatttatataggggaatgattaataagttaaaatggggcattgttgagcaagctggctcatgacttttaggaattgaagaactcagtggaatacaaaagcaaatcagacaaccagctctcaggttatttgggaaaatgaatctaaaggagatggcaggaaattgaaattttcctgtatactttgtgttgtctgtgacatagggagtgaggtcatttagtgatggtgaaagcaggtgattatttccttctgctttctcttagagctattagtaagtctcaaactcagaatttggcctaaattggttattgtttgtatatcattggctcagttgtttttacatatataatatgtttcctcaggaactgtctcccatctccttagaattgaggaaatcttaaggcaactctttaggaggctcagaaattattttgtaaggttgtatttggacctggaagatcaacaggcctactccttctgcaagggttaacctataactcattgtgcctggaactggctatgttgcccctggcctctgttttccattatcttaactctgaggtaagagatctgagctaaaattctcactcctgttgttgtgagcaagatgagaatgggaagaattcaaaagtgcggtggtttcttgtttccacctcttcaccttgactctttttcagtgatacagtgctctgcattagggcatgactgttgaaacagacattgcaagatgaacttcaccttgcttttttttgctagagtgagtggatttacatttaaaatgaggaaaaactgaatatttaaaggacttatgacagattcatcattcagaatttcaacttataaggcttgggctatacttttcttatagataaccaaaagcatacaatgttagatattgtatcagatatttgtttcagatattttctaatcacctttggttgttcagttttatgtttagcaagcatgaattcctggtgggtgctgggattgagggaaagagtccaagaagaagaagccagggtacctgctcttcaaaggctcacagatggtcaactcacacattgtatgaagtgtgggtaagggaaaagcagaggatttgtagaggaggttattaacacttgaactgagtattgaagaaccagtatgagttaccaaggcagcaaagaggaaagggcattggcaaaataatgggaatattaatgagaacaagggcataaagactggaaagtaataggtagagggatttggctggaggtaaagttcttaataatcgtctgaccttagcataaccttgttgttattgttgtgtttttagactttattgtttttagagcagctttaggttcacagcaaaactgagaggaaggtacagagatttcccaaataaccgctgcccgtcacatgcatagtttcccccattatcaacatctcacatcacagtggtacagttgttacaattgatgagtccacactgacacatcataatcacccagactccatagtttacattcccattcactcttggtgttgcacattctgtgggtttggacaaatatataatgacatgtatacatcattatagtatcacacagagtattttcactgatctaaaaatcctctatgttccacctattcatcattctccacccctctctccaatccccttgcaactactgatctttttactgtctgcataattttgccttttccagaatgttacatagttgaatcacagtatatagctttttcagagtggtttctttcacttaataatacacatttaagattcctccatgtcttttcatgcatggcttgatttatcatttcttgttattgctgactaatgttccattgtctgatgtaccacagtttatccatttacctattgaatggcatcttggttgcttccatgtttgggcagttgtgaataaagctgccataaacatctgtgtatagctttttgtattgacataagttttcagctcttttgggtaaataccaaggagtgtgatttctggatcatatagtaagagtacatttagtaagaaactgccaaattgtcttccaaagtggctgtaccattttgcattcccaccagcaaagagagagttcctgttgctcctcatccttggcagcatttgatgttgtcatattttggattttgactattctaatctagtaggtgtatctcattgttttagtttgcatctccctgatgacatataatgtacagcatctttacatatgtttttctgtcatctgtatatctttgctgaggtgtctgttaaggtctttggctcatatttaagttaggttggttgttttcttactgtcaagttttaggagttcttggtatggtatggccgaaagtcctttgttagatgtgtcttttgaaaatattttctccagctggacacggtggctcatgcctgtaatgccagcactttgggaggccgaggtgggcagatcacttgagggcgggagttcgagaccagcctggccaatatggtgaaaccctgtctctactaaaaacacgaaaattagctgggcatggtggcacacacctgtaatcacagctacttgggagactgaggcaggagagttgcttgaacccaggagacagaggttgcagtaagctgagatcacgccactgtaactccagcctggtcgacagagtgagactccatctcaaaaaaaaaaaaatattttcttcaaatgtataacttgtttcattctcttgatgcaatttttaaaatgtattattaagtaagttggcctataatacaagaataactaaaatctgtgtagccccggcaacctacccaagtatgttcacactgttactttatctgatccttttgacaatagcatgcaataagtaggtgaggtgttacccacattttatacctaagagtggaggcttagaagtgtctaatgactttcccaaagtcacaaaactactaattgttagccaggatccaaactcagattttaagactctaaaatctttcattagcttatagtctttatttattcagtacgactttatttcacttctgtttgacaggtactgagctaggtgctgaatatgcaaaactgaatgaccttcttatcttcaaggatctttgtctagtgcgggagccagacatgtacaaaggggctatggactgttggggtgggggtgggaatagggaggtggccaagaagttatggccaggtgataattcgtgaaggtgatattttaaccaagtcttaaagggaaaatagcagtctcttaggtagatacagtgggaaagaatattttaggtttgtatttaacattttttgtgctatagatacctttggcagtctggaaaagcttacagatcctttctaaaaataaaggttttaaatacatacagaaattaaaatacatagacttataaatgaaaccaataatattgaagtataatttcagtatcaaaaattttaaaaagcaaatttagatacagtaatatgtgctttgttatttaacacattaaataagatctatcagattagtcactactgtgatttcaaactatgattataaatagtattttgaggttatctgcaacaatgctagtatgatatgaaaaatccgtgatttcttgtagagacaaagtcacatgtaatacaaatatgacggtttgctccctgccatcataatagaaggaaatgctaatttttagtagaggttagtgaaaataaagatgtaattttgttcccatccagcttacagaccctatgagttgaccctgttccaggtagaggacagagcctggacaagggtgatgagcaatgagagaagagcaacaagctcaggagactgtgctgtttagctgaaggaaaggatgtgtgggggctggccagtgaggaagacagctggtcaggacagtgtcactgaagggcctgtgtaccatgcttaggagtagagactttctctttaggcactagaaagccattaaaagagtttgaatatggaaaaatcagatttgaatttcaagagcgaattgtggcagcattgtcaagggtgcattgggaaggggagaatcaagagctgagagcactctgggagtgttgtgaattcatatgtaataagagaactaaagcagggacagggaatagaaagatttaagggctattcaagagttggaatggaaagaatttggtgaacatttgggctgggagagcaattgagagagaagaaaaaattaagaatgactcctaggtttctggattggatgacaaatgattagtggagctgttaactattttaggaatgtagggagaggagcatatttagggaggtggagcagagaaagataatgaatttaatcttggatattttgtttccttcacaggcacatccaggagtaagtgtagactaatcatttaggagctaaagcctacagtttaggagaaaaggcttggctgaagccatagacattcttgtcacagttggtagttcacccaaggagagcatggagactgagttgatcagaaaaccagtttttaaaagtttggcaatccaggaaatagaggttcagagcctcagctcatggtactgaaacagggatattgaactctggaaaggtagatcgaaccatgttattaaaataagtagctggcatcgattggtgtctgacaatatgtcaggcactgtgctaagcattgccatggattgcctcattcactcttcacaactcagtggtggaggaggtactattgttgtctccattttacagaagttgcttagagagactaaattacatgcccagagtcttgcaatctcatcattaaactagtctaaatgttaaccccaggaacctgacctcagaccccacattctcccttctagttatgaagattcttacttaccaaactcaagagtttggattgtatcctataggaaacaggtttcctataccaggcagtattttgtttgttttagggctttacatttttatttgaatcagtaatggattcacacaattcaaaattcagggaaattcaggccaggtgcggtggctgacgcctgtaaccccagtactttgggaggctgaggcaggaggatcacatgaggccaggagtttgagaccagcctggccaacatgatgaaatttcatctctactaaaaatataaaaattagccgggtgtggtggtgcacgtctgtaatcccaactacttgggaggctgaggcaggaaaatcacttgaacccgggaagtggaggttgcagtgagccgagatcatgccactgcactccagcctgggagacagagcaagattatgtctcaaaaaaaaaaaaaaaattctgtgaaattcaatgaaaaattttctcgtctgtctagttccttcccctttctccaagtggcaacctttaactagtaatttccttatgtctttttgtatgaaatatatatatatttttttatttatctctaccattttatacaacatatggcacactttgcatacttctgcatcttactttttttacttaatagattctagaaatctgtatcacatcaaaagagcttcattgttttgttgtttcacactgcaaagtgttccattatttgggtctaccataattcatataaccagaattttattggacttaaatgttgttccaatattttgtttttacaaccagtgttgcccagaattaccttgtatatatgtcaggggtccccaacccctgggctgcggaatagtaccagtctgtggcgtggtaggaactgggttgcacagcatgaggtgaggggtgagcgagcattaccgcctcagcaccagctccttgtcagatcagtggcagcattatattctcataggagcacaagccctattgtgaactgcacatgtgaaggatctaggctgcacgttccttatgagaatctaatacctgatgatctgacatggagcagtttcatcctgaaaccattcccacccagtccccaacccctgtctgtggaaaattgtcttccactaaaccagtccccagtaccaaaatggttggggaccacccatatatattatttcacacatgagcaagtatttctagaaatgggagtgctaggtcaaaaggcacgtgcattttaagttttgataggtgtggccacattatcctatatagatgtttaaccagtttatattcccatagaaatatataagagtgactctttctgctagcttcaccaacaatatgttattaaacttttggttttgtgtatttcatatgaagcatctttttgtttgagagctatttgtattttctgttaactgtctttgcatatcctttgctcattttctgctgtgtcattggtcttactgacgtataggagttcatccctagggcatatagtcttttgtgacatgagatgtaaatactctttgtcgaattttttttttttttttttttgagatggagttttgctgtgtcacccaggctggagtgcagtggcacaatctcggcttactgcaacctttgcctctcagattccagtgatcctctcgcctcagccttccgagtatttggaattacaggtgcacatcactgctcccagctaattttgtatttttagtagagacagggtttcgccatgttggccaggctggtctcaaattcctgacctcaagtgatcaggagttcacttagtatgatagttgccaaatggtgattttttttttgaaatgctctctctcagcagtatcaccaagtgatgatttttgtatttttatttttttgagacagagtctcaccctgtcacccaggctggagtgcagtggcacgatgatggcccacagcagcctaatctcctggtctcaagcaatcctcctacctcagcctcctgagtagcttggactacaggcatgagtcaccatgcctggctaatgtttttacttttagttttatagagacaaggtttcattgtgttgtccaggctggtctcgaattcctgggctcaagtgatccttctcccttggcctcccaaagtgctgggattacagatgtgagccacagttcctggtcatcaaatgatgatttttttcattctatcgttgcttctagacctattagttgtcattctgttgaagggtagaactcccctgcctccacacattcatgtgcttatttatatcaccatgaactcactatgagctacaatttatgaccattataattatttggatgcttaaactgttccacatttggccggtgggaacctctacaaactagttcctgtgtcattttggaatggtcccatcattctccaagcactttcttgcaactggcataacactgtggtccaggatcattaaaaggaaccctagttccttttttttttgagatggagttttgctctgtctcccaggctggagtgcagtggcgcaatctcggcttattgcaaactttgcctctcaggtttcagcgatcctcctgcctcagccttccaagtagttggaattacaggtgcacaccactactccttaattccttttaatgtggtactaaaatggtatttaggaactaccctgtgggcgctaggtgtgctcattacttcctgggtgtcattgtttccatgttctcttgaaggacagaggtgggaaatatatgtatatgtgtgcatacatacacacaagatacatctgtatctttttctgaatctgtgtgtattaaaaccatcaattcaggcctggcgcagtggctcacacctgtaatcccagcactctgggaggctgaggtgggtggatcatgaggtcaggagttctgagaccagcctgtccaatatggtgaaaccccatctctaataaaaatacaaaaactagctggcatggtggtgggcgcctgtagtcccagctactcgggaggctgaggcaggagaattgcttgaacccgggaggcggaggtttcagtgagccaagatcatgccactgcattctagcctgggcaacagagcgagactccatctcaaaaaaagaaaaacaaaaaacaaacaaaaaaaaacacccatcaattcaaactagtatttccaattctaattcaccactgcaggatttatcctggctttctctctttctgtatgtttaactctcttctccactggtgagaagcctggttactgttacactcagtctgtctacttatttgctcaacttccccccttcctctatacctacccctctatgtaaccaaactcttggccacacttggcctagtacatgacctcttttgccctcccctgggcactctggccaagatctcagccctaatgaagaagaggaggatgttactttcctttaattaaagtgagttcaggccgggcgcggtagctcacacctgtaatcccagcactttgggaggctgaggtgggcggatcacctgaggtcaggagttttgagaccagcctgaccaaatggtgaaaccctgtctctactacaaatacaaaaattagccaagcatggtggcatgcgcctgtaatcctagctactcaggaggctgagacggaagaattgcttgaacctgggaggcagaggttgcagtgagccaagatcttgccactgcactctagcctgggcgacagagcaagactcagtctcaaaaaaaaataaaatagtgagtttagaggccagacatggtggctcatgcctataatcccagcattttgagaggctgaagtaagaagatcacttgagtccaggagtttgagcccagccaggccaacatggcgaaaccccatctctaccaggaaaatacaaaaattagctgggcatggaggcatatacctatagttccagctactcaggaggctgagatggaaggatggcttgagccccagaggcagaggttgcagtgagctgagatcacgccactgcactccagcttggatgacaaagccagaccctgtctcaaaacaacaacaacaacaaaaacaaaacaaacaaaaagtgaatttgaaggatactggtgatggtagttgacaccagagagtgagactgggtagaaaggacacaggaggcaagggcaacttacttttgggtgtatatccttttgtactttttaaattttttttacaccgtgtgcttttttattacctatacaggaaaataaatgtttgaaatatagtaaactcagggtctttgttttcaccatgcttatttacttagctactctgttctgcattatttgggagagaacttggagtcttcgttatttccttaagtgtatttggctcctttctgtcctttaggaaacaatacttgtttttatgggaagtgctattactgccgagaaacagaaccagcttgtgctgatggagacataatggagggatctgtcacactttggcttccagatgtgtggcctctgcagaagcaccgtcacccatggggcaggacttaccgagaaggcaaattggccaggtaaatgctcctatgagccattacttaattctcccctgtgcctagccaggtgccaactctgtaaaggagccagcagttctgtccaagagtgaaaagaactgtggagtcaggggcaaaccggtaggtaggaactctgtcttctctttgtgtgtaacagtgccaggtacagtgttgtcactcagtgctgtgatgctgcttgtctccaggtgggagtatgatgagagctactgtgatgctgtgaagaaaacgtccccttatgactctggcccgcgcctcttggacatcattgacacagctgtctttgattacctgattggcaatgctgaccgccatcactatgagagctttcaagatgatgaaggcgctagtatgctcatccttcttgataatgccaaaaggtgagaccagcaggactgtccttgtcagggaggggtttctgtatatgaaagaagggcattttccagagcatcctggagaatatccagaatgcaattgataggcaacatccttcttttttccacttggagtctttgctattggtacctgtccttctctccactctcagggatctcagtacttcaaaaaatggaaaagtggttcttccaaggagtctaagactctatgagcctcttctcagacaactggaaaaccagggttattcatttgttactacctacatcgttctttcttggctatgatatttggtgtttagaagcttgagaaatacagatgtaatagagtgtggaatttgtcgttaaatttattctatccaaaggttcctgcaattaaaaaattaatttatttggtaggatgttttgctatttttgtgtttgtctaggtccatacttttgctgtcatgggaagaagagctgttgcctagtgtggattttatatataacaacaagttacttttatcttgaataaaaaatgtttattcattttgaataaatagaatgtccttatttttttaggcctgagctatttggagagcacattgtatttccatttcatattctgcattctctgtggtggccggtttagtgccacactttttttttttaagtttttttgagacggagttttgctctgttgcccaggctggagtgcagtggcacaatctcagctcactgcaacttctgtctcctgggttcaagcgattctccaccctcagcctcctgagtagctgggactacaggcactcgctaattttgtatttttagtagagacagggtttcgccattttggccaggctggtcttgaactcctgacctcaagtgctctgcttgcctcgacctctcaaagtgctgggattacaggtgtagtgccacacttttagttgcatttaataaatatttactaaatagaacgcttccttggagtacagaattttccaggccctaggtcactactgttaacattggttgttggaaataagctaagacatggctcctcagcaaagagtgcttgggcagcatttgctaatgatgccaccatcttatactgaacaccaggcgcctcatgctttgtgaaagatggtgatggccacagaaaatgaaaaaccagcaaacactgcctctccctctaccccattacagcaaagcacatagcactcatcctgtgatacacatgatacttgatatttggtgtatatcaaaatagttgctttgtccactcgggccgctataacaaaatcccttagactgggtaattcataagcatcagaaatgtattgcttacagttttggaggctgggaactctaagatgaaggtgccagcagattcagtgtctggtgagggcctgtttctcagatggtgccttctatgtgtcctcatatggcagaagaggtaaacacgctccctctggcctcatttataagggcactaatcccgttcatgtgggctcttccctcgtaacttaatcatctcccaaaggccctacttcttaatactgttggactgttgtaccagggattacgaatttgagggggacacaaacattcagaccatagcagttgcctttgccttgaattactggaggacctacactgtactaagggctaccagactctagctattaaggtgagcctctggaaagattgaaaggttgacagttttccagaatcagttctccttttctaactctgaattgctgctaaggtaatttttgcatctgctgtgaatcagtttcctcagcctgtgtgaacagtttttccaatttgaatctgtacttatacttacttgtagggaataggatattgctaatttgggagtagaataagtgtgacttctttttacaagtaatggtcatctgcttgtttgtgggaatctaatataaatggcttttatttctctctggtccttaaaacctttgcaggcttaggggtcttcaagttctccctttggtctgtgaactccgcactgtgtttaccatagttatgctccagcaccttgtgatatcttggcaggaaaatgtcccttgtaaatgagtatatttgttgtcagatattttagtacaggctgttagggatcctgtgtctaattggtcatgccccgcttgttttcaatttaagtgatttgagctgctggagcgtgaaattatctaggatcacatttgagtttcatatagaatttgctattgctttgatgctaagaagagaacagtacttccacctaattcactaagttttaatttaattttcagctttgggaacccctcgctggatgaaagaagcattcttgcccctctctatcagtgttgcatgtaagttatgcacagcaaatacatgtgcctgcattgccttcttttccaggctcaggtaacagtacatccattttcttggccctcaaactttctgatacctgagcagtgcttttcatacttttccaacctagaacctctggacatcctgaaatgggatatcagggaaaggtgaaggcagataacccaaagctcagaattcatctcaaattttgtatgaatagaagattgtatgtatacagtaatacatgtttggtttaatagcagaatataatcacccacactccccaccccactgccaaaaaaaaaaaaaggagtaaaatctgtatgcctggttactacacctggttaccttgtggttttaactcctctcactcacccatcacataccacctggggtttacatatttcagtttgaaattgtctaaaccaaaagtaactttttccataaataataatttattttggccaggcacagtggctcactcctgtaatcccagcaatttgggaggccaaggtgggtggatcacctgaggtcaggagttcgagaccagcctggccaacatggcgaaaccccgtctctattaataatacaaaaatgagccgggcatggtggcaggcaccagtattccagctacttaggaggttgagacaggagaattgcttgaacctggaaggcagaggttgcagtgagccgagattgtgccattgcactccattctgggtgacagagtgagactctgtctcaaaaaaaataaataataaataataattggtttttttaaaaaaactctgtattagagaacagagaaattgaaaaatgagtcaagaaaatgagataaagtctaagctctaagaagcttcttttcatcattcctgaaataattttattttactattttattttttattttattatttttttgagacagggtctgcatctgtcacccaggtaggagtgtggtggtgcgatctcagctcaccgaaatctctgcctttcgggtttaagcaattcttgtgcctcagcctcccgagtagctgggactacaggcacctgccaccaaacccagctaatttttgtatttgcagtagagacgggtttcactatgttggccaggctggtgtcaaactattgacttcaagtgatccgcctgcctcagcctcccaaagtgctaggattacaggcgtgagccaccgcgcccgaccatcctggtgtaattttagagcacgcacatcaagaattagctgggatccaccttgcttggctacacagaaaaacaagttcaagagcaccatttaatactggtcagctcccagcaacatcatatcacataagactgccatagcaactggtctcttaatcttctttctttttttaaatgacaacaaaattctgtagaaagtaacagtttacccaggctgtccttcattgtctttattaatgataggaagcagtgatatatacaatcaagaattgtgtgtttgtctttgaagatgtttttgtggtagtagttttatcagtctggttcaatcaggagatagcaaagacatagtgtgataaacagaggtgtttaatataaagaattttttttttttggagatggagtcttgctctgtcacccaggctagagtacagtggcgcgatctcggctcactgcagcctccacctcccatgttcaagtgattctcctgcgtcagcctcccaagtagttgggattacaggcgcccctcactgcatctggttaatttttgtatttttagtagagacggggtttcaccatattggccaggctggtctagaactcctgaccttgtgatctgcctgcctcggcctcccaaactactggcattacaggcgtgagccactgcgcccgacccaatataaagaattattaaattccaataacaggagtgactataaaagggctgagggagagtacccaaggaaggacaaacttggaaggggatcccctcccaatgactggagtttggacttcagtggagaaggtttagctcagcccaccagatagcagagaagtttgctagtttgcaggtgctgagcaagccagaagcaactctccagagtgcaggtggggtgtacattcaagccacagctggtgtcatgacatgcagaaggagagagagaatccagtagaaaccttctgtgctgcaagtgaggcacacacgtgggccttcagagggaattggggccagggtgagcagatggccttcaaagctctggtttccatgttgagagggcctcgggagagttatccccaggccaggctaaggcttcaggtcaccaaggacccacaccttctgggagtgtggctagacaggtcttcagtggatgacctctctgcctccccaacaccaccattgacccacagaagcatttggagagccccttcaccctgcagtgtcccctttgcgccctctagtgagaatgctttaggggaaagatgcttaaaggaatcccatccgtcgtagcagagcacattttgtttttgtttttgttttgttttgttttttgagatggagtctcgctctgtcgcccaggctggagtgtagtggcacgatctcggctcactgcaaccgctgtctcccgggttcaggcgattctcctgcctcagccttctgagtagctgggattacaggcacctgccaccacgcccagctaattttttgtatttttagtagagatagggtttcaacatgttggtggggctggtctcgaactcctgacctcaggtgatccgtccgccttggcctcccaaagtgctgggattacaggcatgagccaccgtgcctggccagcagagcatattttgaagagtgaatttggagctgagaggaagtaacataataacttagtctcacagaggaagctctgatgctctgggcatttgtaatatatttaagaataaaaataactgtactccgtctgtgcctgtttgtcccttccatgcatgctttatgcagcagtctttcttaccttttaaattgctgcacaacaggctgccttttacagaatcagacatttctgcatacccagtaattcctcctttacccacagagagctaagaacttttcttctcagattttaaataaaagatatgctatagttggtgtcttagttcgttttgtgctgctgtaacaggataccacagactgggtaatttattttattttattttatcttattttgagatggagtctcgctctgttgcccaggctggagtgcagtggcgcgatcttggctcactgcaacctccgcctcccaggttcaagtgattctcatgtctcagcctcccaagtagctgggattacaggcatgtaccaccatgcccgggtaatttttgtatttttaggagagatggggtttcaccatgttgcctaggctggtctcgaactcctgacctcaagtgatctgaccccttgggcctcccagagtgctgggattacaggcatgagccaccatacctggccagactgtgtaatttataatgaacagaaatttattggttcacagttctggaggctagaacgttaaatatcaaggtgccagcatctgacaagggacttattgctgtgtcatcatgtcacatggcagaaggcaaaagggcaaagagagagggcaacaaggggctgaactcgcctttttttttttttttttttttttttttgagatggagtcttgctcttttgcccaggctggagtgcagtggcacaatcttggctcactgcaacctccacctcccgggttcaagtgattcttgtgcctcagcctcccaagtagctgggattacaggcacctgccaccacgcccagctaatttttgtatttttagtagagacggggttttaccatgttggccagcctggtcttgaactcccaacctgagggcccacctcggcctcccaaagtgttgggattacagatgtgagccaccgctcctggccttgaacttgccttttaataacagcattagttccacccatgagggcagagcactcatggcctaacgacctcttaaaagactctaccttgatactgttacaatggcaatttcaacatgtattttgtaggggacagacattcaaatggtagcagttggtgaaataaggcagataggtatccttaaaataattttttccgccaggcgcagtggctcacgcctgtaatcccagcactttgggaggccgaggcgggcggatcacgaggtcaggagatcaagaccatcctggctaacacggtgaaaacccgtctctactaaaaaatacaaaaaaattagccgggcgtggtggcgggtgcctgtagtcccagctactcaggaggctgaggcaggagaatggcgtgaacccgggaggcagagcttgcagtgagccgagatcacaccactgcactccagcctgggcgacagagcaagactctgtctcaaaaaaaaaataataataataattttttcccagtagaaattattttagacttcagaaacacatgaggaagaaggaagtttatagtacccattaacgtctaaggattaacgtctaaggatcctcttgttaacactgtagtgtacttcattccaatccatgtatgtgtattttgttttcctacagatttgaagtgaaacaggtttttcctccaacctcttccccccacccagcatcatatcataggcattttcccattccactaaattttcaaaattgtttttaacagctgcatggtattcctttcaatggatgaaccataatttaactatttcccagtcgttagacattcagattgttcaacatttttcctgttataaacagcactgcagtgaacatgtgtgtaaatctctgtatatatctctgattagttacacagaaacaatatcaaaacatagtaatgtggcttactcattatgtaggggtaattctaaagcaaaaattatttagttaaaggattttacctaattattttttcacttttgtctgcctagcaggctttcaactccgtttgataatgagaagttttatgtaccttgttctataactttttctcccagcattcgggtgtccacctggaacagactgaactacctaaagaatggtgtgctaaagtctgccttaaaatctgccatggcccatgaccccatctccccagtgctctctgatcctcatctggacgccgtggaccagcggctcctgagtgtcctggccaccgtgaagcagtgcaccgaccagtttgggatggacacagtactggtggaagacaggatgcctctctcacacttgtaattctcgacacaaaataagtgaaacttctttttacaaagatagagaaacagcacaatcaattccaaatggtatgagatggattggaagtggccagcagcaagttctggtgacgggacagagtggccttggatgtctttggtattttctgtagtagaaactaaagcaaagaccacaagtttcagagcatggagacattcctgctgaatcgccttctcacctcctcggcaattgctcattctagggttgggcatcatagttggtcagtcttaattcccatgccaaaggacaaacaggtgtgacatttggatagatgaatactgggattggctctggagcatgtgttttgagttgaaccttgcagtcctttctctacgcccgtggattttgtggaaacactttgcaatctctttgtcttttttttttttaccagaactagttacattggaatgcttactgtcctacagagtggcagcaaataaaaccttgcattccatcaagccaaaatagcacactctgttagaggagatacatgtttaagatagaattggagggaaggacaaaaacagaaaaatgtttgggcttttaagccattgggtagtattgttttgatgatcttagaggagggaagaagagagagagacccaatggtagaaccagaatcagggagatgactgaactactgaaaaacaggttcccttgtatttaggatcttaaggtgtataaaaagcaaacatgactttgcacctaagtaaattctgcattctcatagttgtgtcccaattaaccaaaaagttgtctctagagaaaatactattacaatctaagcatgattctctgtggagactaattttttccccttttgccaaaagcagtccttcccaaattaacaaagcaaactgaaataataccttgaataacaggttgcctgtggtctctgtcatcctcgtttctcttctgaaatgaatttccacctctgcctttaaggcatttttgtcactgaagctgctgttcccaagagatcggcaacctttttgtccctttctcataagaaagggacactcctacaggtgagagtgtataccttactctctcagataagtggctggacttatcttgtgatttggggccatggaagattggaaacaaagattttaagccttcttctttttttcttttttcttttttttttgagaccaagtctcactctgttgcccaggctggagtgcagtggcacgatcttggcttactgcaacctccgtctcccaggttcaagcgattctcttgcctcagcctccagagtagctgggattacaggcgcccgccatcgtgcccagctaatttttatatttttagtggagacagggtttcgggtttcaccatgttggccaggttgatcttggactcctgaccccaggtgatccacctgcctcagccttccaaagtgctgggattacaggcatgagccaccgtggccggccaagattttaagccttctgagccttgaaattgaggaggttaaaaggaagagccttaagattttgatttatgtcaaatcctaattctatcattcagtcttgtttggagttctgaacccatgatgttgtattatgcttctttctcctcttagcactctcaaatttcaggtttgtaaaacacagtttttgttttgtgttctggcaaagtgatctcaacatgtaagtagttgcagtaaaacacaggggcaaaggaagacaggcctgatgtgcccactcatctatggactcagagctgtgtgctttgctcctgcatcttgttgaggtgctgttccagctttgcatttctgtcaagtagaggcgaatatataaacagtgtggttgaatacatttaatgccagccattggaaactagttttaggcaaccactctcaaaaacagctttagaatttatgcccagttttcttgcattgaaagataactgagtaataacctgtaactatttttaaatggcatgaaattaggaaacttttgtacattttatatacattttgagatgaacagaacaatgggctgagttataaaaagcgtgtattgaatttaagaagacagactagcacaaaacacagaattcgtgttaaccaaaggaggcattgatttcagttttaaggctactcagtgttgtgtgtccagggaaattcacagctcagtatgagaataccttggttagtgctcacccacaagcttccaggagccagctgggaggagacaataggaagagatgtcatctctgctctccctgtaaatgttagttgaactaagttatggatttgtggtctttcaaatacatgacgcctttagtatgccacactgaaatgaataagaagtcttctgaaactgggaacttcataacattgaaggcagaagattctgctaaggaaaaaagcaggcaggaaagaaaatgtctcatcctttcttgaaagcatttgcagaaaatatatcatttcattttattcccatctgttttcaaactcgtgatcttaaaaggcattctgatgataaatttagaattttcatctataaaatttagaactctaatccataaagttagaattgagctaatagagtggtatgacatggcactaaaaatataaatttttgttgtaagtcaggattggagtaagctggaaaagtatgtttaggcaaatcttggagaaaaccaaccataaacttacagctctaaaattcagaaagccctaaaatttcaaacactgtttgaaagaagaggtgggggccgggtgccgtggctcatgcctgtcatcccagcatttgggaggctgaggcaggcagatcacctgaggccaggagttcgagaccagcctggctggctagcatggtgagaccgtctctactaaaaatgcaaaaattaacagggcacggtggcatgcgcctgtagtcccagctactcgggaggctgaggcaggagaatcacttgaatccaggaggcgaaggttgcagtgagctgagattgtgctgctgcactccagcctgggagacagagcgagactctgtcttaaaaaaaaaaaaaggaggtgaatttttttttaagttttgtaacactgtcctactttatttattagaatctaaggctgttacaatcaagtcgttgcagggtttggatcagctgtaagttaggtatgcctaccaaacatccaaaggtagacgtggagacattttaatactacaaaactaggaaaatcagaactcatggccatttcctgccctcctccaacttgttaaaacatgtttattctaaagttcgaatggataaatttgagtataaaggttttgttataaaactgttctttagtgtaaggctgcattgtgggtttgggggaaatgtaaataattttctgtgtaaaacaaattcataggatctgatttgctcagagtattattcaagaatgtattaataaggcattgccccctgtttgcactcagggttaatatgtcaaatgaaatttaagaaggaaatggaagaattcaggtacattaattgcatattattttgggaaagatgagtcctatacgtggcaatttttcaatgtcatctgaagccagcattatcttccaaagaaatcgatcttttttttctaaaaaaaaaaaatgcttttgccttcccttcccttcccatccgccatatttcttcagcctttcttctcgatcacccgtgtattctttgaccagtaaatgaccacacctcaatgatggtaaaacagcatcatcagtaagctatcttatatgcctcatcctgtgagtttgagcttcaggaaacatgagtaaaagtatatgtaatgtatatagtcgtatatgtattctagcaagaaaaacatatttattttgacaaaggggaacactgactttctgaaggattcagaaagaaccttagtgaaaggttctcagtctctgagagtggaccctaattaacataaagaccattcatcagcgaataactactgagcaactctagtgtgccagcacaggccagacatactagtgagccaggcacatctggccttgggaaactcatcctacaggggaaggccagtttttttcccttcaattcctcaagtctgggtggtgacaaggtaggggctaggtactggactaccacaggtttttaggaactaaggtgtttctcataaacacaaaatgttgggtgaaactgggaacaactactcagaagctcatttatttgcttaaatggaaagtgtgggagccactaccctctcttttgatctgccaaggatttcctctcagagctgttgcacagacagagattgtacttggtaagataccaaacaagacagatatggatctaaatttctaatgtgttctatgggtttcaattctgaaaaaagaaaatgaataaagattttaataaatattgatatctgatcttttcctcttctttcccccttcaggatatttccagtggttcctatttcagttcccttcctgttaggggacagaagaagattacatcttttgtgaccacttaaacttccacagggatagtttcacagtacctggtggattagacctttaaatttcctcaccagtcttcaaacggttcatactagattgtaactataccaaaattttctatgggatcattgttaggtgctttttgtatatttaatagttaactccatttttgcagcttccttagaggtaaagaattatgaatcctgaagaacttgtcagcacactacaaaaaaggcaggaagagaaggccagttgccctagtagtacttcttaggacacctagatttttaacttttgaaatagaatccagcatcataaacctcaaccaggtaactgagcaaagctcttaagatttttattgggccaggcaccatggctcatgtctgtaatcccggcactttgggaggccaaggcccgtgggtcacctgaggtcgggagtttgagaccagcctggccaacatggtgaaaccccttctctactaaaaatacaaaattagccagcttggtgcatgcctgtaatcccagctactctggggaggctgaggcaggagaatcgcttgaacccgggaggcggagagctgggattgtgccattgcactccagcctgggcaacaagagtgaaactcagtctcaaaaaaagaaaaaaaaaaaatagctgggtgtggtggcaggcgcctgtaatcccggctactcggaggctgaggcaggaaaattgcttgaacccagcaggcggaggttgcaatgagccgagatcgtgccattgcactccagcctgggcaacgagcgaaactctgtctcaaaaaacaaaacaaaactattttatttgcagcatatttgtgctagtttttgtcatttgtacatgaatacatgtgggaggctagtagagcctggtctgtgtatctcactgactcaccacatacaaattcttttccaggctagagttcaggattcatttgcttctcaagcatatcagaacgactggtattagagtttgaaaaaggtagctgggagtagggagggcggggctctgttactgggtaattttacctagtatgtttgcttattagactgatttttagcagtcgtcattttgtaaaatagataggaatggtcatagccactggatttccatgggtagggttttgtaaggtaagttgcagtataagccccaaggaataatggtattaaatcacaatttgtgagatatttggtctgtcatcagtagatctgtgggccattgtgctgcaagctgtggtataccattgagatgatagtgactgtcttttccctcaaagagcctgcagtctagacctggcagaaaaaaaaaaaaaaaaactgcttgtgaggcaccaagtccctcaggaattatataaattaattgcagtgagaataagaaacgggagaggatcatggaggtggggattgggagggattgggttaggcagtacctacattgaaaaagtggagattgatccaggcttgacgagaggtgtgtatagaacccctgactccggaaatgcagaactatttgcttagtggatacagggccttctgggcctgtattttcagtatctgggtcaccagaaatatcgcttcaggggtttctcattgttctaggaaatgtttgtgtgccctgtctcttgaggacattctcagtccacctcaagtgctgtgtagatgttaaaggaacatagatgagctgggaaacattcctgtaatgtttctcatgcagggttatttaaatatggagacttgcgtttgttttgtttttgagaccaaatctcactgcagtgcccaggctggagtgcaatggcgcgatcttagctcactgcaacctccgcttcccaggttcaagcaattcgcctgcctcagcctcccgagtagccaggactacaggcgcatgccaccacgcccggctaatttttgtatttttagtagagacggggtttctccatattggccgggctgattggtctcaaacacttgacctcaagtgatccacctgccttggcctcccaaagtgctgggattacaggcgtgagccaccgtcccaggccgagacttgcttttgacccagtgtttccaaggcagatggttctgattgccttcagtggggttgggaagatgtctgttgtaaagctaggtcatactcagatgacatcaataaaggtgttgtgtgccaattttgccttaggcttttttttttttttttggattgacagtcttgctttgacgcaggctggagtacaatggcacaatcatcgctcattggcagcctcgaactcctgagctcaagtgatcctcctccctcagcctctcaagtagctaggattataggcgcgcaccaccacacgcagcttattttttatttttatttttggtagagacaaggggtctagccttattgcccaggctggtctgaaactctggcttcaagccaccctcctgcctcagcctcccaaagtgctgggattacaggcatgagccatggctccctgcacagaaagaccttcttaaagtgcaggttttgattcagtatgtctggggcctgagattctgcatttctaacaagctcctgggtgatgccagtggtggggttctggaaccgcccttgagtagcaccgtaatgcttgctaaagaatcatagttgtctgtagttttcccagcagcctctagatgtccccgtcatccatgagatactgcttccagaactttaaaacgagcacagcctccaaggcctgagacggaaaggagtatttattgaaatactcaagagacgtatctagaaactggtgtttggatttattgtttctacacgggactgactctttggtgaaaaagtgtttacctcttttttttttttttttggaaaaaaacggccaggcgcagtggctcacgcctgtaatcccaacactttgggaggctaaggcaggcaggcagatcacctgaggccaggagttcgagaccagcctggccaacatggcgaaaccccgtctctactaaaaatacaaaaaaaaaaaaaaagcgggggcgtggtggtgaacgcctgtaatcccagctacttgggaggctgagggggaagaatcgcttgaaccggggaggcggaggttgcagtgagccgagattgcaccactgcactccagcctgggcgacagagcaagactccgtctctaaataaataaataaataaataaataaataataaaaatgtcttgaaatcaagttggttgtcgaagaacttcccagctaacctactttatttacaatgggatgctaaatccaagtcagaacatctcagaaatgagcaggtagccctacataaaagttccaagtacagataattctttccacaacatcttaaaacttggattgttttaataaactattgttactattaattttttcaccataaattcacttataaaccaccagtattaactaaagccatccttgcctcctgtgtagaccacccagagcctcctccatggctgggtagatacaaaaataaaactggccacgtattgagtgttgatgatgtgctcattgaatgagctcatttaatcttcacagcagccctatgaggggactattttattatccctttttataggtgaggaaacaggcatgggacggttcagtaacatccatggttgcacaaatagaaaatggtaatcactatattagactgccattcagactcaaacgacatttgttgagtgagttaattttttttcttttctcttttttcttttttttttttttttgagatggagccctgctctgtcacccaggctggagtgcagtggtaccatcttggctcactgcaacctctgccccctgggttcaagccattctcctgcctcagcctcctgagtagctgggattacagatgcgcaccatcacactcagctaatttttggatttttagtagagactgggtttcaccatgttggccaggctggtctcgaactcctgatctcaggtgatccgcccgcctcggccttctaaagtgctaggattacaggcgtgagccaccacaccctgcctaattttttttggttttccaataagcactgtggtgtatgtgctgttaactgtctccggtggactccgcaagctcgatgtacacagtgctgggctggtgccatggctgctactctatgaaaagtttttgacatcacactgaatggctgagacactataaacattttacttttcaactcaacctcgtgtcctagcaaaggtgctttatctagtttctgtagctgttcgaatgaaaggcacaaatttggtgagctgctgagaatggagcagacctggcacggtgtgatctaagagactggggtgaacagatgaatcaagttgcctgtgacatttgtgagacacggtgctagtattcaccttactgtctggagggcccaacctcaaagccactaaacatatgacattagccaagtcctctgatctctctagacagcagttttctcctaagacaagacggctttttggcttggctgaagtaagccaggtgattttaaacaatgcattaagctcttctgaaatctaacagccttgttccatgctcctcgctatggagaagagagcgctgagggaaagaggccctgtagtcttttgccaatggtgtcggatgggctgctcagtcccagcacttgaaagttgcaaacctagtcaagaaagtgcacacagtacttcggatttttatgagatataattacattgacatgacgcctcatgatttacccatgactcggggggtttccaaactccaaacacctccagactggagcccgtcaatcagcttcctcttgcttttcgtggactcccactgtggaaacctgctggtgttttgctgcggtttcatttccctctgcggaggagccagggcaaccttggccagacaggcacctagctgtgcccttctttccagggcctcttctccactcagctgactcccctggcctgcctaacgctcctgactgagacggttttccggcttcctctaacaggttggcgggaagttgatctgcaaacgcgaggcaaagcaggagtgtccttattcggtaggcctctgatgggccaccagagccgtccgaaagtcctagcggagtgattgacagtggccatttcaaagcctgagttcaccttctttgcaaccacaatcacgacccagaataaactgcttcctggaagccggctagttgcacctggcctgaagtcagtctcgggactgcctctcaggaccccagacagggtttcgggttcccgcctgggtcacgtgtacggaggctgccagtgcgcactcagcccggcccgccggtcgccacagcaacaccaatcccgggttcaagcccgcctaggaagtgcggtccccgcctgaccgccccgggcttaagggagcctggctaggccggcagccggatggtcccgcagctcggggccggccatgcttcgcggtccgtggcgccagctttggctctttttcctgctgctgctcccgggcgcgcctgagccccgcggcgcctccaggccgtgggagggaaccgacgagccgggctcggcctgggcctggccgggcttccagcgcctgcaggagcagctcagggcggcgggtgccctctccaagcggtactggacgctcttcagctgccaggtgtggcccgacgactgtgacgaggacgaggaggcagccacggggcccctgggtaagaccccgtccccacggtccgccgttcgctgcggagcagagtgcgatccgggcgcggctgtggtcgcctcgctcctgtccggggcgacatctgggcccgcagtcctgctctgctcagccgccggtaccggctgtgggcgggggcagcctccgcggagcagggagccccatcaccgcccttgccccacccgcgtcccctgcgcacccccctggcgcccggcttcccgtcccccgcctcccctgatccctgcgctttccagattctctcgcaccgcctgtgccatctcccagcccaccctggcgttattcgaaggtgttgctcttgacagctctagggaacgtccgctgtggacatgcctggtgactgccgtctcccttgctgcttttggacgcagggcttgaggaccgacagtgggccgagtcggggctgggagttagaatctgagttctgggtgtccctctgtccctgacaagttgtgtggcaaggttgccgcatcactgtagagcaccggatggtctcctctcggtctcacagctcctccttttccaggctggcgccttcctctgttgggccagcggtacctggacctcctgaccacgtggtactgcagcttcaaagactgctgccctagaggggattgcagaatctccaacaactttacaggttggaccccgcatggcttcaaggggctagggatctgtggggaggggaggcagtccaggggaaatggttagatgcaagggacctttcgtcatcctgcccagcctcccgacaagcaggaactgccacagtctgactgaagttttgagggagtaccaaatgcttgagtcagggaagcccctttttagcctctagcttaatagcccaggcaaactgagctgtttgtcaggcccagagcaccttgtttcactctaaacagaaacagctacctctcagagggggctggaactatccagagagaactgatcagaaattctgggttgtgattaaaagtgtccctttcttttcttttttttttttgagacggaggcttgctctgtcacccacactggagtgcagtggtgcgatctcagctcactgcaccctccgcctcctgggttcaagcaattttcctgcctcagcctcccgagtagctgggattacaggtgcctgccaccacgtccggctaatttctgtatttttagtagaggcggggggggggggggggggcgggtttcaccatgttggccaggccggtctcgaactcctgaccttgtgatttgcctgccttggcctccaagagtgctgtgattacaggcgtgagccactgtgccatggcaaccagtattttctaattagtttgggttgcttgatttgcatatcgcagttgatttgcattttcactctcacaaggtgatctttggccccatgttctgaaacctgttcagacatttgcaaaagcttcctctcttttccccaggagcaatttcttgcccagagttctcctgatcccaggtccttcaatctccataaatgaggcccaccccagatatttaggtcacttaattggccaaaatgccaataacacagtgtgtttagggacacagtctggctcaggacagaagataggactacatacaggtcaggaagtcttaatttggatcactgctgacttggataagaccctaggtagcaattagcaagttactttctgtcctcaacatgccttttgttcgacaagggcctggatatccaaattgttctggccagcggattggagacccctcctagtcgtgcctcttccttgctggcaaactgaagagtccttcccaagtgtccgtttcctcagctgtattttattttattttttttacttatttattttgagaccaagtcagtctctgtcacctaggctggagcaatctcgtctcactgcaaactctgcctcccaggttcaagcgattctcttccctcagcctcccgagtggctgggattataggtgcccaccaccatgcccagctaatttttgtatttttagtagagagggggttcaccatgttggctaggctggtctcgaactcctgacctcgtgatccgcccgcctcagcctccccaagtgctgggattacaggcgggaaccacctcgcccagccagtttcctcagctttaaatgggaataataatgcctgccttgcagaattgtgaagaccaaatgtaaaacacttagcccagttctgaatacagagaaaaaagcagcaaatgttaattttcttctatgtcactactacttttggagcaatgtgtgatgccctcaatgttttatcatagaccttgacctcccaaggctccagaatacctgctctgggctggcttaaggtgttcaaccaattgatcctggatttggaatgctcacccacagctggccgcacaccctgcctgtctgcactgtggctcctggtagcgccatctccaaatcctacaacctcacgtctgacaggaggaagttagacctagcagaaggctttgcacactggagctgctctcaccaagtgttggctgaattgaatgagaatgaataaaatcaggcttctccataaaacagggagccctgaaaccaagggatgggagcagcctcaggacatgggggacagggttcctccctagacaggtctgtgccaactgttctgggtatctgcaggatctttaggaagggtgattccactctccctgagtcccatcttgatttcccaaactcttcaaaaagtagtccttgaggctgggcgcaatggctcacgcctgtaatcccagcactttgggaggccgaggtgggcggatctcgaggtcaggagttcaagaccagcctagccaacctggtgaaacccgtctctactaaaaatacaaaaattagctgggtatggtggtgggcacctgtaatcccagccactcgggaggctgaggcaggagaatcgcttgaaccagggaggcggaggttgcagtgagctgagattgcgccactgcactccagcctggtgacagagcaagactccatctcaaaaaaaaaaaaaaaaagaaagtagtccttgaatcccttctgctacaaagtctgccctctggcttcttattctaaggccaaacaggaggagtaactgattccacccgagagagattcagagttgcttggtttcttggctgttggctgttgtggtggatgggcctgtgtgtgccttttgctgtgactacctctttcctgtccttaggcttagagtgggacctgaatgtgcggctgcatggccagcatttggtccagcagctggtcctaagaacagtgaggggctacttagagacgccccagccagaaaaggcccttgctctgtcgttccacggctggtctggcacaggcaagaacttcgtggcacggatgctggtggagaacctgtatcgggacgggctgatgagtgactgtgtcaggatgttcatcgccacgttccactttcctcaccccaaatatgtggacctgtacaaggtgaggccgaccagggctggggtgaggcctctgtgctgggagggagacccttctgccagcccttccttgcaaggaaatgggctctggagaagcctggggaggtggggacaggtggcagaacctgtgacagagccactccgtccactcctcacagaggcccttagcatgtggaagagactggcgttccaaacaccaccccctgctctgtccccaaacagccacactcgagaggctggacactgcgcttaagcagggacgctgcatgttgatttttgttgtagaagtagtgaaatgtcacagactgcagtgtagaacagagaaaagcaaaaactgtcagactctccacacggacacacccttgtcagctaaggagaggtagcattttatgtgctgcctttggtgcggaacctttccccaggctctttgtcattgtaactacgttggacagaaagttgtatatcctgcctttttaaaatttaaacaggcctattggccggacatggtggctcacgcctgtaatcccagcactttgggaagcctaggtgggtggatcacgaggtcaggagatcgagaccatcctggctaacacggtgaaaccccgtctctactaaaaatacaaaaacttagctgggcgtggtggcgggcacctgtagtctcagctactcgggaggctgaggccggagaatggcgtgaacccgggaggcggagcttgcagtgagccgagatcgtgccgctgcactccagcctgggcaacagagcaagactccatctcaaaaaaaaaaaaaaaaaagaaaacaggcctatcctatcttacctgattttttcatgtcttcccgtatcagcttattatataccagcaagtggatggacagaaatttccttggctgttctgccattttgggtttcttctactaattcagccatgtagtattgaaaacataccagaagagcctgacagaattgactgcacctgtctttggctttgttttaatatttggaatgtgttcacctgggaagattatgtcagcgtctgaggacagaggctgcccgcgtctcctggcagtttccatatggctttggagggcaggggcaggctgagctgggcctttccgggcagaactgtctggcttagactgtggcaagactgctgcagccccagcttccctgctgtgaaaactggcatgtgcatagccctgcacctcacactttcccccacaaaggtgcacccagtagacagggagtgtgggctctggagtgggagctggccctgaataatatattccctgcccatttcgatgaacattgcaaagaaccatcttttcacttgagggtttactgtcttgcctggggcaaggaaccctccacagcttgcaggagccaagttggatgttggttaggcccagtcagtccttcagcaggcttgctcccctgagatctcggcccagccagggtgccagtgggcgctctgcacccgggagttcagggcaggggactgtggctgcctctgggcacatgagcctctctttgcccatcacccctgtatgaaagctcaactcctatccaccccctgggaatagaccctgagcttaaacagggaagttgcgcctggatgtttttgttgtagaagtaatgaaagtttgcgggtgtgtggacattgttagctcctggatgggatgtagtcctggagaggagagagcagggctggagcaggggccccttctcaagggcagaagggcttctccccactcctttctaggctgtctcctagtccccaagctggcttcaccttgtttagctttcaaccctcagggtgcttgagaatcacctgggagctggtcagatgcaaatgtcctaggccctccccaagagcttggatttgataggttctggcggggggcggggggtggggaacccagccccagggggtccattgcacatgcccaggggaaaccacgccctcaaggccggaggtggaaggagtcaccacttccccagctgctccctatatcccgtgcaggagcagctgatgagccagatccgggagacgcagcagctctgccaccagaccctgttcatcttcgatgaagcggagaagctgcacccagggctgctggaggtccttgggccacacttagaacgccgggcccctgagggccacagggctgagtctccatggactatctttctgtttctcaggtgggttctggggaacaatagtcaggagggctgggggaggggaagatactagctggcagtggaggcttccacacgcccccaggttcagaacctttcctcaagaaaaaacagcacaaactctggtacagaaagcagcagccagctgtggtagctcacgcctgtaatcccagcactttgggaggccaaggcggccgggtcccttgagtccaggagttcaagaccagcctgggcaacatagtgaaaccccatctctacaaaaaatataaaaattagctgggcgtggtgacatgtgcctgtagtcccagctactcgggaggctgaggtgggagaatcgcttgaacccaggaggtgggggttgcagtgagctgagatcgggccactgcactccaatctgggtgacagattttttgaaacaaattcccaaggcaagcctggttagagaacggcatagtctcccatcaagtccaacacggccatttcttccttcagccttggaagtctgtttcttctccgattatgagaagtcatcactggcttaaaatccagggtatacgggttgagaaacccatgcttcctctgagtacctgtagttcagttgtgacgctaccatgcgggacccagtcactgaatcactgcactgaggttcctgtgctttcactgacatctttgcgtgaagccaacttagaaataacttgtccaacctcatctttttgcggaggctccaactgggggagtcctgtgtctgttacaagtcaagtttgggcccttgttgacccaatttgaaggcttcacaacagctcgagcctcggtaggagtcaaactgagtgctcccttcccaactccactccggcctcgccttccccaggccagcacttgcttgctgggggggtttcccaagctgcagggctcactttgtgaggatggggaagttggtgaggggatgctgattttcatcctagcttggggagtgtggaggggtgaagggttgagctgtggagggcagatctagtttcggtgtggattcacaagaggaagtgaaacagggtccaggcgcagtggcctgtctactaaaaatacaaaaattagctgggtgtggtggcaggtgcctgtaatcccagctactcgggaggctgaggcaggagaatcacttgaacctgggaggcagaggttgcagtgagctgagatcacaccattgcactcccgcctgggcaacaagagagagactccatctcaaaaaaaaaaaaaaaagtgaaacaggtttaagccgttgctgggttgggatttctgtgtagtggcgggaagcccttcctggtctgcaggtgaagctgtggagacttaaaatgcattgcgaagtgggaggggagtggggcgcccagggtgatttttaagaggagagagagatctcttgcatgatttgggtgcccagaaccaggaggtgaggacaatacattcttccagttagttcttattcccacgttctgtactatttcccaccttcggaggcagccgggctcacctctcctcctgttacagagagtcaggtgggggctgcaacatgcagatgagcctgaacagaggagtcacctgggtgctctgtggtttctccacctgagccctgggtcgggcagtccaggggaccctctgaaccaccgatgcggaagccagtccttcccacggcacggagcaaagcatggtgacccagcctcgggcctccctttcgcttccacagggatctcccttgcgctgagtgtcttcctcctccctctccccgagacaggggtctcccggcttcaggtggtgccggccacacagactctcccacccacttctcagcttggaaaacctcacctgggccgtgattgcctaacgtgacccggtgaccctgcagtctccaggcaaatgttatagcctttgtagtattttggccgtcgacctgacagcaccaccctccagctgcctgtcctttcccccagtgacccagcctgggtggactgtccgaggccatgtggttgtgggcggggtgggggggggggcctgcgggaaagccgcctgccagactcctccatgccacagcagggtggggaggtcccccaggagcctttgacaaggatgtaaaggacacattaaaaagatgtggccttaggttctagtggctctggtgtgggcctgtgcccagcctttacgagccagaggttcccccctccagccgagggctgggctgacatagtctgtgcccaggctgtccagggcagcagcctctcaacctcaagttctcaggtagtccagcgatggctgggaggagacacactgttgatattggagtctctggacttgatttctataatgagcttcagttaatcatccactgtaataaggattctagggcatgaaagaaaagcacagctgattcaaactgctagagtgaccaagtcatactgcacgaaggaagagcagcagtttaattctgaaataaatcctaaacctgctgtttgaagtgagttgccagtggctttgctggcttcctcctggtggcaggagagaggaggtgggagctgaagcagggagttgagtgtggtgctcagagcctgtccttcatccgggccagggaattggggccggtgactgggcaccaggaaagtaacattacttcgcccacttctgccaagtatagaaggctgcaggaatgggctcacgtggcaggacaggaacgtggaactctgcgatcggagctgctgccctgttctcatgggagctgtaaagtgacaagggtaattaaagcctgtttcctttgtgctgttgtcaggaaaaggatagaaaggactttgaaaaattacagaactacacaaatagaaattatttgtggagtcgtggtgggccttgcgctccagcccccagtgtctgttggctgcctgttgactaatgagaggacagcttagattggtccagtcctgactttataaatgagaacactaaggctcaaagagatgactgagtctatgtctgttctttaggaaagatgtagcagttagcatggggtttgttaccagtaacaaccagttctctggtgaggcctgaagtccccttcctttggggtcttagggataagggctggtgagatcagccctggcaggggctggagacgggggatgagggctggttgagagaggtgccctctggcccctccagacttcccagcgatccaggtcagcagatgagctgggaggctgtggaagggaattgaggtgctccagaaagacagtgtctgtctaaaggtagaacgctggtgccagagtggtggtcctgaagatgggaggggcggcgcgtagagaggggccccaggttatgggtcagtgggtgagattaaagcctctaaagtggtggaagaaaagggactttggctatgggacaaaacaggagagattgagaagggtggagatggtgcagacacatgtgcagagatgtccctttatttactaaaggctgagtgcctgatacgcacatagcactggggttacagaaataaacaggccaagcttcctgccctatgtgatgggacctctctttacccatgtaagcaccttacagaatggagaaggccctggagtccatgcccctaggatggggtgagggagtatcactctgtggggtttcacagcaccctggatcctgccttccagcccctgccaaggtaaacagtgctgcctgcctcctgtggggaatgcaggatggggcaatgccctggcagcagggtcttgcctcagctgatgcaactgtggctgctcctgtgtgcacagatcatgtgcctggaaggccttcctgcagcaggggcagtgtcagaaagtggaagagtggtgtgagcagcttccccggggaaagcctggctgagcaactgaccttgagcaagcactgcagatggcccttgttcctgccgggctcctccagctgggagctctcagcccctggtaaattctggcagtgaaagacacattagcacctccccctacaatgaggcacctagctagacaacttggctgtccgggcttaacctgcgtggcagggaaggacgcctgcccagccttagcctctacgcaatggtggaggcagggagggagagaaccacacagctcccctcatttcccagcagcccccatggagcctagtcaacagggtgtggtcacaggctaaatgagcaaagatgtgagctaatatactggtaggtgtcatgggggctttcagagctgggtaaggagggaaagagatggagatactggttccccactccttaacctgccacctgccttccctgtcctttaccctccctcattctgctggacctgaggaaaatgcaagggaggctaggcctagtggctcatgcctgtcatcccaacactttgggagactgaggtgggagaatcacttgagcctaggagtttgagaccagcctagggaacatagtgagacctcgtctctacaaaaaaattttaaaaatagggccgggcacacctgtcatcctagcactttgggaggcgggcagatcagctgaggtcaggagtttgagaccagcctggccaacatggtgaaaccctgtctctactaaaaaaattagctgggcattgtggcatgtgcctgtaatcccagctacttgggaggctgaggcaggagaatctcttgaatccgggaggcagaggctgctgtgagctgagatcacgccactgcactccagcctgggcaacaagagtgaaactctgtctcaagaaaaaaaaaaatagctgggtagggtggcatgtacctgcagtctcagctacttaggaggctgaggtgagaggatcagctgagcctggggaggtggaggctgcagtgagccaaaatcacgccactgtactccaatctgggtgacagaatgagactgtctccaaaaagagagaaagcacggggaagtgcaagtgcattgatgtggagctgggcaaggacttggtttaaggagctggtggagctggctgaggctgcagcccagggaggggaggagagtgcagtgaggtgggggagaagtatgcctgggccgtggagggcggaggaaggccaagtctccactacctctgtcccagcttccctgctggtggctctacggttgcatcttctctccttatcccctttctccctcctatctctaacccaaatcactttctgtgcttcagacccgtaattccagttgcccagtgatatcacaacctgagagagatcacaggcaagcaaaaaacactcaaacgtgtgtaaaaccaaactttcatctccacctttccgtgttccacctctgttaagggggacagcaagcagcttgaggctaacgcttgttctttgatgacctcctttcacttctcacatccatcagttacagggtttttcaattgtgcctccataactaattcttaaatcctcctttccatcccttctccatgatccataagctcaagtccatgcaaatggcagacaaagccctcctcaccttagccagcctttcatgcctgtgctcctctggcgcacccaggcctagacacttcccttccttgccaagtgtctgatgatcactacttggtctttatttgctcagaagcctcatcctccctctgaagccttctccttagaactcagtcactcctccattagcgcttgccacactgtcctggaaccgaatacacggatgcccctcagaccacacagaagaaccatatcttaccatcttttcatgctccctgcacttagcacagtctcagtaagtgtgtgtcaagagggaacaagagagaatgggtggcaaaactggacagtgtcttttctgtgcagacctttgggttgtgaggttgtgacaaggtctcctggaagagagagaaggtccacgcccctcagcctctattcttccaggcactaatgcattggtttcagcggtgagataacatatatggaagctaaatataaacaaatgggttaacaagctcttgtctctttcagtaatctcaggggcgatataatcaatgaggtggtcctaaagttgctcaaggctggatggtcccgggaagaaattacgatggaacacctggagccccacctccaggcggagattgtggagaccataggtgagtaactgactcaatatgcctctggtgagagccacagctggtgataattaatgtgtttgttggaatgtgtgtttatgaagcagacagggtacttgggaacacagagggcagactataatcatctcacattgtcaaccctgggtgggcacaagaaaagtggtaagaaagcctgagctccttttctgggaatgcttggaaatagtagtttgcactggccccaagaacaggcgcagtggtctctgaagcctgagactctgagtctcactgcaagactttacctaaaggagacacagctgtagagttcttgacatacgctgcagacagcactcctggagttgagagatgagatccaacctcacccagccccttcagtcggggccaggtgtggtggctcacacctgtaatcccaacactttgggaggctgaggcgggcggatcacttgagtccaggagatctagaccagtctggccaacatagtgaaaccccgtctctagaaaaaaaacaaaaattagccaggtgtggtgctgtacgcctgtggtcccagttactcacaggctgagatgggaggatcccttgaacctaggaggtggaggctgcaaggagcccagatctcaccactgcactccagcctggttgacagagcaagcccctgtttcaaagaaaccaacagcaacccccacccccgctaaattccagctaggttcagtcctaggtcagactccatgtaactttggtcttgctttcagacaatggctttggccacagccgtcttgtgaaggaaaacctgattgactacttcatccccttcctgcctttggagtaccgtcacgtgaggctgtgtgcacgggatgccttcctgagccaggagctcctgtataaagaagagacactggatgaaatagcccagatgatggtgtatgtccccaaggaggaacaactcttttcttcccagggctgcaagtctatttcccagaggattaactacttcctgtcatgaaggctagaggaagacttcctggaactgcctttcttccactaacaggaccctgggacctgtaggagcaccccgtttgggactgtgaggtgtttgagggtgtggactggcatccagcagccactaacaaacacacaactggtgtgtaaaaggcaggccttacattagaagccaagccaatcctttttcttttttttggaggtcccaccgagatagataggaacttggattgctgaattcaaaaacagagcccattcttaagatcacttggtgccttaaagacacgcattccaaagtggaatgtggttgaagaaagtgggccaggtggttgaagaaagccatgtgggagctcagcaaatcccaagggcttattatgacactccagatggtctccttagcatctcagctcttctgcaaggaagagcttgggtgttaggcctcagaggctgtagggtccttgggttacagagccggggagaacgaagttctgtgacccaggggtggagaatacactctaggtttgcaggctggtgggctttcaaattggtacttccagaggaaagccaagctgcttctgttgtgagcgaatcagccaagagcctgaggctgaagggaaaagtacacagaggaagatattttacaaaccaggtcagtgtaggccaagacttatggtctacagattttggcgggggaggggggaccttttcaaagacaatagggggtcttgacatgtttgttgtatgtaaagatgataagattaaaatttttgattttcctaaaatccttggcgtactttctgtttctgagacctagcaaaatttcaaacaaacactgggtaatcctgataaggcctggcagagacagggcagatgctggcacacacggagctggcagggtatgtgactggtctgcctgtctagtctgtttgctttgggaccacatcagtgaagccaagtcatgccaacctcctactgaagagactcccatagcacattgctcttatatatgaaaatgttcactcctgtactctcagctataaaaagaaaagcagtcattctgaggattaaatggcaccgtgtagacatgtagcactgtgcctagacacagtgttcattatgtgcctttccccttttcttggttcttctcccatgcctctggaataacacaggtcttttttccctatgttttttatatattaattatacacacatatagagtcagggtgtcactatgttgcccaggctggtctggaactcctgggctcaagctatcttcctgcctcagcctcccaaagtgttgggattataggcgtgagccactatgcctagccttttctgtcttttctatttgtcatcatctcacgtcctttcttaatttaagccttcctcacattacctgacatcaggagttcaagaccagcctggctaacgtggtgaaaccccgtctctattaaacaaaaattaaccaggtgaggtggcatgcacctatagtcgcagctactcgggaggctgaggcaggagaatcacttgaacccaggaggtggtgggtgcaatggtccgagatcgcaccattgcacttcagcctgggtgacgagcgaaactccatctcaaagtaaagtaaaaaaaataaaagccttcctcaaccctctctcctttagttcttgtaaaatgaaacacttctgtctctcccacattcaacagtttattgtgctcatttactcaactgcctcaaggttagaaagtacctaccctgagccacattaggtactcaaatattcaaatcaatgaaataaaagggttttagtttcataccactcaagggataaaagagaactcggcctaatggtacaatcagatggagccagtgttagcttcctactcacaggtcttccgtctcaagcatcccagaagatgtaggccacccgatgctactgcacaccaggctctaaacacactgaatacactcgttgaagggtaagtctcagagacagaacacgtggcattcgttccagcactggccagctgtgtttggtgtcataaatagaggatctgaacctcccaccttgggaaccaagacaagcagcagaatatagtggttccaaacacatgctctggagccagactaccagggctggagccatggtttcatcacttagcactgtgaggccatgggaaagcagtttagcctcagtgtccactccagcataactacctacctcacagggttgctggatgacatgagttcatctttataaagtgcctatgaaaccgcttttgcaaaattcataaactgagaaaattatgagtgacatatcagacctaactgaccccatcttgttgctaacctctaaactgtccttgttcattcctgggcataggccaaactagctttgggaaggatttttgtgtatagtttgtcaaaaaatagcccttcccaaaaggctaaacagttcttgtaaaacaaatgaaaggccaccaagtcaagatgagagaaactggaattcttaccagccattattctagaggtcctaagatttgcaacttccccaattactcctgaaggttaacgtcactattgtgagcctaagattggcctttttgatgtcttttcaggtttttcatttctaacaaccagatggccccacctggacctgccaaccagttctgtggcccccacctaggaactgactcagcagaagagaacagcttcgattccccatgcttttactcaccaaattatccttaaaaactctgatccccaagttttctgagactgatttgagtaataataaaactccggtctcccgcacagccagctctgtgtgaattattctttcgccatcgcagttcttctgtcctgataaactggctctgtctaggcagtgggcaaactcatcccagcactttggaaggccaaggcgggtggatcacttgaggtcaggagttccggatcagcctagccaacatggtgaaagcccatctctactaaaaatacaaaaattagctgggcatggtagcacgcacctgtagtcgtagctccttgggaggctgaggcagaagaacggcttgaacccgggaggtggaggttgcagtgagccgagatcgtgccactgcactccatcctgggcaacagagggaaactccgtctcaaaaaaaaaaaaaaatatcgtaagtcaaatcacattgagagattcttggaagattctaaaaagactttctagacaaccaaaaatgagtgggagaaaaggactgaaggtggagtgtgaggaggtgtataaactgctgcttaccagccttttcaattgggcctcacgtacgcacaacttgtgtatattctacccatacctgttaaggttacttctaaaagcaataactcattcataaaaacttgaagaccacgtcaaacaagaatagaatgttatcctgtgaggttctacacctactctataaatgctacattgactcaaaacacttagaacttacttggagttgtcctcagaatggattttgagagaattaaatttcacttgacagtcgcttcatgttgaagacacacaaacaaacacattacccaccttatgcacagcagcctggactccagatgatgccttttgttcacttccaaaaatgtaatccatcaaaggatgagtattcacccccgcagaggacattctaaagaatgtgccaggctgggtgcggtggctcacacctgtaatcccagcactttgggaggcagaggtgggtggatcatgaggtcaggagatcgagaccatcctggctaacatggtgaaaccccgtctctactaaaaatacaaaaactaagccgggcgtggtggcgggcgcctgtagtcccagctactcgggaggctgaggcaggagaatggtgtgaacccagggggcggagcttgcagtgagccgagatcacaccactgcactccagcctgggcgacagagcgagactcagtctcaaaaaaaaaaagaatgtgccagagggatgcttgctaaaatcagcttctttaccttatagacatctgcattacagagagagaaagaaatgctgcttagactccctgtcaactaaaatcatgggagaaacactccaatctccagcctactttgccaataaaatattgcaacctttatttaaaacagaacacaacttggcaaaccttgtgagaagacagagaaagcagtccctttcaagggcctcatttatttacatcaagtttaacactgttagcagttcacagtcagacaacagacggagagaattaaattagaaaaacaactgaaaatatattacaataacaattaagaacaaatacctgacaacactggtaatgtgacacacaattgatgtgtttttagttaaatcccacattgtctcactatgtccccttagcaatgcacacagtgcccgatgctccaaacctcaccctctgtgcaccacatacacacctgcggggcgggacctttggtataaacgttcaacgagttttaaagaattgaatccgcatgtttggggacaaagttctgtcaaccaccatgaaatgtttaaacttatttttactaagcgagcataaaactggggtttgtccagtgacagtcattagagaatcagactgcagagaaggaaaagtgcagggtctgggaggagatggaggttctgagtatacatcaacagctaagtcagcctgttcacagtcaacacaccaaccacgccactgctgttcatccaaggaaagatctgagcgattcctcttttacttactccccctttcttaatgggcacgacagcaatgcacgtgtatttatggacacaaacacacaccagctatacagatctgagatgagagggttttcaaacatatgccctaaagaaccataaacaagtatctcattgttcctttctagtttctgaatactgattgacaattggcctaatcatggtgatttctcaagatctcaagaaattcctctcaaaacataatggaggccaaatggccacagtgagaaattgctgtcaaaatggaggagagctggtttctctggccctcatctaccagaagcatctctgtcccctggcgttcttgccactcacaatacctggtatggtacttctgaactgtgcagaacagtatctgaggtttactgttgatgacactaaagcgttcccagtcctgcaaaaccttctgaaacaacaataaatttgcatattctttttattgtgtcataattgctgaaaattaagtatacatatatgtgaaaatttaagtcccattctccaactaaagtttatggtgcctaattccgaagcaaatttaagcaagttctgactacacaaactccttatgtttcctttcataaagcctgctagcattcgacagtttgcctccagcttttctaaaaggttacttagcatgctcggagacctcagcttctgagggttcagctcagtagcctggagtgacaggtaggaccctgaaaatttgctgatgcgagtacagtgatgaacaaagaaccttccagaatagacacagatgacatgtatcacaattctgctccaaatatggtgtggtctgaggacagtgctgtgcctggcctttccccttcaacacgccagctgctccctcagagtgaacagggctttgctactctgctcaggtcaaaaggtaagatttttgtaggagaggtaaacaatttcaaagttagagatgtacatggggatgaagaagtaagtgcacttggagggaagggtccggcgagacctcgccaggtgggcttgagaaacgcagcaactgccaagtgaacactctcaggaaaccacttcaagtggcggattctcacagatggaggatctggccactgccatctgagaagctcgtgtctaccagctctagttcaataatcatgcaaaatcaacccagctgacaagtcttttcctctctgagactcatgaaacccatctttgaaggcacaaatctccaagggtggcctctcagcagtttctgccattccaagtcagctttgaatcatgacctgccacaggcctccttacctgggaagaagtatgggatcttcttatcttgatcccacagggtgaagccttccctaatttcctgtcctttggatctaggatattgagtcagaaatgaaggtatccttttttttttttttcttcttaacgtgtctcactctgtcacttaggctggagtgcagcggcgcgatctcagctcactgcaacctccgcctcctgggttcaagcaattctgcctcagcctcgggagtagctgggattacaggcatgtactacgcccggctaatttttatatttttagtagagacggggtttcaccatgttggccaggctggtctcaaactcctgacctcaagtgatccgcccatcttggtctcccaaagtgctgggattacaggcacgagccactgtgcccagccaaaaggtatcatcttgtacatactcaagatagaatacatacctcagagaggtggcatgaaaagcagaaattcatccgtctgcacattgcaataaacttcccaagagacaggctggactgagtcatgggggtgagtgctccagagtaggctgtagcttgtcccacagccctgaatcccacccatgcttctcagtaagagaggagcgtagggaatgacagtgaagttcaagtgaactacagttgtgctgaacagagcaagtaacaataactattactactggcttgctggaaaaaagaaaaaaaaagcaagctttgcattagaatttctttttttttttttttttttttttttttttttttttgagacggagtctcactgtcacccaggctggagtgcagtggcgcgatctgggctcactgcaagctccgcctcctgggttcacgccattctcctgcctcagcctccccagtagctgggactacaggcgcccgccaccacgcccagctaagtttttgtatttttagtagaggtggggtttcaccgtgttagccaggatggtctcaatctcctgacctcgtgatccacccgcctcagccccccaaagtgctgggattacaggcgtgagccaccgcacccggccagaatttctttaagtctagtagttggaaaaccagaactctatccaaatgatatagatatcttgactgttaagagttggagacgtttcagaagttctcacatactcctacggatcccagactgaagcaggaatttacaaaggaagtgagctactactggcttgatacaagctaagacagagacacaaaccgctgaagcctatcaatacttaagccagagacccacctacaaaaagaggattggtcatagcagatccttagggcacagagatgaaccccaaatccaggtggaacaatttaatgcaagatgatgaaactcaaaaatcaagaacaaaaatatagataaccctgtcaacccaatatgaactcctggtaggggcacagcctgataacaagaatgacatcagtagcaaaatggaagggagcagaaggggtcataatgaaatgtagctataattcagaagtggccaccaaggcaatttacttttgagtattttataataggtgaattaaattcagctattctttttagaaaaagaaaaaaaagatgacaaatgtcaatgtcatttataactggtaggaaatcctagaaaagtaattcccaaagtgcactggttttatggccatccacctccccctgtaaacctaacaaaactatgcaggacataggttaaaaaagacactgtcaacatcaacagataacaagtctgttcccaccacatttaggataaatggccaagcatatagtggataaatggagaagagtaagaggacctcatccttcttgagtcaaggtccttggaagagcttctgtgttctggagtgagtacacattcttactttttaaagatcggaattaaaccaaaaagttggtccctagtttcctgaagtaattcagctctgaactacaactttagcaaacacttgctatacctgctcatacctgttaagcagtttcatatttctaaatgtgataaatgacagaggtgattcttacgtatctacattttcaggtaccccacagggtccatccacattgaatcaactgtcttatcttttaaacagacaatactacaaccttatagttatattacagttgatattttctcaaagtgctgtcatatacattatctcatagtcattaaaataatgtgaacaataaagtttttttaaagaaaagggaatgtaggactaactaggttttttaaaaactcaagttggtatttctttactacaaagtgcacattgctgtgttgctgaggtctgtccaaaagaatgaccccattgaattttttaaatgactgaattctactgagtgcttgcttggcttgtgttcccattactccacattcaagccccttaaggtcaaagtgtcaggagcatgtgctttagaaagtgtgcagcaccacacaggctgattgtcggtctgagccacttttttgcagtgtctcacatggcagcaggtatggtggaaagaccccacaaccataaagtcaagagacttgaattctagccttggctccgccactaattaaccgttctgagacaataggcaagtaatttaaccatcttctgctagtttcttaatctataaaccaaagggttggggtactgatagttttcagatagcactgggtgtttcaaacacctggcgagattactggagaatacactggccgagaccccagcccactgggaattctgatctggagtggggctattccgtcagttttttttgtttgttttgttttgttttttaaccctaggggattctgctgtgcatccctagactaggtgttaataatagtagtagtagaacatttatagagtgtgctcattgtgtgccaggcacttgagctaagtatcttacaaatatcatctcatttaatccttacaacaaccctgaggtagttactattattatccctgttttacagtgaggaaactgaggctcccaaggttaaatgacttgccttttagactggcattagagcctaaacactaaaccactatctatactgcatcctttaaactcaaaatctccatgactttagttaaataaaaatgggcagtgccttagcaaagtcagcatgctttattacctgggacatattttactagccaattttcagttgaatcctaagaaactttctatctaatttaaatcttaaaatgctgctggtaaagggtctccactataatgacctctatgtacagaggtcatctagaaaaacactgaatgaatttcttagggccaaattttttaaggctaatctctgaaaatcatgatggaagtctacaggaaaatatatacatttaaattttacacaaagccttttaagaaaatactttaggtaaagcacaatcaagatgtattcaacacaaatcactgcattttaaagtgggaaatctaaactgaagtaatattatctgtacatgaaaggaatcaagcagtggcagcccaaaggcatgcatccagatatacagcataaccactttagaaaactgtcaacgccttggacaagggcctttgctcattgcttttagaatagttaaaattaaactacacttttccagaagcagttctctctgctttggattcaaaccttttaatttacataagggtttgtgtttaagctggtccttttcaagtagaaaaaggaaaaacctaaattatgcttgtatagtaagttgacagtgcctctttaacctgaaccttgagccctcaacccctgaagttacatagttcaagcatcatgtggacggggtatgctccaatagtcaatgccactcttgacagtgttgacaaaaatcaattccatttatatatcgtgcacccactgtaggcaagacagctaggtgctgcccctgagtaagacacagcccccaccccctacccttcagattgttgttgggcaagaaaaccagggagcctaaatacactcagtagtgtttctccaaggcttagttccagaactctcaagggaaaatagagcccttgcttaaaaaacaaaaaacaaaaaaaccctgaaaaacaattacaatcccttaacttgcatcccacaacaacagggataaacctaaaaagcagcataataatattctacagttttagctttcccatcctcgaccttaaaaggctagctgctaaaacgagttcttcaagtttacggacatgtatgatgagcaagaatggcaggaaagaaggtgggagaaggaccgctggtttttacatctgagttctcttccaaccactaggaggagatgaacccaatcactagctgcctgtgctgcaactgcaggtgacatactgcggggatcacccaacacctgccaggaatgcccagcaccgcgtgtctcctctaatcctcttaacctgggcctcctttggagcagcaatggtaagaatcaaggtttcctttctttccaatgaaaactcaattttgttcttggtcaaaaatgccaaactctattcaaaacatagttttccccttagtattctagcccaattcatataatctcaacaattcttaatttcattaatattttctactcagcaaataagataatggaaaacaattcagcataacaacactgcactaaaagagctttccagagaggctctccaagggatagctgcacacttacttgcaacacccttgagaacatgtactgttaccttaattaaatagattaagagatgaggctgtggtttgacagtgtatcaatgacagagccagaagaaaactcagtgctcaatgttgaattcctatcttcctaagctaatattcctttgagagaataaaataaaaatatgtgtgtattgagctctccttggggaggatgggggaagtattcataaagctttaaaaagagaatgaagcagtaactggaattagacccgataccaagaacattaataatacctaactcattagattcacttccatgccatcctaattagcttccacaattataactaaggaaggaaggaaaaggcctatttttcaagggcaaaacatatttaaggattaagtcattaggttcccagggtctacctggtgctccctgaaaatgactactccttcaattatgcattcttaaaatagaagtgaaatgacaaaataaataaagaatattcccaataagatctgtacaaataagcacaatgattgggtgggggagtagggaggaatatgtgagagaagaaaagcatgtgagaataatgtgggggtgattcacttcaaatccatagagaggagaatgcctctccctattctctacctggattgggcttaaggtggtaagggaagggaaaggtacagagaaacagccattaggaccaagccagcttttcctctcagtcacatacaggccactgggtttcaagaaaagaccatctccctggtattttagttgataaagaaaaatacacagctgctcaagtccttggccagtatgtcagtatgcaggggaactgtatcagaacaggattcattcctttgtgagaacctgtagggactactattttcaaaatcaacactggttaatgctctgaatgctaccaggaataaaggaagacagcctgcccagcaaaagacagagaagaaccactagggagacccctgtacttatggtttacagatagccaccaacaggcaggaagccgatcctggtccattatgaaataatataccagactgggagaagtgacttctgctaatctgtgtaagattttagaaggttttcaatttctgattcaactttccagcattctttacttcctgcactaactgatacaccagcggaaaataggacagaggcattcagatagggccatatttcccagttttatgttgtagcactctgatttgcagttcttattacagcagcaatggccttgtggaagcaaggctaacagctacttccaacagccctgaacaaaaatttctcttggtacattcccagctttcttttctgctgtcccaccaaattcattaggtaaattggagcataatcaccattagtacctaccagagccccaggtctgggtgcagctgctgcagtcttgctgagagcagccctgcctagcacttcccagcattccaggtagggatgggctgcattgccttccttatgacatacacatgttccctattttccagtgcagtttccaaccctgtccactactgccttgcccccacttaatttacctctcctatacttatgtggtttgcttcttatttttgagatgaaactgtaaacgtgagaactcagggatctgaggtacttcacataaacacactcaagtatgagtctttcattttctgaaaacaagaacacaggaaaacaagtccttttccctctcccctcagaaatgtgtgcattttcccaccaggctaacagtggctacctctgcaccacatcactgatttcctgtacacatgacaataagttattaaggacagggtttgtcccgggcacaccagcagctgctgaagaaacctgtagctcctgcaggctgagttccagtttgctcacagcctctcggaaggcaaatttgttgcgagtttgagggatgcagtccacatagcctgagcagtagtcaagcagctggtgtccagtgtctaccagctggctgttgggcacaggttccgtgagtgcactggacagtaggtcagcacattccagcagggcctctttgctgattttgtctgctgagattttctcagcggcctgtttggtttttctcagagccactttagtacctgctgtgccattggccattttggctggcgagatggaagatgtgggcagaggcacttgaggtggaggcatcactggcctcccagctttcccactgatgggcactgcgcccagagctgccttctttcctccttcctgtgtttctgatgtggactgtcctgcagttagggcagttggctcttctgtagggtctgagcagatggacggatgctgcagtagtctcatcactggtggtgggggtggggcacactttggttttacccgtcggggtcggtccttgtctccagaggatgtgacctgatgctcagataagagcttgaatttattcccctgagagtctgtgccaatgagctgcacgtcagctggagtgtgtttcagagtgggtgagataaggactggcactttgtggttgtgagtggttgggaggacgggggcagccttggctggagaaggccagcccggctgctctccatcctctggaactccagccatcccaagtcgtgccccaccattcttctctttacccttgggggcagctgccactccagccactcccacccctggagggtccttctctgtaatggctagatccccagagggtgttctgagaggaagagctgtggctcctctgggcaataacttggcttttggtctctccctgcttggagcagcactttcctctgatttttttggaagcatgtcattggccctgtccacattctcttctggctgagaagaggtggacactgtcctttccagctggagtttggacctctggcagttcctgggaagggtcattgccatcctatcctgctctggaagccctgaggacatggaagatgtagagtttgaccttggaaaaggcttggaagtgtcatcactggctgtgggtttacctgctcgtaagcccagtgtctttttgattaagcgtggtgtaaagaagcctgtgatgccagaccacccacccccagcagtgccactgcccccacccccaccaccgtcgtcattacagaggttcctctgtgcaaagctccccccatagcacttgggtggcaccagattcgcctcttgctgggcaggagtgaaagagaacccatcagcatgctgtagagaagcaacagatgagaagttacccgtgagttcgtatttcttatggggctgattctccatttctcggaaggagctgctgcgtttggggggtgtaggagcatttctcttcttcatgaaggagctgaagaagccccccttcctatccctggtgaagcatgtctctttggcatcttccaagaggctgctgggtgacttgtctctttgctttcgaggcagtgctggggatccactagaggcctgtgcacctctgatgaaccctgatgagaaatggccgatcagtaacttaaaagacaagaagtgaatctattacatttgagttaccgaggctgcattatgagttttgtcagcatttatcaaaggtcaggtgttggcaggactcagaagcaaataataaatacattatcatttcacagtgactggtgttccaaatgtgatgtaagttaaaaatgggggaagatagggggccgggcacggtggctcacgcctgtaatcccagcactttggggggctgaggcaggaggatcaggaggtcaggagatcaagaccatcctggctaacacgatgaaaccccatctctactaaaactacaaaaaattagctgggcatggtggcgggcgcctgtagttccagctactcggaaagctgaggcaggagaatggcgtgaacccagaaggcggagcttgcagtgagccgagatcgcaccactgtactccagcctgggcgacacagtgagactccgtctcaaaaaaaaaaaaagaaaaaaaaaatgggggaggagataacctgctggttattacctcaacactcatcctttctttaaaaaccactgcgcaacctcttctcctctatcgggttcagcgaagtttttgatggaactgtaaagatgatacatacttgggaacccaggggtacggagatgaaactggagccagagttgtatgttggatggatagccaagggcttcatgggtgggtaaaagagacgccatgctttccccagggaggacgggcatgaaagtggacataaaagcctcacaccccatgcttctttaaacaaggcagtttctattttgattctacctgctaagggacccatacctggtgctaaactggaagcagaattttctgtggcatcttgtgccccttcaatgttctccttgttctccacctgtttcttcagtgtccgagtcttggaaggaagtataggtagccggggcaggtatggaacaacagatgacgaggaggcggctctcccaagctcctcagctacctctgtgaggaagacaaggggaccaataaaaagaacaatttcatagcagaaaaagggagttcttttcagaaaaggcacacaactgagaaagaactggcaagtagagtactaaaatacatacacggaatcataaagtatgtactcttttgtctggcttctttcgcccagtatcatgtgtgtagattcgtccacgctgctgcatgtggcaggggttctcattgctgtgtagcatgccaacgtgtgaatataccacaatccacctgttctcctcttgatggatgtttaggttgtttccagtttggagttactatgaatacgttgctagcatcatcctgctctgtgtcttttggttcacaatgttatgcacgtctgattggcacaatgtaggagaactggtggatcacagggtatacgtgtattcaggtttattaggccctgccaacctgtccccaaatagcgatactattttgcatgaaagctgtggttactctgcatgcttgctaaaatttgatgttattatttttggcttttttttttttttttttttgcagatggagtctcaccctgttgcccaggctggagtgcaatggcgtgatctcagcacactgcaacctctgcctccgatgttcaaacgattctcctgtctcagcctcccaaatagctgggattacaggagcccgccaccacacccagctattctttgtattttcagtagagatggggtttcaccatgttggccaggctggtctcgaactccggaacttgtgatccacccgcctcagcctcccaaagtgctgggattacaggtgtgaaccacagtgctcagtcttttttttttttttttttttttgacagggagtcttgctctgttgcccaggctaaagtgcagaggtgcgatctcagctcactgcaacttccgtctcccgggttcaagtgattctcctgcctcagcctcccaagtagctgggattacaggcgcctgccatcacgcctggctagtttttgtatttttagtagagacggggtttcaccatgttggccacgctagtctccaactgctgaccccaactgatccacctacctcagcctcccaaagtgctgggattgcaggtgtgagctgctgtgcctgccaagcctgattttaattttagcctttttggtaggctgtggattggtatctcagtgtggttttattttgtatttctctatgatgagtaatgttaaacatggttattggacatatggatatcctcgtttactaagttaaagttaacctttaactactttccatgtcaatatatccaagaaggccgggcgctgtggctcacacatgtaatcccagcactttgggaggctgaagtgggtggatcacttaaggttaagagttcgagagcagtctggccaacatggcgaaaccccatctctactaaaaataacaaaaattagccaggtgtggtagcaggtgcctgcaatcccagctactcaggaggctgaggcacaagaattgcttgaacttgggaggtggaggttgcagtgagctgacatcgcaccactgcactccagcctgggtgatagagtgagactccatctcaaaaaaaaaaaagtatatatatgaatatcattttaaatattcagctatatggttgtaataatttacagtccactagatatataagttgttcataatttctcactattataaactatactctgatgcaatgtccttgtagttaaaagttgaaacagagtctcaaactccacaaaagttgatcatttttgaaatgtaattaccattatcaccaccaccaccactacccagaattagtcaccaacagtaaatccaacagattctcaccttcagaaatgctggagtcatggaacatggtttcaaaagcttggtgtgtttcagcaaaagagggcctatcggcagggctccacttccagcctatgtaacagaagaaaaaatattaaaaactccttgcagaaattcaatatccagtggtgtatctaataatgagttctatgcatcatgatgcacacttattaaagtacagttacagcatgttcacaatgatagcaacatagccacttctcagctctagactcagaggtataatgaacccactttctaaattccttttttctttttctttgagacagggtcttgctttgtcacccaggctggagtgcagctggcatgatctcaactcactgcagccttgacctcctgggttcaaacaatcctcctgcctcagctccccaagtagctgggactatgggtgtgcaacaccacgcccagctaattttttttttttttttgagacggaatctaactctgtcacccaggctggagtgcagtggcgcgatctcggttcactgcaacctctgcctcctgggttcaagtgattctccagcctcagcctcacgatttgctggggctacaggcgcacgccaccacgtccggctaatttttgtatttttagtagacaggggtttcgccatgttggccatgctggtctcgaacccctgacctcaggtgatccacccgcctcagcctcccaaagcgctgggattacaggcttgagccactgcacccagccaaagtgagcacacacagcgatctcaagctactgtattttttatgtagatgggattttgccacattgctcaggttgcttttgaactcctgagctcaagcaatctgctcacttcggcctcccaaactgctagaattacaggtgtgagccactgcacctggccattaacccactttctaaggctggtgcttcgatttagcctcagctcctattccctctaataaaaggatgaggagaaacaaggagatgttttagaaaatggcaagttttcttccacttgaaaataaaagaagtaaaacctgtcttcaaactcagcctaaccatataatcaactgtatacattcagaaatataaaaggatatcaaaaaggaggatctcagagttcatttttgacaaagtgcaacagagaacgtcaatgttcaatgaggaaacaacacagacttatgttcaaacacaactgcaattaggaccaggcctggtggcccacatctgtaatcccagcattttgggagactgagtcgggcagatcacttgagaccaggagttcgagaccagcctggccaacatggtgaaaccttgtctctactaaaagtacaggccgggtgctgtggctcaagcctgtaatcccagcactttgggaggccgaggcaggcggatcacgaggtcaggagatggagaccatcctggctaacacggtgaaaccccgtctctactaaaaaatgcaaaaaaaattagccgggcgtggtggcggccggcgcctatagtcccagctactcaggaggctgaggcaggagaatggcatcaacccgagaggtggagcttgcagtgagccgagatcgcgccactgcactccagcctgggcgacagagcgagactccgtctcaaaaaataaataaataaataaataaaaatacaaaaattaggccgggcgtggtggctcatgcctgtaatcccagcactttgggaggctaaggcgggcgcatcacttgaggttaggagttcaagaccagcctggccaacatgccaaaaccccatctctaccaaaaatacaaaaattagccaggcgtggtggcaggtgcctgtaatcccagcaacttgggaggctgaggcaggagaatcgcttgaacctgggggacggaggttgcaaagattgtgccactgcactccagcatgggcaacatagtgagagtccgtctcaaaaaaaaaaaaattagctgggcatggtgtgcgtgcctgtggttccagctactgaggggctgaggcatgagaatcacttgaacccatgaggtagaggtttcagtgagccgaaatcacgtcactgagccgaaatcacgtcactgcactccagcctgggcaacaaagcaagagtgtttgaaaaacaacaacacaaataaacaaacaaacaaaaccctgcaatttaatccactattcactttcctactttttcatctgcaagcaatcggatgcttctgtttatctacacagaggttatttttatgcattcttcatagtctttagcaatcatttttatctagttgaaaacggccctccccaccttaatctctaaactcaatttcttatcttattaaatatgcaaatgtctctgagaaactgggtcaatatttgggccataacaactatgcttaggattccaataagattcaatggtatatgatatgacaactggctgatcaatgataaactctttaaatgtcatgaggctggattcaatctaacaacactgagaggagaaatgtttctaattttgttaactattctgaactgctagcttatgccttcaaaattaaaacatgcaaaaatactcacatgctctcataagttcataaaccttaggggggcatccctcaggctgttccattcgatatcctttttctagtaggtcatagacctgagacaggtcaatacctggatatggtgacattccataggtagcaatttcccacaacaataccccaaaagctgcataaggaattaaaaaaagcacttagtgtttcttctccgattatttattttacataattaggtgattctctttcatattttaggtaacattcacacactgttacaacaacattaatatataatatactcttttatcaattcttacagcatgatttgtccttctaaaatatttgcaacaggaatgctgcagataatctcaagacagttttatttaaatttcccaatttttatttagatttcctttactgtaatgagggaaaataaactatgaccatttaagaagtctgaggcataatccaactattcttagaaaagtttttgatatacagtagtcaccccttatccatggtttcacttcccacagtttcagctactcacagtcaaccaaggtccaaaaatattaaatagaaaattccagaaataaacaattctaaagttttaaattgcatgctgccttgagttgcatgatgaactctcaagccatgcttctccttcccgcctcgcacataaatcatccttttactcaacatatccacaccgtagatgctacccaccccttagtcactgagaagacctctcggttatcagatcaaaaaaagattatatatccagggcttgacactatccttggtttcaggcatccaaggggggggtcttagaatgtattctcctgaggataaaggagaaccactgtatctaagaagcataacagttttaaagtcagctaatacaagaggagtaaaataaaaatggattcatttaaaatcagataaaattcagagcagaaaaaaatttgtttgattttgtctcagttatttgagtttgagatttccagtaaaaaatgacttagaatcctatgactatttctattggtccacaatccaaatttccagattgttcagtctactaagcttcaaccaaatcatattacctctcagagcagtagaaaccagatcaattaggacaaattctagaggaaacctcatcacacttccctcgaaaaaaaaaaaagctctgtgtcacagtactctgcaaatgagtgataaaacctaatgtgaggctgagcacagtggctcatgcctgtaatcccaacactttgggaggccaaggcaggtggatcatttgaggtcaggagttcgagaccagcctggccaacatggcaaaacctcatctctactactatgacaaaaattagccgggcatggtgatgcatgcctgtagtcccagcaactctggaggctgaggcaggagaatcgcttgaacccgggagatgggaggttgcagtgagctgagattgtgccactgcactccagcctggacaacagagcaaggctctgtctcaaaaataaaaacaaaaaacctaatgtggaaataggatgctttacagagggaggatcattagcactccaatgcaataagagatctagactgtgagctctctgaaggcagggacttcttttttaaaactttcttctttcttttttttttttgtttgagacagagttttgttcttgttgcccaggctggagtacaatggtgtgatctcagctcaccgcaacctccacctcccgggttcaagtgattctcctacctcagcctcccaagtagctgggattacaggcatgcatcaccacacccagctaattttgtatttttagtagacacagggtttttccatgttggtcaggctggtctcaaactcctgacctcaggttgtccacctgcctcggcctcccaaagtgctgggattacaggcatgagccactgtgcctggccctaaactttcttctttatacttttttttttttttttctttttagacagagtctcgctctattgcccaggctggagtgcagtggcgccatcttggctcactgcaacctctgccctccaggctcaagcaactcccctgcctcagccttcagcctcttgaatagctgggattactggcgcatgccaccacgcctggctaatttttgtagttagtagagatgggagttcaccatgttggccaggctggtctcgaactcttgacctcaagtgatccacccatctcggcccaaagtgctgtaattacaaacgtgagccaccacaccaggcctatacttctgtttatgcaaagttcttaacacagtcctggcagataggaggtatttaacaaatgcttgctgaataactgaagaagaatggcaggtaaaggtagaggatactgaacatcgtaagagaagctctaaagaatcaaggcatttataaaaaaaaaaataaaatgacacagaaaaaagtccctttcaaccttaccccagacgtcagatttaattgagaaggtattgtaggcaagactctctggtgctgtccacttaataggaaatttggctccagcatgagcagtataagtgtctccagtcatcaatctacttaagccaaagtcagccacttttaccacatggttttctcccactaggcagttacgagctgcaagatctctgtgggaaagagaaccctaatgtgattccattcagatggtgaggaaaatggtcatgaacactctattcttggttttgtgttataggcagagttaagtaagtgctgacaaatatggtagtatcttctgataatcactaacaccttagaaagccactaatgatatgtcactgtagagcactcatcaagatgcggaataaagcaggctttataaaatgggtccctatttataagtacactgggataggtgagcaatagatgcttttctacaagactaagttatttactaatgcagaaaactattttaaaaattcagccactaggccaggtgtggtggctcacgcctgtaatcctagcactttgggaggccaaggtgggtgtatctcctgagctcaagagttcaggaccagccggggcaacatggcgaaacccagtctacaaaaaaaaaacaaaaaacaaaacaaacaaaaaaaaaccataaaaaaacacttagccgggcatggtggcatattcccgtaatcccagctactcaggaggctgaggtgggaggatggcttgagcccaggaggctgagggtgcagtaagctaagatcgcatcactgcattccagcctgggcgatggagccagcctgtctcaagaaggaaaaaaaaaaaaagccactagatggtgaaaaaggatcttggatacagtgctggcaaaataaagagagctctctggccaggcatggtggctcacacctataatcccagcactttggaaggctaagatgggaagatcacttgaggccagtagtttgagaccagcctggtcaacatagcaagaccccatcactattgcaatttttttttaagagagttctcaggtttgagaatagcaaaggccccttattaatactttaatcagagtacagtaggtttacaagttagaagtgacattataatgggccctgtgaaatacaaaattactttcttgaatcatctgaaattttctgggaaggttctcacattcttccttttatctgcatgtcaagcaagatggcttcatggttggtcagaggtaagttttacacacctatggatgaaattcttcttctctaagtactccattgcagaagaaatctgagtggccatgtagagcagcacaactgcagtcacctcttctcggttgcattctcggaggtaatccagcaaattcccgtatggcatgtattcagtcacaatgtaaaatggtggctccaaagtacacacacctaaaagttgaacaatagtgcttgtttttattagtgtacattcaaacactccaaaccactttggccaagaataatttgacaatttttcaggtctagttcggcaataatcttttcccttcttaccaccacgttctctgaaattaataagcaaaaactaagaagctggtttataagggagtccttgctttcactgtattgttctctgggaattcatatgcctctgagtatttgtctatttgagcaaaagcagagttacctgtattaattctataaggggccaagaaaaccaacatgtctagagttttgcacatggcccatgattctcctcctcaactgcaggctaacaatacttattcaaaagtaatagcaattgtgcacaagctgaagactatgtgagccccacctgcaatactgctttgcaaaatgattttcatcatttccatagtcaaggatttcactgatatatggcagattccttataaaaaaccaacaagaggccgggtgcagtggctcatgcctgtaatcctagcactttgggaggctgaggcaggcagatcgcctgagcccagcagttcaagacttgactgtccaacatagcaaaaccctgtctctactacaaaaattagctgggcgtggtgatgcacgttgaggcacgagaatagcttgaacctgggaggtggaggttgcagtgagccaggatcatgccactgcactccagccagggtgacagagcaagactctgtatttaaaaaacaaacaaaaaaaaacaccccccacacccccccaaaaaaaagaaaaaaaaaaaaacctggccctattttaaaaaaggaaaaaggcttagtaagagagttctttgtgacctgaacagtgaaaagccaatctaacataataaagcatttcctgcttgtaaaggtaaactgaggggtgtggtcacagtttttaatttgcttcttttcaacagtcctttgtttaatgtctctttattttgcaaaaacatttcatgagtttttggttcagtaccattttgaacacttctgaactttgaatctggggcaattagtacaaactgagcagttttatacaccaaaatcaatccaggccactgtttcttgggctatttttaaacacagaacctactgattaaaataactatttattgaagtggattcaaacctgcagcttccaccatggtgactaaaaagaaaggtttgggggaggaaaaaaggcggggcatggtggctcatgcctgtaatcccaacatattgaaaggcccaggcggaagtatcacgtcaacagcagcctggacaacagagcgagaccctgtctctatatttaaaaagaaaaaaaaaaagcatttggagataacaaggggttaaagggcaagcatttttggaggaaatctatggttcaggcaaggttcctcacctaaaagttgtaccagattaggatgcttgatttccttcattactgcagcttctttcaggaattcttctacctccatggtatcttcctagaaaaagggaaaggtaagaaagaagaaaacgagagggacaaaccctcaacttaaaatcatttcattcatctcacaatcataaagcttcagctttaaaagtaaaaactatcttttgaatattttagcatattacaagatataattattattttaatttaatttatttttttgagacacggtctcgctctgttacccaggttggggtgaagtggtgtgatctcactgcaacctccgcttcctgggctcaagcgattctcctggctcagcctcccaagtagctgggattacaggcatgcacgaccatgcctggctactttttgtatttttagtagagacggggttttgcaatgttggccaggatgctctcctgacctcaggtgattcgcccacctgagctccccaaattgctgggattacaggtgtgagccaccacgcccagctgaaataattattattttaagaagtccaacttagaaagaaaaatgccctggagtaaaaattagcaggaggagcaaaattcagcaatcgtgtcctataatcaactgaaacacaggagccctgtattggatagttcagttctctcatagtttacttctccagatatctcaggctttgaatgacgagacagagcacttagctgcttacatacttatcttcttgcttagatcataatcttcttagagagcaaggccttatatattgcttcacactcagaatatgcttaagaaacagaactgactactacttaatagctaaagaatagcccttataagtgatacaccagagacagttactgaggctctgggagaatcaagtgagtgaaaaccttataatttgatactgggatgtttaaaggagacatatagtagtactacgaaaaagatttctgttaatattaagaagtgatctaactgcaaagagacaggaggaacaagaacaagggagagaaaggacagaacaagaaagaggcccatgactctcagacaagaattctgtccagcaggggtcgttctcaggaagcaaaagagcttattttgcttttataacttttaatttgcttctaacctcccactaagatctacatcaacagcctccagagacagacctttcccacatgtgggtttcactgcattgcagtgaataatctacctctgcattgtcagcaatggcactagggttaatcatctcaatttgctgatgtgcttggcactagtgggtggaaagtgttccaaagttaaagaagggcatgctgatatttccaagtgattgagcacaaaagaaaaagatgcctgaaaactgtaattctcagcaacccaccttcaatgttttcacagcaactgtaaggctgtatttcttccagacgccaacgtaaacctctccatactgaccgcccccaagtttgtgcttcatggtaatatctgttcgctccatttcccatttgtcgtggatgggggacacaccatagactgtaggcttattacacttgggtgctgggtagtgtaatgttgtcaccagcccatcagccactgtggagtgatggtgtacaagctctgccaaggtgctgaagcggctctcagcagtcacatacacctggtaagaaaaggagaaaagctaaacaacttgaaaagagattaagaattttttttttttttttttttttttttttgagatggagtcttgttctgtcacccaggctggagtgcactggcgcgatctcggctcactgcaagctccgcctcccgggttcacgccattctcctgcctcagcctctctgagtaggtgggactataggcgcccgccaccatgcccggctaattttttgtatttttagtagagatggggtttcaccgtgttcttgatctcctgacctcgtgatctgccggcctcagcctcccaaagtgctgggattacaagcatgagccaccgctcccggcctaggaattcttttttaaagcccatatttcactggcctgaagatatagatagaaggctatctttaaaaaaaaaaaaaaaaaaaaaaatattctgcagacagggtctcactattgcccaggcaggtctcaaacgccagcctcaagcaatcctcctgcctgggcctcccaaggtgctgagattacagactgggccagtatacccagcaagagtttatcttaattgaggtaaataaaaaagttaaatgatgattgcaagggtaaacacagaacttcagtttaatcttatttaatctgcctttcctattcttcctaaattagtcttgggaaaaaccttccaagatctctctctggataatcaagctcattggtcttcaaatatatatatattttcaaatatatatatactcaaattatgtattttttcaaatatatatttctttttctttttttcttttttttgagacagagtctcactctgttgcccaggctggagtgcagtgtcatgacctcagctcactgcaacctccgcctcctgggttcaagtgattctcctgcctcagcctcccaagtacctgggattacaggcatgcgccatcacgcctggctaatttttttctttgtatttttagtagagatggggtttcaccatgttagccaggctggtcaggaactcctaacctcaagtgatctgcctgcctcagtctcccaaattgctgggattacaggtgtgaactaccacgcccggccagtcttcaaatatttttaagccacaagactatggactcaactacagaagatacgtaaagaagagataccctgatcaaagtaggcaagcaaggacttggcctcttgtagaaatttacaaaatatcaccttaatcttgtaaaaaacataacttgaaaaccactgatttagatttttttggtaagagtattcaaatatctcataattttaattatgacgtgcagttgagtgtctaggccattaaagaaagagccacaatttaccggtggccaggagcagtggctcacacctgcaatcccagcactttgggaggctgaggtgggcaggtcacttgaggtcaagagttgagaccagcttggccatcatggtgaaacctcctctctactaaaaatacaaaaattagccaggcttggtggtgcgtgcctgcagtcccagctacttgggaggctgaggtgggaggattgcttgaacctgggaggtggaggttgcagtgagctgagatcacgccactgcattctagagtgggtgacagagcaagactccatctaaaaaaaagaaaagagccacactggatttcacagaattgcttacaaaatagttgttaatttgctaagcaataagaaatgtaaagtaagagggaattc

SEQ ID NO: 2 - DNA, 3543 bp, Homo sapiens
agaggtatggtccttgggacagttctccttccacctaatacttatggcagagatcaggacacttcactttgctgcctgtgcactgaggcctcagaatctgctctacccgacttaacagaagctttgcatcgtccctatggttgtgatgttgaaccccaggcactaaatgaggctatcaggtggagctccaaggagaacttgctcggagccactgagagtgaccctaatctcttcgttgcactttatgattttgtagcaagtggtgataacacactcagcatcactaaaggtgaaaagctacgagtccttggttacaaccagaatggtgagtggagtgaagttcgctctaagaatgggcagggctgggtgccaagcaactacatcaccccagtgaacagcctggaaaaacactcctggtaccatggacctgtgtcacgcagtgcagctgagtatctgctcagcagtctaatcaatggcagcttcctggtgcgagaaagtgagagtagccctgggcagctgtccatctcgctcaggtacgagggacgtgtgtatcactacaggatcaataccactgcagatggcaaggtgtatgtgactgctgagagccgcttcagcaccttggcagagcttgtacaccatcactccacagtggctgatgggctggtgacaacattacactacccagcacccaagtgtaataagcctacagtctatggtgtgtcccccatccacgacaaatgggaaatggagcgaacagatattaccatgaagcacaaacttgggggcggtcagtatggagaggtttacgttggcgtctggaagaaatacagccttacagttgctgtgaaaacattgaaggaagataccatggaggtagaagaattcctgaaagaagctgcagtaatgaaggaaatcaagcatcctaatctggtacaacttttaggtgtgtgtactttggagccaccattttacattgtgactgaatacatgccatacgggaatttgctggattacctccgagaatgcaaccgagaagaggtgactgcagttgtgctgctctacatggccactcagatttcttctgcaatggagtacttagagaagaagaatttcatccatagagatcttgcagctcgtaactgcctagtgggagaaaaccatgtggtaaaagtggctgactttggcttaagtagattgatgactggagacacttatactgctcatgctggagccaaatttcctattaagtggacagcaccagagagtcttgcctacaataccttctcaattaaatctgacgtctgggcttttggggtattgttgtgggaaattgctacctatggaatgtcaccatatccaggtattgacctgtctcaggtctatgacctactagaaaaaggatatcgaatggaacagcctgagggatgcccccctaaggtttatgaacttatgagagcatgctggaagtggagccctgccgataggccctcttttgctgaaacacaccaagcttttgaaaccatgttccatgactccagcatttctgaagaggtagctgaggagcttgggagagccgcctcctcgtcatctgttgttccatacctgccccggctacctatacttccttccaagactcggacactgaagaaacaggtggagaacaaggagaacattgaaggggcacaagatgccacagaaaattctgcttccagtttagcaccagggttcatcagaggtgcacaggcctctagtggatccccagcactgcctcgaaagcaaagagacaagtcacccagcagcctcttggaagatgccaaagagacatgcttcaccagggataggaaggggggcttcttcagctccttcatgaagaagagaaatgctcctacaccccccaaacgcagcagctccttccgagaaatggagaatcagccccataagaaatacgaactcacgggtaacttctcatctgttgcttctctacagcatgctgatgggttctctttcactcctgcccagcaagaggcgaatctggtgccacccaagtgctatggggggagctttgcacagaggaacctctgtaatgacgacggtggtgggggtgggggcagtggcactgctgggggtgggtggtctggcatcacaggcttctttacaccacgcttaatcaaaaagacactgggcttacgagcaggtaaacccacagccagtgatgacacttccaagccttttccaaggtcaaactctacatcttccatgtcctcagggcttccagagcaggataggatggcaatgacccttcccaggaactgccagaggtccaaactccagctggaaaggacagtgtccacctcttctcagccagaagagaatgtggacagggccaatgacatgcttccaaaaaaatcagaggaaagtgctgctccaagcagggagagaccaaaagccaagttattgcccagaggagccacagctcttcctctcagaacaccctctggggatctagccattacagagaaggaccctccaggggtgggagtggctggagtggcagctgcccccaagggtaaagagaagaatggtggggcacgacttgggatggctggagttccagaggatggagagcagccgggctggccttctccagccaaggctgcccccgtcctcccaaccactcacaaccacaaagtgccagtccttatctcacccactctgaaacacactccagctgacgtgcagctcattggcacagactctcaggggaataaattcaagctcttatctgagcatcaggtcacatcctctggagacaaggaccgaccccgacgggtaaaaccaaagtgtgccccacccccaccaccagtgatgagactactgcagcatccgtccatctgctcagaccctacagaagagccaactgccctaactgcaggacagtccacatcagaaacacaggaaggaggaaagaaggcagctctgggcgcagtgcccatcagtgggaaagctgggaggccagtgatgcctccacctcaagtgcctctgcccacatcttccatctcgccagccaaaatggccaatggcacagcaggtactaaagtggctctgagaaaaaccaaacaggccgctgagaaaatctcagcagacaaaatcagcaaagaggccctgctggaatgtgctgacctactgtccagtgcactcacggaacctgtgcccaacagccagctggtagacactggacaccagctgcttgactactgctcaggctatgtggactgcatccctcaaactcgcaacaaatttgccttccgagaggctgtgagcaaactggaactcagcctgcaggagctacaggtttcttcagcagctgctggtgtgcccgggacaaaccctgtccttaataacttattgtcatgtgtacaggaaatcagtgatgtggtgcagaggtagccactgttagcctggtgggaaaatgcacacatttctgaggggagagggaaaaggacttgttttcctgtgttcttgttttcagaaaatgaaagactc

SEQ ID NO: 3 - DNA, 3849 bp, Homo sapiens
aaaagcagaatctgtgagtcgcctggaggcagcgcggcggctgccgtgaggaggccgggtgcggagccgccggtggcccagccgctcagggccagggcctgggctgggagggagagaccggagcagcgccaggagcccgaggccggagccgaggaggaatgtgaccaggggtcggcgggggcgcgggagtacgcgagagcagggatggggcagcaggtgggccgcgtcggggaagctccggggctccagcagcctcagccccgcgggatccggggcagcagtgcagccaggccctccggccgcaggcgggacccggcggggcgcaccacagagaccggcttcaatatcttcacccagcatgatcactttgccagctgtgtggaggatggatttgagggagacaagactggaggcagtagtccagaagctttgcatcgtccctatggttgtgatgttgaaccccaggcactaaatgaggctatcaggtggagctccaaggagaacttgctcggagccactgagagtgaccctaatctcttcgttgcactttatgattttgtagcaagtggtgataacacactcagcatcactaaaggtgaaaagctacgagtccttggttacaaccagaatggtgagtggagtgaagttcgctctaagaatgggcagggctgggtgccaagcaactacatcaccccagtgaacagcctggaaaaacactcctggtaccatggacctgtgtcacgcagtgcagctgagtatctgctcagcagtctaatcaatggcagcttcctggtgcgagaaagtgagagtagccctgggcagctgtccatctcgctcaggtacgagggacgtgtgtatcactacaggatcaataccactgcagatggcaaggtgtatgtgactgctgagagccgcttcagcaccttggcagagcttgtacaccatcactccacagtggctgatgggctggtgacaacattacactacccagcacccaagtgtaataagcctacagtctatggtgtgtcccccatccacgacaaatgggaaatggagcgaacagatattaccatgaagcacaaacttgggggcggtcagtatggagaggtttacgttggcgtctggaagaaatacagccttacagttgctgtgaaaacattgaaggaagataccatggaggtagaagaattcctgaaagaagctgcagtaatgaaggaaatcaagcatcctaatctggtacaacttttaggtgtgtgtactttggagccaccattttacattgtgactgaatacatgccatacgggaatttgctggattacctccgagaatgcaaccgagaagaggtgactgcagttgtgctgctctacatggccactcagatttcttctgcaatggagtacttagagaagaagaatttcatccatagagatcttgcagctcgtaactgcctagtgggagaaaaccatgtggtaaaagtggctgactttggcttaagtagattgatgactggagacacttatactgctcatgctggagccaaatttcctattaagtggacagcaccagagagtcttgcctacaataccttctcaattaaatctgacgtctgggcttttggggtattgttgtgggaaattgctacctatggaatgtcaccatatccaggtattgacctgtctcaggtctatgacctactagaaaaaggatatcgaatggaacagcctgagggatgcccccctaaggtttatgaacttatgagagcatgctggaagtggagccctgccgataggccctcttttgctgaaacacaccaagcttttgaaaccatgttccatgactccagcatttctgaagaggtagctgaggagcttgggagagccgcctcctcgtcatctgttgttccatacctgccccggctacctatacttccttccaagactcggacactgaagaaacaggtggagaacaaggagaacattgaaggggcacaagatgccacagaaaattctgcttccagtttagcaccagggttcatcagaggtgcacaggcctctagtggatccccagcactgcctcgaaagcaaagagacaagtcacccagcagcctcttggaagatgccaaagagacatgcttcaccagggataggaaggggggcttcttcagctccttcatgaagaagagaaatgctcctacaccccccaaacgcagcagctccttccgagaaatggagaatcagccccataagaaatacgaactcacgggtaacttctcatctgttgcttctctacagcatgctgatgggttctctttcactcctgcccagcaagaggcgaatctggtgccacccaagtgctatggggggagctttgcacagaggaacctctgtaatgacgacggtggtgggggtgggggcagtggcactgctgggggtgggtggtctggcatcacaggcttctttacaccacgcttaatcaaaaagacactgggcttacgagcaggtaaacccacagccagtgatgacacttccaagccttttccaaggtcaaactctacatcttccatgtcctcagggcttccagagcaggataggatggcaatgacccttcccaggaactgccagaggtccaaactccagctggaaaggacagtgtccacctcttctcagccagaagagaatgtggacagggccaatgacatgcttccaaaaaaatcagaggaaagtgctgctccaagcagggagagaccaaaagccaagttattgcccagaggagccacagctcttcctctcagaacaccctctggggatctagccattacagagaaggaccctccaggggtgggagtggctggagtggcagctgcccccaagggtaaagagaagaatggtggggcacgacttgggatggctggagttccagaggatggagagcagccgggctggccttctccagccaaggctgcccccgtcctcccaaccactcacaaccacaaagtgccagtccttatctcacccactctgaaacacactccagctgacgtgcagctcattggcacagactctcaggggaataaattcaagctcttatctgagcatcaggtcacatcctctggagacaaggaccgaccccgacgggtaaaaccaaagtgtgccccacccccaccaccagtgatgagactactgcagcatccgtccatctgctcagaccctacagaagagccaactgccctaactgcaggacagtccacatcagaaacacaggaaggaggaaagaaggcagctctgggcgcagtgcccatcagtgggaaagctgggaggccagtgatgcctccacctcaagtgcctctgcccacatcttccatctcgccagccaaaatggccaatggcacagcaggtactaaagtggctctgagaaaaaccaaacaggccgctgagaaaatctcagcagacaaaatcagcaaagaggccctgctggaatgtgctgacctactgtccagtgcactcacggaacctgtgcccaacagccagctggtagacactggacaccagctgcttgactactgctcaggctatgtggactgcatccctcaaactcgcaacaaatttgccttccgagaggctgtgagcaaactggaactcagcctgcaggagctacaggtttcttcagcagctgctggtgtgcccgggacaaaccctgtccttaataacttattgtcatgtgtacaggaaatcagtgatgtggtgcagaggtagccactgttagcctggtgggaaaatgcacacatttctgaggggagagggaaaaggacttgttttcctgtgttcttgttttcagaaaatgaaagactc

SEQ ID NO: 4 - protein, 1146 aa, Homo sapiens
MVLGTVLLPPNTYGRDQDTSLCCLCTEASESALPDLTEALHRPYGCDVEPQALNEAIRWSSKENLLGATESDPNLFVALYDFVASGDNTLSITKGEKLRVLGYNQNGEWSEVRSKNGQGWVPSNYITPVNSLEKHSWYHGPVSRSAAEYLLSSLINGSFLVRESESSPGQLSISLRYEGRVYHYRINTTADGKVYVTAESRFSTLAELVHHHSTVADGLVTTLHYPAPKCNKPTVYGVSPIHDKWEMERTDITMKHKLGGGQYGEVYVGVWKKYSLTVAVKTLKEDTMEVEEFLKEAAVMKEIKHPNLVQLLGVCTLEPPFYIVTEYMPYGNLLDYLRECNREEVTAVVLLYMATQISSAMEYLEKKNFIHRDLAARNCLVGENHVVKVADFGLSRLMTGDTYTAHAGAKFPIKWTAPESLAYNTFSIKSDVWAFGVLLWEIATYGMSPYPGIDLSQVYDLLEKGYRMEQPEGCPPKVYELMRACWKWSPADRPSFAETHQAFETMFHDSSISEEVAEELGRAASSSSVVPYLPRLPILPSKTRTLKKQVENKENIEGAQDATENSASSLAPGFIRGAQASSGSPALPRKQRDKSPSSLLEDAKETCFTRDRKGGFFSSFMKKRNAPTPPKRSSSFREMENQPHKKYELTGNFSSVASLQHADGFSFTPAQQEANLVPPKCYGGSFAQRNLCNDDGGGGGGSGTAGGGWSGITGFFTPRLIKKTLGLRAGKPTASDDTSKPFPRSNSTSSMSSGLPEQDRMAMTLPRNCQRSKLQLERTVSTSSQPEENVDRANDMLPKKSEESAAPSRERPKAKLLPRGATALPLRTPSGDLAITEKDPPGVGVAGVAAAPKGKEKNGGARLGMAGVPEDGEQPGWPSPAKAAPVLPTTHNHKVPVLISPTLKHTPADVQLIGTDSQGNKFKLLSEHQVTSSGDKDRPRRVKPKCAPPPPPVMRLLQHPSICSDPTEEPTALTAGQSTSETQEGGKKAALGAVPISGKAGRPVMPPPQVPLPTSSISPAKMANGTAGTKVALRKTKQAAEKISADKISKEALLECADLLSSALTEPVPNSQLVDTGHQLLDYCSGYVDCIPQTRNKFAFREAVSKLELSLQELQVSSAAAGVPGTNPVLNNLLSCVQEISDVVQR

SEQ ID NO: 5 - protein, 1182 aa, Homo sapiens
MGQQVGRVGEAPGLQQPQPRGIRGSSAARPSGRRRDPAGRTTETGFNIFTQHDHFASCVEDGFEGDKTGGSSPEALHRPYGCDVEPQALNEAIRWSSKENLLGATESDPNLFVALYDFVASGDNTLSITKGEKLRVLGYNQNGEWSEVRSKNGQGWVPSNYITPVNSLEKHSWYHGPVSRSAAEYLLSSLINGSFLVRESESSPGQLSISLRYEGRVYHYRINTTADGKVYVTAESRFSTLAELVHHHSTVADGLVTTLHYPAPKCNKPTVYGVSPIHDKWEMERTDITMKHKLGGGQYGEVYVGVWKKYSLTVAVKTLKEDTMEVEEFLKEAAVMKEIKHPNLVQLLGVCTLEPPFYIVTEYMPYGNLLDYLRECNREEVTAVVLLYMATQISSAMEYLEKKNFIHRDLAARNCLVGENHVVKVADFGLSRLMTGDTYTAHAGAKFPIKWTAPESLAYNTFSIKSDVWAFGVLLWEIATYGMSPYPGIDLSQVYDLLEKGYRMEQPEGCPPKVYELMRACWKWSPADRPSFAETHQAFETMFHDSSISEEVAEELGRAASSSSVVPYLPRLPILPSKTRTLKKQVENKENIEGAQDATENSASSLAPGFIRGAQASSGSPALPRKQRDKSPSSLLEDAKETCFTRDRKGGFFSSFMKKRNAPTPPKRSSSFREMENQPHKKYELTGNFSSVASLQHADGFSFTPAQQEANLVPPKCYGGSFAQRNLCNDDGGGGGGSGTAGGGWSGITGFFTPRLIKKTLGLRAGKPTASDDTSKPFPRSNSTSSMSSGLPEQDRMAMTLPRNCQRSKLQLERTVSTSSQPEENVDRANDMLPKKSEESAAPSRERPKAKLLPRGATALPLRTPSGDLAITEKDPPGVGVAGVAAAPKGKEKNGGARLGMAGVPEDGEQPGWPSPAKAAPVLPTTHNHKVPVLISPTLKHTPADVQLIGTDSQGNKFKLLSEHQVTSSGDKDRPRRVKPKCAPPPPPVMRLLQHPSICSDPTEEPTALTAGQSTSETQEGGKKAALGAVPISGKAGRPVMPPPQVPLPTSSISPAKMANGTAGTKVALRKTKQAAEKISADKISKEALLECADLLSSALTEPVPNSQLVDTGHQLLDYCSGYVDCIPQTRNKFAFREAVSKLELSLQELQVSSAAAGVPGTNPVLNNLLSCVQEISDVVQR

SEQ ID NO: 6 - DNA, 84539 bp, Homo sapiens
gatccgccctcctcagcctcccaaagtgctgagattacaagtcaccacgcctggcttccgtatttaagtattaggtggtgactccatacaaaatatttaacagcttgctggttaacatttaaatctttgttgctaagaccttttctatgtttgacagttattccaaacacgttccatataggaatagtttgccaaatagaagaacacgggatattcttctcccatggctgatgtgttgtgattctaggtgcatatttctgtttccaagaacacggacctgttctcaagttcaaacactgcatcaagagactgtgccccatgtcttttcacttttttccctctctctttctctccccttcccctccttgatttaatttcttcagcaggtcagaattgatacagcttccagaagaaatagtagaagtaggcaaaagattagtttttaattcttgttctgtcaaatactttttgattgaaattgtttttattacaaaggtagcatttgctttttgaaaaacaaaataagaaaaatctttttaatcacttcactattaagtttgttgaatattccctcataactttttctcctggctttttgagtatttgagacatttctttccacattgcagttttgaaacattctaccccacactcatacatgaaagtgaaggctctgcttctcagagtacagtttaatttactttgtggacatcattaacaaaaggcagagaaagacattggaccatactaattgtgggacttagaaaagctatattagtgatggttattaagtatcgcttgcagtggagtctgtaacatattcctatgaagttaatctaactactgagatagtgggagtgcagaggaggcatgctgtgagatggagtggtattttccagcatggcttttctgtttcttgaaaaccctacagcactttgccatggtttctcatctggatgaaataaagcacctgtaccttagtatgtcctgccatgcctttgtatggcatacttggctttgttctccaggcctcttcccacagtaacaacggattctttaggaattggaagggggggccgggcatggtgactcacgcctgtaatcctgggactttgggaggctgaggcgggtggatcacgaggtcaggagtttgagaccaacctggccaatatggtgaaaccctgtctctactaaaaatacaaaaaagaattagctgggtgtggtggggcatgcctgtagtcccagctactcgggaggctgaggcagaagaattgcttgaacccaggaggcggaggttgcagtgagctaagatcgtgccactgcactccagcctgggtggcagagactccgtctcaaaaaaaaaaaaaaaaaagaattggaagggaatggaggaagcaaggtggttaaacaaaaatcatctttacacacaccacccactctatgatgtatatgagttcagaagagcactttgctttatccttaatagaaatatctgtaaagccaggggagtcataaagctgtaggctaagtaaggtgccatgagaaaaggaggcttcacttacgccagtatttggagcagcgtttggcctcttggaggtgtttgcacccatcggagatgctccatagtgagatttaggataagtgcaaggtgagctttgcttttgtaaaatgcatgaagtgtgaccacgaaagggccatgggaaagaattattgatgggttacaggtgcatcgtttcaggcagattgacactaggaaagagcaaagagcaggtagaggtttgacagtttactcctttaaagtaatccatgtccaataacccatggaaagtcttcatgtacttctaaggggtagacataccctagtctaggtgctaatctaggttctatcatgtgttagctgtgaccctgggcaaataactttatcctatggcctcaatttcatcttgcaaaaaaatacggattagataatggcacttccttaaaaggtttgatgggatttcaatggaatattgcatagaaagtgcttgaggcattgtaagtacacagtaagtgaagtgttgaccattattaatatcatgatgataaagtgtgtaacttattttataagtaccatataattgaaatttgccaatagtaactcctcggagacttccagggaactctgtcaatcaaaaaaagctttgtgtaataatggacgctaccttgacagagtgtgttagtcaagagaggatacttatttataaattttgggatatttatacttttttttttttctttagagatggagtcttgctctgtcgccaaggctagagtgcagtggcgcaatctcggctcactgcaacctctgcctccccagttcaagtgattctcctgcctcagccttccaagtagctaggactacaggcacctgtcaccaggcctggctaatttttgtattttttaggagagacggggtttcaccatcttggtcaggctggtctcgaactcctgacctcgtgatccacacgcctcagccttccaaagtgctgggattacagacgtgagccaccgcacccaactataaaatgtatttttatagatacttattgaattttaggatgtgggctgggtgattattttttaaagtacttttaaaaaaattaaggtattatttacatataatatccacccatttttaagtatacattttggtgaattactgtgtgtcaatcatgtaacctcaaacacagccaagattataaaacagttctctacgctgctttggtgatggatacaccaaaatctcagaaaccaccactacagaacttattcatgtaaccaaacaccatgtgttccccaaaaacctattgaaaaaaaaaaagaacccaaaaccaaaaaacagttctctcaacattaaaagttttcctgtgcctttttccagtggattccttccctcagcctctggccccagacaaacacttatccactttctgtgtctgtagttttgtcttttcaagaaattcctatgaatggaaccatataaaagttatataaaaatcatagacttttgtatttgatttcttttactcttagcatcatttttttgagatttactcctgttgtatgtatttacatttcctctttttcttggtgagtaatattgcattatatgaatatctcacaatttatccattttactagtggataacattagattgcttccaacttgaggctattatagtacagctgctatgaacatttgtgtattcaccattgtgtgagcatatgattttccctaccttcttttctatttcactttcttttcctttttctttccttttttttttttcttttttttgagacagtctctctgtgtcgcccaggctggagtgcagaggcacgatctcggctcactgcaacctctgcctgctgggttcaagcaattctcagcgtcagcctcccaagtagctgggattacaggggcccgccaccacacccagctaatttttgtatttttagtagagacggggtttcaccctgttgaccaggctggtcttgaatgcctggcctcagtgacccacctacctcagcctcccaaagtgctgggattataggcctgagccacagtgcccggccctttttcctttctttttttgagataggatcttgctcaatcatgcaggctggagtgcagtggcacacttggctcactgcaacctccacctcctgggttcaagcgagtctcctgcctcagcctcccgagcagctgggactacaggtgcgtgccaccacgcccagttaatttctgtgtttttagtagagacggggtttcaccatgttggccaggatggtctcgatctcttgacctcgtgatctgcctgcttcagcctcccaaagtgctgggatgacaggcgtgagccaccaagcccagctgacaatggattttctttgttttctccttcatctgaagatgtcttccctttgccttcatttttaaaggatattttctctgggtatagaattataatccgagagtatagaggttgacaagttttttggtattggttttagttttgggttgtgtctttttgttgttggcttttgttttgtttttgccccttatagatgtttcactgtcttttggctcatgtgttccgatgagaagtcagcccttcattgggtccttgttctcatctgtttaatacctgttcccatacctgccctttctctctcactgcttttaagatttgctctttatctttggttttggagtatttgactatgaagtgcaagctgctttgtttcttcttggtgtacattcagcatcttgcatcttcaagtatctgcgtatcattaagtttgggcagtttttggctcttgtttcttctaaattttttctaccccattctccctgtccctctccttttggtattgtagttacccactggaacacgtgacattatgtagcaagtctctgagtctgttcatttttttccttcagtcttttttttttttttttgaaatggtctctctctgttgcccaggctggatggtacagcggtgtgatcacgactcactgcaactttggcctcctgggtttaagccatcctcctgcctcagcctctttgagtagctgggaccacaggcacgtgccaccacgcctggctaattttttgatttttactagaaacagagtcttgccatgttgcccaggctggtcttgaactcctgggctcaagcaatccacctgccctggtctcccaaaatgctaggattacaggtgtcagccactatgccagccttttcaattttttatctctcttcttcatcttggttaatttctactcatcttttttgaaatgcacatactgtccttttctttgtccattcagctgagtatatccagtgaattttttatttcaaaaagttaatttttttcagttctagaattgccatttaattattttttgttgttcctatttcccccattgagatttcctacttcttagctgagattttcattcattggaaacatgccttatactgcttcattgagatgattataatagctgcttaaaatctttgtctattccagcattttgttgatctgcaggttagactggcagttgaactaatttgtcccactgtcttgcttctttgtatggtgggtagttctggattgtttactggactgtatgaatgtcaaattctggatattctggattctgttacttttctctaatacttgtttggttttatcaggtggttttcttggctgggtttgaacacaaatgtgtttctctctgagatctttagtctgtagctgagctgctttgtgtctgtcccatgcatgagtggctcaagggtcagtcagggatgtgattgtacggggattggagatcttcccctgcctttttcccttctaggattacttcagtctatttatttatttgtttatttatttagagacagaagtcttcctctgtcacccaggctggagtgcaatggcgcgatctcagctcactgcaacccctacctcccgggttcaagcgattctcctgcctcagcctcctgagtagctgggattacaggcccgcaccaccacgcccggctaatttttgtattcttagtagagaaggggttttgccatgttggccaggctggtcacgaactcgtgaactcatgctctacccgcctcagcctccccaagtgctgggattacaagcgagagccactgcgcccagccggctctctttgttattcatagtttcctggcttctcttttctgagccacccagccaaaaatgctttgactttttctatgcatgtttctgtcatgtctgtgcatgacataaatcataatatcctaagacacaaaattgttaaaggcaataatttggaaacatacaacaataaataaaatggttatagagatgtaactttaaaaagttgaacattttgttaaaacgattcctgaatatgctggaaaattcagatagttaacaaaatattttgaggataaaaaggaaaggaatctgttatctggaggtagaataacttggccattttacagaaaggaaacaaattctgatttcactttttgtatatcatccatttgacaattttcaataaaaggtttattgaaaacggaatacataaggctggtgtttggacctttttcctaattagaaattgtccatgtctccaaagattattaactcagtttattatcagctaaagagatcttaaagttgactaaacacctggaaattaaaacttaggccaacacatcaaagtctttatgacttgaagtgttttaagaattaatgaggcaaagcacacacctgtggtcccagctacccaggagactgaggcaggagaactgtttgagcccaggagtttgaagctgcagtgagctatgatcatgccactgttcttcagcccaagagactgagtgagacctcttaagaaaaaaaaaaaaatgaccggacacagttgctcacgcctgtaatctcagcactttgggaggctgaggcgggtggatcacgagatcaggagatcgagacaatcctggctaacacggtgaaaccctgtctctactaaaaaaatacaaaaaaattagcagggcgtggtggtgggcgcctgtagtaccagctacctgggagcctgaggcagaaaaacggcgtgaacccaggaggcggatctcggcagtgagctcacaccagtggctcacacttgtaatcccagcactttgggaggccgaggtgggcagatcacctgaggtcaggagttcgagacaagcctgaccaacatggagaaaccccgtctctactaaaaatacaaaattagtgggcgtgtggcgcatgcctgtaatcccagctacttaggaggctgaggcaggagaatcgcttgaacccgggaggtggaggttgcagtgagccaggatcgcgccattgcactccagcctgggcaacaagagccaaactccgtctcgaaaagaaaaaattcatgagactaaatcattttaaaaagatgtcttttattacaatttctttcaattaaacagttttatatttttgaaattttaaacaatttgtgcaaacgagatcctactggacctataaaactaatgtagttaatttagaaggtttaaatcacgagaagttaagcccaaatcttgggttgatttaattgttgtgttaacattgtttttatatcataataaaatcagaaggatgcatagacttgtttttaagctaatattaacttctatttctagcccaaagagtcatctatcaaaagccagctcatattaagtagggggaccaagagggaggtctctcagttaaacaaagttattgacttgtctttagacaggcaagacagagtatctcctcataaggttttcttttaacctttttgttctgaagttaatatgtcttttaagtgaataatcttgtttataacaacattttcttttctgttttgtttgtttgtttgtttgagacagagtctcgcactgtcacccaggctggagtgcagtggcacagtcttggctcactgcaacctctgcctcctgggttcaagtgattctcctgcctcagcctcccgagtagctgggactacaggcacatgccaccactcccatctaatttgttttgtgtttttagtagagacggggtttcacaatattggtcaggctggtctcgaactcctgacctcgtgattcacccacctcggcctcccaaagtgctgggataacaggcatgagccactgcacccagctataacaacattttcaaagtggcacattttaatatgaataaaccttctacaatcacaggcctagaaagtttttctttttcttacggaatttcttgactgtttacttgtactagatatttcttcggtatattcataatgacagaacaattgtttatgattgattgatctccaaagtttcccaagaaaagagaaaaagtaggaggggaaggaagaaagaagtgagcagaagttggcttcagagagataggaagggccagttcccttctctgtttaaggaggggagaagacttagatgcagggaaactcattttgacatgccaactcaaaccacagaagccagagtcagttcagctggtttcagtagtaactcaggaccttgacaattctgttaagccatgtcctttaaaagccagctcttattaagtagggggatgaagggggaagcccttcagttaaacaaagttactgacctgtcttttagacaggcaggacagaggatctcctcatgaggtttttttaacctttttgttctgaagttaatgtgtgttttaaatgaataatcttatttataacaacattttcagcgtggcaactgcagtttcagaatggtggaattataccagtcagagagagatgcaaatgatttaaaataggaagaaagcaggtgtctggcccagaggaccagattaagaagaccccatgagagttacaatagttagtgaaaatggtgcttctgcaaacctcatgtctacagaagctggtcaaagaccagatgccaccaggtagaatcccagcaaactcttcaatggtagatatctgtcaccatccttaggaggtaggtctgtggaagctcactataataccatttgcttaacagatatacaaacatggatcagatataatttcaacccacaaggagctcacagtctggctcttcttttgaggcaaaaagactaaagctgttgcctttatggtaagataaatctgccttggagtcaatccaaggtgatttaattactgtaggtagttgtaccaataaggcttgtatcccagagaactccatattgcatttaagcttgttagtaagccaggccagctctatttcctgtgaggactttaaaaaaaattgcattcacacatttaaaaattagcataagcagggtgcagtgatgcacgcctgtagtcccagctacttggtaggcagaggtgggaggatcacttgagccccagaagttcaagaccagcctggacaacaaagtgaagtgaagactgtctcaaaaaaattaattatgacataaaatgttatatgtcttaatttacacaaatataagtatatacttttaaatcccattcttacacacaaaagctagcatactgtgtaaagtattctggacctacacacctttatttttttaacataacaatatgtcttggatacattttcctatttgtctatagaaagtttcatcattattttaaaaaatttgtttttggctgggcacagtggctcatgcctgtaattccagcactttgggaggccgcggcaggcgaatcaggaggtcaggagtccgagactagcctggccaacatggtgaaaccccatctctactaaaaatacaaaaacttagctgggcgtagtggcgggcgcctgtaatcccagctacccgagaggctgaggcaggagaatcgcttgaacccgggaggcagaggttgcagtgagccgagatcctgccacagcactccagctggggcgacagagtgagactccatcttaaaaaaaaaagtttgcttttatatttcatacctgcatggtattctattgtgtgagtaggccatcatccagtggtcttcaaactttcctgtgtactccgtaacagaatttgttgttttgacatgcagtctcactctgttgaccaggctagagtgcagtgccacaatctcagctcactgcagcctccacctcctgggttcaagagattctcatgcctcagcctcttgagtagctggcgcatgccaccatgcttgactaatttttgtatttttagtagagacgggatttcaccgtgttggccaggctggtcttgaactcctgacttcaagtgatctgcccgccgtggcctcccaaagtgctgggattacaggcttaagctaccacgcctggtcttctaatagaatttttaaaaactgtatttacccccttgcacatttaaattgacatctaagatttttattataagtttaaatagttgtaaaggatgtattttccattatattttatacattccttaattttatatatatatatatatatatatagttttttttttttttttttttgagatggagtctcactctgtcacccaggctggagtgcaatggtgcgatctcggctcaccgcagtcactgcctctagggttcaagagattctcctgcctcagcctcccgagtagctgggatcacaggcacccgccaccatgcccggctaatttttgtatttttagttgagatggggtttcaccatgttggtcaggctggtctcgaactcctgacctcaggtgatcccccccacctcagcttttcaaagtgttgggattacaggcgtgagccactgtgcctgccctcccttaattataatttaaaaaatttttgcagatgtagatttatataaattggcaagccagttctctttatcaaattaactgatcaaaaaaaaaaaaacagattaactggtcaaacatagatttactttctgtcagtaaaattttgacttcatttttttaaatcaaataagcatgttaacccggtattgctgtgacaacctttatcctaatttcttttcttcttcttcttcttcttcttcttcttcttcttcttttttttttttgaaatggagtcgtgctctgtcacccaggctggagtgcagtggcacaatctcggctcactgcaacctccacctcccaggttcaagcaattcttctgcctcagcctcccgagtaactgggactacaggcatgcaccaccacgcctggctaatttttgtatttttagtagagatggggtttcaccgtgttggccaggctggtctcgaactcctgacctcaggtgattcgcccaccttggcctcccaaagtgctgggattacaggcatgagccatcgcacccagcttttttatcccaatttcaagatgggtgggcaggtaggtaggtaggcaggtgggtagatagatagggccttcagacttgcctcgcattcatcttttacataaaataagacagtttcaccttgaacacttctgtggtgagactgtttagtcttgtagctctaaactccagccatactctgatgactcccacatttataaatcccagatttgtatactcaactacttatctaacaggtcaaaactgaactcttaaaattcttacatttgtgtccacacatgattggctaagctaatggggctcagatttaccctgtcacctgaaacaactgtaaaactggagaaaatatgtcaaactctgaagttattaggtctaaggtgattaagaacagcgaatcctgagacctgagaaatagagccctatgattgctctgatttaatgctcggagaaagtttccagaccgcggtgcggggagaaggaacatcagcaggccccagcagtctctcctcattgaagaggtaaaatgaaaatccagggcagctggggcagctagagttcacaggcagagaaacaaagaggcaagagatgcacagagaagagaacactggcctgcagatgcccagggagcccactcgagtattcggttgagtactgatcagtgcacccatgtgaggaaaccccttagttcagggaaggagccactcaaaaggattagagagaaccatttacggagttcacacagggccagaaatagttcctgttcccagtagcagagtagaacatttcatgattcaggggtattgattggagtactaagaagagttgtattagtgaaggttcttgagagagagagagagagagagagagagagagagagtgtgtgtgtgtgtgtgtgtgtgtgcacgtgtgaagaaatttattgtaaggaattgccacgtgattatggaggctgataagtcccaaaaatctgcagggcgagtcagcaagatggagacttaggagagccaatgatgtggctatagtccaaaggctgacaggctcaagacccaggcagagccaatgtttcagttcaagtccaaaggcaggaaaaaagttgatgtcccagttcgaaggcagttaggctggaagaattctcttttactcaggaaagggtcagattttttgttcaattcaggcctttaacagattgaatggggctcacccacattagggaggccaagctgctttacgcattttgccagactaacgtttgaccaaagatctgagcacgtatggcccagtcaagttgacacataatgttaatcatcacaaaagtcttgcctcaatagttgggaaaaattaatcctagaataaatgctattctggtcttgcttagcaaaggttaagagcaagaccaaaaagatcaaaccttttccaaacaaaagctgaagaatatttttagggatacaaaaccatctaggaccgagtgtggtaaaatgcacaatgtttaccagccattccagaattactaggtatgagcagaagtagtaacatacaaccaataactgaccagaaatgagacagattatataagtagtacattacagacattagtcattataattgtattccatgtgttcagtaagctaaagaaaagatttaatatgttaagtaaagacatgaaagatgatggctgggcatggtgcctcacgcctgtaatgccagcactttgggaggccaaggtaggtggatcacctgaggtcaggagttcaagaccagcctagccaacatggtgaaaccccacctctactaaaaatacaaaaattagctgggtgtggtagcgtgcacctataatcccaactatacgggaggctgaggcaggagaatcacttgaacctgggaggcagaggttgcagtaagccaagatcgtaccactgcactccaacctgggcgagagagcaagattccgtctccacaaaaaagaaagatacagagatgaaaaatacactgaagactgtcagaataaacattgcacaagtaaagatttgtgattttgaagacttaaccatagaaactatccaaaatgaagcagaaaaaaaaaagaaaccctgaaaaaaatacatgaacacaacatcagtaaagtgagggacaacatcaggtagccaaatgtgtgtggaattggagtccctgaaggaagggagggtatgagggaccaaaaaatatatatagagagagaaaaaaaaatggctgaaaaatttccaagtgtaatgaaagcagtaaacacacagatccaagaatttcaaggaactccaagcacaggaaacatgaagaaaaactatatactaaggcacatcttaagcaaattgcttaaaaccatggtttaaaatggccaaagaaaaagatacattatatacttaagatcagggtgacaccagatttctcattggaaacaatgtaagccaggttaacagtggaacaatacatttaaagcaaagaaagaaaacagttgtctacctagaattcttttctcagtgaaaatacctttcaaaaatgaaggcaaaataaggtctctttcagactttaaaaaaaaaaaggaagaatttattaccagtagatatgcagtacaagaaatatcaaaggatgtaattaaagcagaaagagaatgataactggtagaaaactggagccaccaggagtcccaggctgcagtgagctatgatcgcaccactgcattccagcctgggtgacagagtgagaccctctgtctaaataaacaaacaaataaaagaaaactggagctataaaaataaatgaagacaatgcaaagggtaactaagtggataaatacataagatattttttctttttgtttcagtctcattaaaagatatgtcttagaggtaattacacattgtagcatttgtaacttacatagaagtaaagtttatgacaatagcagcacaaagactgggagtgggagaacaaatatactcttgtaagctttttacactacatgtgaagttgtataatgtcaattgaaggtagactgtgataagttaaagatgcatatgataaaatctatagtagcagagttatagctaatgagccaacaacaataaaaagttgggatataatcccaacactttgggaggccgaggtgggtggatcatgaggtcaggagttcgagaccagcctgaccaacagggtgaaactttgtctctactaaaaatacaaaaattagccgggcatggtggtggcacctgcccgtaatctcagctactcgggaggctgaggcaggagaatcacttgaacccaggaggcagaggttgcagtgagcggagatagtgctactgcactgtagtctgagcgacagagtgagactccgtctcaaaaaaaaaaaaaaaaaaaaaaaaaagttgggataaatatctttaaataatccaaaagatggcagaaaaaaaaactaaacaggaaaagcaaacagagcagatgggacaaatatgaaacaaagagcaagatggtaggttaaaattcatatcaataattaattcaccattaagggccaggcagagtggctcatgcctgtaatcccagcattttgggaggtggattaggaggatcacttgaagccaggagtttgagaccagctttggaagccaagtgagactccatctctacaaaaaaaaaaaaaaaaaaaaaaattagccaggtgtggtggtgtagttccactcaggaggctacggtgggaggatcacttgagcccaggagtttgaggctacagtgagctctgatcacaccactgcactcctccagcctgggtgacagcgagattccatcttaaaaaaaaaaaatcctcattaaaagcagacattgtgagattggataaaaaagcaagacctatttatatttatatgctgtctacagttatcccactttgaatataaaaatacaaatacgttaaaagcaaaagagtggaaaaagatgttaatatgttaacattaatgaaaagaaaaccttagtggcatattaaacaaactatattttaaaatgaatgtcaccaaggataaaggagaattatttcataataataaagagatacattcatcaagacataattctaaatgtttatgcatgtattaacagagattcaaaatacaaacttttcaaggagaaataaatctgtaattatagttgaaggtttaacaactctctcacaaaaactgatagaacaagtagacaaaaatcagtgaggatatagaagatttggacaatataatcaactgacttgacttaattgatgttcagtagaacattcgacccctaaacagcagaatacacgttcttttcaagtgcatatggaactgtttccaagacaaatcattttctgggccattttaaaagcctcagtagacccggcggggtggctcacgcctgtaatcccagcactttgggaggccaaggcgggcggatcacctgaggtcaggagttcgagacaagcctggccaacgtggtgaaaccccgtctctacgaaaaataccaaaaattagctgtgtgtagtggtggatacctgtaatcccagctactcaggaggctgaggcaggagaatcacttgaacccgggaggcagaggttgcagtgagccgagattgcactactgcactccagctgggcaacagtgagactccgtctcgaaaaaaacaaacaaacaaaagcctcagtgaatgtaagctgattcaatttgtagaaagtatgatctctggccaggtgcggtggctcacgcctgtaatcccagcactttgggaggctgaggcgggcggatcacgaggtcaggagatcaagaccatcctggctaacatggtgaaaccccttctctactaaaaatacaaaaaatcagctgggcatggtggcgctcacctgtagtctcagctactcaggaggctgaggcaggagaatcacttgaacccgggaggatgaggttgcagtgagccgagatggtgtcactgcactccagcctggctgagagagcgagactccatctccaaaaaaaaaaaaaaaaaaaaaaaagaaagtatgatctctggccacaaggaattaaatgaggaattaataacagaaaggtatctggaagatctttcaaatatttggaaaataacataattttaagtaaatcatgggtcaaagaaaatcaacagcgaagtaagtaagtatctggaactgaatgaaggtgaaagtgccacataaaatttgtgtgatggagctaaagggaaatttatatagtgcagatggtccctgacttatgacgtctcaacttacatttttcaattttatgatgggtttttcaggatgtaacccaattttaagttgagcatctgtatcagaaaagaacaaaggtcccaaatcaatgaccttggggtcccacttttagaaattagaaaaaatgaagactagtgaaaccccaattaagcagaagaaaaagccagtgtggccaggcagggaggcccatgcctgtaatcccagcactttgggaggctgaggcgggcagatcacaaggtcaggagattgagatcatcccggccaacatggtgaaaccccgtctctactaaccatacaaaaattagctgggcgtggtggcgcttgcctgtgtaatcccagctactcaggaggctgaggcaggagaatcgctttaaccagggagtcggaggttgcagtgaaccgagatcgcgccattgcactccaccctagcgacagaacgagactccgtctcaaaaaaaaaagaaagaaagaaagaaaaagaaaaggccagtctggaaatcaatgaaacagaaaaccagtacagaaatcatgaaaccaaaagctagttatttgagagagcaatgaaactcataaacctctacccagactgatcaggggaaaaaagggaagacaataactgtaatatcagcagtgaaagagatgacatcacacagattctaaagatattgaaagtgtaataactgaatattgatcagcaatctagaagaaacagattccttgaatgacacaaactaccaaagttcactcaagaagtaaccatctgccgggcgcagtggcttacgcctgtaatcccagcactttggtaggccgaggtgggcggatcacgaggtcaggagaccgagaccatccttgctaacacggtgaaaccccgtctccactaaaaatacaaaaaattctccgggcgtggtggcgggcgcctgtagtcccagctactcaggaggctgaggcaggagaatggcttgaacctgggaggcggagcttgcagtgagccgagattgcaccactgcactccagcctgggtgacagagccagactctgtctcaaaaaaataaaataaaataaaaataaagtttagtaaaataaaaaaaatcagtaaaatacaaataaaatttagtaaaaataaagctgattctaaaatgcatgtgcaacagaggatctagagtagccaaaataagctcctctgtctgacttaaagtcttaattaaaataatcagacaggccgggcatggtggctcacgcctgtaatcccagcactttgggaggctgaggcaggtggatcacctgaggtcaggagttacaaaccagcctggccaacatggtgaaaccccatctctactaaaagtacaaaaattagtcaggcgtgatagcacacgcctgtaatcccagctactcaggaggctgaggcaggagaatcgcttgaacccgggaggcggaggttgcagtgagctgagatcatgccaccgcactccagcctgggcgacagagtgagactctcaaaataatgaaataaaaataaaaaactgaacttcatatcttgtccccagatctgatatagtcacttcttcctttgttcagttaaacccagcttcatgtttctgcttgcccagggccaataactttgggatcttccttgactcttctttttctgtcaaaccccgtaccagtctttcaggtaaccgtaacttcagagtctatccaggtcacctctgcttgtccaagcccctgtcatctctcacttggatgatttcattagcctcttggccaggatccctgcttctgctggtatttccttagtttcttctccctacagcagccagagctaatcattttaaatgtcatatttctgtctcgcctcagctgcaaaccttccagtaccttcccagctccttctaaataaacaccagaggccgggtgcagtggcttatgcctgtaatcccagcactttgggaggccaaggtgggcagatcacctgaggtcaggagttcgagaccagcctggccaacatggtgaaaccccctccctactaaaaatacaaaaattagctgggcgtgatggcgcctgtaatcccagctacttgggaggctgaggcaagagaatcgcctgaacccaggaggtggaggttgcagtgagccaagactgcgccactgcactccagcctgggcaacaagagtgagactctgtctcaaaaaatataaataaataaacaaacaagcaccagagccctggcagtcacctgtgaggcttcaccccgtctccccacccactataacctctgacttacctccttttacactccttcttgctgactctgttccttccacattggcctctttgctcttgcttgaatgatccccagtcatcctccctgcccagggcctttgcacttgctgttctctgccaggaaggtgcttcctccgtagttcactcttcacctcctttgggtctttgctcagttgtcatcttcagtgatggctcttctgatcaccttatttaatttttattttttaattaaaaattttttttgtggagatgtcttgctatgttgcccaggcttatctcaaactcctggactcaaacgatcctcctgcctcagcttcccaaagtgctgggattacaggtgtgagctaccacacccggccccgatcaccttatttaaaattgcagctcccagcccccaaccccagcatacccattcctcttttttttttttgagacagaatcttgctctgtctcaaattccctccagccatgagccctgaaatgaaacaagttatctatttccaaaacacaacagtggggcaggcataggatagacattcccattccaaaagggagcaataggcaagaagaaaggggtagctggtcccaagtaagtccaaaacctgagacagagtcttgctctatcgcccaggctggactgcagagtggtgcgatctcagctcactacaacctccgcctcccgggttcaagcgattctcctgcctcagcctcccaagtagctgggattagaggcatgcaccaccacacctggctaatttttgtagttttagtagaggcagggattcaccatcttggccaggctggtctcaaactcctgaacctcaggtgattccccccgccttggcctcccaaagtgctgggattacaggcgtgagccaccgcctggtctccctcattccttttgttatcagtcatccctcccagccctggcaactgctgctcactttcctgaccttagaattttgcttattacagaatgtcatataagtagaattataaagtatctaagcttttaagtcctaatgcatttaagatctgtaaaaagtaaagtagaggttcctcttcaaagactttcctccccatctaattaggaataaatagtaacttctcttagaagaaaaatttattcaaagacctgtgctaacattcttaaatatctgctagccataataaagaaatcaatgtactttatattcttagctctcacaatttagcctaaatatttgccctggcatgcttatactggtccaagcaagcattaggtcatggcctgttcctcttccttatttgaaggtgtttttacctttctcagcactccacaagttacttcctccttcctttgttctcctctgcctttgcctcttttaaaaagttctaagttcctagccaatcaggacaaatacagaatgtgaggtcctgttccagccaatggaaactggacacagcagtagggtggacgcgtcaggttgtaaatgaccctgtctcctttgttcagtgtactctcatggcaaaactgctggtgagtgtaccctttctgcagaaagtaaaaaatggccttgctgaggaaattaaatttatgttcaagtgctttttcttcagggcaccagggaacaagcatttctaacagatccatatgtgtcgctgcatgtaatcagtggttcattcctttctattgctgagtagtgttccattgtatggatgtacccgtgtattgatccatccatccactgaaggatatttgagttgtttccagtttttttgtgattatgaataaagccactgtaagcattcacatagaggctttggtgtggacataagttttcatttctcttcggtaaataattaggagtagaattgctaggtcgtaggataagtatatgcttaacttaggagaccctgccacagtattttacaaagtaagggtgctattttgcattactaccagcagttcacaccatatgtattcacaccagttgctccatttggtcttcgttctgtgacttgtctctattctcttaacagtatctttagaacagcagaagtttttgattttgatgaaattcagtttatcgggccaggctgggtggcttatgcctataatcccagcactttgagagaccgaggagggtggatcacttgaggccaggagattgagaccagcttggccaacatggcgaaaccctgtctctactaaaaatataaaaattagccatacatcgtggcgcatgcctgtagttccagctactcaggagtctgaggcaggagaatcacttgaaccctggaggaagaggttgcagtgagctgagatcatactgctgcactccagcctgggcaacagagcaaaactccatttcaaaaaaaaataaaataaaatttcagtttatcagtgatttctcttaagtttcatgcattgggtattgtagctaagaaatcttcacctaactcaagtttacaaagattcttctgttttttttctagaagatttataagttttaatttaacttttaggcttataattcatttcacattaaatggtgtatgtggtgccaggtattgctcgagagtcatcatcttgcataggggtttcggatgttctagcaccatttgttcaacagatagcctcttttcattaaattgccctttcacctttgtggaagatcaattcagcatgcatgtatgggtcacttgtatgtttttaattgatttctaaattctgaaaggaaaatgataaaaaggaagattctttcactatatctgcatttccaatttctcccccaagaacgctttcctaacacctgcaggcagaactaaccactctgggcccctgtagctgttgtgcgtatgtttattattgagtttttctggcctttgcagttctttcagtgtctgattctccctctctctcagcttctttctcaagactgtgcatccttggaggtcagaaattgtctcatcctgccgggcgcgatggctcatgcctctaatcccagcactttgggaggccgaggcggttagatcacaaggtcaggagttcgagaccagcctggccaacatagtgaaaccctgtctctattaaaaatacaaaaattagctgagcgtggtggcacatgcctgtagtcccagctactcgggaggctgaggcaggagaatcgcttgaacccaggaggcggggaggttgtagtgagctgagattgtgccactgcactccagcctgggcaacagagcgagactccatctcaaaaataaataaataaataaaagacaaaataaattttctcatccttttttgtcatcttcatcacagctggctctctaatgttcgttgaatgaaagggccattcccaaatctctgataagtcaagcatttttaggagagctaaaccagccaaatgagaccaggaagtgaagaaaaatacctttttttttttttttttttttttggagacggagcctcactttgtcacccaggccggagtgcagtggcacgatctgagctcactgcaacctccgcctcccaggttcaagcaattctattgcctcagcctccccagtagctgggactacgggcacacaccaccatgcccggctactttttttttgtatttttagtagagatggagtttcaccgtgctggccaggctggtgtcaaactcctgacctcgtgatccgcccgcctcagccttccaaagtgctgggattacacgcgtgagccaccgcgcccagctgaaaaatacctactttttttttttttttacagcaggaggtgtttctgcttctactttgctgtgacttctgtttctccaggcattcctggtatggacatttataccaggtgttttggtcattggtgggctctcagcttcagaggacacttggcttctctataccctatctttggtgacttctgcttgttggacttcagtcactgcagttcacaagtttcacactcatgaaatatttattgttaattaattccaatatttaattgattgcttctctttctcagcactttggaaacatgtttactacctagacattttcctacagtagatttttaattcttaacattttgatcttaagcaatttaccatgatacttggtttccatgagagatttaacagttggaactgtactgcccataagatgcaggtggggtcatggtatgtagttgggaactctacaaatggcagcccttcatgcctttgcaaaggctgcaggttggaaagctgccattacttgtgagggcgtccaaatgcttcctgtgaatggaagatgtgtttctgtgcctcactgtcttcactgtgctggattgctggctttctcagatgtctttatttgagtcaacctgtattccatctccccactcccacccagtcttgatcatctcatttgtgtgtatacaaccttcccctcacacacacctctttgcagagtttcgttgcactgggcgttgggaggagaggcgtcagagttgggtggaatcatttcaggctgactgtccagacatagctctcattagaagcatcagtaaataagctgaaaaccaacaacacgtacagcacagtctttctgtgtttgcagcttggaactgagcagccctcactgggtgcatttctaatcctttgtctcatcatttatatgacatccacttagaaaaaggtgttaaaatcatataacatattgctttttccccttttttattagcagttttactgaggtgtaatttatatgccataaaatttaccatttgaatgttcaattcaatgatttctactaaatttatagagtgtgcaccataaccacaatccagttttattttttatttttttttcgagacagagtctcactctgtcactcaagctggagtgcagtggtgcaatctcggctcactgcagcctccacctcccgggttcaagcgattctcctgcctcagcctcccgagtatctgggactacaggcacgtgccaccatgcccagctaagttttttgtattcttagtagagatagggtttcaccatgttagccaggatggtctcgatctcctgacctcatgatccgccagcctcagcctcccaaagtgctgggattacaggcgtgagccaccgcacctggccccacaatccagttttagaacatttccagcatccccaaaagatcccttttgtccctttgcagcagtcctacccccacttccagccccaggcgaccattcatttgctttttctatagatttgtcttttccgtacgtttcatgtaaatgaagttttatacaatatatgaccttttgtgactgttttttttcccagcatgtttttgaggtttatctgtattatagcacgtctcagtactttgttccctttttatcactgagtattattccattgtacggacatacatttttctctattcactagttggtgaacatttggattgtttccagttttggtcatcattaacaatgctactatatatatccatgtataagtctttgtgtggatatacattttcatttctctagggtagatttctagaagtaaaattgctgagtctttttttttttttttgagatggagttttgctcttgttgcctgggctggagtgcaatggcataatcttggctcactgcaacttccgcctccctggttcaagtgattctcctgcctcagcctcccgggtagctgggattacaggcatgtgctaccacacctggctattattattattattattttttttttttttgagacagagtctcgctctgtcacccaggctggagtgcagtgcgcgatctcagctcactgcaagctctgcctcccgggttcatgccattctcccgcctcagcctcccaagtactgggactacaggcacccgccaccacccccggctaattttttgtatttttagtagagacgaggtttcactgtgttagccgggatgctctcactctcctgacctcgtgatctgcctgcctcggcctcccaaagtgctggcattacaggcgtgagccaccatgcctggcaaattttgtatttttagtagagacgaggtttcaccatgttgatcaggctggtctcaaactcctgacctcaggtgatccacccaccttggcctcccaaagtgttgggattacaggcgtgagccaccacgaccggtcaaattgctgtcttatagtaagtttatgtttaactttttaagaaactgccaaactatttcctaaagtgactgtaccatttacattcccaccagcaatgtgtgagtgttccagtttttccatgtccttgccaatagttggttttgtctgcctacctatccccgcccctgcttttttttttttttttttttttttaagagacagggtcttgctgtgttgttcaggctggcctcaaactcctgggctcaagtgctcctccctagtagctgggactataggcacacaccagtgtaccaggggttgtctgtcttttgattatagctattggaatgggtgtgaagcagcatctcgtgattttagtttccatttcttgaatgactaatgatactgaacatctttttatgtgcttattagccatttgtatttcatctttggtgacctgtcaattcaaatcttttgcctatttttttttattaagtttgtgcactgtcttattatcgagtggtaagagttctctgtattgtgggtacaagttcttttttttttcgagatggagtctcgctctgtcacccagggtggagtacagtggcacgatctcagctcactacaacctctgcctcctgggttcaagcgattcttctgcctcagcatcccaagtagctgggactacaggcttgcaccaccacacccagctaatttttgaatttttagtagagacgggatttcaccatcttggccagacgggtctcaaactactgacctcgtgatcctcccacctcggcctcccaaagtgctgggattacaggtgtgagccaccgtgcccggccttccaagttctttatcaggtatatgatagcacatgtagctttttatcttccttacaccagtgcaagtttatagaaaggaaaagggatagttaatagtaatcataataatgtttaatatttgaatgttttttaggagctggacactgggctgcatgtttcacggggatgatttgggtcactcttcaattttctaaaggttacaggactgtttcaaacacctctccttctattaacagttttcaatggagaaagaaaacaaaagattgatttggggaattcttaggttaatgcgagttagaactgaccttttaacattattaccataaggactaaatggctgttacagacccaaaataggagcaattcaaacaagcgagatgcttctctctgtaacaggctgacagtcgcggtcctggaggggcacgatggctcctcagggtcagacacctcctgtcccttactctgtaaggcacagcttctacctcaggatccaaaatgcccacgccagctgccatggcccctcacaggcagtggacaccagaaagaggagagggagtgcgtgcctgttgctttgaagaacctgacccagaagtcttacacagcacttttgcccacataccatcacttagtcatgtggtagtgcctggccacaagagagactgggaaatggagtctttattctggggtgatgtgtcccatctgcaactgaggggttaaggcctaaaacttagggattgaaggcaggagaaaaaacagatgtcgagagccagtgttgggattgcagggtcctgtgtgggattcctgttcattttcatcaaagaaagtctagttgatatgcaattgttggcctaagtgttattgaggagttaattttactgttgaattacaccagaacatttgtcatgtgaaataactataaatacgactgattattgcctagtgtattacagttctctccagagacacaggaaacaatagggtctgtctatcggaatagatatcctaaaagtgtatcttttgaagagattgattataaggtattggctcatgagatgatggagcttaagaagtcccacagtctgctctctgccagatggagacccaggaaagctgctggtgtagtttgaaggcctgagagctgagggccagtgatgtggattccagtctgaaggtctgagaacccagagggctgagggcaggagaagatccatgtcccagctcaagcaggcagagggagggtaaatccaaccttcctttttctgtttttttgttctaatcaggccctcaacggactggaggatgcccagccaaagtggggagggccatctgcttaggcagttcaccaattcaaatgctagtctcttctagaaacaccctcacagacacacctggaaataatgtttaagatctgggcatcctgggggtctagtcaagctgacacatacatttaaccaccacacttagggaatatgaaaataacttagacattaaatttgattttggtgttgctgtaaagaatacagatcttaggcaccagctcgtaggaggggtggtaaacaaggaggacagcttttaccaggttgcctttggaagcaaaactctgctttgctgttttgttttcaagcggctgcatggccactgagcgagccctttcctgccatcaccacagtgccacttagtgaacagtttctccaaggttaactgtagctgctcttgttcagttaatatgtagttctggattaatatgccacatctgtcactttaaggaagaaaaagagcagttccgtttagagatcccattgctcctctaaggacagcagtcatttctaacaagcagagggaagaaatcttctggttccatttgtcctttgtgtgtgttgaaaaccttgacaacaaaacaaattggtactgaatggtttgaagaaataaacattaactcccctttagtatttcatttctagggtttccagccttggtaacaattaccaagaactttgctgtcatggggtacagctctaggctgctgacaggatcttcatactggaggagactatacttttatagccaattccaactgccccagagataacttggataaacactggctcctccactacctcctaatccctgctgggttttacactgtggggaggtttagacaacttgttttctttcccttttagcccttctctctacactcccctccagctgccttcttcccccacccccaaatgtaacgtgatctcctaggcctgctctgctgcttttgcagattcttttagtcttagagaaagagaggaaaacattaaccattggtttaccaattcaaagttaacaacattttaaacaaaaggagcttgtcaataaccagggcttgggcttttcttttatcagttcctcctacttggctgagtttacctcatcataaatcttatcacacagataataatttgacagctttagggtttttgcagcattgctattttctattgcaacaaagctgtaacattgctatttttgatgtatttgttttatagatatcttaatcttcaatattcagattaaaaacaaacaagacttagtgatttaaaattatttggtaaaaaggtaaagggacagagacatctcatgaaaacatggcaaaatctctttaagtttaattcaagcacttagagtttaataagaatatcccaaaccatttttgcttaataatatttctgtttataattaaaccttaaggtacagtgttgtaactggatttaagaatttagagttttgttgtttcttcgccatcagcgaaattaagatttaaaacctatagtttgcaacaaattttagtttttcagcctagtgtcctaaaacttgtctgtcatgggtacctagtaggtgtcatgtcacaggaaagggatctaacaagggagtttgaagataggggttttccttgctctgccactaacttgctatgtgacctagggcaagttatataacctatctgtgtcacagttgtctgaaatgcaatatggggaaaataaattcttaaaatgacaataaaaagatttctttttttttttgagacagagtcggcgcaatttcggctcactgcaacctccacctcctgggttcaggcgattctcctgtctgagcctcctgagtagctgggattacaggcacacaccaccacgcccagcaaatttttgtgtattttgtagagatggggtttcgccatgttggccaggctggtctcaaactcttgagctcaaacgatctacctgccttggcctcccaaagtgctgggattacaggcatgagccaccacgcctggccaaaatatatatatacatattttaagtgaagatgtgtaaggactaaaggagcccaaaggagacagtaggggataagaaaggcaaagagagtggtaatagatttagcagaatcaaggatgccaaaacctcttctgagagagaccttgattaagagtgagctggtttgcctgtaagaatctgcggaaaggtcagaaattagagacattagtaccacagaaagcaggggatgaggcctgaagctgaaatcaaggggatcaattgagagcctataaaaggagggtttagattcttcaaacccctcccaaccccacagagtcagatgaccacctctcctaaccacctacaggagaccagaggcattatattgagaaatgcccggccgggcgcggtggctcacgcctgtaatcccagcactttgggaggccgaggcgggcggatcacaaggtcaggagatcgagaccatcctggctaatgtggtgaaacaccatctctactaaaaatacaaaaaattagccgggcgtggtggcgggcgcctgtagtcccagctactcaggaggctgaggcaggagaatggcgtgaaccctgaaggcggagcttgcagtgagccgagatggcgccactgcactccagcctgggcaacagagcaagactccgcctcagaaaaaaaaaagaaaagaaatgccccgtagaggctctgcactcagaagcctgacccccagcatgggtccgggtaggccattggccgaaaatagtggagtgggcaaagagcagattttttaaaagtctgcatactgaatagtaagattcccagccctcttctaatacctgctagccaggggcctaataaggaagagattgaaatattcttttctagggggaaaaagttgaatgaccctagagaaaagccctacagataccaacatttagggattctcttgaaaaagcgagtttaccatctcctttcactgaagcttaccagtggctgtgccccttccacgcatacaaaactaacagtcatcttttagttgctcattcatagagagtgagtggcctgccaaatctctctaggcattagaggaaggtctctaacctgatattaatagataaagggtaaaacaaatagcagaaaacaggaacttggtggaaataaagacaatgcagtcaacagaggaagagtttaaaagaactattcagcttgggcaccatgactcacgcctgtaatcccaacactttgggaggctgaggtggacagttcacctgcggtcaggagtttgagaccagcttggccaacatagtgaaaccccatctccactaaaaatacaaaaattaactgaacatggtggcaggcacctgtaatcccagctattgggaggctgaggcaggagaattgcttgaacctgggaggcatagcttgcagtgagccaagattgcaccactgcattccagcctgggtgacagagtgagactctgtctcaaaaataataaataaataaataaataaataaataaataaataaagctatgcatttcccacaaatactgcttaaaaaaaaaaagaactattctttgctatccttggagagagagattgcaaatgtcagaaaacaggatgcttttaaaaacaataacaatcagaataagaataaaaatgaattatttttcttttatttatttatttatttatttttgatacatggtctctgtcacccaggctggagtgcagtggcaagatcacagctcactgcagccccaacctgcctcagccccccaagcaactgggactacaggcacgcaccaccacacctggctaatttttaaattttttgtagagagatggggtctcactatgttgcccaggctggtcttgaactcctgggctcaagtgatcctcctgcctcagcctcccaaagggctgggattatgggcatgagccaccacaacccagcaagaatgaattgtttaatattaaaaacattatagcccaaatttaaaatccaacagaagatgtggaaaattgtctcagaatgtattgggttggtacaaaagtaattggggcttttgccattttaatgacaaaaacgttgattacttttacaccaacctaatagaacataaagacaaagacatggactgttgagagaacaataagagagaacaggtaagaaaattagaaactctcgggaggctgagacaggataatcgcttgaacccaggaggcagaagttgtagtgagccgagactgtgccattgcactcctgcctgggcaagcaacaagagtgaaactgcgtctcaaaaaaaaagaaaagaaaattagaaactcaatcccagggctttaatatcctttttgtaggaattccagaaagaaaacaaaaaaataatgataataacgaagaaacaaatggaggagaagttaggagacgttaaatgtaaggaaaaatttcagagtttagtgaagtgagtattttccaaaacagaattatataaatctctggatttatgagtgcccagaataatatatgaaaggccatatcattgtgaaagttcagaacaccaagaatattagaagatcctaaaagtttccaaagagaaaaaaagcaagtcattttaaggggtcaggaaccagaatgggattgcatctctaaacaatacttactggaaactagatagtagcagataaataaatgccttcaaagttcatttgggaaaattattttcaacctagaattctatacccagacaaactgtcaagatagaagaaaggtatttttttagacatgcaccctccctccccacctctgtgaagctactggatgatgaattccagcaaaatgaagaagtcagtcaataagtagaactgagcccgggcacggtggctcacacctgtaatcgcagcactttgggaggccgaggcgggcagatcacgaggtcaggagattgagaccatcctggtgtaacacagtgaaaccccgtctctactaaaaatacaaaaaaattagcagggcgtggtggcgggcccctgtagtccgagctactcgggaggctgaggcaggagaatggcgtggtgaacccgggaggcggagcttgcagtgggcggagatcgcgccactgcactcccgcctgggtgacagagcgagactacgtctcaaaaaaaaaaaaaaaaaaaagtacaactgagaatatttgaaatgttttagtgattggaaaaaatatcactagatgtttgacagatctttgaagtatttgagggaaaatggtgataggtatatagaagactaaatgaaaacaaggcaatatctctaggataaacaaagatgtataataaaggaaacattcatagtatattattggctaagcagtgaagaatatttatatcatcctaataatgtaaacactaactattgatttatcccccaaattgtgattttattgttttggggggttatggatggtgatagtataatactgctgtcataagttaatagataatggataaatctaatgaattcagaaagagcaagatgatgttactagctaaaaatctgaaggaggctgtctctgcggaaacatacaggggaaagggattgctgttttttattaccagatttttacaagactatttttagccttttttacaagacttttagtactgcttgagttttaaccatgttatgtatattactttgattaaaaagaaaaattaatttaaaaaaatgagcatactaataccattaattttctccttctagagaaaaatgtttaacagttaggtttagacttgttaattataaaaatatagtagtcttactgtaatgagattttctagaaagcggatttactctaaggcagttcagatttggtccccagctgagaattatagcctggaaataccaacagaaaaatcagtgtcatttgaaggacagtcatctgtgcagcctgtgcatgaaatcatgggtctgaattaggcccccattcaagatgcgggggtgtggggtttgtgttttgctgaagctgtggttgcaaatctttgctttaggatgaaaggtggggctctctgttacatttgaaccttagttgctactcctacctcacagctagtgtttctttctcttcataaaataaattgatttcctggtcccttggaaaacctccagactgctttctggaatcattttcatatagcatgtttgttaatttgagctcttgactgagtcccagtgagatgtaggcaggaacacagacctgatacagaaatagtcctcttttggaaataacaatagctactgtttatcgagcaccttctgtgctttgggcactctccctacatgattcctaatcttcataataaccctgtgagataactactcttatgactacctcccatttttcagatgagaaaattgaggctctgagaggtgaagtcgttgcccatggccccccagtgagtggcagatctgagactcagcgctagcctttgtgacacataactcgtgaccagcaggtagtgctacccgattgaaaaaatgtgagacttaaagggatgcatgtgtccctatacagtggcaaaaactggtatgacagaaatgatgttttaagctatgttatcctttagttggacatacactggacccacagagctagagtacctggcaggctcttgctggacaatgtttgctgaatcagagaaagtgggctttgttggcatttgcatgtgaaatactgtgcacatacctcaagatctttgctgttccccccctaaagctactagctatgctggaaacagtgaaggggtcagccagacctggggtcaccttgtcctggggtcccaagccaggcctcctgtcaggttcatctgtctctgttccacctgcactcacacccagtcttgctgctgagagatggagcatccacatcctgaatcctgcctccttcccttgacacacttctatggtgggtgcctgcccaactttttcttaataggactaaattccaaattaaaaaaaaaattcagtctctctcctctaatcatccaagaagagtcctcttttggtaacttgttctagaactatgcctatcttaaattcagaaattgctttcaaatgaattaaattttagaaatatccattcctgttcgtcctattcccagtgaaaataagcaactcttccctcttccaaaacagtggatttcttttctttttttctttttttgaaatgtagtcttgctctgctgcccaggctggagtgcagtggtgcaatcttggcacactgcaacctccacctcccaggttcaagcgattctcctgcctcagcctctcgagtagctgggattacaggtgtgtaccaccatgcctggctaatttttgtatttttagtagagacaggatttccccatgttggccaggctggtctttaactcctgacctcaggtgatcgcccaccttagcctcccaaagtgctgggattacaggcgtgagctactgtgtctggcccccaaacagtggatttccatcttacatatgcatacagatcatctgggagcttcagatccagcccattgcacatattctgattctagactagaaaccaagactccgcattttaagcaagccccacattattcaaaatagattgtccatggattatgcttttagaaacattgttcagatcccatgctttaatgttggggagtttatcctttccttctaaatattttttttttttttgagatggagtctcgctctgttgcccaggctggagtgtggtggtgcgatcactgctcactgcatcctccacctcccaggttcaagcaattctctgcctcagcctcccgagtagctgggattacagatgcccgctaccacgcccagctaatttttttgtacttttagtagagatggggtttcaccatctcaaaaaaaaaaaaaaaatcttaaatagctctagttccctgaattattttctcctaagacatgttttcccaacagttaatgttacttatttcatcttctgatcttactcatattatccacctttcacttaaattggaaattttctgccattttacaaagttcaagactttcctaccttcctacctcctggctgtgtgaacttggacagaatacttccccctctcccttcagtcagttttcttacttgtgaaatggaattaaactatacaaaatcattaggttactgtgaggcttaagagtgcctggcacatattacatgcacattaaaaatgtatttcttcccaaactaatgcaacatagcaaacaagttagttcacagtctttaatttctttccttttccctttctttttagctaatagcttttgggtcttaggctgattagaaggaatccttcttggggtgttcttctggtaagaagattgaagctctcctttatttgcttgtaacagaatgctcttcgtatctttcaaaagcgtcaaaagctggatatggaaagtataggccaggtttgatacagaagtgtagtttggcagttttttcagtatccattgccactaattcagtattttgttagatctgttgatgtacggttttataactaaaattaaaaagatttgggtacagtttataagcattattcattttaagaggatttaagaggataagaccattttagactcgagcagaatattagtaataataattagcttaaaaaaataaacatataccaatataaagaacagcacaaggcaagcaaatatctgggcctatattaacagacggtattcaaggatatggctagaatttccaaaatgcctaataatgtgtaattcttggcattaagccgccaatttgaatatggccctttaatatgaaatgctatataatgacagactttattatagtctgatccaaggcccctgaactttccaaaagggcaacaaaacagacagatactgtattttttcactagttggtactggtggcacccttttgagtgttatttttggcattacgtgtagaggttgacagattcaagacaacactaaacatgttagcagtcaccacacgcaggctgtccagcagaatgacagcgcctcccagatcctagaaagcatccagagtcactgcacccatcccctgggggccctgtagtcctgcatctccagatgatgggagctgcaaacttccctgatggtgccctcttggatgctcatgaggcctgatctggtaccacgactctagcagtaggtccattcaatggcagtaaaaatacatgttgtatgttgatcagaatttggcaggacacatgtggaaagttaatgacatcctggccgagaacccctgtgatatacactggttacatcctgtccaactggagtttttatgtgctgtttttggcagagccaagtgaagatcttgttacttagccattcctgaggtactgaagatacccgggtttttgtcattacaggataggctagaaagtagccagggtctcataaccaaggctttctctgaaacatataatgacaatgctagttatttgaccaaagatatccaatgctttaatccacttggaatttattcttgatgtgaagggtcagcatcatccactggtcgggagcctgatccttggagccaggcagacctgggttgagtccatctccgcctgtttccagctgtgggcaaggtgtctgacttctctgagcatctgtggggtttgtttgtttttaaacctgaagaattgggataatctgtttcctcacagaatcattacgcagatgcaatacagttaatgaaggtaacatggcacataggaagagcacaatacacgtcagctcttactcttcatggctagaagatgtggactaaacttccccctttacagttttgtgattaacaccatttatgaaagaaataaatgaaataaattccttttcctgtcatgaggggaacttacctgattttattgaattatttggtgccattgagttccttattgtttccctctgacctagatttctgggtgtgcgccagtaacacgactgtgattgttgctgctttataacattgtctgtggtcagtccaaacaagagtttaaaataattgaatgaaatggattttaaacaatggtccccccaccctcaccccgtggcactcggccaaaggaagaggaaaagttctctgttaaagaaaatgaatcagagaagaggcccagaccgtgcctccgaaaattctcattgtagggactaagtcctctcactctgaactcacacccgtctgttcgcagcctcacctcatatcctggtctggatgtgcggcctcaccggggtgtcgtgcgcacgtgtggttgtcctgtgggtgccagctctgacttcctccccagacacccactgcctcagccttaatcacaggacgcgcgttgagtagaaatgagaccagttagtattggtaactgcagaggaatttgcattttcaccagcgttctcgggtcagagggtttgcctggcaccgcgtactgggaaactcgccaaaagcggtgcaggttggagacgccccaggccgcggtggagttgcgcgcggcttctaaagtggagtggagcaggcctgcaccctccccgccggggctgggacggcgcttccaggcggagaaagacctccgcgggccgcgcgcggccttccccctgcgaggatcgccattggcccgggttggctttggaaagcggcggtggctttgggccgggctcggcctcgggaacgccaggggcccctgggtgcggacgggcgcggccaggagggggttaaggcgcaggcggcggcggggcgggggcgggcctggcgggcgccctctccgggccctttgttaacaggcgcgtcccggccaggcggagacgcggccgcggccatgggcgggcgcgggcgcgcggggcggcggtgagggcggctggcggggccgggggcgccgggggggcgcgcgggccgagccgggcctgagccgggcccgcggaccgagctgggagaggggttccggcccccgacgtgctggcgcgggaaaatgttggagatctgcctgaagctggtgggctgcaaatccaagaaggggctgtcctcgtcctccagctgttatctggaaggtaagcccgggccgcacgggttgggctgagtagccgcgcgccctcccgctgctgctgggcccttcctaggcctcgccgcccgcgcgctcccgcctgcgccctccccgggtcttgtcttttttttctttcttccctcttctcttctcttctcttcagttctcttatattctgtctctctttctttctctctgtgtctgtctcttttctcttctcttgtctctctctttttctctctctctgtctctttctctttctcgcgatggcccctaggcgccgccggcggagcgtggcccccagccccggcaccagccccggtagagccacgccggatggtgacggcggcgtccgggaccccacagtgcgggctcccccgaaaaagtttgagaaagccaactcgccaggccttaacatccctgggatcccacgttgtggaatttccaccgttaattgggactgtgtgttaaaaagatcgacccgtgtttgtgaaaacatgcgatttccattaagcatcatgtacacaaggagcaccactctaccatttatggatacagaatttcaagtgatctttccatctcccccacccccatcttatctacagaccacgaagaggtcttaggaattcgctctcttttcaggggaggggagcccatttaagaaggcgtttccaaggagtcccccagagtactggggtgtcatgttcgtttccttaagtgaagctcagggccaggagggcttcacttttgcgttctttctacctactctccttctctaattacatctgttttattttatttttctttcctgttgtagtctttagttttataaatggaaaaaaagtaaattaagggttatgggtcttcactttcgtagcttctaggtgggtatgaaattgatctgatttgagactggtgctcccagaccattgccttcagcagaaaggctatctgtgcttcagagatggagttgcacttggacttctctgttgttttcaactaagaatataagagtaaaaatgtggccgggcgcggtggctcaagcctgtaatcccaacactttgggaggccgagacgggtggattatttgaggtcaggatttcgagaccagcctggccaacatggtgaaaccctatttctactaaaaatacaaaaaattagccgggcgtggtggtacacgcctgtaatcccagctactcgggaggctgaggcaggagaattgcttgaacccggcagggggaggttgcagtgagccaagatcgcgccactgcactccagcctgggcgacacagcaagactcggtctcaaaaaaaaaaaaaaaaaaaaaagagtagaaagaaatgtgaagctcactttagatcaagtaggatttctgctagaatgccatgtttttctttttaaaaaagcaggagaaataaacaggcctcatttattttctaacctatttttctttccttaacctctgactctaccctgaaggcctgcttggagtcacatatgcagatcgcactagtgatgtgggtgaaacctgtttgcccttcttgccacagtttgtgtacctcactcctcttctgtctctgggattccagggggctgaatggcccagagaaggctcatctaggtggagtgggctcacagagcctagcatttggagggtggagattgtttatttgtccctaaaagttgatgtctgagcattccaaatcatcttcaattaatacatcgcttattttttccaagttgctcaaaattctgagcacattctcatttacatctgtatcgagttagtgttaggcatcatcatctccactttgcagatgggtgcctaaggcggggatgaatggacttttgtaagtgcagagctggagcaaagtttaggtcacagctttgccaagtcattgttttgttttcctgggagtaaaccggagatttagtatggacctggctggtttaattattgttttgggaaaaaaatctaaaactcattccaacatgtcttgaaatgaattataatgttgactctaaagtctcaaaaaaaaaaaaaagttctagtacagcagactgccttgggttaaaccttcttctaggaaggtgtcactctaggaattctttcttactgagttttcttcttttcgttgttatagttaattagagaaagtaaatggaattatcattttgtagttggtagactttaacaagcctcagcgatagtcttttaaagactggaagtgctatttgtcttttgggaaaaatagttcctaacatctggatgagtaatccaggctttccactttcactgacagcctaaatcagtcaatcagtgtcgatcagtcagtcagtgtttattgacgcccactcagggtcccaggcattccctgtcagggagagaattgaatgagatcaagtttttcattgttctatcaaagaagcttccaccataaatgtggtttgatggtgcatgctacctgctactcagcctagctgtcaaagcaacactggtaaacataaagatggctagcaattattaaatgcttactatgtgccaaatactgttttattactctatgtgatctgatatagttatatttcatgatagcactatgattatttccattttatagatgaggaaactgagtcaccaaaaggttaggtaacttgtctagggtcacagagccagtgagtgggggtactggaatttgaactcatgctatctggcttgggagagcccacttttagccactggatgctctactgatgtctcttttggaattcactgtgattttcaaaatggtatctccagaaactcctccaattaatccctgaaatgtaagggtagtctctttttcttttctagaaacctgatcctggcaaaatgattagctttaacagtaggattaattttttttaaatgtatataaaaatacttgtatgtaagaaatcctaaaagggtatttagtgaaaagtaattccctctcctattcattttccccacctagaggcaatcaccctggagttttatgtgtgtccttctagaaatactgtgtgcacatataagcatttattataacatgtgaattttgctctccagctttttgtgaatatgcatgtttatagcagtatctattcaccaggaccgcatttagctttgcaatattcggtctactttgaacactgaattttctgattatttctttcccctggagacgtcaaagaaaacatgaacactagccttttgaattcagggttttcttttcattttattttcatttgttcatgtgtttgtatttacatacattgtgcctgaatattgtacatgtatgtgacagttgtgtactgtagaatcaaaaactaatgtctgtgaactgaactcttcttgaactttttgttgttgttgttattgttgcttttggagatagggtcttgctctgtcacccaggctggactgcagtggcacaatcacagctcactgcagccttgagctcctggtctcaagcaaccctcccacctcagcctcccaagtagctggggctgcaggcatatgctacctgactaattaaaattttttttttttttttgtagagacagggtctcactatttttactagtttgcccgggccaagccagtgttgaactactggcctcaagtgatcctcccaccttggcctccccaaagtgcatccctacaggcatgagccactgcactcagcctgaactttcgaaatttattttaagggcccacttttaaatgcttcttttcagcagctaactttccagcggatgcttcatgtggtgccagccatacagatacgcttttagaacttgagctttggagaagcttatgcttgctttctgctctctcctgaggtcatcagatacttcccttgttcagtaacaaagaaagtgagactctttctgttacctaataaaaggccagtctgtccatttcattttgggtgctattaacattgttgaccatttacaatgtcacaggccctggcactgagcacatcatgtgcagcctcacttaattttcacaccagtgccctgaggtttctagaaaagaggaagggccccagtttaggtggcagtacttttcaatttggaggcaggagatcccagttttcctctgacctctctgagcttccgtgtttttacctctaagacagagagaccaatctctgttcagccaccttagaggattgttttgagggtctataagatgattgagggaaagctctgaaactggtaagagttaaatgtatgtttgtgttatgttgatacgagattcctctgagatgctctttagtccaggcaggtttatggctcacacagcgttcctagggtctcattcttctgcacaggcaggcagctcccgagccagccagtctggacccaaatgccacctgtgctcttggctggtcttgcgcattcaccgaggctcactgtaccttggtgttcccatttctaaaataaggcctgtagtaatccctacctcactgagttgttgagagacttcatgtaaattgtttagcagggagcctggcacagaacaagagcctagcagaaaagtaagctgttgttactttaagcaaatatttagcagcctaaaatggtggctttaccagtttctgtaatggttgaagtgatgacagaattcagccaatcccacccatagtttttcttgctaataaagcagtttgtttctaagtgtgttttgatgcgtataattaatacttcctgtccttgatttttggctgaaaaatgtcttctcttcctcgcaccctctgttattcccgtagtgaatattttcattgtggaagagaaattcccattggctcatggttggcattttatttctcctgtccagagacaatatttccacagcctcaaacgtgaaaactgaaaatctggtgccacttaccaccttgtaaaatcaagtcattgtaattgtgccagtataatcattgatctgaaataaaagcagaaaataaatcaatgggtagctttcttaaatacataagaactaaagatcaaatgggatattggaagactttaacttttcctccataatggtggatcttaattaaacacacagaagaacaatttataaaataaattcataggtttgtttgttcttttttagtgtcaatttttaaaactatctcttttttaattaaaaaaaatagagatgggtctcactatgtcgcccaggctggtctcaaactcctgggctcaagcaatcctcccacctcagcctcccaaagtgctgggattacaggcatgagccactgcacccagcctaaaactgtctcaaaagtagacagcataatgaactcccatgtacttatgatgtggctttaacaattatcagcttatagtcagtcttgttttcttacataataaagttacagtttcaaagccattaggaagaatagcttgctaattataattacttagttttgatagtgtaactttgtcctcagatgggtagaatcgttactctggaacaggggtgtccaatcttggcttccctgggccacattgggagaaaaattgtcttgggccacacgtaaaacatactaacgatagctgatgagctaaaccaacaaaacaaatcccacaaaaatctcatttctttttttttttgagacaagagtctcactctgtcgcccagactggagtgcagttgcacgatctcagctcactgcaagctccgcctcctgggttcacgccattctcctgcctcagcctccctagtagagggtactacaggcgcccgggggtttcaccatgttagccaggatggtctccatctcctgacctcgtgatccgcccacctcggcctcccaaagccctgggattacaggcgtgagccactgcaccgggccaaaaatcttataatgtttttaagaaagtttgcaaatttgttaggccacattcaaagccgtcctgggccatatgcagcccgtgggccatgggttgaacaagcttgctctagaaattggttttcaggccgggcgcggtggctgacgcctgtaatcccagcactttgggaggccgaggcaggtggtcacctgaggtcaggagttcgagaccagcctggccaacatgctgaaaccctgtctctactaaaaatataaaaaattagccaggcgtagtggggagtgcctgtaatcccagcactttgggaggctgaggtgagtagatcacttgagaccaggagttcaagaccagcctggccaacatggcaaaaccccgtctctactgaaaatgaaaaaaaaaaaaaaaaaaaaaacttagccaggcgcagtggctcacatctgtaatcccagctactcaggaggctgaggcaggagaatcacgtgaacctgggagctggaggttgcagtgagcctagatcgcaccactgcacttcagcctgggcgatggagtgagactccaactcaaaaaaaaaaaaaaaaaaatttcaattggcttctattaaagcttttttatgtttgtcattgttttctagtaataacaacaatccacattttttgtgagaaatataccataatatagagaagtataaagaaaatgtaaggccagacacggtggctcacgccgataatcccagcactttgggaggctgaggcaggcttatcacctgaggtcaggagttcgaaaccagcctggccaacatggtgaaaccccatctctactaaaaatacaaaaaaagtagccgagtgtggtggcgggcacctgtaatcccagctactcaggaggctgaggcaggagaattgcttgaacccggaaggtgaaggttgcagtgagcagagatcacgccattgcactccagcctgagtgtcaagaacgagactccgtctcgagaaaaaaaaaaaagaaaagtaaatttaaagcacttaaaatcccaccaccctgagttaactactgttcacagtttggtgaaattcctcctcgtcttctattttttcatagttgggaccatgcctatatactggaatgtaatataaacacttaatatattattgcgagtatattctcaaacttaaatattttttccaaaaatattttgagtggcttttaaaatatatcctattttatgcatgcagcataaactaacttatcctgtgttgatagatattttgtacttgcccttttctgtgctgttgtttaaaattagtgtatagaaataattgtgttgatcacttaggataaattcctagaggtaaatcagatattaaattctttatagacttctggttcatattccagttgccctcaatttatacacctactactagcattgtatgagagtgcccgtttctctgaaatcttacccacactggctgtcagcgtcaaaaagatgtatctgcagatttggcaaggaatttgcatttcagtgattattatcaagcgttcacacactttccatgtttaatggtaattttttttgtttttttttgagacgatgtctcactctgttgcccaggcagagtgcagtgccgcgaccttggctcactgcaacctctgcctcccaggttcaggtgattctcctgcctcaactccttagcagcttggattacaggcatgcgccaccatgcccggctaatttgtgtatttttagtagagacggggtttcaccatattggccaggttagtctcaaactcctgacctcaagtgattgcccgccttggcctcccgaagtggtgggattacaggtgtgagccaccgcacctggcctttaatgataatgtaaattttttgtgaattgtttattcgtggctttgccccattttttccatagtggagggatttttctttttcttgttggtatgtaaagtccttgtgtgtcaagaatgctctgcctcacagtttttctaatataatgacagtggaagctcgtaactggcttccacttgactgacttgctaaattgaccggcactcttcacttccttataaaacatgcctccatagtccctgtgccccatggcacatgggatggggtagccacactaccactgtctgctgctgcatatgttcctgtccacaagagccagtcatgtttgtttccaagcctggttctttcagatatagctgttgttgtaattacttggttgatatgaaaccaatgaaatgcaagtgtgaaaagagaattgtttctaggaaaattaagatgaatgctttagaaagactcagtaaaggaagataaatttaacaaattactacagaattaggtgtaagacaacagtaaatgattggaatgaattacagaaatctagaaagctttgacagtgggaatgcgtagtgaatggctctaagttctccctccatctgtaggaaattgaagctggaagtgagatgatatattcattatagggaggacttacagaagaaaacatcaagctctaatcacatgtccacatttcgttttgtttacttattttttgaaatggagtctcactctgttgcccaggctggagtgcagtggcatgatctcagcttactacaacctctgcctcctgtgttcaagcgattctcgtgcctcagcatcctgagtagttggggctacaggtgtgcaccaccacgcccaactaatttttatatttttagtagagacggggtttcgctatgttggccagcatggtcttaaactcctgacctcaggtgatctgcctgcctcggcctcccaaagtgctgggataacaggcatgagccaccgcacccagccccacatacctacatttcaaaggacagtctttggccctacagtcaagactaccaaaccaatgtacactaaatgggggtggggatcacgttgttgttttttgtttgtttttgtttgtttgtttgtttgtttgtttttttgagagagtctcgctctgtcacccaggctggagtatagtggcgtgatctcggctccctgcaagctccgcctcccgggttcacgccattctcctgcctcagcctcccgagtagctgggactacaggtgcccgccaccacgcccagctaatttttttgtatttttagtagagacggggcttcaccgtgttagtcaggatggtctcgatctcctgacctcggggatcacatattttaataaaattgataatccctaatagtgatggcgctaggtgtacttttagagccaatgtaaatagtcgagaaagggggccaaacatatgactaagcttcttctacactctaggagatgtcagtattgcaagatcctgttccttgaaattatctttgccccccgccattcagaaaaattaccaaatattgtaataatgtaatttaggacttaccctttcagagaaacagttcctgaaggttgacttcagttagtagtacagaagcgagacttgaagctcttcatggtttatgttctgttggtttatcccaaagagatgttttagagcacattcatttctgaaagttctcggtcatgtttccggaagtttgatcaggtgggttctttttctttttttaaaagataattttttttttaaagataattcagaatcagtcccacccctgagatggtattattacccaggaaagaatgcgtgaggatcctctaaatccatagagaaggaaaactaaaacaattttgttaccatttgtttggctcaagcatctggtagatccttcagttcttttcaaattagaattttcctctctttttcttttttttttttttttttgagatggagtcttactccatttcccaggctggagtacagtggcacaatctcggctcactgcagcctcacctcccgggttcaggcaattctccggcctcagtctcctgagtagctgggattacaggtacccgccactaattttcatatttttgcctggctaatttttgtgtttttagtagagatggggttttgccatgttggccaggctggtctcgaactcctgacctcaggtgatctgcccacctcagcctcccaaaatgctgggattacaggggtgagcctccacgccgggcctctctttttaagataataaaaattactaaacatcataatgacatgatttgggattactctttcagagaaacagttcctaaaggtttacttcagttagtgatttttatttttattatttatttattttttgagatggagtctcactctgtctgccaggctagagtgcagagacgagatctcagctcactgcaacctccgcctcccagactcaagcgattctcctacctcagcctcctgagtagctgggactagtagctggaactacaggcgcccgccaccatgcccggctagttttttctattttagtagtgacggggtttcaccgtgttgcccaggttggttttgaactcctgagctcaggctcaggcaatctgcctgcctcagcctcccaatgttctagggttacaggcatgagctactgtgcctggcccagttagtgatttttaaattgtagttccttaaatgagtattttggatttcactccacaatgttcttcaactgtaagatctacttgaggttatttgatttgctggtttgcaagaactcgtctacacgttgtctttatcacaaagaaattctggagcatttgagccctgttcaaaaaaaaaaggcaagaagaaacttcaggaacaaataacctatttgaccctgtaatgaataatattcacacattttctaatttaagagctgttgattgatttccaacttttagaatctgtttcagacaaaacatggaagtttagtaacagttttttctattctgtaataataattgccacaaggttaaaaaaaaaaatttacagaagaacatatttgagggtggtcgtgggggcaagaaagctggaaggacgggtggggtgtgttaatagccttgtcttgtgaagtaagtcctcagagacgctgtcctagttggaagaacaggcagtgaaggtgtgcgtctgttacggagaataggactccagtgatctagctggtacttagagaggtgggggtgatcaccaaaaaaacagctctgcctctgggagcggagaggactgggatagaaagtatttgtctttcattttaagcccttctgtacgatttgacttttcttttttttttttttttttttttttgagatggagtctcactctgtcacccaggctggagtacagtggcacgatcttggctcactgcaacctccgcctcccaggttcaagcaattctcctgcctcagcctcccaagtagctgggactacaggcacctgccaccatgcccggctgatttttgtatttgtagtaaagacaggatttcactgtgttggtcaggttggtcttgaactcctgacctcatgatccacccccggcttttttttttgacacggagtttctcgttacccaggctggagtgcagtggcgcaatctctgctcactgagacctccacctcctgggttcaagcgattctcctgcctcagcctcctgagtagctgggactacaggtgtgcgtcaccatgcccggctaatttttgtatgtctaatggagacagggtttcaccatgttggtcaagttggtctcaaactcctgaactcaggtgatccacccaccttggcctcccaaagtgctgggattacgggcgtgagccaccatgcccggctaaagcaagaaattttcattgcatattttaagcaaaggcaaatgcatatgtggatagactgttttaatttgactaaagtcatattgaatccatgaattttagaagctcaaactattggggaacaataattaccaccttggagtgaaaatacttaatttccacaagatttagtaaaggaagagttttttaaaaaccaccttaatgataatagtatgtacagatgttaagaaatgaaataggaatgtgtaatgttggaaacacaaatatttttgcttctgagaataaaactaattttttctcccaattttctcttcctttttcttttttctgttcccccctttctcttccagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtaaaagggttgtgggcagctagtggtggttgcaggagatagaaatctgggaattgcggtttgacctaccaccctttgctcgttaaaggagcagctttgaaatctggactgcagggatatccaaaacaacaactgcatgtttctaagggagtcgactctccttagaggagttcttgtacaatagccctgggcaaaaacagaacttgccctattttttatactgaaaaggacagctggacaaaatactgaacgcaatttttcccctaagaaaaagcattatttccctaaaatgtcttatattaggaacagagcacttgaataaacataattgatttataaaaactgaggctatacacttacctatctgttcagtacaaacaggaagcttcaaatgtaaacgtgaattctcatacacttataaatgcatatctttatgtggacttgttaaaatgaaattggtatttaggaatttggagatttttagtagttacacaagaatcaatgaaaaagaacgaagctggtttccaaagctgatatgtctgatttggttcctttcttctcaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaaggtaggggacccttggcagggggcgctgatgggcccagggcaggggaaccagaggtcctgctgtcggattgataaattattgcaagaaagctcaaccaagaagatgtttaaagaatctttcaggtgggagtcattccattagccttatgaagaccctttattgaggatccgttctgtgatattacaagttcctgggactggtatgattctcttattgtcttgctagagttttgttgttagcaagttacttaaaataggaggaatatctgttgggttttggacacattttttacaataagattcttcctttaaaaaaaatattttataatgatagaaatcatccccaacgagaaaaattcaaataatagataacctttcttagagcgaaagaccactttactatcccctcccccacagacgcctggcacgtgggcttcttcctgcggatgaacacattcatggagaggtgtgtggacatacgtgcatacacgcatgcagttttcagtgaaaaccgggatcaaacccacagcttaccaaacccaaagtgtggtgttgtaagggaggtggcctatatgattttgtggtcttcatataaataatccttaagagtccataaagagaagggaggagttaacattgactgaggtattgctataatacctgcatctttgcattaaagaaagggaggcataaagagatcttgcccgatgtcaacctagtgagtcactgatggaataggcttggatcccacagtttattctactgcaccaggctgtagttgaggtacagtgttgataggtggtcccgatggtgtatgtttgtttgtttgtttgtttttgagatggagtcttgccctgtcacccaggctggagtacagtggtgcgatcttggctcactgcaacctctgcttcccatgctcaagtgatcctcccaccttagccttccgagtagttgggaccacaggcgcacaccaccatgcccagctgttttttgtttgtttatttgttttgagatggcattttcgctctcattgcccaagctggagtgcaatggtgcgttctcggcttactgcaacctccacctcccgggttcaagcatttctcctgcctcagccttccaggtagctgggattacaggcatgcaccaccacacccagctaattttgtatttttagtagagacagggtttcaccatgttggtcaggctggtctcaaactcctgacctcaggtgatccacctgcctcagcatcccaaagtgctgggattacaggcgtgagccaccacgcctggccgtttttgtatttccagtagagacagggtctcgccatgtcgcccaggctagtctcaaactattgagctcaagcaatctgccctcctcggcctcccaaaatgctgggattacaggcacgagccactgtgcccagcctatattttttaaatagtatgtaatgcatgcatatggttaaaaaaaatcaaaagaacaaaatgaattacagtaaaaagtaatcgtctcctaattcacttttcctgcccagaggcaagcactggtactagttccttctagagttacctggacacagatggatggtatatagatgtttatgtggatgtttatattcaaaaaggcacgcaaatgatacatgcacgcgaatgcatcatgcttctcccgtgtgataatacaccttcgagattgttttatggcaacaggtttagagctgcctcattcttaccagtgactgtgtaccgtggatggactgtcacttatttcagtagtcccctgctggttgacttttaggttgtttccaatctttagttattgtaaataatgctgatatgaggtggctttcaaaccttttcaaaaatctggttctgctgtaagaaaaactcattttacaccaccacccagccctcacaatataacaaaagtttcatgaagtcttacctacccttactgtatgtaacgcactctgatagtttgtatttcattctgttctatttcattttttaaaaattctggttgtaatccactaaatttatttcatagactgactagtggcccatggcctggtttgaaaatgcgttgcacgtgcttctttatgtgggtgtaagtgtgtgcagataagttgccagaactgactgaaggttatgtgcatcaccaggtcaccctctatagaaattgtacaaatttgttctcccacaaacaatccatgaaagggcttgtttccccttaacccacaccaacagtttattattagacatgtaaaaacacctttgtcactctgactggtaaagaatggcatctcattatgttgaatatccatgtggcatattttgggtcatctttatgccatttagtaaattcatctttttcctacatgttagaaatataccaagtgtatatgtgtatctaggcctgttgacctctctgttctcttccattggtctgtctatccgtatgccagtaccaaacttgtaattgctatggcttcataccatgttgtggtatctgataggctagtccccacttacctttttaactttttttttttttggctgtcttcacacattcacttgtaccatatgaacatacatatacttcctttttaatgttcttatttttcttcacatcccaaagtcttctggctgaaatcctattgcccatatctgatgctttccaccactgcagaattctttagtaccaacactgcagtgtgcgtgacggacaaaagagcctgttatccctgtgtgactagacgtgggcctgtcagcttgctttttgggttattgttcttatcccagaggtggagctgcgttcttccattctgagggacgtacttcaggacattgggccctttgaaagagccttcagaccacaagggtacctttctagtgaatcagaacaccagggtgcaccgcccatggggggtgattttctggagcagacagcttgcttcccatgtgtgctatttatccaaggcaagtcatgctgcaccgagatagtcacttgaggtttgcaccgaacaggataatggtttcttttccctagagctgtttaatcagcaaccttggaacagatgcaggcaggcatgatctagtgtgacagctcattagctaggggttgatctaaaatactgttcaaatgggcaaggaaaggcctgcgtatgggatatgtgatttgcagaatttgagattcatccaaagagggagaatctgcctaagatgcctctgtctaaaagctagtatgtgccaggtgggtttgtttcattccaggtagagcacagggagcttatgtgaccaggcagtgtccaccattgggcaacacatcacacttcccttggcgtcttgcctcctccctccccgtctggcacagactctcccatcagccgtttgggcacctggggacttctgagcccacagcgtcatccagcagaccgtctgcggaggttccttcctgtgagggccgcactcgagggggtgtattgggtgggatcgtatcactggaatgagtgaacatcgttagagggagctcccacagctatgcgtgtgaccgtgtggctcgccgctgaaaaatgctgctcacatagcagaacttcgatgcaatcctttttggatttctaaggggaaattttaagaccgataggaggaataggctggggttgaataagtttggttcttatttttaaattagctcatctttagaaaacaactgaatttagaagattcttctgtaaggccccaaaggatttctttctgcctccccgctcttggctcttatttgtgtctctcctcgtgttctgagcttttcagcagcccttgtgggtcatcagccgaggaccatcagctcggggcccagagctggggactgggagttatgagctggactcttgaaggggccaatactaaaccgagccgttgccatttcctaggagccttgacaggctcaaacatctctgggtacccggggatttgactgtcattgttctgatcctttcccaaagttgtaaccctctgttgacatagcggaattgaagattgatttttcttcaccaccttctgcctaaagttcttagttctgaatgtgtccacacctcattgacttctatgagatgcccctgtcacgttcctcgattccttctagtccttaatagagtctttccttttcttgaaagaaatttgcccagttgacatttattttcatcgtctgctgaagtgacagatccaaggccctaagcttcagcagagttgcaggaagggaccatttacacagcctcttagatgtttaaggtttacccgccctgacagtggctcagcaggcgtaggcctcctggtgaaaagccgagcacatggctaacctgctctggtggaggggctttggaccgttcttgtgtactgcgaggcttgccttcctgctggtgtgtgagcagctctcgggggaacttgcttcctggctgagcaacctgtaaacagtatgtctgaggtgggactcccgggcccaaagcagccaaaggttgcacaagtctaagctccgttaggcctgtcagtgtggctgtcaaaagaaacgctgtttcttttctttctttcctttttttttttttttttttttttttttttgagacaggctgtcacccaggctggagtacagtggcacaatctcggctcattgcaacctgcgcctcccgggttcgagcgattctcctgcatcagcctctcaagtagctgagactacaggtgcccaccagcatgcccggctaatttttgtatttttagtagagatggggtttcaccatgttggccaggctggtttcgaacgcctgacctcgtgatctgccctccttggcctcccaaagtgctgggattacaggcgtgagccactgctcctggcaaaatgctgtttctaagcgtttctttgacaagtcccatccttcaggttcatgcatgattagggagaggcagcagccctgactgagcacctgagtgttcatggaaccaggtgcatagcgtaatgacatgccattcatgacactactttcttccctgaacatgccatgaattcctgatgattacacagttccccaacaagtgctgctcaaaacctggaactaatcagaaggaagcaccagccttgactgttagtgggacacggggaagatggctgtgaaccgaatatgcggtgcctttgggggagaatggttttagtatttagtaagtgtcagtatgtaagttttatgttgaaaaatgaactaagtcacaacaaggtctataaatttaattattgaaaaggataaagttgggccattcagtgaaacctcctgtaactgaagtctctaacacagtagccaatgggggaatcaaatgagcccaagaagaaattctgcgtaaactaggacttaggggagcccttagcattccactttggagtgaaggaacccgatccgccagttggggaacgctcctttgaggcccatgtgcttcctgctccgacgggagagagggataatgagacttgctcccaggtgccctgccctggagcccagcaggccacacgggctctttgactctgtactgtgccagtgatttccaagatgggagtggtgctctgattgcaggaatgtcttcttggaatggcttttctcacttctctagagtccttggtagggagcacatcatcagctgagctaacgtgagaacaggggttggagcacttgccaccggcagtgaagtaatcggacagctgacagtcttttccagagggtcggggattgggagctgaccccaggtcccgctccgttttctttgtgtgcagcacttgtgttggtgaccagcagtgaagagagtgcgtggacgtcaccgtgtggttatgaagctaacggcagccctcagcggtgccgacttccgctccccgacacattcctgtgtgcacgttcgtgggtctgagcgtgactcatggtgtgtgtccctagagcagttcactcagaggcttctcttccgtgctctcaagaataaaagcagggccgccacatcctgataagtcttgccattcagcggtcctgtcttgcagagggataagccagtgtccccaggccacagtccttacaccttgagcaagttccccagagtgaagcaatctttctagacaagttttgcgtttctcagaattttattttagtgatattgttaccaaaagattgtaaccccaaaatgtgtgcctttggcttgcctgttttaggaattcacttgtgttatctccccaagcaaaacaaaagaaagagaaaccttcctgttgacgaatcctttttaccttcttgagagttgtcaatgggactaacttgtatatttctttgctaccttcttttaaaaacatttttcttatttactattacagacgtatagtaatggtgcctgacataacccacagccaaaggccttggccttgctgtgtgtatttcaggctttttcttcccttttctttttttttaagtaataagatattacagttatagctgaagcctgatcctgttcccttttctccctctcgtcccaagcatcaccactgtcccaaatctgctccttcttcccacatctttgcagtttgggcaagtcttctgaatgcctagtcatgattttaagtcttctttccaccttcagtgatggttggctaccctgagcaagccagtagattcagttatgtgcctggagtccagatgttttatgtggatttttcatgtggaattgtttttggtaaggaagtattcccttgccacaccaatgaaaagagaagaaaacaggtagctggcacccactgcattgttgctttctgccagcctgctccttctccttcctctccagctctgttgtgtccagatgcaaactctacatgcagggtttgaccctaggtcctcacctgctgtagctctcacacggggaccgaatgctcttgtgtcgtaaatccttccttttgcttcctcgtgttggatgtgtttagtggttttcatcttctggtcactatttcattctaggtttatcccctttaaattataaattaaatgaattcttcgcttttcctaccagcaactacccaccaagttctaccatgtgttaatttaatcaacagttatttattgagtacttattacatgttgatttcctgttctcaacattggaatgaagacagcttagggttctgctttcatgaagcttgcatgctcttaggtggagacagacagtaattaagcagataaacaggaaattatgagctagtagtgggtccattgctgagaagtaagacaggtggtaggatgacccgtgccgtgtgatgactttagattgggtggttggggaagacttcacggaccttaaaactggccttggaacgggaagcgagaactgggcacggaagatgagggagtgggagatttcaggcagagggagcagcagcaggtacagaggccctgaggccttttattgtgtctttttgcttgagcgagtaacttagagcacacgtagagaaagacagcagaagtgatcttctaaacactctgtcctgtgtggagagctccttatgtgagattttgctgtgtagtgaattaaggctcagccaaactggctcacgtgagctctttgagcttgcctgtctctgtgggctgaaggctgttccctgtttccttcagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggtaggctgggactgccgggggtgcccagggtacgtggggcaaggcgtctgctggcattaggcgatgcatctgcctggaagtctacctcctgcctgctgtccgagggcttcattggcgccacggaattgacttttccgtcttatatcattcctgtgtctttgtaggagtggaatcattctcatagtccgagtgtgtttccacatatggtgagagctgacaagcatggaggggttttggtgtaaaaagattagtcatttggagaggttttctcattttatggcaaggttcttttaaagccgtggatttccatgctgttcgtgcggcatggagatcacttcctaccgagagttaaggaggaaaaaaagatctctgagttttgaaagaagatttaaccaaaatgcatttgactcttctgtggatttttgttggctgatttggaaggcaggtgccctgggcatccccagtgggttccaattctgcagctgcccagactcctgcaggcagaggtggaagtgtccccgctagaaaggcatccaggaaactcgcttttgaccaactcagatacagtctggtctttcttgttaactgggactttaccctctccactaatgaatagtgtttgtagtttccaggcaggtttttttcttcttttgaagttctttgaaacccttaggacgccaagggaaggaagttttcattttcagccctttgcattcttcaaaatgtgatccaggcttttccctgtggcgagggtgtcccctagtgttgacttacattatttccacttgttttaaatgaactttgattaccttgacctgtgcacagaaatcaagagaggcagcccctttttagagctgttagaaaagaactgaaacatccacctctgggaaaaatagagtttagtttgtgcatgtgtctcagaagtcaggtgcacacattgagactggtgcagccacatgcctgccttttagggactccttgactgacctgatgccttcacccaaaggcatagtgaaatgggaagtatgatttctgactggtatgaagtggctgctttagagaaaagatcacattctttagtcctagaaaccaaaggtggcttcctgatgctcttattcagcacgtgcctgtctttgaggcaggactggcactggggtgggggcatggtttggggttaccttaaggaccctggatatttctcagataggactttttcttttctttttttgagacggagtcttactctgttgcctaggctggagtgcagtggcatgatctcagctcactgcaacctccgcctccccaaattggcgattctccttcctcagcctcacgagtagctgggattacaggcacccgccaccacacccagctaatttttgtatttttagtagagatggggtttcgctgtgttggcctggctggtctcaaactcctgacctcaagtgatctgcctacctcagcctcccagagtgctgggattataggcatgagccaccacacccggccctggactttttcttaattgaataaatatttatcaagttcttcacttctatttgacggttcctgggctcaacactggattagaaaatgaatgggacagagttcttcatctgagacatagggtggtccaggtagtaggaggaagaaggattgcagcgcagcagtcctcgggacaccaccagggggtgaacgaaggagggacgggggagctgtgcaggtgccccagctccttgggttgcacagggttgaagaccagacccaagaaatggggccttgaccagctcagcaaactgagaatggcctagatttcagcacactttcaccaggatttgtcaaatgaaggaagattccaagcgcaaaataccctagatttcaaccagtaaaagcttgtctgaaaacttccaagatcgggttgtacaacttgacatttgaggaaacgctacttcagtagatggtgctgtggcattgttttctgaacaactgaaggaggagactccagagaagggtacctgctggagacctgtgtggtttgtggggtgggtttcttatatcactgaatctaaagtgggtgcctttcagaatgtgctttactaataatgtgttactctgcttttaattatacttctaataattttccccaagagcctaattaacttacgatctcacagttggtaaagaattacaggatgtcattgcgtggatttctgtgggcagtcacccggagtggtatctaattagggaaaaggcagtatgtccctttctggaagtgtgttaactgaaaatacggtactaagcaactgctaaataaattagccctccttcctcctggccgtgcgcagaaacatccctgcagtatgttgcctatttgtcaaataagattagtgtaaatatggggcatttacctttgctatagaagctgaaattgccaggcacggtggcttacgcctgtaatcccagcactttggaaggccaaggctggtggattgcttgagcccaggagttaaagaccagcctgggcaacatggcaaaactctgcctctacaaaaaataccaaaaatcagccaggcgtggtggtgcgtgtgtattcctagctacttgggaggctgacatgggaggattacctgagcccgggaggttgaggctgcagtgagccgtaatcgtgccactgcactcctacctggggagcagagtgagaccctgtctccaaaaaaaaaaaaaaaaaaaaaagctgaaatccttgagtggattctaaatcatggcagtggctaacattcagcttcatcgtgcccttgctgtgtgcctggcagagacctctgtgcttactacatgtcgcatctcattgaatcctcacaaccttaagatccttccaggttgagcatcccggaccctaaaatcccaaatcctaaatgctcccaaatccaaaactttttgagcaccggcatgcagctcagaggaaatgcatactggaacatttcagattttggattttctaatttggagtgctcatctggtaatatagtgtggatattccccaatctgcaaagatctgagctccgaaacacttgtagccccaagcctttcagttaagggacattcaccctgtatcaacattttaatgctgagaaaatggaagagagagaggttaagtgactgacctggcatcacacagccaggagtggcagcatcaggatttgaactggggcactcagctccaaagctcagctcttcttttttaaaattgaacccagattcttatttctcaaataccataaaatatttacataagccacagttatgccttttaaaaaaaaatcaccttttccccccactctcccatttacttcctttgcagtttgtggctacgatctaccttcctcttcaaaacccttgaaaaatccttgcagccttgcccaagcgtccttccctcagccgatttttgggcctcagtttcctcccatccagcctgtttcctatcgctggcttctccaagggcagagccagagtcctcttttgccagcagggggagcaagggcctacttttaaccgcaactccatagtgaacaagtcaagcaaccttgggtcattcatataccttccaggcagaaggaagacagaagagaacaggaaaaaagcaggacactaggctgacccctgcagatgggaagctggaattacagcaaaggctcattgattgggaactgcacccttgaattttgatagtattgggcaggacatagtcttgctatctgtgttaaagggaggacgtgcttaagaaaaccaacagtcaagtacaagaagctctaattttttttgaggtgggggaacggggtctcactgtgttgcccagctggcctcaaactcctggctcaagcagtcctcccacctcagcctcctgagtagcagggatcacaggcttgtgccactgtgcccagctaagaggctgcaattttttaatgaactgtattcagagtttgtacatcagtgtcttttttttctatgaatcagttctctaagtaatagaatttggaaaagaccgctggtgatctagctgtggcatttagattgactcatgagctaactgacaaatttactacttaaacagtggaaaataatgtgcaggatatatttttttgaataggttgtcccaccctcgagttctgtgtctttcctgagtccgcatatgtgaaccttgttttaaccaggtgacagcatcccataggcacggatctctgtcttctgttgtccacatcatgtgtcttggcagttgttccttattgatatacataaggttgtcttgtttttgtatggatatattttaacatgtaccagtgttcagtgggcctttctcttatttccaatattttgctgttctaaacagcagcacagtgattcccctcatttgcatgtatgtgaaactatcctggtgggtccgcttccagaaactactggatcagggaccatgtgcatttatggccttgaccatactgccagagtaccttccaaggagtttgtgtcatcaatttgcactccccagtggtgttgagagagtgcctgtttcctccacccagcacagtgtgtttcggttttatttctcccaggaaatgggtgttagttggggagctccagaaagaagccgagaagaagggcagtcgtacccttggcagttgtacgcagtggctctctggccgcacaaggagggctcggagcctgctgttgccaggttgcacttgctcttgcaagacaggcattccttgcagtgggttctcctctggccagggagatgagttctgggggctcctctacgaggtggtctctcctgggctttcctctcgagcttcaagtcccacctctggagtgtgaatcgccttcctcagtctaactctatccctccagtggtttttttttttttgctttttgtttttgtttttgtttttttgagacagagtctcactctgtcacccaggctggagtacagtggcgtgatctcggctcaccgcaagctccacctcccgggttcaagcaattctcctgcctcagcctcccgagtagctgggactacaggtgcctgcgaccacgccggctaattttttgtatttttagtagagacagggtttcaccatgttagccaggatggtctcgatctcctgacttcatgatctgccctcctcggcctcccaaagtgctgggattacaggcgtgagccaccacacccagcagtctcttcagtgtatttagctgtgttgatcctgattcatctgatgggtgaaggaatattggaaataaggataatccctgtggtgtttcactgtgcctgtaacctctctcacctagcatttggttaaccagaaacagatgtggaaactgatctatctgtagataatggccctgctcagggagggggtccctgctgagagcgagacattgaaagcctgctacacagaaactgctggaaggccaagccatttcttttttctttttctttttttttttttttttttttcagacagagttttgcccagtagttcaggctggagtgcagtggcatgatctcagttcactgcaacggcctcccaggttcaagcaattcttctgcctcagcctcctgagtagctggcattacagactcctgccaccacgcccggctaatttttgcatttttagtagagacacggtttcaccatgtggccaggctggtctcgaactcctggcctcaagttatctgcctgtctcggccccccagagaggccaagccatttttaaaaacagtgttctttttcatgctgtagaatacctgtgcttgggggtgagcaaaaaggaggaaactgattagaaaatacctatgagggccgggtgcagtggttcatgcctgtaatcccagcactctgggaggccgaggagggcagatcacaaggtcaggagatcaagaccatcctggttgacatggtgaaaccccgtctctactaataatacagaaaattagctgggcgtggtggcaggtgcctgtagtcccagctactcgggaggctgaggcaggagaatggtgtgaacctgggaggcggagcttgcagtgagccgagatcgcgccactgcactctagcctgggcgacagagcaagactccgtctcaaaaaaaaaaagaaaatacctgtgagatgttgagtctgggagaatgtatctgtgagaagacaaacaaccctagtattcattaaagaaccagcctttgttttctacaagctatctgttcggtctactcaggaatagagataaactgccttaatagtaataaaagcaagcaccgctgagtgagtgctggcctcatgccaggaactgctcttggcactctgtgcacaagggagatactgacattctttctgttgtgcagatgagaaaacaggctcagagaaggtgagtaacttaccatggtcacacagcagcagagctaggtcagtataatccacaagtcattctcttaaccgctccgccgtaccgcccagaatgttcatctgcatctgggccagcctcgtcagtccgtgcaaagaggccagttgacccatctccctccctaacaagcacagaagagaccaggctgaaaattcatagctttcactcaaagtgtttccttcacctcattcatctgagttagctttattttactatataaagagaattttcttcagcaaaatgataggaatttttttaatgaaagtaaaggaatgtcacagaattgcgtcagggatatttccagtttaacacaactacaaacgtaggctgttttgttgttgttgttttggttttctattttgagatgaagtctcactctgtcgcccaggctgaagtgcagtggtgcgatcttggctcactgcaacctcctgctcccgaattcaagtgattctcctgcctcagcctcctgagtagctggaactacaggcatgcgctaccacgcctggctaatttttgtatttttagtagagatggggtttcaccatgttgaccaggttgttccggaactcaggtgatccaccttggcctcccaaagtgttgggattacaggcatgagccaccacactcggccagctgttgttaatagttaaaattataaattggacaaggtctgattccagcagggggagacaaattcacattgatttttctataagaaaaggaattatttgtactttcctcccttgtgtgtttgtgtgtatgtaagagagacagatgagaagagggctgcctttgagagccatgttccttttgcatgtacatacctctcaagattattacatgggtcttttagaagagggtcagtagctataaaagcaaatgatatttatgaaatgatttcattgtatgtggtttattatgtagtcttcaaaacagaaaaaccccacttgcttcgaaagcttcctttgagagctactttttaaccatattgttgatcaagcaagattcttaaagccccagtcctatacgtgagcagcattgaatccccggatactggcaggcgttcactgtccagcctttgacattagcatgtcaccctggtaaatcagagcactccatgccaaaaaacagcatggtctgctgagcacattaaggagcagagagggctcagaagaattaaacggccaatggggtggtgacagaggtgagctctttccccctcggacagcttggcatggtgactaccaatgaggtttaattgacattaagagcagctcaccatttttaaagagcgataaattgtttccaagtttcagcctcacggagccctgagctaagagataatgggctgacacatagcctcattggtcacgcttcacttgtaaacaggattggccaaagcaagaaggctgtaagtaaaggttggcccgtcctctggaagcctcccccacagcagggtggggaactggggcagccatagcaagcaggaccatcaggacacggtgtctcccaaacgggccggaacatacaaggttctacgtttaatagaaagttgatcaagccttcctctttggggagcctaatttttaagccattcactctctgttcccctgcagtcttttctctccaaactctgttgaaacttggaggcatctcgcttcctcctttggcctttgtagccaggttttcgcagcaaacagaccatatcatttaacacccacctgcttcttgttctgatagtcagttcagtagacagtagcattttttaagtcaaaaagaggcttagtgatgtaggccaaggtcaaggctctgtgtcatggggctctccccagggctcagtgccacggcacttcagacttgaccccagactgaaaagatgagaggacctgtagaagtcacggcctgtctgtcccgggcccatgctcctatctttttctcccccttggcatttagataccatggacttgttaaaatgtgacatgtactgatgatccttatgaatttgttggtaggggagctgctggtgaggattattttagactgtgagtaattgacctgacagacagtgatgactgcttcattaagagcccacgaccacgtgccagaatagttcagcatcctctgttgctactgtactttgagacatcgttcttctttgtgatgcaatacctctttcttgtcatgagggtctcttcccttaaatcaggtacatttcagattcttcaggtgatgttttttaggctgagtgtgttagcaccccttggaaaccaggttccctggggaaagtgtgtcacgtttgtggtgtttgcccgtatagctctcccacacccccacgagctccaagaggtggggaagcacatgtggcctgcctagcaggcaccagaagcaggttgtcggcacatgcaagccagctttgccctgttgcctgggggagaattgaaaagtttggccccaaaggggaaaattctttctgccatcaagttgctggtcagctgtcatggaacctgtctgcagcaatgtggctgtcacaaaacgcagcccaggacgagtatgcgctgaagctccattttgcattaactagtcaagtacttacccactgaaaagcacttcctgaaataatttcaccttcgtttttttccttctgcaggaggacaccatggaggtggaagagttcttgaaagaagctgcagtcatgaaagagatcaaacaccctaacctggtgcagctccttggtgagtaagcccggggctctgaagagagggtctcgcgccgcacccccagggtgacacaggcgctggggaagacgcacgggcggctcactgcacaaaacctcgttggaatatttgtgctctgccgacgttcagccgcgggtaaaatgaggcctgtatgggatgggtgtgtgcgtgtgtgcacatatgcacatgtatgtatgagagggagaatgtgattattttaagtggatacctaaaagcagtcaaatgcaaatctgaaattagtttctgaaacttgggcattttccagagttttctcactgaagtgattctgtaagtagacacataaccatcagacctaaccattcaggggtaaactgacggtggtgaaggtcatttgaggtggggccaggtctgcgtctgaattctgtggcagcctctccctgcgtaaattcaagttcactggcttgagaagaagaaaagagcctggccatgtccctcccacacgagcacagtctcaggatgcaggtgcttgggaccatgttggaagttgggcccaggactgaggagcagagtcagaatccttcagaaggctttttctttagacagttgtttgttcagttgggagcggagccacgtgttgaagtcctcgttgtcttgttggcaggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacaggtaggggcctggccaggcagcctgcgccatggagtcacagggcgtggagccgggcagccttttacaaaaagccccagcctaggaggtctcagggcgcagcttctaacctcagtgctggcaacacattggaccttggaacaaaggcaaacactaggctcctggcaaagccagctttgggcatgcatccagggctaaattcagccaggcctagactctggaccagtggagcagctaatccccggagtaaggaatatttcatttttgaaccattttgggctaagtggaatattaaatgaagttcataaaaggcgtccttcctaataatcacagtcccctctgaaagagagtactcattgttcctccggggccagcgcccagcagtaatggcctcttcctgggagccgctggcattgcttttacgcaaactcatgggaacaaccagtacatggcttttgttttttagtgtgtgtgtctttttttcctgctttctctttattatcttttcattctccacttaacttgctgtcctctgattcagggatgtttccacaattgtcagaattgtgtatgtggaggtctaaattaagtgtctttgctgttttaaaccctgaagtgttttgaccttcaaatgtgccacaattatcttggtcttcaaattctttgctggtggaaatggcttcccagcaaagcgacggcctgtgcaggacagagcctgatgggctttgctggcgtcttttatacgagatgttctccacaccaccactgtgacctttcctacccaattagctcaagtatgaaacctctacatgagatttcatagcaaggaataagggtagcatgtgtgaaatgctggcggctccttgtaagacagattttctatgctccatgagagcctgatccagatactcctggctttcaggaaagtgaatcctctgatgctgcaaattgagattgcttgacttctggtttgcctggcacggccgggtccgttccttcttcattcaagcgaacttaaaatgctttatcaaaatcctcttaggcacaactcttctgtatttaatttcactcttgttgctgacgatcaggctatctcacgcagccatcacctcaagctgctgccccctctcattccgctcttcccgtttgccttgtgatttcatggttaatgttcatgaaccaaagattagactcctacctccctgggagaatcaggaagtgagaggaagcagattatacaggagagagttttttaggatacatttgggtacacctcaggaagaaagagggaagtaggaaacacagtttgggtaaagaatgccatgggctttgttttattttgtcccatcaagtctgaataggacaagtggagatgcactctgatagaagtttcttgcaatcaaggactgggctcagtgtttgtctccgtgcctggcatagagtagcaataactgcacttactattctcgacacttcaatcaaagcacttagcatattaattaattacactcacagcaaccctaagaggtaggtgccattattatcgtcttcattttacggatgaggtaactgagtcccagggtaattaacttgctccaggtcatgaggccagggagcccgctcccagcaccagcctctccccatgttgggatctcagggtgtgtttgctgagaggctggcactgtgttaattgccgtgggcattaatacaaacttccagggcattggactcaatctttccattgtcagcattgcacctttgctcagcagtggtggatttgtgaagtggaaggttggccaggagctctcatgggtgaacattttcctttcttagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctggggtaagggctgctgctgcactgaagtggtccttcctgactacaggagggtttttttctgcctctttcttgctcttccctttcttttcttcctttctttttgtttttttgagacggagtctcactctgtcacccaggctggagtgcagtggtgcaatcttggctcattgcagcctctccctcccgggttcaagcgattctcctgtctcagcatcttgagtagctgggattataggcacccgccaccacacccagctaatttttggtatttttagtagagatggggtttcaccatgttggccaggctggtctagaactcctgacctcaggtgatctgcctgccttggcctcccaaagtgctgggattacaggtgtgagccactgtgcacagccacccttttttttttttttttaactgtgtatttggcctgaggtccaaatatggtgcacagtgtatctgctgatctgttaagtctcctctataagccccctctccatgtctttatttcccttgctgtttattgaagaaaccatccttagagtttcgtacgttctgggttttgctcattgtctcccatggtgtgatttaatatgttcctctgacttcagtattcctagtaatgggtggctggatctggaacggcctcatcacattcaggtttgactgtttctcttttagtaagagtggatcaggggagatggtgtgcccttccatcagtagatacaaaatgcttcatttatttccttccttcctttcctcccctctccctcccctcctctcttctccttctccttccgacggagtttcgctcttgttgcccaggctggagtgcagtggcacgatcttggctcactgcaacctctgccttctggtttcaagcgattctcctgcctcagcctcctgagtagctgggattataggcgcatgccaccacccccggctaatttttgtatttttagtagagacggggttttaccatgttggccaggatggtctccaactcctgacttcgtgatccacccacctcggcctcccaaagtgctgggattacaggcgtgagccaccacgcctggccccaaaatgcttgatttgctatcgtttttttgtgggggtttttttttgagtaatttttggcagttactgatatacagtgcctagatccaatagtttgctaggtattgccaggtaggaattctctaattctgtcattcctgttaagttactagtggaaacttgtatgaggagaaacagttcctcatctgtgtggttactcagtggtacagttcatgtaggaagagcagggtcaattcttgcttctttgtttttatttaccagtttttaaaataacaaattactttttcttttttttttttggagccagagtcttgctttttctggggtacaaagtacaggctggagttcagtagcatgatctcggctcactgcaacctccgcctaccagattcaagcaattcttctgcctcagcctcccaagtagctgggattacaggtgcctgccaccacgcctggctaaatttttttgtatttttagtaaagatggggtttcaccatgttgcccaggctggccttgaacttctgagctcaggcagtccacctgccttggcctcccaaagtgctgggattacaggcgtgagccaccatgccctgccacaagttggtttcttttttttttttttttttttttttttttgagacagtctcactctgtcgcccaggctggagtgcagtggcgcgatcttgatcttggctcactgcaagctccacctcctgggttcacaccattctcctgcctcagcctcccgagtagctgggactataggcatctaccaccacgcccggctaatttttttgtatttttagtagagacagggtttcaccatgttagccaggatggtctcaatctcctgacctcgtgatccgcccgcctcggcctcccaaagtgctgggattacaggtgtgagccaccgtgcccggcccctaatttttgtatttttagtagagatggggtttcgccatgttggccagggtggtcttgaattcctgacctcaaataatcctcccacttcagcctcccaaagtgctgggattacaggcgtgagccaccgcacccagccttgtcctggtcttctgatgataaagagcctggtaaaatgtcagagcctgggctgctgctggggccatcccttctgaggtctgctgcaaaggtaactgattttaaatgtagtgtagtgaaatgctacacatcttgaacagcctttctctttcggttttctttcagcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgtaagccttcctcagcctgttctcacgagtatatgtgggcattccaggaaattcaactgtgcaggagtgtgtacacaaagttgaaagtttttccatgagctctctccattccagttcttcagatgcagctaatgtagccatttgatacctattgacctttatttacagataaatagtatgtgcgtgacttgtcttttaaagcaaaaatggtattgatagataccaaacctgggtgtattcctaaatacagattcctgggccctgctttcacagacattctgctatagtagctaagctcatgaggtgattttttttttttttttttttgagacggagtttcgctcttgtcgcccaggctggagtacaatggcgcaatgttggctcacttcaacctccacctccctggttcaagcgattctcctgcctcagcctcccaagtagctgggattacaggcatgcaccaccacgcccggctaattttgtatttttagtagagatggggtttctccatgttagtcaggctggtctccaactcccaacctcaggtgatctgcctgcctcggcctcccaaagtgctgcgattacaggcgtgagccaccacgcccagctgcgaggtgatttttatctggtcgttttatactgattacatatgtgttatctgtactatgcacacacaggatgttttcatatatcttataaggtatttatatggccatttcttacactgttttcccacacatgtctttccacgtccatatattcagatctctccctctccctacctcttatttatgtatagctgcccagtactccatttcactcattcatccggtcctcttttgatgtgcatttggttgttttacacatttgtttggtttttgcccttataaacaaagcagaaacaaatattcatgtacacgaatctctgggcactttggctggtatttctaaaagttgaactgttggttccaagaactgtgtgggttttaaattttgatacattttaccaaactgttaaaaaaggttgtgccattgtatgctccagtcagacgcatatgggagtgactgttcctggtccccagtactaggctttgtcagtctgtttagtcctcatgttagtctcattttcatgagaatttctttacttcagactttgataaccgtgaagaaagaacaagatagaaggtgagctgtttggcttagtaattttctacacctactagagcgggactgggaaaaatatatttgtaaatgcagttcttgctgtcactgtctctctggggttttacaatccatattcctgccagcatctaacgtcttttcaaattcttaatgtctataacaggacatgatgacattcatcgttttgacttgttgcagcaaaagatggttagcaggattggaatgttgctttcattctagacttttccttgagaactgctagccccgtattgctagccagatctcatggatgatctgacttgggtttcatctgtccaggttggcagtggaatccctctgaccggccctcctttgctgaaatccaccaagcctttgaaacaatgttccaggaatccagtatctcagacggtaaagtacccatcccggggtacctgcagtggggtgaaagggcagccatgtgggactgcagcctgggtcattcggttcacttcctggtgaaagttcacagaccagcctgtcctgagaccagaaagctgggcagaggtgtggagtattgtgctttcttgtctgctgcagccctgcagagttctaagaaatgctaagggctgtttctccggtatccacgtgccttttctttagttgtatgcagatgagcactgttaccttacaaagaaagagaaccaccacaccaagccaacaccagtactgatggctgctggatttttgtttctgtccctgtatgattcttagaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcggtaagtcccccgcttcccccaaccccactgctcttcccttccctgccagaggctacattcaggccatcataggccaacgggaagctgtgaatggagcccgcacagaagggcagccatggcctttgtcaatggttcagcttcggaaggaggaaggttctcctctccccacctgcctcctatcccctccctctgagagtccccgaggagcataggctccagcagtgagttcagtcctgtaggcagaggtgcttctgaagcccgccaaggagctagcccatctcccacctattacccgcggcatctgtggttgctgtctcagagcagattcaacaatagtaagcaccaggctgcgtgacaagcagctcacggtgaaggcactgatgtgagaagctcgctgcagccaggtggaaccaggacacgcacatggacagaacccgtgtcctggtggtctctgggaagagcatctttctggtgccattgcctaggcgagcaagggagttgggatcagctcaggagggggtctcaagacactagcaaaaataataatggccaacatttatgtagtgtgcctactacaggccaggctctgttctatgttcatggttgacccccagaaccagcagtccaggttctgctgcagaccagctgaacaaaagcattgaggggtgtattagtctgttttcatgctgctaataaaggtatacccaagactgggtaatttataaaggaaagaggtttcaccgactcacagttccacatggctggggaggcctcacaatcatggctgaaggtgaatgaggagcaaggtcacatcttacttggcggcaggcaagagagcttgtgcaggggaagtccgctttataaaaccatcagatctcgtgagacttattcactaccacgagaatgtgggagaaacctccccatgattcattgatctccacctgaccccaccgttgacacgtggggcttattacaattcaaggtgagatttgggtggggactcagccaacccatattggaggtgggggataggccaagcatctgggttcatagaggatcctggggatggcaatggtggactccattctaatggactgcagactgggtacatgtgaagggactggacaaagccctacttcttgaagatttatggtgggatgtttactgacagcagaatctataaacctgagttttctttatcacgtactctcattagtaaaaaaaaaaaaaaaaaacagaacatgcagcccatatatatggtatataaagtacgtatgtccactactgtaggagtagagtgcgtatgttatgtaccaaaacgcacatttaaaaggcttgcatgctaaataagtccaaatttgcaatacttcccgccccgctgggccatctcactcacctacttggaggttatggcgtatagactctccagaatcttccacccttcctcctcttatcggttttcctgatttcaggtggctgagcagtagggaggagaggcctctgggaacgctgtgcttattaagaacccagtgatcagcagtcatgttcatttacgcccgtcagtttcatctgaggggtattttgatagtgatctcttggtgagccgagggtgtcaattaactgctcagtgctgaaactgagtggtgcataattcattgcaatttcactctcctggcctccttccactagattaaagttttgggagggctttggtgttccctgattcagatgtcccgcgtgctgccttgggttctgataaatgcatactggtacctctgttgagttacaggtgccaacagaaaacctcttcttgcaagctgacctctgagggagaattctgttactcactagcaggagcttggcaacaccaaaaagatttccttttttttttgagatggagtctcactctgttgccaggctggagtgcaatggcatgataccagctcactgcaacctccacctcctgggttcaagcgattctcctgcctcatcctcctgagtagctgggactacaggcgtgtgccaccacacccagctaatttttgcattttttagtagagacggggtttcaccatgttggccaggatggtctctatctcttgacctcatgatccgcccacctcagcctcccaaagtgctgggatgacaggcttgagccaccgcgcctggctaccagaaagatttcttatgagctatgattcatatttaaagagtgcccattaaaaaactgttggccgggcgtggtggctcacacctgtaatcccagcactttaggaggccgaggtcagcgaatcacaaggtcaggctcccgtggcacaagctgggggctgcccccaccccatgctccccagaggagcacccaagcgtctgggctcagacaggcctggatctgataaccggctctgatgccaccatccctgaggccaggcaagtcccagccgggctacagcactgttgtgagaatgcagatgaggttgtaaaggtgaagcacgtgtcacgcttctctcaataaagccatcattgctgttaggattgtcattaggaaaggctttgggccgggtgcagtggctcatgcctgtaatcccagcactttgggaggccaaggcgggtggatcacctgaggtcaggagtttgagaccagcctaaccaacatggtgaaaccccgtctctactaaaaatacaaaattagccaggcgtggtagcgggtgcctatagtcctagctacttgggaggctgaggcaagagaaccatttgaacctgggaggcggaggttgcagtgagctgagatcgtgccactgcactccagcgtaggcaacaagcgagactccaactcaaaaaaaaaaaaaaaccacacacacgctttggtctctttccctgaagatgggaacaaaaagctggacggccctggcctcttcatccctgcactggtttacttgaaggcagcagccccccacccacccacatcactctctagtgagtataaaaacgatgacaaggccaagcaaagggtcaaaacctgtggctctcctgccagccagctagccgagaggcctatgaggagctctgggaataaggggctgtgtcccacagtggggaagggacaatgggccattatgcacaggagataagaagggatgacctttgacaatttttttgtttgtttgtttgtttgttttgagatggagtctcactctgtctcctgggctggagtgcagcagtggcactctgcctcccgggttcaagcgattctcctctgtcagcctctagagttgtctggagttgtcagctcttccccttgcgtttcagatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccggcggccactcaggacttcagcaagctcctcagttcggtgaaggaaatcagtgacatagtgcagaggtagcagcagtcaggggtcaggtgtcaggcccgtcggagctgcctgcagcacatgcgggctcgcccatacccgtgacagtggctgacaagggactagtgagtcagcaccttggcccaggagctctgcgccaggcagagctgagggccctgtggagtccagctctactacctacgtttgcaccgcctgccctcccgcaccttcctcctccccgctccgtctctgtcctcgaattttatctgtggagttcctgctccgtggactgcagtcggcatgccaggacccgccagccccgctcccacctagtgccccagactgagctctccaggccaggtgggaacggctgatgtggactgtctttttcatttttttctctctggagcccctcctcccccggctgggcctccttcttccacttctccaagaatggaagcctgaactgaggccttgtgtgtcaggccctctgcctgcactccctggccttgcccgtcgtgtgctgaagacatgtttcaagaaccgcatttcgggaagggcatgcacgggcatgcacacggctggtcactctgccctctgctgctgcccggggtggggtgcactcgccatttcctcacgtgcaggacagctcttgatttgggtggaaaacagggtgctaaagccaaccagcctttgggtcctgggcaggtgggagctgaaaaggatcgaggcatggggcatgtcctttccatctgtccacatccccagagcccagctcttgctctcttgtgacgtgcactgtgaatcctggcaagaaagcttgagtctcaagggtggcaggtcactgtcactgccgacatccctcccccagcagaatggaggcaggggacaagggaggcagtggctagtggggtgaacagctggtgccaaatagccccagactgggcccaggcaggtctgcaagggcccagagtgaaccgtcctttcacacatctgggtgccctgaaagcccttcccctcccccactcctctaagacaaagtagattcttacaaggccctttcctttggaacaagacagccttcacttttctgagttcttgaagcatttcaaagccctgcctctgtgtagccgccctgagagagaatagagctgccactgggcacctgcgcacaggtgggaggaaagggcctggccagtcctggtcctggctgcactcttgaactgggcgaatgtcttatttaattaccgtgagtgacatagcctcatgttctgtgggggtcatcagggagggttaggaaaaccacaaacggagcccctgaaagcctcacgtatttcacagagcacgcctgccatcttctccccgaggctgccccaggccggagcccagatacgggggctgtgactctgggcagggacccggggtctcctggaccttgacagagcagctaactccgagagcagtgggcaggtggccgcccctgaggcttcacgccgggagaagccaccttcccaccccttcataccgcctcgtgccagcagcctcgcacaggccctagctttacgctcatcacctaaacttgtactttatttttctgatagaaatggtttcctctggatcgttttatgcggttcttacagcacatcacctctttgcccccgacggctgtgacgcagccggagggaggcactagtcaccgacagcggccttgaagacagagcaaagcgcccacccaggtcccccgactgcctgtctccatgaggtactggtcccttccttttgttaatgtgatgtgccactatattttacacgtatctcttggtatgcatcttttatagacgctcttttctaagtggcgtgtgcatagcgtcctgccctgccccctcgggggcctgtggtggctccccctctgcttctcggggtccagtgcattttgtttctgtatatgattctctgtggttttttttgaatccaaatctgtcctctgtagtattttttaaataaatcagtgtttacattagaattcttggcagattgcttcggcctgtgatcctgccctgttgttctgagcctgtgcggtggcggggccgggatgtagtgggttctggtttcccgacggggctggggcccggcatcaggtcttacagagcagaacggtggcaggttgcccccagctgcccagggcctctgcggcctgggtgcccacagccatctttcaggcacttgccctgctatgctcggaccctttctgtacccttggcgcttacctgctggcagcctggccccaccggcatcactgggagtgggctgtgccaccatacaagccacatctgactggcggtatcaggctgtcgcagcgacgtggaagtggggtgaggaggtcacgtcccgtgcttcttgctggctgagggcaaatccctccccacgtgcgcctgttctctctccacctttccgccctaagcccccaggtgcccttactcactcatgttccttgagccaggtgtcttaggtctttgtgtggatgaggaggagcggaggtgtgaggtgtgctgggccttcagggcctgggaaggccatcccagactgagccccaccgccctttcccagctcagtccccaccgagctgacagcttggaaaggggcggttgggaagtgagtagcaccctctgataatgatactgatcaggccagcaggtttgtcaggggtgcacatcctgctcagcagagcatatgaagatcacagcttggtgaagctccactcgagagacctggagcgagactgctgtgcggtgggtgacaggtggaactccagccgggccggggctggtggagcccactgcgagcagtgccaggcacaagattctccccctctcatttcacagggacaaaaccaggctggggcccccaatttccttgccggttgaaccatagctggccaccagatccaccaagcctggggcttctcttgtcctttgaatggctgcttctcccaggtctgaacgaagacataggacaactctggtgacagccacagctggtggccgcttcctcgtgtcagatgtggacggtcaccccgccctcccagcgctaagcgctgagactgcgtgtccagagtcggctcctgcacgtgaggccaattggtcacactgggccagtgtggaggaagatgcctctcatctcccaaatggggatctttcatgggtgtcacctgagcttatagagcttagtttagtctagccattccaagacagtggtttcttaaccttttctaggtcacaaaacctcctgagaacctaatgaaaactggatcccagccccagaaaaatgcacctgtgtgcacacacccagcagtttccaggtcatttcgagtcctgccttcgaggtccattgttattatttacaaaacaccagcctaaggtattggacattttcttaaaaggccaggcaaggtggctcatgcctgtaatcccaacactttgggaggctgaggtgggtggattgcttgaggccgggagttcaagaccagcctgggcaacatagaccccatctcacaaaaaatacaaaaacaagctgggtatggtggcacctgtagtccagctactcaggaagctgaggtgggaggattgcttgagcccaggaggtggaggttgcagcaagctatgatcgtgccactggattccagctccagagtgagaccctgtctcaaaaaaaatttttttttcttttttctttttgagacagagtttcgctcttgtctcccaggctggagtgcaatggcacaatctccgctcactgcaacctctgcctcctgggttcaagcgattctcctgcctcagcctcccgagtagctgggactagaggtgcgcaccaccagacccggctaatttttgtatttttagtagggacggagtttcaccatgttggtcaggatggtctcgatctcttgacctcatgatccgcccaccttggactcccaaagtgcttggattacaggcatgagccagcgcgcccggcccaccactttttttttttttttttttttttaagacgagtctcacactgtcgcccagtctgtagtgcagtggtgccatctcggctcactgcaagctccgcctcccaggttcata

SEQ ID NO: 7 - DNA, 5384 bp, Homo sapiens
aaaatgttggagatctgcctgaagctggtgggctgcaaatccaagaaggggctgtcctcgtcctccagctgttatctggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtagcagcagtcaggggtcaggtgtcaggcccgtcggagctgcctgcagcacatgcgggctcgcccatacccgtgacagtggctgacaagggactagtgagtcagcaccttggcccaggagctctgcgccaggcagagctgagggccctgtggagtccagctctactacctacgtttgcaccgcctgccctcccgcaccttcctcctccccgctccgtctctgtcctcgaattttatctgtggagttcctgctccgtggactgcagtcggcatgccaggacccgccagccccgctcccacctagtgccccagactgagctctccaggccaggtgggaacggctgatgtggactgtctttttcatttttttctctctggagcccctcctcccccggctgggcctccttcttccacttctccaagaatggaagcctgaactgaggccttgtgtgtcaggccctctgcctgcactccctggccttgcccgtcgtgtgctgaagacatgtttcaagaaccgcatttcgggaagggcatgcacgggcatgcacacggctggtcactctgccctctgctgctgcccggggtggggtgcactcgccatttcctcacgtgcaggacagctcttgatttgggtggaaaacagggtgctaaagccaaccagcctttgggtcctgggcaggtgggagctgaaaaggatcgaggcatggggcatgtcctttccatctgtccacatccccagagcccagctcttgctctcttgtgacgtgcactgtgaatcctggcaagaaagcttgagtctcaagggtggcaggtcactgtcactgccgacatccctcccccagcagaatggaggcaggggacaagggaggcagtggctagtggggtgaacagctggtgccaaatagccccagactgggcccaggcaggtctgcaagggcccagagtgaaccgtcctttcacacatctgggtgccctgaaagggcccttcccctcccccactcctctaagacaaagtagattcttacaaggccctttcctttggaacaagacagccttcacttttctgagttcttgaagcatttcaaagccctgcctctgtgtagccgccctgagagagaatagagctgccactgggcacctgcgcacaggtgggaggaaagggcctggccagtcctggtcctggctgcactcttgaactgggcgaatgtcttatttaattaccgtgagtgacatagcctcatgttctgtgggggtcatcagggagggttaggaaaaccacaaacggagcccctgaaagcctcacgtatttcacagagcacgcctgccatcttctccccgaggctgccccaggccggagcccagatacgggggctgtgactctgggcagggacccggggtctcctggaccttgacagagcagctaactccgagagcagtgggcaggtggccgcccctgaggcttcacgccgggagaagccaccttcccaccccttcataccgcctcgtgccagcagcctcgcacaggccctagctttacgctcatcacctaaacttgtactttatttttctgatagaaatggtttcctctggatcgttttatgcggttcttacagcacatcacctctttgcccccgacggctgtgacgcagccggagggaggcactagtcaccgacagcggccttgaagacagagcaaagcgcccacccaggtcccccgactgcctgtctccatgaggtactggtcccttccttttgttaacgtgatgtgccactatattttacacgtatctcttggtatgcatcttttatagacgctcttttctaagtggcgtgtgcatagcgtcctgccctgccccctcgggggcctgtggtggctccccctctgcttctcggggtccagtgcattttgtttctgtatatgattctctgtggttttttttgaatccaaatctgtcctctgtagtattttttaaataaatcagtgtttacatt

SEQ ID NO: 8 - DNA, 5881 bp, Homo sapiens
ggttggtgacttccacaggaaaagttctggaggagtagccaaagaccatcagcgtttcctttatgtgtgagaattgaaatgactagcattattgacccttttcagcatcccctgtgaatatttctgtttaggtttttcttcttgaaaagaaattgttattcagcccgtttaaaacaaatcaagaaacttttgggtaacattgcaattacatgaaattgataaccgcgaaaataattggaactcctgcttgcaagtgtcaacctaaaaaaagtgcttccttttgttatggaagatgtctttctgtgattgacttcaattgctgacttgtggagatgcagcgaatgtgaaatcccacgtatatgccatttccctctacgctcgctgaccgttctggaagatcttgaaccctcttctggaaaggggtacctattattactttatggggcagcagcctggaaaagtacttggggaccaaagaaggccaagcttgcctgccctgcattttatcaaaggagcagggaagaaggaatcatcgaggcatgggggtccacactgcaatgtttttgtggaacatgaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtagcagcagtcaggggtcaggtgtcaggcccgtcggagctgcctgcagcacatgcgggctcgcccatacccgtgacagtggctgacaagggactagtgagtcagcaccttggcccaggagctctgcgccaggcagagctgagggccctgtggagtccagctctactacctacgtttgcaccgcctgccctcccgcaccttcctcctccccgctccgtctctgtcctcgaattttatctgtggagttcctgctccgtggactgcagtcggcatgccaggacccgccagccccgctcccacctagtgccccagactgagctctccaggccaggtgggaacggctgatgtggactgtctttttcatttttttctctctggagcccctcctcccccggctgggcctccttcttccacttctccaagaatggaagcctgaactgaggccttgtgtgtcaggccctctgcctgcactccctggccttgcccgtcgtgtgctgaagacatgtttcaagaaccgcatttcgggaagggcatgcacgggcatgcacacggctggtcactctgccctctgctgctgcccggggtggggtgcactcgccatttcctcacgtgcaggacagctcttgatttgggtggaaaacagggtgctaaagccaaccagcctttgggtcctgggcaggtgggagctgaaaaggatcgaggcatggggcatgtcctttccatctgtccacatccccagagcccagctcttgctctcttgtgacgtgcactgtgaatcctggcaagaaagcttgagtctcaagggtggcaggtcactgtcactgccgacatccctcccccagcagaatggaggcaggggacaagggaggcagtggctagtggggtgaacagctggtgccaaatagccccagactgggcccaggcaggtctgcaagggcccagagtgaaccgtcctttcacacatctgggtgccctgaaagggcccttcccctcccccactcctctaagacaaagtagattcttacaaggccctttcctttggaacaagacagccttcacttttctgagttcttgaagcatttcaaagccctgcctctgtgtagccgccctgagagagaatagagctgccactgggcacctgcgcacaggtgggaggaaagggcctggccagtcctggtcctggctgcactcttgaactgggcgaatgtcttatttaattaccgtgagtgacatagcctcatgttctgtgggggtcatcagggagggttaggaaaaccacaaacggagcccctgaaagcctcacgtatttcacagagcacgcctgccatcttctccccgaggctgccccaggccggagcccagatacgggggctgtgactctgggcagggacccggggtctcctggaccttgacagagcagctaactccgagagcagtgggcaggtggccgcccctgaggcttcacgccgggagaagccaccttcccaccccttcataccgcctcgtgccagcagcctcgcacaggccctagctttacgctcatcacctaaacttgtactttatttttctgatagaaatggtttcctctggatcgttttatgcggttcttacagcacatcacctctttgcccccgacggctgtgacgcagccggagggaggcactagtcaccgacagcggccttgaagacagagcaaagcgcccacccaggtcccccgactgcctgtctccatgaggtactggtcccttccttttgttaacgtgatgtgccactatattttacacgtatctcttggtatgcatcttttatagacgctcttttctaagtggcgtgtgcatagcgtcctgccctgccccctcgggggcctgtggtggctccccctctgcttctcggggtccagtgcattttgtttctgtatatgattctctgtggttttttttgaatccaaatctgtcctctgtagtattttttaaataaatcagtgtttacattagaa

SEQ ID NO: 9 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 10 - protein, 1149 aa, Homo sapiens
MGQQPGKVLGDQRRPSLPALHFIKGAGKKESSRHGGPHCNVFVEHEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 11 - protein, 1142 aa, Mus sp.
MGQQPGKVLGDQRRPSLPALHFIKGAGKRDSSRHGGPHCNVFVEHEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTIYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVSAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKRGTRGGAGSMLQAPELPTKTRTCRRAAEQKDAPDTPELLHTKGLGESDALDSEPAVSPLLPRKERGPPDGSLNEDERLLPRDRKTNLFSALIKKKKKMAPTPPKRSSSFREMDGQPDRRGASEDDSRELCNGPPALTSDAAEPTKSPKASNGAGVPNGAFREPGNSGFRSPHMWKKSSTLTGSRLAAAEEESGMSSSKRFLRSCSASCMPHGARDTEWRSVTLPRDLPSAGKQFDSSTFGGHKSEKPALPRKRTSESRSEQVAKSTAMPPPRLVKKNEEAAEEGFKDTESSPGSSPPSLTPKLLRRQVTASPSSGLSHKEEATKGSASGMGTPATAEPAPPSNKVGLSKASSEEMRVRRHKHSSESPGRDKGRLAKLKPAPPPPPACTGKAGKPAQSPSQEAGEAGGPTKTKCTSLAMDAVNTDPTKAGPPGEGLRKPVPPSVPKPQSTAKPPGTPTSPVSTPSTAPAPSPLAGDQQPSSAAFIPLISTRVSLRKTRQPPERIASGTITKGVVLDSTEALCLAISRNSEQMASHSAVLEAGKNLYTFCVSYVDSIQQMRNKFAFREAINKLESNLRELQICPATASSGPAATQDFSKLLSSVKEISDIVRR

SEQ ID NO: 12 - protein, 1182 aa, Mus sp.
MGQQVGRVGEAPGLQQPQPRGIRGSSAARPSGRRRDPAGRTADAGFNVFTQHDHFASCVEDGFEGDKTGGSSPEVLHRPFGCDAESQALNEAIRWSSKENLLGATESDPNLFVALYDFVASGDNTLSITKGEKLRVLGYNQNGEWSEVRSKNGQGWVPSNYITPVNSLEKHSWYHGPVSRSAAEYLLSSLINGSFLVRESESSPGQLSISLRYEGRVYHYRINTTTDSKVYVTAESRFSTLAELVHHHSTVADGLVTTLHYPAPKCNKPTVYGVSPIHDKWEMERTDITMKHKLGGGQYGEVYVGVWKKYSLTVAVKTFKEDTMEVEEFLKEAAVMKEIKHPNLVQLLGVCTLEPPFYIVTEYMPYGNLLDYLRECSREEVTAVVLLYMATQISSAMEYLEKKNFIHRDLAARNCLVGENHVVKVADFGLSRLMTGDTYTAHAGAKFPIKWTAPESLAYNTFSIKSDVWAFGVLLWEIATYGMSPYPGIDLSQVYDLLEKGYRMEQPEGCPPKVYELMRACWKWSPADRPSFAETHQAFETMFHDSSISEEVAEELGRTASSSSVVPYLPRLPLLPSKTRTLRKQGENKENLDGGLDAAESLASSSAPAGFIRSTQASSGSPALPRKQRDKSPSSLLEDAKETCFTRDRKGGFFSSFMKKRNAPTPPKRSSSFREMENQPHKKYELTGNFSPVASLQNADGFSVAPSQQEPNLVPAKCYGGSFAQRNLCADDDSGGGGGSGTAGGGWSGITGFFTPRLIKKTLGLRAGKPTASDDTSKPFPRSNSTSSMSSGLPEQDRMAMTLPRNCQRSKLQLERTVSTSSQPEENVDRANDMLPKKSEEGAAPARERPKAKLLPRGATALPLRAPDPAITESDSPGVGVAGVAAAPKGKERNGGTRLGVAGVPEDGEQLGWSSPAKAVAVLPTTHNHKVPVLISPTLKHTPADVQLIGTDSQGNKFKLLSEHQVTSSGDKDRPRRVKPKCAPPPPPVMRLLQHPSTCSDPEEEPTAPPAGQHTPETQEGGKKAAPGPVPSSGKPGRPVMPPPQVPLPTSSISPAKMANGTAGTKVALRKTKQAAEKISADKISKEALLECADLLSSAITEPVPNSQLVDTGHQLLDYCSGYVDSIPQTRNKFAFREAVSKLELSLQELQVSSTAAGVPGTNPVLNNLLSCVQEISDVVQR

SEQ ID NO: 13 - DNA, 6021 bp, Homo sapiens
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccggaagggccatggccagcccggggcggacgccgagaagcccttctacgtgaacgtcgagtttcaccacgagcgcggcctggtgaaggtcaacgacaaagaggtgtcggaccgcatcagctcccttggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatcatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatgacgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctgaagtccactaaagcgagtgagctggacttggaaaagggcttggagatgagaaaatgggtcctgtcgggaatcctggctagcgaggagacttacctgagccacctggaggcactgctgctgcccatgaagcctttgaaagccgctgccaccacctctcagccggtgctgacgagtcagcagatcgagaccatcttcttcaaagtgcctgagctctacgagatccacaaggagtcctatgatgggctcttcccccgcgtgcagcagtggagccaccagcagcgggtgggcgacctcttccagaagctggccagccagctgggtgtgtaccgggccttcgtggacaactacggagttgccatggaaatggctgagaagtgctgtcaggccaatgctcagtttgcagaaatctccgagaacctgagagccagaagcaacaaagatgccaaggatccaacgaccaagaactctctggaaactctgctctacaagcctgtggaccgtgtgacgaggagcacgctggtcctccatgacttgctgaagcacactcctgccagccaccctgaccaccccttgctgcaggacgccctccgcatctcacagaacttcctgtccagcatcaatgaggagatcacaccccgacggcagtccatgacggtgaagaagggagagcaccggcagctgctgaaggacagcttcatggtggagctggtggagggggcccgcaagctgcggcacgtcttcctgttcaccgacctgcttctctgcaccaagctcaagaagcagagcggaggcaaaacgcagcagtatgactgcaaatggtacattccgctcacggatctcagcttccagatggtggatgaactggaggcagtgcccaacatccccctggtgcccgatgaggagctggacgctttgaagatcaagatctcccagatcaagagtgacatccagagagagaagagggcgaacaagggcagcaaggctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatccgctgagcagcgggatcaatggcagcttcttggtgcgtgagagtgagagcagtcctagccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctagtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctagagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgaccgttcccaggtgtatgagctgctagagaaggactaccgcatgaagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgactaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgtctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagcctggaaaggcagtgccttagggacccctgctgcagctgagccagtgacccccaccagcaaagcaggctcaggtgcaccaaggggcaccagcaagggccccgccgaggagtccagagtgaggaggcacaagcactcctctgagtcgccagggagggacaaggggaaattgtccaagctcaaacctgccccgccgcccccaccagcagcctctgcagggaaggctggaggaaagccctcgcagaggcccggccaggaggctgccggggaggcagtcttgggcgcaaagacaaaagccacgagtctggttgatgctgtgaacagtgacgctgccaagcccagccagccggcagagggcctcaaaaagcccgtgctcccggccactccaaagccacaccccgccaagccgtcggggacccccatcagcccagcccccgttcccctttccacgttgccatcagcatcctcggccttggcaggggaccagccgtcttccactgccttcatccctctcatatcaacccgagtgtctcttcggaaaacccgccagcctccagagcgggccagcggcgccatcaccaagggcgtggtcttggacagcaccgaggcgctgtgcctcgccatctctgggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgtcagcaggcagtggtccggcggccactcaggacttcagcaagctcctcagttcggtgaaggaaatcagtgacatagtgcagaggtag

SEQ ID NO: 14 - protein, 2006 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGIMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDDEGAFHGDADGSFGTPPGYGCAADRAEEQRRHQDGLPYIDDSPSSSPHLSSKGRGSRDALVSGALKSTKASELDLEKGLEMRKWVLSGILASEETYLSHLEALLLPMKPLKAAATTSQPVLTSQQIETIFFKVPELYEIHKESYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKSDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYPLSSGINGSFLVRESESSPSQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPSFAEIHQAFETMFQESSISDEVEKELGKQGVRGAVTTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSVSCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAWKGSALGTPAAAEPVTPTSKAGSGAPRGTSKGPAEESRVRRHKHSSESPGRDKGKLSKLKPAPPPPPAASAGKAGGKPSQRPGQEAAGEAVLGAKTKATSLVDAVNSDAAKPSQPAEGLKKPVLPATPKPHPAKPSGTPISPAPVPLSTLPSASSALAGDQPSSTAFIPLISTRVSLRKTRQPPERASGAITKGVVLDSTEALCLAISGNSEQMASHSAVLEAGKNLYTFCVSYVDSIQQMRNKFAFREAINKLENNLRELQICPASAGSGPAATQDFSKLLSSVKEISDIVQR

SEQ ID NO: 15 - DNA, 6096 bp, Homo sapiens
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccggaagggccatggccagcccggggcggacgccgagaagcccttctacgtgaacgtcgagtttcaccacgagcgcggcctggtgaaggtcaacgacaaagaggtgtcggaccgcatcagctcccttggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatcatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatgacgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctgaagtccactaaagcgagtgagctggacttggaaaagggcttggagatgagaaaatgggtcctgtcgggaatcctggctagcgaggagacttacctgagccacctggaggcactgctgctgcccatgaagcctttgaaagccgctgccaccacctctcagccggtgctgacgagtcagcagatcgagaccatcttcttcaaagtgcctgagctctacgagatccacaaggagtcctatgatgggctcttcccccgcgtgcagcagtggagccaccagcagcgggtgggcgacctcttccagaagctggccagccagctgggtgtgtaccgggccttcgtggacaactacggagttgccatggaaatggctgagaagtgctgtcaggccaatgctcagtttgcagaaatctccgagaacctgagagccagaagcaacaaagatgccaaggatccaacgaccaagaactctctggaaactctgctctacaagcctgtggaccgtgtgacgaggagcacgctggtcctccatgacttgctgaagcacactcctgccagccaccctgaccaccccttgctgcaggacgccctccgcatctcacagaacttcctgtccagcatcaatgaggagatcacaccccgacggcagtccatgacggtgaagaagggagagcaccggcagctgctgaaggacagcttcatggtggagctggtggagggggcccgcaagctgcggcacgtcttcctgttcaccgacctgcttctctgcaccaagctcaagaagcagagcggaggcaaaacgcagcagtatgactgcaaatggtacattccgctcacggatctcagcttccagatggtggatgaactggaggcagtgcccaacatccccctggtgcccgatgaggagctggacgctttgaagatcaagatctcccagatcaagagtgacatccagagagagaagagggcgaacaagggcagcaaggctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagatgatgagtctccggggctctatgggtttctgaatgtcatcgtccactcagccactggatttaagcagagttcagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatccgctgagcagcgggatcaatggcagcttcttggtgcgtgagagtgagagcagtcctagccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctagtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctagagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgaccgttcccaggtgtatgagctgctagagaaggactaccgcatgaagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgactaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgtctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagcctggaaaggcagtgccttagggacccctgctgcagctgagccagtgacccccaccagcaaagcaggctcaggtgcaccaaggggcaccagcaagggccccgccgaggagtccagagtgaggaggcacaagcactcctctgagtcgccagggagggacaaggggaaattgtccaagctcaaacctgccccgccgcccccaccagcagcctctgcagggaaggctggaggaaagccctcgcagaggcccggccaggaggctgccggggaggcagtcttgggcgcaaagacaaaagccacgagtctggttgatgctgtgaacagtgacgctgccaagcccagccagccggcagagggcctcaaaaagcccgtgctcccggccactccaaagccacaccccgccaagccgtcggggacccccatcagcccagcccccgttcccctttccacgttgccatcagcatcctcggccttggcaggggaccagccgtcttccactgccttcatccctctcatatcaacccgagtgtctcttcggaaaacccgccagcctccagagcgggccagcggcgccatcaccaagggcgtggtcttggacagcaccgaggcgctgtgcctcgccatctctgggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgtcagcaggcagtggtccggcggccactcaggacttcagcaagctcctcagttcggtgaaggaaatcagtgacatagtgcagaggtag

SEQ ID NO: 16 - protein, 2031 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGIMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDDEGAFHGDADGSFGTPPGYGCAADRAEEQRRHQDGLPYIDDSPSSSPHLSSKGRGSRDALVSGALKSTKASELDLEKGLEMRKWVLSGILASEETYLSHLEALLLPMKPLKAAATTSQPVLTSQQIETIFFKVPELYEIHKESYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKSDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEDDESPGLYGFLNVIVHSATGFKQSSEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYPLSSGINGSFLVRESESSPSQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPSFAEIHQAFETMFQESSISDEVEKELGKQGVRGAVTTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSVSCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAWKGSALGTPAAAEPVTPTSKAGSGAPRGTSKGPAEESRVRRHKHSSESPGRDKGKLSKLKPAPPPPPAASAGKAGGKPSQRPGQEAAGEAVLGAKTKATSLVDAVNSDAAKPSQPAEGLKKPVLPATPKPHPAKPSGTPISPAPVPLSTLPSASSALAGDQPSSTAFIPLISTRVSLRKTRQPPERASGAITKGVVLDSTEALCLAISGNSEQMASHSAVLEAGKNLYTFCVSYVDSIQQMRNKFAFREAINKLENNLRELQICPASAGSGPAATQDFSKLLSSVKEISDIVQR

SEQ ID NO: 17 - DNA, 4221 bp, Homo sapiens
atgtctgagactcctgctcagtgtagcattaagcaggaacgaatttcatatacacctccagagagcccagtgccgagttacgcttcctcgacgccacttcatgttccagtgcctcgagcgctcaggatggaggaagactcgatccgcctgcctgcgcacctgcgcttgcagccaatttactggagcagggatgacgtagcccagtggctcaagtgggctgaaaatgagttttctttaaggccaattgacagcaacacgtttgaaatgaatggcaaagctctcctgctgctgaccaaagaggactttcgctatcgatctcctcattcaggtgatgtgctctatgaactccttcagcatattctgaagcagaggaaacctcggattcttttttcaccattcttccaccctggaaactctatacacacacagccggaggtcatactgcatcagaaccatgaagaagataactgtgtccagaggacccccaggccatccgtggataatgtgcaccataaccctcccaccattgaactgttgcaccgctccaggtcacctatcacgacaaatcaccggccttctcctgaccccgagcagcggcccctccggtcccccctggacaacatgatccgccgcctctccccggctgagagagctcagggacccaggccgcaccaggagaacaaccaccaggagtcctaccctctgtcagtgtctcccatggagaataatcactgcccagcgtcctccgagtcccacccgaagccatccagcccccggcaggagagcacacgcgtgatccagctgatgcccagccccatcatgcaccctctgatcctgaacccccggcactccgtggatttcaaacagtccaggctctccgaggacgggctgcatagggaagggaagcccatcaacctctctcatcgggaagacctggcttacatgaaccacatcatggtctctgtctccccgcctgaagagcacgccatgcccattgggagaatagcagactgtagactgctttgggattacggtggtgataacacactcagcatcactaaaggtgaaaagctacgagtccttggttacaaccagaatggtgagtggagtgaagttcgctctaagaatgggcagggctgggtgccaagcaactacatcaccccagtgaacagcctggaaaaacactcctggtaccatggacctgtgtcacgcagtgcagctgagtatctgctcagcagtctaatcaatggcagcttcctggtgcgagaaagtgagagtagccctgggcagctgtccatctcgctcaggtacgagggacgtgtgtatcactacaggatcaataccactgcagatggcaaggtgtatgtgactgctgagagccgcttcagcaccttggcagagcttgtacaccatcactccacagtggctgatgggctggtgacaacattacactacccagcacccaagtgtaataagcctacagtctatggtgtgtcccccatccacgacaaatgggaaatggagcgaacagatattaccatgaagcacaaacttgggggcggtcagtatggagaggtttacgttggcgtctggaagaaatacagccttacagttgctgtgaaaacattgaaggaagataccatggaggtagaagaattcctgaaagaagctgcagtaatgaaggaaatcaagcatcctaatctggtacaacttttaggtgtgtgtactttggagccaccattttacattgtgactgaatacatgccatacgggaatttgctggattacctccgagaatgcaaccgagaagaggtgactgcagttgtgctgctctacatggccactcagatttcttctgcaatggagtacttagagaagaagaatttcatccatagagatcttgcagctcgtaactgcctagtgggagaaaaccatgtggtaaaagtggctgactttggcttaagtagattgatgactggagacacttatactgctcatgctggagccaaatttcctattaagtggacagcaccagagagtcttgcctacaataccttctcaattaaatctgacgtctgggcttttggggtattgttgtgggaaattgctacctatggaatgtcaccatatccaggtattgacctgtctcaggtctatgacctactagaaaaaggatatcgaatggaacagcctgagggatgcccccctaaggtttatgaacttatgagagcatgctggaagtggagccctgccgataggccctcttttgctgaaacacaccaagcttttgaaaccatgttccatgactccagcatttctgaagaggtagctgaggagcttgggagagccgcctcctcgtcatctgttgttccatacctgccccggctacctatacttccttccaagactcggacactgaagaaacaggtggagaacaaggagaacattgaaggggcacaagatgccacagaaaattctgcttccagtttagcaccagggttcatcagaggtgcacaggcctctagtggatccccagcactgcctcgaaagcaaagagacaagtcacccagcagcctcttggaagatgccaaagagacatgcttcaccagggataggaaggggggcttcttcagctccttcatgaagaagagaaatgctcctacaccccccaaacgcagcagctccttccgagaaatggagaatcagccccataagaaatacgaactcacgggtaacttctcatctgttgcttctctacagcatgctgatgggttctctttcactcctgcccagcaagaggcgaatctggtgccacccaagtgctatggggggagctttgcacagaggaacctctgtaatgacgacggtggtgggggtgggggcagtggcactgctgggggtgggtggtctggcatcacaggcttctttacaccacgcttaatcaaaaagacactgggcttacgagcaggtaaacccacagccagtgatgacacttccaagccttttccaaggtcaaactctacatcttccatgtcctcagggcttccagagcaggataggatggcaatgacccttcccaggaactgccagaggtccaaactccagctggaaaggacagtgtccacctcttctcagccagaagagaatgtggacagggccaatgacatgcttccaaaaaaatcagaggaaagtgctgctccaagcagggagagaccaaaagccaagttattgcccagaggagccacagctcttcctctcagaacaccctctggggatctagccattacagagaaggaccctccaggggtgggagtggctggagtggcagctgcccccaagggtaaagagaagaatggtggggcacgacttgggatggctggagttccagaggatggagagcagccgggctggccttctccagccaaggctgcccccgtcctcccaaccactcacaaccacaaagtgccagtccttatctcacccactctgaaacacactccagctgacgtgcagctcattggcacagactctcaggggaataaattcaagctcttatctgagcatcaggtcacatcctctggagacaaggaccgaccccgacgggtaaaaccaaagtgtgccccacccccaccaccagtgatgagactactgcagcatccgtccatctgctcagaccctacagaagagccaactgccctaactgcaggacagtccacatcagaaacacaggaaggaggaaagaaggcagctctgggcgcagtgcccatcagtgggaaagctgggaggccagtgatgcctccacctcaagtgcctctgcccacatcttccatctcgccagccaaaatggccaatggcacagcaggtactaaagtggctctgagaaaaaccaaacaggccgctgagaaaatctcagcagacaaaatcagcaaagaggccctgctggaatgtgctgacctactgtccagtgcactcacggaacctgtgcccaacagccagctggtagacactggacaccagctgcttgactactgctcaggctatgtggactgcatccctcaaactcgcaacaaatttgccttccgagaggctgtgagcaaactggaactcagcctgcaggagctacaggtttcttcagcagctgctggtgtgcccgggacaaaccctgtccttaataacttattgtcatgtgtacaggaaatcagtgatgtggtgcagaggtag

SEQ ID NO: 18 - protein, 1406 aa, Homo sapiens
MSETPAQCSIKQERISYTPPESPVPSYASSTPLHVPVPRALRMEEDSIRLPAHLRLQPIYWSRDDVAQWLKWAENEFSLRPIDSNTFEMNGKALLLLTKEDFRYRSPHSGDVLYELLQHILKQRKPRILFSPFFHPGNSIHTQPEVILHQNHEEDNCVQRTPRPSVDNVHHNPPTIELLHRSRSPITTNHRPSPDPEQRPLRSPLDNMIRRLSPAERAQGPRPHQENNHQESYPLSVSPMENNHCPASSESHPKPSSPRQESTRVIQLMPSPIMHPLILNPRHSVDFKQSRLSEDGLHREGKPINLSHREDLAYMNHIMVSVSPPEEHAMPIGRIADCRLLWDYGGDNTLSITKGEKLRVLGYNQNGEWSEVRSKNGQGWVPSNYITPVNSLEKHSWYHGPVSRSAAEYLLSSLINGSFLVRESESSPGQLSISLRYEGRVYHYRINTTADGKVYVTAESRFSTLAELVHHHSTVADGLVTTLHYPAPKCNKPTVYGVSPIHDKWEMERTDITMKHKLGGGQYGEVYVGVWKKYSLTVAVKTLKEDTMEVEEFLKEAAVMKEIKHPNLVQLLGVCTLEPPFYIVTEYMPYGNLLDYLRECNREEVTAVVLLYMATQISSAMEYLEKKNFIHRDLAARNCLVGENHVVKVADFGLSRLMTGDTYTAHAGAKFPIKWTAPESLAYNTFSIKSDVWAFGVLLWEIATYGMSPYPGIDLSQVYDLLEKGYRMEQPEGCPPKVYELMRACWKWSPADRPSFAETHQAFETMFHDSSISEEVAEELGRAASSSSVVPYLPRLPILPSKTRTLKKQVENKENIEGAQDATENSASSLAPGFIRGAQASSGSPALPRKQRDKSPSSLLEDAKETCFTRDRKGGFFSSFMKKRNAPTPPKRSSSFREMENQPHKKYELTGNFSSVASLQHADGFSFTPAQQEANLVPPKCYGGSFAQRNLCNDDGGGGGGSGTAGGGWSGITGFFTPRLIKKTLGLRAGKPTASDDTSKPFPRSNSTSSMSSGLPEQDRMAMTLPRNCQRSKLQLERTVSTSSQPEENVDRANDMLPKKSEESAAPSRERPKAKLLPRGATALPLRTPSGDLAITEKDPPGVGVAGVAAAPKGKEKNGGARLGMAGVPEDGEQPGWPSPAKAAPVLPTTHNHKVPVLISPTLKHTPADVQLIGTDSQGNKFKLLSEHQVTSSGDKDRPRRVKPKCAPPPPPVMRLLQHPSICSDPTEEPTALTAGQSTSETQEGGKKAALGAVPISGKAGRPVMPPPQVPLPTSSISPAKMANGTAGTKVALRKTKQAAEKISADKISKEALLECADLLSSALTEPVPNSQLVDTGHQLLDYCSGYVDCIPQTRNKFAFREAVSKLELSLQELQVSSAAAGVPGTNPVLNNLLSCVQEISDVVQR

SEQ ID NO: 19 - DNA, 4158 bp, Homo sapiens
atgtctgagactcctgctcagtgtagcattaagcaggaacgaatttcatatacacctccagagagcccagtgccgagttacgcttcctcgacgccacttcatgttccagtgcctcgagcgctcaggatggaggaagactcgatccgcctgcctgcgcacctgcgcttgcagccaatttactggagcagggatgacgtagcccagtggctcaagtgggctgaaaatgagttttctttaaggccaattgacagcaacacgtttgaaatgaatggcaaagctctcctgctgctgaccaaagaggactttcgctatcgatctcctcattcaggtgatgtgctctatgaactccttcagcatattctgaagcagaggaaacctcggattcttttttcaccattcttccaccctggaaactctatacacacacagccggaggtcatactgcatcagaaccatgaagaagataactgtgtccagaggacccccaggccatccgtggataatgtgcaccataaccctcccaccattgaactgttgcaccgctccaggtcacctatcacgacaaatcaccggccttctcctgaccccgagcagcggcccctccggtcccccctggacaacatgatccgccgcctctccccggctgagagagctcagggacccaggccgcaccaggagaacaaccaccaggagtcctaccctctgtcagtgtctcccatggagaataatcactgcccagcgtcctccgagtcccacccgaagccatccagcccccggcaggagagcacacgcgtgatccagctgatgcccagccccatcatgcaccctctgatcctgaacccccggcactccgtggatttcaaacagtccaggctctccgaggacgggctgcatagggaagggaagcccatcaacctctctcatcgggaagacctggcttacatgaaccacatcatggtctctgtctccccgcctgaagagcacgccatgcccattgggagaatagcagactgtagactgctttgggattacgatggtgagtggagtgaagttcgctctaagaatgggcagggctgggtgccaagcaactacatcaccccagtgaacagcctggaaaaacactcctggtaccatggacctgtgtcacgcagtgcagctgagtatctgctcagcagtctaatcaatggcagcttcctggtgcgagaaagtgagagtagccctgggcagctgtccatctcgctcaggtacgagggacgtgtgtatcactacaggatcaataccactgcagatggcaaggtgtatgtgactgctgagagccgcttcagcaccttggcagagcttgtacaccatcactccacagtggctgatgggctggtgacaacattacactacccagcacccaagtgtaataagcctacagtctatggtgtgtcccccatccacgacaaatgggaaatggagcgaacagatattaccatgaagcacaaacttgggggcggtcagtatggagaggtttacgttggcgtctggaagaaatacagccttacagttgctgtgaaaacattgaaggaagataccatggaggtagaagaattcctgaaagaagctgcagtaatgaaggaaatcaagcatcctaatctggtacaacttttaggtgtgtgtactttggagccaccattttacattgtgactgaatacatgccatacgggaatttgctggattacctccgagaatgcaaccgagaagaggtgactgcagttgtgctgctctacatggccactcagatttcttctgcaatggagtacttagagaagaagaatttcatccatagagatcttgcagctcgtaactgcctagtgggagaaaaccatgtggtaaaagtggctgactttggcttaagtagattgatgactggagacacttatactgctcatgctggagccaaatttcctattaagtggacagcaccagagagtcttgcctacaataccttctcaattaaatctgacgtctgggcttttggggtattgttgtgggaaattgctacctatggaatgtcaccatatccaggtattgacctgtctcaggtctatgacctactagaaaaaggatatcgaatggaacagcctgagggatgcccccctaaggtttatgaacttatgagagcatgctggaagtggagccctgccgataggccctcttttgctgaaacacaccaagcttttgaaaccatgttccatgactccagcatttctgaagaggtagctgaggagcttgggagagccgcctcctcgtcatctgttgttccatacctgccccggctacctatacttccttccaagactcggacactgaagaaacaggtggagaacaaggagaacattgaaggggcacaagatgccacagaaaattctgcttccagtttagcaccagggttcatcagaggtgcacaggcctctagtggatccccagcactgcctcgaaagcaaagagacaagtcacccagcagcctcttggaagatgccaaagagacatgcttcaccagggataggaaggggggcttcttcagctccttcatgaagaagagaaatgctcctacaccccccaaacgcagcagctccttccgagaaatggagaatcagccccataagaaatacgaactcacgggtaacttctcatctgttgcttctctacagcatgctgatgggttctctttcactcctgcccagcaagaggcgaatctggtgccacccaagtgctatggggggagctttgcacagaggaacctctgtaatgacgacggtggtgggggtgggggcagtggcactgctgggggtgggtggtctggcatcacaggcttctttacaccacgcttaatcaaaaagacactgggcttacgagcaggtaaacccacagccagtgatgacacttccaagccttttccaaggtcaaactctacatcttccatgtcctcagggcttccagagcaggataggatggcaatgacccttcccaggaactgccagaggtccaaactccagctggaaaggacagtgtccacctcttctcagccagaagagaatgtggacagggccaatgacatgcttccaaaaaaatcagaggaaagtgctgctccaagcagggagagaccaaaagccaagttattgcccagaggagccacagctcttcctctcagaacaccctctggggatctagccattacagagaaggaccctccaggggtgggagtggctggagtggcagctgcccccaagggtaaagagaagaatggtggggcacgacttgggatggctggagttccagaggatggagagcagccgggctggccttctccagccaaggctgcccccgtcctcccaaccactcacaaccacaaagtgccagtccttatctcacccactctgaaacacactccagctgacgtgcagctcattggcacagactctcaggggaataaattcaagctcttatctgagcatcaggtcacatcctctggagacaaggaccgaccccgacgggtaaaaccaaagtgtgccccacccccaccaccagtgatgagactactgcagcatccgtccatctgctcagaccctacagaagagccaactgccctaactgcaggacagtccacatcagaaacacaggaaggaggaaagaaggcagctctgggcgcagtgcccatcagtgggaaagctgggaggccagtgatgcctccacctcaagtgcctctgcccacatcttccatctcgccagccaaaatggccaatggcacagcaggtactaaagtggctctgagaaaaaccaaacaggccgctgagaaaatctcagcagacaaaatcagcaaagaggccctgctggaatgtgctgacctactgtccagtgcactcacggaacctgtgcccaacagccagctggtagacactggacaccagctgcttgactactgctcaggctatgtggactgcatccctcaaactcgcaacaaatttgccttccgagaggctgtgagcaaactggaactcagcctgcaggagctacaggtttcttcagcagctgctggtgtgcccgggacaaaccctgtccttaataacttattgtcatgtgtacaggaaatcagtgatgtggtgcagaggtag

SEQ ID NO: 20 - protein, 1385 aa, Homo sapiens
MSETPAQCSIKQERISYTPPESPVPSYASSTPLHVPVPRALRMEEDSIRLPAHLRLQPIYWSRDDVAQWLKWAENEFSLRPIDSNTFEMNGKALLLLTKEDFRYRSPHSGDVLYELLQHILKQRKPRILFSPFFHPGNSIHTQPEVILHQNHEEDNCVQRTPRPSVDNVHHNPPTIELLHRSRSPITTNHRPSPDPEQRPLRSPLDNMIRRLSPAERAQGPRPHQENNHQESYPLSVSPMENNHCPASSESHPKPSSPRQESTRVIQLMPSPIMHPLILNPRHSVDFKQSRLSEDGLHREGKPINLSHREDLAYMNHIMVSVSPPEEHAMPIGRIADCRLLWDYDGEWSEVRSKNGQGWVPSNYITPVNSLEKHSWYHGPVSRSAAEYLLSSLINGSFLVRESESSPGQLSISLRYEGRVYHYRINTTADGKVYVTAESRFSTLAELVHHHSTVADGLVTTLHYPAPKCNKPTVYGVSPIHDKWEMERTDITMKHKLGGGQYGEVYVGVWKKYSLTVAVKTLKEDTMEVEEFLKEAAVMKEIKHPNLVQLLGVCTLEPPFYIVTEYMPYGNLLDYLRECNREEVTAVVLLYMATQISSAMEYLEKKNFIHRDLAARNCLVGENHVVKVADFGLSRLMTGDTYTAHAGAKFPIKWTAPESLAYNTFSIKSDVWAFGVLLWEIATYGMSPYPGIDLSQVYDLLEKGYRMEQPEGCPPKVYELMRACWKWSPADRPSFAETHQAFETMFHDSSISEEVAEELGRAASSSSVVPYLPRLPILPSKTRTLKKQVENKENIEGAQDATENSASSLAPGFIRGAQASSGSPALPRKQRDKSPSSLLEDAKETCFTRDRKGGFFSSFMKKRNAPTPPKRSSSFREMENQPHKKYELTGNFSSVASLQHADGFSFTPAQQEANLVPPKCYGGSFAQRNLCNDDGGGGGGSGTAGGGWSGITGFFTPRLIKKTLGLRAGKPTASDDTSKPFPRSNSTSSMSSGLPEQDRMAMTLPRNCQRSKLQLERTVSTSSQPEENVDRANDMLPKKSEESAAPSRERPKAKLLPRGATALPLRTPSGDLAITEKDPPGVGVAGVAAAPKGKEKNGGARLGMAGVPEDGEQPGWPSPAKAAPVLPTTHNHKVPVLISPTLKHTPADVQLIGTDSQGNKFKLLSEHQVTSSGDKDRPRRVKPKCAPPPPPVMRLLQHPSICSDPTEEPTALTAGQSTSETQEGGKKAALGAVPISGKAGRPVMPPPQVPLPTSSISPAKMANGTAGTKVALRKTKQAAEKISADKISKEALLECADLLSSALTEPVPNSQLVDTGHQLLDYCSGYVDCIPQTRNKFAFREAVSKLELSLQELQVSSAAAGVPGTNPVLNNLLSCVQEISDVVQR

SEQ ID NO: 21 - DNA, 46235 bp, Homo sapiens
gaattcggatccacccacctcccaaagtgctgggattacaggcatgagccaccacgcctggctatggccggcttctttaccgcaagctgttttatcagcaaggtgtttatgacctgtattttgtgctgaccgcctacatcatcctgtgacttagaatgcctcagtcatctgggaatgcatcccagtaggtttcagcctcattttacccagctcctattcaagatggagttgctctggttcacacgcccctgacaattacacagccattgatacggtatgtaaaccaaaagtgactcttaagcacccccaaccaactgaatagacccttcccccttggccaagaggatgccaaagaaacctgaaaaactggccaggcacagtggctcacacctgtaatcccagcactttgggaggctgaggcaggtggatcacgaggtcaggagtttgagaccaacctgaccaacagggtgaaaccccgtctctactaaaaatacaaaacttagccgggcatggtggcacaggcctgtaatcccatctgctcaggaggctgaggcagagaattgcttgaacctgggaggtggaggttgcagtgagtcgagatcatgccactgcactccagcgtgggcaacagagtgagattctgtctcaaaaaaaaaaaaaaaagaaaagaaaatctgagtccacctatgccctgtaagcccccgctttgagttgtcgcgcctttccagatggaaccaatgtatacctcacatgcactgattgatgtcttatgtctccttaaaatatataaaccaaagctgtaacccaaccaccttgggcccatgttctcaggacctcctgaggctgcctcatgggtcatggcccttgcatttagctcagaataaatctcttttaatactttccagagtttggcatttttttgtcaatatgtatatgaaatatacccttaggttttctgtctaatatgttctatgctgtcccctgctcccaccccacgatcatctgctatattttcttatccgttgctgtttaaagacccaccccaaaatgtaacagtaaaaaacaaccaagatttattgcttcttttgattcagtgggttggcaatctcggtgacttttttcctttttttttttgtagatggagtttcactattgtcacccaggctggagtgcaatggtgatctcggctcactgcaacctccacctcccgggttcaagtaattctcctgcctcagcctcccaagtagctgggattacaggtgcccgccaccacacccagctaatttttgtatttttagtagatacggggtttcaccatgttgcccaggctggtcttgaactcctaacctcaggttatcctcccacctcgtcctcccaaagtgctgggattacaggcgtgagccacagcgcccagtcccattcccattttttttaaatcagagtcactctgtcacccaagctggagtgcactggcatgattatagctcactgtacccgtgaactcctaacacctgggcctaagggatcctcccacctcagccttctgagtagcagggactacaggcatgagccacatggcctggttctaaacataattttaaaatcatactcatgttctacaatataaaattcaactttaataattcatgttcccattgttcacagtattggctttcttatttattttatattttgagacagggtctccctctatcattcatgctggagtgcagtggcgaaatcatggttcattggcagccttgacctcctgggctcagctgatcctcttgcctcagtcttccaagtagctgggactgtaggcacaggccaccacacccagctaatttttttaaagttttgtagacacagggtcttcactatgttgcccaggctggtcttgaactcctgagctcaagcaatccacctgcctcggcctcccaaggtgctggattataggcatgagtcactgtgcctggcctttggctttcttatttagtattcatttttacaatccctttggaacttacattctaggttcattttaattaggaaatgtttcattttctctcttcctcactgtgtgtgacgctgtctgtctccctgttctgcagttgcctcaacctctctgtattagggtgggctaaacatctggaacaaatggactccaaacgcaatatcttaaacacaacaggattttcttttgctttcacgtaacagggcagggtgtatgtccaggtcaggcagaggaggagaaagggatagtaaggaagatctgttccacgcagtcttggagggatgcaggctggtgggctctgccatcctcaaaacagggcttccaaggttgtacctgtcacctgtagtaggctgaataatggcctccaaatatgtctgcatcctaatagtcagaacatgtgatgttgctttacatggcaaaaaggagttagcagatgtcatgacattaaggatcttgagatgggaggatattctgggtgggtctgaaatgggatcccaaatgttcttgtaatagggaggcaaagagacatatccgtacagagagaaaacaggtgtgaagatgaatgcatgggtctgagggatgcaagaagctagaaaggccacagatggttcctcccctagagtctccaaaaggaacaagccccactgacaccttgactttagcccatgaaactgatttcagactctggcttccagagctgaagagcatatatttgcattgttttgaggcactgagtctgtcataatgtgttgcagcagcctaggaagctggtccacggtaggaggcagccccggggaggaatgatggtccaggctcagaagccatgcatgtcacctcccctcatgttccatgggcgagaacctggccacatggcagctcccaactccaagggaagctgggaaatgtagtagagctggacacccacatgtccacttagtaccgaggagggtgggggcgctgggtggacagctacatctccgtcacacgtgttgtattgagtcctacagccctgcgtggtggtgcaaatgccccaggtcccatctgcactctaagtcagggactggtttgctcaggtcctggacataaggctgtctggacatccctctttggctgcatgggtttttaaaatttttctttatttttatttttttgagacaggttcacaccatgttgctccagctggtcttgcactcctgggctcaagtgattcctcccacctcggcctccccaagtagctgggaccactggtgcgcccctggctgcgtgcttcttttacaagcgtgggtgacccaggcccagcagctttgcagttgcaggcccactcagcactttgcatttctcttcatttctggctcacagagatatcacttttgttttgttttgttttgtttttttgatagagtcccgctgtgtctcccaggctggagtgcaatggcgcgatctcagctcactgcaaccttcacctcccgggtttgagcgattatcctgcctcagcctcccaagtagctgggattacaggcgcctgccaccatgcctggcttatttttgtatttttattagagacggggtttcaccaggttggtcaggatagtctcggactcctgacctcaagtgatccacctgcatcggcctcccaaagtgctgggattacaggcgtgagcaaccacgcccagccaccagttttgtttttgtttttgtttttattttttgaattttagtagagatggggcttcaccatgctggccaggctggtctcaaattcctaacctcaagaaatccgtctgccttggcctcccaaagtgccgggattataggcatgagccacggcgcccggcttcgacctatttttgagcacagctatacttcttgctttgcaaattttacactgtatgtgtaattcctgtggccagcctcagtctctgccacacattctcctggggagcacaatgggcatgtggaagagtgtgctgcccctttctctctgggagccactctgaagcaccctgctcttcaccggcagcagactggtctttctaagccacgaacgctgccctcctgctgaaaagtctgcagtgagctttgtctttccttctctgtaaaattagcccagctaatttttgtactttaattttagagacggggtttcaccatgttggccaggctggtcttgaactcctgacctcaggtgatccacctgccttggcctcccaaagtgctgggattacaggcatgagccaccgcgcctggctgctatattttctttacatgttttcttgaataaaatacattttcttcaacctaaaagttcatttttccaattttaatagaaataagaacatttccgtgggcgcccaaagatatgtccacagcctaatcctcagcacctatgactatgacctcttatggcaaaaggtgtgacacagtgagggacttgagaggaggggtttgtgctggatcaatacagtcacagggctccctctgagatggacaccaccggctatgcctgcttttttgactagatgatttcatacctctgtagaggcagaaattaacctttaaaaaacaggcagcaatgtggcctggcaatgtggtggctcacacatgtaatcccagtgcaggagggtcacttgagcacaggagttcgagaccagcattagcaacacagcaggacccagtatctacaaaaatattaaaaaattagaggccgggcatggtggctcacgcctgtaatcccagcactttgggaggccgaggcgggaggatcacctgaggtcgggagttcgagaccagcctgaccaacatggagaaaccctgtctctactgaaaatacaaaattagccaggcaaagtgacgcacgcctgtaatcccagctactcgggaggctgaggcaggagaattgcttgaacccgagaggcagaggttgcagtgagctgagatggcgccattgcactccagcctgggcaacaagaggggaactccatctcaaaaaaaaaaaaattagctgagtgtggtggcacgtgcctgtagttccagctacagctacttgggaggctgaggtgggaggatcacttgagcccaggagatcgaggctgcagtgagccttgattgcaccactgcactccagcccctgtctctaaagattaaacaaacaaacaaaaaaaaacagtcagcaatgtaaaaaaaaaaattttttttttcagagatggggtctatattgcccaggctggtcttgaactcctgggctcaagcaatcctctggccttgccgctcaaagtgctggtattacaagcctggccaacaatccttgtgcacagaaaatgcaaattagctgtgtccagttaaatgcaattgacctttgcactagagatagagcagtttttgcaaattcatttcagcattcgtcttccgttttttttttttttgacacagggtttctgttgcccgtgccggagcggtccttttgcctcagcctccagaatagctgggaccacaggtaaacgtcaccatgcctggctaatttttttcttaaaaaaaattttttttttttggtagagacagggtcttgccatgctgcccaggctggtctcaaattcctgacctccagcaatcctcctgccttggcctcccaaagtgttgggattacaggcgcgaggcactgcatctggccaaattctttaaacaatttgtaacaacttatgatgtattcattatcgagttaaataaaatatttgacgtgttcattttatatgtgtatgaaggtcatgattttacctttaaaaaaatggtcaacacctgacatttccttccgccacccgcacttccaattcatcctcacatctgacagtttagctttctaaatgcctcttgcatctatccacttctccccatcttacctgccaccagcggtctgcccgaattggctacattaggctctacctggcccttctcccatccatcttccacacagcagccagagtgatctcattacaattcaaacttcattatgtcttatttatttatttagcgatggcgggcgggggcgagggtctcactatgctgaccaggctggtctcgaactcctggccttaggcgatcctcccatgtcagcctcccaaagtgttatgattacaggcgtgggccactgcgcctggcccaaacttcatgatgtctcttatcagcttaacttcctccgaggacttcccgtgtctcccagaataaagaccgaggtcttcgctgtgctctatcggcctctgcatgacctggcccctctcacaccacagctccgctgcaccactagccctctaccatgctgagctgagcctcctccagttcccctgctgcaagaccgttcctgccttgggacctctgttggccctgctgttggaacctgctgttggcctgcctggaaaattcttccctccttaccttgtttactcttatagggccttcacatcttggctaacatgtgaatcgcccaaagaaacctctggtctcccaggggttcaggtcctaccgtctccccaatacgatgttatgaaactctcacttcttggcatttatctcatttcccaggtgagtacccatgtggtttaacgcttgactcaccttctaggctgctatgtccctgtggccggggctcgcgtccctggcacgcatgtggccctttcttggaggggaggtgcatggtgagcggacgaggacggctgagggcgcgcgcggagggaggatggagagatgaagaggctccccgctcccggactcagggcacgtgcgtctcctcggctggcttccttcccccggccccgggcagcgccgcggatcctgagaccccgccgcgggctgtgtgctgctcagcgcccatcctctgcgcgcgcaacgctacttccgggcgagctctgcagactcgccacaggccgggctagcgcttagccagctgcgggcggacccccgggactcttgcgggccaagacgcgggaggaggcggggcagggggcggggtcgcgcggggacccggatgcggccccgcccccgccctcggaaccggaagtagagcctggtgcctgggagcggctggcgcggcggaatccagggccgacccgggccggaccgaccccaggcggcggtgagcgagcgcggcgtccgcccggggtgcaggccgggctctgcttcttccctcctgtggcgtcgcctggccgcgaaaggggaaggaagcggcggccgcagcggtctcaccgctcttccgcttgcggtttcgggggccggggcagagtggcgaggctttcccggcctggggcggggcagggcgagggccgggggtccgcgagggccacgaggacgggtctgggcccaaggaggccgggcagggggcaagggggcacggagggcttggggctggggtccgcggaggaaggttagaaaggggccaaattggggtatcagctctgaggggagggtccctgggcgccggggggctccagggcaggagaggggccaggctgggaggggtcttgggtccgagggaataggagagggggatgggctccgagggggagggtattagggcccgagggagaggggctgggcctgtggggttcgggggtctgagcggagggggccggggttcgaggggccaaggaaggtgggtgtggggggtctcaaggaaagggcgaggggtctgagagggaggtgtctgggtgcagagggaaagggatgaggtccgaggggaggagattggggtctgacgggaaggggtccaggcgcagagctactgggatggggtccgagggaagggaattggggttcgaggggaaggggtcggggctcggaggaaaggtatagggtccgaggggaggggattggggctccaggggaagggatccaggcgcagggaaaagggatggggtccgcggggtcaaggcggttggggtgcgcgactttgaggggctcgctgtgggacgtggcggagcagctggggatccgcccggcctgcacctgagcggaggttttgcaatcaggtcagtggaggcagcgaggcgcggctgcagctagtcgtgaggctctaaatttggaaccgtccctccttcggggtgtgtttaaaatcaccagaacggtgctttggggctttctgttacgtttccatcacctggatagtctaagggatgcctgatccctgaagctggaaggccttgggtgagaagggacagggcttcctcccgctggaatctgtggacgtttggggccccttggggaggaattcattttctgctgaaggaagctccggaggcatccggtgagcgctccctggaatcccatgcgctccctttgggctcagtgttgacttggctgcgcaggctctgtcggggggacttcacccactaggtccttcaggcatgatccagccctcaagcccggccgggcccaaggccgtggcgcaggaggggctggccgcctgcccctgtctccattcacgtgtttgaccgtgtgggtgagtcactggctccagagcactgagtcagtcctgtttctcctctggttagagaagtattcgccagatactcaaggaacaaggaagagtcttttaagtgggactcaataaaaagttggggaggcagggttgttttgatattaggtcccctagaactagaagctaaccttctgctatatcttactgaaaagaaatttcttttgacctggggcaggggtgcggtggctcacacctgtaatccctgcactttgggaggtcaaggtgagaggatcgtttgaggccaggagttccagactagcctgggcaacacagggagaccttgtgtctacaaaaaaagaaattcgctgggcatgttggcttgcacctgttgtcctagctacttggggcgctgaggcagaggatctcaagcccaggagttggaggctgcattgagctcgatcgcaccactgtactccagcctgggtgacagagcgagaccctgtcacccaggctggagtgcagaggctcttcacaggtgccatctagcacactgcaaccttgaactgctggccttaagcgatcctcccacctcagcctcccaagtagctgggactgcaggcccagctaaagctgacttttaattgtagcattttgatggacttgtttttaaaacacttttaaaatgacagcacacataatcggcaattcagatcttcagtctgtgccacgcagtaggtgctcagaaaaggtgtcctgagagtgtgaattgctgtgagcgctggttgctgccttgtgttgcagtattcccatggcctgggccagccaagagcaggggactgtgctccaggagcaggcttggctttcactgtggcccaggcccaccccatctgctcacgcactgctttgagtaaacggagtaaatgttgggggcggggtttcccttgagtaagtgtggtgtagggagtgtgcctgtgatggtgattttggctacgcaagtgtctgtttcctttctcctcctgctctgccatagatgctgaggctaatggaaagctgatggtggacggatggttctgtcctgtcatttaaaacattcttctgccaggctcggtggctcacacctgtaatctcagcactttgggaggctaagggcaggtggattgcttgagcccaggagtttcgagatcagcctaggcaacatggggaaaccccatctctgttaaaaatagaaaaattggccgggtatggtggctcacacctgtaatcccagcactttgggaggctgaggtgggcagatcacgaggtcaggagattgacaccatcctggctaacacagtgaaacaccgtctctactaaaaaaaaaaatacaaaaaattagccgggcgtggtggtatatgcctgtagtcccagccactcaggaggctgaggcaccagaatcgcttgaacctgcgaagtggaggttgcagtgagccgagatcacactattgcactccagcctgggcaaaagactgagactccatctcaaaagaaaaaagaaaaagaaaaattagctgggtgtagtgacaggtgcctgtagtcccagctactcaggagggtgaggcaggagaatcgcttgaacccaggaggcagaggttgcggtgagctgagatcacaccattgcactccagcctgggcaacagagtgagacttcgtctcaaacaaaaaaaaacaaaaaaaaaaagagagagaaaaatcagcttggtgtggtggtgcattcctgtagtcctagctactcgggaggctgaggtgggaggatcacctgaacctgggacacagaggttgcagtgagctgagattgtgcctctgcactctagcctgggccacagtgcaagaccctgtctcagaaaaaaaaaagaaaaaaaaggctggtggggggcttttaggcgcggtggttcgtgcctgtaatcccagcactttgagaggctgaggctggcaaatcacttgaggccaggagttggagatcagcctggaccaacataatgaaaccccatctctactaaaaatacaaaaaattagctgggtgtgatggcgcatgcctgtaatcccagctactcgggaagctgaggcaggaaaatcacttgaacctgggagacagaggttgcagtgagccgagatcgcgctgttgcactccaacctaggcaacagagcaagactgtctgaaaaagaaaaaaaaagttattgtgtagaggaatttttcactctgtctagacagcaagattacttcacgtttgtgcaatcttgatttgtacactgacgtttgcaaggaaggtttaatgttgaggatggacagagtcattatcttcttttgaaccgtattgtaacagctttatcgagatataattcacataccatacaatacgcctgtgtagtgtgtactctccactggtttttagtatcttcatgcagttgtacaactgtcaccacagtcaatttcagaataattttatcacctcaaaaagaagcccttgtaccttttagccatgacctccctatcctcaccaccaccatccgcccagccctgggcaaccagcaatgtatcttttgtctctgtagatttgtgaactccagacatttcctagaaattgaatcgtataatatgtggtcttttgtgcgtggttttcctttgcgcaatggatttgaggtccctccacgctgtagcaggtgtcactgctggatcctctttgtggcagaataaaatgcctttgtgttaggtagaccacatgttatgtatctatcagttgatggacatttggagtgtttctgttttttggctgctatgaatagtgctgttctgaacactcacgcacaagtttttgtgtgcttatactttatttctcttgggcatagacctaggagtggagttgctgagtcatacagtacctgagtttaaccttctgaggaactgccagaccttttccatttaacatccctgtcagcagtctatgaggcttccagtttttcaacactgttgctgagatttgtcattggctgttttattttagtcatcctagtgggtatgaggtgggttctcattgcagttttgatttgcatttccttggtggttaatgacattgaacaaacttttcctgagcttgttggccatttgtatatctcctctgaagaaatgtctcttcagatccttcaatcatttttaaatggggttattcgtcctttttattgaattataagagttcctttttttttttttttttttttgagatggagtctcactctgtcgccaggctgcagtatagtggcgcaatctcggctcactacagcctctgtctcccgggttcaagcagttctcctgcttcagcctcccaagtagctgggactacaggcatgcgtcaccacgccctgttaatttttgtatttttcgtagagacggggtttcaccatgttggccaggatggtctcgatctcttgacctagtaatccgcccgcctcggcctccgaaagtgctgggattacaggcgtgagccacctcacctggccaagagttctttatatattctggatctacagttcaaaatacttttctctcaggatcgtcttgtcaccttcttgatgaagcacaaaatttttaattttgtacaatttatttttttcctctagtgttttggtgtcatatctaagaaactattgccaaattcaaagtcacaaaaatttacacccttttaggattttagttcatttttgagttaatttttctgtatggttttgaggtagggctctagaggctttttatctttggatccacaaaacaatcaaacttatttcctattggttatttttgtgattgttttttaaatttccaatattgagagtataaaattgtggatctgagcctatccttagctgctcatgagtagatttttattatttactttatgattgatttatgtatgagacagggtctctctctgtctcccaggctggggtgcagtggtgtgatcatggctcactgcaaccttgagctcctgggctcaagcagttcccccacctcagcctcctgagtagctgggactacaggtatacacgaccatgcctggctaaatttttaggcagagttgtatagatgcttgccgattacactaataactgcagtaaccagaatgtagcaagcaatctgtagaagttaggattgccgaagggaactggggagagagatggagcaaacagttgggtcagaaaccagataggaaattaattaattaccaggagggccagccaggcaccatcccctagttcctaatgcgtgagaaaccactctgcctgtgaccggcttgttcctagggacaaggggatttcattcgtccatgtgtgttgtctagcagtatggtgtccagttgaggctgatgtgtggtcctagggaaggggtcgtgtgcttggccaccctctggggagctggagtcttgctccctgcgcggtaaccgtgtacgttcattaggaatgcagcccgcctcctcctcggctctccagctgccagtctcatgtcttgatagacagtcaccatactggtttttcctcccggccatatcatgatcctctcaaggagtgtttttcttatattattgttttaaaaagctcacagtaacaattttattttataatgtcattaattagaatgaataaaaagtgaaaatctctaagaattatggcaaacagtaaatgcatactagcaattaaggcaataatttctgtttgcattcataagaaacatcatgtaggccgggcgccatgcctcacacctgtaatcccagcactttgggaggccaaggcaggcggatcacctgaggtcgggagtttgagaccagcctgaccaacatggagaaaacccgtctgtactaaaaatagaaaattagctgggcgtggtggcgcctgcctgtaatcccagctacttgggaggctgaggcaggagaatcacttgaacccgggaggtggagtttgcggtaagccgcgattgtgccattgcactccagcctgtgcaacaagagtgaaactccatctcaaaaaaaacaaaacaaaaaagacaaaaagtcatcatgtagttagaaatcttttaaaacttatttctttagtttctctctgttgaacttttttattagtaaaatgcaagtcttcccatcctcctctcagatttcattacaaaaccttgttacgtttgggaagatttctgaagttttctaaaaagatctccacacagaagggtttgcccagggagctgctcaacatacatccttctcagctttttacccttaatctttttacagacggaatcagtccccaatgcctggaaattcctcattggattactgtgttttaaacagaatttcgtgaacagccttttatctccaagcggaaagaaaggtacttgtgcattgtgaacatcctaacattcttttcctgtggatggtaagggcaaaaatccctttaacaggaaacgggggaggttgctgtttctgtggatttactgggagagagatgataatattctggaaggaaatttcattgtttaaaaaataaagtcaggtttctgttgcatatgacttagaggagacaattgcagaaaactctggaattgaggaagagaaccattcctgtcctccctgaggggaaatctggccccttccccaccccatgaacatgggtggggcccccaggcacaggattgtgaagctcgcaagtgcccaccccgctcgctggtgaagctgtgtccgggaagatgagcagagactgtgctctgctaatctctccccgatgagcctgaaaaagtaccaggcaaagacacctgccgcctttctcctttgccttgggggtgaaggtggcctcgccagccctgcagggactcccatcctttcttgcagccaccttctcagtggtgggaaccatgggcagctgaagtcctgcaggcgggcagccccctcgccagggcagcatggtgccatggggcggccggagaccaagcatcccgtttctctttcctgcagccgttgcttcatttataaatgggcgcgttgggacaagatggactggttgtgcctttccgttctgagattctagttcatctgttttctaaaatcctggcaaaagcagatcactctggaaattcttgccatgctttgggcattgacagaaggttccaggggcactgttagtcctgctacaccccttggcagacggttcttccccaagagacaaagtcgcacctagaaatttgagagcacgctgccctgattggtccctcactgtcacattggatttggacttgagacccagccaggctggagggagaggagccagatgggacattctcaagctgcgaaggggttttgtccagtcctgattcagggcgctagagtcccaacatggcacctgagaaaaagtgaattgggtgtcctcagttcagacccctgggcacagcttatggttgtttgatttaaaataatctctcagttgctctgtggcctatgtgtaattgtattactatttctgtggaaaaggaatcacttctttttatacatagtttgaaaaaaaaaaaaaaaccttaaaattttatattttaccactgcagttacaagaattgtcttgttagggtttacagatgtgccattgttctgtataacagagaaaaccttccagtcttcaagggctgagggagaaggtggctgcccacttgcagagctgcctgatagaggaaaaggtggcattttatattcctgttttcttccaagtagagagtaaagcaattctccaataaactacttgaaacatttattaaattgtaagtctgaattaggaaaattattgttatggtttcagtaagcacttgaaatcaaaatgggtgcactttttaaaaatgtgtgataaaatacacataccatgagatttaccattagtaacatttagtatatttataatacataatccaaaatggtgcttttatttttggttggaaactatcatcatatgattgaacagaacaggaatatgttcttaatgaataaataagtgcacacacgacttttttattttccagaaaattttctcactcaaggtgaagccaaatagtatttccagtatatttctgacgtcaggtgacggcagtgttagaaccccgaggtgaaatttcttttactcctgtggttgtcagtcaggcttatggatcatgaaggattggctggtcctgcagcctcgtcctcctgggttgcctagaatctcggtagttttggccaaagggctcatctgctggcctctgtggatggcgccctgaggcctccgtaaccctccccggctgctctttcagatgtggaaagcttcagcaggccacgctgtgtccatcgcccaggatgacgcgggggccgatgactgggagaccgaccctgattttgtggtaggagccgccagcctttgctttcctctttcatgaagtggaagtggctctcctgggtttttctttgatggggtggtggtttgtctgtgtcacaatccaggctgtgagatttactggtcgcttttcttttcagaatgatgtgagtgagaaggagcaaagatggggtgccaagacggtgcagggctccgggcaccaggagcatatcaagtaagaggcgtcgccaccaccctcccgagggcccctctgcggatggagccccaggtgcaacagggcccatggtcatctgtggagggacagcccgtgtggaaactgcatttatcttcttgacctgatacccatgcagtaaatgaatataagacacacacatatatgtgtatttgtgtaatgtgtgtgtttatatgtgtatatttagatacttctgtgctcagtaatttttaaattcccattgcaaacaatttccgtatttataattttagagtgtccgcttctcagtattgtggcttttatcagggaacatttgtccctaagctctaccttacaagcgagaggtttctaaagtatgagatgtctgaataactgagaccttaaatgccctgtccctttttttattatctggaaatgattatgtgttaatagtgaggtactgtgagctttgtgttcttatcgaaagcccagagttgggcactgagtgaaccctgtcattgtttctcacttggcactatctaaaatgccagctcttggggcagttttttttttcgtcagaaagtgtagataaaaggaacttttaaaatatgctcagacttttattttaggccacgctgggttcagagtggatcctcctttccctggctgtgaaggcagcaccactgacccggtgaccgcctcagtatcccgggaagactgtcccgcttgaccacggtcccttcagctgagaaagatttagtccttcctccagaaaagaaggcacagcctgcacaatggctttttattttgttgttaaagatttatttatttatttatttatttattttgagacagggtctcactctgtcacccaggctggagtgcagtggtgcaatcatagctcactgcagctttgacttcctgggttcaagcgatcctctcacctcagcttcctgagtagctggaaccatgggcgcacaccacaatggctagctaagtgcttttatttcttgtaaagatgaggtcttgcttttgttgctcaggctggtcttgaactcctgggctcaggtgattctcctgcctcagcctcccaaagtgctgggatcacaggtgtgtgccgccgcacctggcccccagtggctttttatacacaagttttagtccctctctggcttgctcagcacactcccttccacagaagcagcttcggaactccggtggccgtgcggctgcccaggacaagagactggaatggggctgacttgggagtccactgtagccttgggtatttgctatggcgttaagcagactcctgaattcgttgagtcatcatacctcttctcatctcttctgttccctgtggcttcaaaacatgtctcggagggtggcttggagaaaaaacaactctccagtacaaaacccttgggctggttaggattcagagaggaacacatggggaataagcccattcttcatgctctggggccttcccaggttttaggagtggtgtctttctttctcctgctcataagggtattgggcggtttccaagcctggtttgtatttcatcttcaattctaggattgtcgtccagatgtcaaaggtggcgtcctggtgtctcctgggcagaaagtccgaatgccaaagtggccttcttatcactctggggtctctcaatgacagctgtgtctggcctaccagatttgagcaaaccttgtcatctgtttcattatctgctgtattaaaattttttaatggcatttagatgttaaattctgaaacgctttcctctcatctttgaatgtataaaaaagctggagtacttcaagtacagacgaatcatctgaatgttcagcaccactggacttctgccagtggccgaggaagggaccatagcctcgtgtttccccctctacctctggatgattacagactagctttcctattttctgcgtgtgaaataaaggtcaggcttcagtagagccatctttagcggggatttgggatgatcccagtgtctgccttcagattcgtgttctgtctcccacagagcatgcgtctgatactgcagactgccgaagtgggtgtaccagccggcagaatagtcaggaaggcacagtgcagggagagaatcggtgaacatcagataagcagatttacaaatttacaaagataatgtcacctgttcttatttatcataccaggaggcaaagctgcacgtctgttttattgatctgttcctattttcatctcttccatcaagcatacacaagctgagggagaatgtctttcaagagcatcagacccttaaggagaaggaacttgaaacaggaccaaaagcttcccatggctatggagggaaatttggtgtggaacaagaccgaatggataaggtaagtggcccgcggctgcctatgccaggctcctggtgtgcttggaccactagactgggtggcagagtcgtccctcagtctttccttagatcagcagttttgagtccatctgctgaggttgcgatttgcccggagtagacccctgttgattggacttactcatgaagtactctagctttcttcaaagctctttaggaatcctcagaggtgctggagcacgggtgttagtggggaggcccagagagggagattggccctcagaggcagccttgtgataggacagaagtccagaagcatcctggctctaggtgtcatcactgtcaatgcctgtggttccagtctgtagcctaagtcatgccagtccgaagaccaaagtcaaatctgtaggtccccatggttcacagcacacacggaatccagccaaaggcctggctaacatccttcatgcgttcagtgaccatcagagcagtctccctcgtgtaaattcggcgcgtaccaggctggggacagtgctcactctattcgagaggtctgggcaagcctcccagcagctgtcattgatgggatgcagactgccacgcagacgttgattgaggacgttggccaaatgcatccggctgcacccgtgctttaggtcctctctaccttaagaaagggagccacgttagctcaggagggctaccttttccttcctggagagaagatggaagagatcgcagtgtgtttgcatgtttcttcagtaaaacagacatcctggaggtgaacttttgagtctgagcaacataagcttgttctgacctgacagatgcttgcctttactctgtggaaagaagtgtgttagccagtgtcggattccagccggtaattccatacccctctcccacctcatgcaccagccagatgaacatttgagctgggcacgggatgatgttgccgtgtcacagtgagctgcaccttgctctgtcccccgcagagtagatgatgtggtgggccggcagggcaccccaaaccacgaccataaatagcctgggggaggagtggggtgagggaccttctgcaggaaagagacatggatgttgctgcgtcactaccacagagatgccctgacaccgagaagagcaatgtgtgcagaatcatcatctttggagtctaaaaaaagtctggtagcttttaggagtgtaaacaggcagacggacccctgagttcagtgcagcgagcgaagcaagtgcacacggacagacggaatccatgtgttcagtgcagcgagcgaagcgtgtgcacacggacagacggaatccatgtgttcagtgcagcgagcgaagcgtgtgcacacggacagacggaatccatgtgttcagtgcagcgagcgaagcgtgtgcacacggacagacggaatccatgtgttcagtgcagcgagcgaagcgtgtgcacacggacagacggaatccgtgttcagtgcagcgagcgaagcgtgtgcacacggacagacggaatccatgtgttcagtgcagtgaacgaagcaagtagataggagcctttcccactgcagactgaggcgagccctgtgcttagggggcttgtggggagttagcgcagagagacatttattctaaaaatggggctccctgctgtgggttgggaaaattacatattggggaaaagaaaggcaacgtgtcaggagagcctggtagggaagctgctgtggcttctctgctgctgctcggcctgcaccttttttcagcgcgaatctcgctcaccctagaacctgcagctcaggccttcccgagtgccaggagctccctggaagagccctcacttccctctctcctgtgcggtctccacagcagcctctgcttccctcatgagctgtccttgcctcatcctgctgtccttcccagggagactcccctccgggctgggcttttcaagggtcgggacttgtcatcctcaccttgtgcctggctcagtgccacgtgccccccccttaggccagtgctctcatggaaccaaggaggcttatggagccttgctctcacctctcagccggcaagttggagagctgtcctagcagggcgagtccagcaagggatgctgctgctgctcagtcccagctggcatcggggccaggttagcgctcagctcaggtcagcaccatgggacagatacgttgtcaaaaataactcagggccaggtacgggactggcacctgtaatcccagcattttggaaggccgaggcaggatgatcttatcagcccaggagtttgagaccagcctgggcaacatagcaagaccctatctctacaaaaacataattaactgggcatggtggtgcacacctgtagtcccagctgcttgggaggctaagctgggaggatcacctgaacctgggaggtggagactgcagtgtcctgtgatcgcaccattacagtccagcctgggcgacagagagagacgctgtctcaggaaataaatagattaaactcagattccaaaccgaaggggaaacggcttaagttatttgtaacttgcccactccagaaactaaaaaaaaaataaaaaattgaaatctctgcgttaagacttgaggaatatgtgctagagacttccagaacaaaagaagacattgaggacaagcacacgcgagaggagcctggacgggctgtcaggattctgtttatgctggggcctgagcacactgcacctttcccttgcggccagcggtggctgcatccatcacgcggggcaggcttccttcttgatcccttgtctccttgcatcccgtagccctgagtctacatattcaggcttcaagatacaatctccccagcgtgattgtttaataacagattttgtcaatgtgaaatcgaaggcaattttggagatgaatccctttcaatcaaaaagctggaaactttgcctagttgtcattctgggcatgtctttagatcagttttcctgtaacttccttctgatgaaaactcatgctggggaatccctttggcacgtctgcctctgccactccttcatctctgttttctgggtgagaccccggaggacacggatgggcagtctctgccacagagggtttggaggtgatggctcatgtggaaagagagtcagctgccaacctcaaaacaaacgtgggaagaagagggcgcagcacaggcccaaggcttagggcaagacctgctttttcaataggggaaatagtggacactctctgagttgcctgctgcgtcagcaccggtgtgggatgctgcttgggttagcaggttagcatgagtggctccacatgctagagggagtgagaagaggggtcccgatgtgccgtggactttctccacgccctctgctgggccatgtgcccgcactgtctgtgaaggagtccttgacccggctctgctgtgaggagggagcctcagaaccctggggcctcggtgtcaggactggagcagggccagtgctttgaggtaggagatcttttccactttgtggaccagagggtctctcaatagaactgccgaacggagttgctgaatcacacaagcagctgtggacagcacacctgtgaatggctgtggctgtgtcccagaggcgttatttacagaagagctgggtgccgggtctggaaagcccaggggaaccttgggctttacctgctggctttggcgaacagaaggtccccaaacctgtgtgcgctgatgggggctgacgacattaatgatgctgaaatgagctggcatgggagccggcagctggctcctgcctgaacagggtgctgctcagcggaaatccagtgtgagcctgtgtcttgcagactatttttgtttatagtctattagaacaaaatagtttgctggcagactattttttgtgccgattctttggtcatgtgattcacagaggtcccacttcagtagatgccagcagtctccacaccagcgggtctcacggatcctgccacaggctgccagctctgtgtaccttcttcatgcccggtcgtgtcagaaatccccactggtggccccgacctgtgtccagggttcgtgtgcagcttccaggagcccaagggctggggtgggccgtgaaagcctgcaccaagggctgggcgcggtgtctcacgcctgtaatcccagcactttgggaggccgaggcgggtggatcacaaggtcaggagatggagaccatcctggctaacacggtgaaaccccgtctctactaaaaatacaaaaaattagccaggcatggtggcgggtgcctatagtcccagctactcgggaggctgaggcaggagaatggcgtgaacctgggaggcggagcttgcagtgagccgagaccccgccactgcactccagcctgggcaacagagcaagactctgtctcaaaaaaaaaaaagcctgcaccaaggacccgggcctcccaggagtcgtgtcgccttccttggctccccagatgggtgtgttaatgtaaggtttccctgcactgaccaggatgtggcctcccccagccctgctctgggaggaagtgaagcgccgcagtgttgttggtgtctaatgctttgtgtttgaaccctgttccagtcagctgtcggccacgaatatcagtcgaaactttccaagcactgctcgcaggtggactcggtccgtggcttcggaggcaagtttggtgtccagatggacagagttgatcaggtgagtgatgtggcactgggactggggcaggttggggcaaggggggcgttccccgtagatctgagccctgttgggccacttgtagtagcaggcaggtgccctccagctctgggggactgcagagaggggcatctgcctcccaggatgctctgagagcctgggctctcagagagaggaagcaggatgcggcgggcttgctgtagcagtccctgcggtcgctcttcactacgtgcctcagtactggatggccctgtgtctgtcccaatgtgccagcagggaccgtttccacccgtgtttctcacatgtgtgttactgtgacagagtgacatcagagcctataccgggggggcaggatgcgctcgtgtccaggacccaggctttgcgtccaggtgttgttgcagtcatcctaggcccgagcctctttgattctcttcggctcgggtctgttggctcttttgtttaatagaggagcgatcaggagcctggagggagcagggagctgatgctggtcgcagattgtggctttgtgacatattagccacggttctgaatttatgtgtctgtgaaaggaggatgatcatacctatctcacgggtgtttgctcagatgcccatcgcagtgcctggctggtgggagtgcacccctggtggccactgctattctcattactaatactaatgttctcagcatgacttatttgagaagcgagtttttgtgaagcccgtggaatgaatcagctgagcattccctgctgccgcatgctggggagggcaggtcaccagggatatggcttggcctgcgccgcccactgggccttgcagtgggcctgtccgtttaggatgccagagctcttgcgtcactgccacctgcacctggcttaggaacccagaggtcacagcatcatgtgtttcatatttttttaaatgtcatgtcatgaattcagagagattgtttcagggacattggaaagtatttctccccactttttcatgtgtttttggtcgtcacagtctgctgtaggctttgaataccaggggaagactgagaagcatgcctcccagaaaggtaagacgcgaaaggtgcagaaagagcccgctccgggggccccgatggggagagtcacagccacctgaagccgtttccccgcgctccgggggccctgatggggagagtcacagccacctgaagccgtttcctgagcggtggctgttgccacaaggaggactctgccctcctcttcatgtttctggatcatctggataccacatgatggatgtattccccgttacacatccccctcggagccctggcatctgctgtcattgtggggcttctcccccttgtagcggaagccgccttcccttcatctgggcagcgctcctcctggaaaagagagctcacagattgatcggcagaaagctaacttccccccaaggctagaaaccagagttgttaaattcttgttttccttatacacatacggtcttaactgctggtgattaatcttgattacatcatgcagtctcttttttgaaagaccaaagcatatcgccaccttaaagttctcagtttattttttgcaagcttatttagttcttcctcctttgtgtcattgcatcatccgcggaacaagctctcagtccttacgcagtctcggtgcagtctaggtggaggtagctgtggtgtgggcaggcggaggatcagggtcttgtctcccatgttcagctgcatggctttcttcatatgaggctttctctttccatttttttcattttgttttgtttgtttgtttgttttgagacagagtctcgctctgttgcccaggctggagtgcagtggcacgatctcggctcactgcaacctgcgcctcctgggttcaaacgattctcctgcctcagcctcccaagtagctgggattacaggtgtgtgccactacccctggctagtttttctattttttagtaaagatggggttttgccacgttggccaggctggtctcaaactcctggcctcaagtgatccgcccacctcagcctccaaagtgctggttctttgtatgaagctttccagcttttacctgagcagttgattttcaaagtgggcatgcagcgtcatttctgactacacaaaggaactgccctgtaacagctgacagggcccagaacccccccatgcacctgttgtgaaacagggtccagcgtcacctgtatggagcagtgggtggcttgttgtacattttaaaatgtgcgcttgatgtgtttgtgagttgtaacccctacacacttttgcttagtttaacaaaaggaacagtgtagttcgtcctcaggcccccgtgctaattgctgccctgtctctccagactactccagtggttttggcggcaagtatggcgtgcaggccgaccgagtagacaagagcgcggtgggcttcgactaccagggcaagacggagaagcacgagtcacagagaggtggggcggaccccacggtctgtaatcacgcgtttgctccagaaacacccacgagggcattttctctctgcacacgtgattgttgtagattttttaactgaaaatgtttggtgttcttttcgtaccagtgtatgtgggcttttcctaattcgaatggctgcacagttccccatcgtatgaggattccatagtccatgtaaccattccttttttgaatgtggaatttaggttatttatttatttatttatttatttttggtagagatgaggtcttgccgtgttgcccggactggtcttgatctccgtggtctcaagcaatcctcccccaccttggtttccttaagtgctgggattacaggtgtgacctgtaaattttttttttttttcttttagacggagtctcgctccgttgcccaggctggagtgtagtggcgtgatctctgctcactgcaacctccacctccctggttcaagcaattctcgtgcttcagcctcccggctagccgggattacaagcacacgccaccacgcccagctaatttttgtatttttagtagagacggagttctgccatgttgcccaggctggtcttgaattcctgacctcaggtgatcgccctgccttggcctcccaaagtgctgggattacaggtgtgagccactgcgcccagctgaaacttgactgcagggaacattcttgtacctgtgttttgttcacaagtgattattcaaaagaatcaactcctagacatggaattgctgggtcgaaggctattgaatgtaaaatttccatagatactgccaagttgtccttggaggctgtgctgagttcttccttccccactgcatagcctgtacaccatcactcagagcgtttcccagggctgcagaggctgtggcctgcctgtttccctgtgcctcttgtttggcctgcacatttaaatttttaaaaatatttgccaacatttgaaaagtgagagcttgactatttaaaaactcaaaaaaaaaaaaaattaccccaaagcaagaaacaccactggaagatccgaccatgctggcttggcatttccctgaggaaagtgccggtggcctggagggcagcttggcttgtagtttgtcgcggcctggcctggccttgccttcctgccggtcaggccttgcctgccgcacagagctgcttgcctctgctctctgcctctttcctgggaacagtccattcctcctcttcccgacaccacacagttgtgagcagcgcatgctccgcgaagctgcatgagtgtgtgactccagggagccagggagcacaggccgagcacctgggcagacgcaaggctccttggatgcttcaggccttctctcccaggccttgatcctctgccttgtgctgttagtgggtccatgagagagtcatgttttggtgtcacagtgacatgctaaatggcttcattctgagtagaactgaagtttctggaggatgctgtgtgtgtcctatttcctggcatccccctgcgtgcttcagtttgtgaagagtggcctttgaaagccttgcccttctgctgcctcctgagcttagagccttatcataaactctcatctcctgctagcttctggatgcaatggatgatgttacaggaattcatctgctgtactttatttcttttttttttttttttgagacggagtctctctctctcgccaggctggagtgcagtggtgcgatctcagctcagtgcaacctcctccttccgggttcaaacaattctcctgcctcagtctcctgagtagccgggattacaggcatgcaccaccaagcccagctaatttttgtatttttagtagaggcggggtttcaccatgttgcccaggatggtcttgatctcttgacctcatgatccgcccaccttggcctcccaaagtgctgggattacaggtgtgagccactgtgtccagcctctgctgtactttaaaaaaaaaaactttttttcttaattctttttttttttttgagatggagtttcactcttgttgctcaggctggagtgcagtggcgcaatctcggctcactgcaaccttcacctcctgggttccagtgattcttctgcctcagcctcccgagtagctgggattacaggtgtgggccaccacgcccagctaattttgtatttttagtagagatggggtttcataatgttggtcaggctggacttaaacccctgacctcaggtgatccacccacctcgacctcccaaagtgctgtgattacaggcgtgagccccctcccccagcctgttttttgaatttttaaaatattagctaatgatttaacttagagatggggtcttgctatgttgctcaggctggactccgcctcctgggctcaagtgatcctcccttctcagcctcctgagtagctgggctgcaggcacacacccccacctctggttaccactgtttgtgtttctgttattacacataaatagcacctttaaatactgtctgtattattcagtggtcctacagtggacctgaaagatttttgtttttttaatcaaaggataaaatacttgcatgttcactgatttcgttgctccttttaaagtagtccttttttgtttgtttttagattactccaaaggtttcggcggcaaatacggtatcgacaaggacaaagtggataagagcgccgttggctttgagtatcaaggcaaaacggagaagcacgagtcccagaaaggtgtcttccgttttatcttaccctccagccagcagctagtaatgtgacaggtggtagccacaccggaaaaggaaaaacaaagcgttattgatagcccttaacgtagatgtctctttttcatgaaatgcccaacttgaaaacggcacatccagaaacacagctgctaaataggaactcgcagcttgagtgttatggcgctccagccccagcggcgttgcttatcgtggtggccacaccccgcacgtctgtgttgcccgtgtgagcctgtgctgtctcatggtgcaggtggaagccgcatgcgtgggagcttccatggggtcctgtctggcttagtcacgatgctgaggtcatagactaggttatttcctgccactctccaaggagggcctcttcatggatgttatctaaagtattaccaaagatccggaagggaagagtagattgagtgaatcatatgcgtttaactgtattgaaaacatactttccacctgtgacctgcactacctgttaatgatgggttctggcctttcattgtgcatgtagactatgtgaaagggtttggaggaaaatttggtgtgcagacagacagacaagacaaatgtgcccttggctgggatcaccaggagaaattgcagctgcatgaatcccaaaaaggtacattcactctgcctgtatgcgagatggttttagaagtttgtttttgttccttgcggggtcagttggtatgtgttgtgtctgcgtgtgcctgctgcacattgttttgtcttcactgtttgaagttgtcctgtgtgcccccccaatcccccagttccctccagctagtgtagtcacaaaccctccctgctgacacggggtggtaccccagagagctgggtttacctggaagtgggttgcacgggccaagcctgcagcagggggctgggtttacccaggagtggggacatgggtcaagcctgcagcggggggctgggtttacccgggagtgggggcatgtgtcaggcctgcagcggggggctgggtttacccgggagtggggacatgggccaagcctgcagtgctcctcgtccaggcttcttgtctataaagtgggccacggtttttggcaggagatacttacttttgaaccgaaagtgttttcttgttggaatgggtgccccttccctggaaaaaggagcctgagggggctgaaagcctctcagtgtgacccagccctgagagaagcaggagcggggctaatggggctgcagcgccctccgaatctgcagggtctctcctcccgtgtacacagatacgcacactcgtctgcagtgtgcgtaggacgctaagaatgaactgcacagccgggagtgcctttactgaattatttgaagtacagttcttaggctgtttccagatcttcgctgaatgttcagattttaagttcattctttaacgttgacctctggagccaccttctggcgctgagcctgggagtaggatctcaaaggccctaagctcaggagagcccttgctttctggaaactgtgtttgatttttgcgcatgctcatgcaattctaacccaactgtgtttcctcttttggttgttttccccaccgttgcttgtggattttcagattataagactggttttggaggcaaattcggtgttcagtcggagaggcaggactccgctgctgtggggtttgattacaaggagaagctggccaagcacgagtcccagcaaggcacagttgccaccagcctcctaccctccccccgaccctcctgtgcggccacttctagcacagacttcagggctcaccgtcagcacctgcaacacagtcctcctgtgtcaccacttgtcagctgtcaggctgttcggcactttaaacccatttctagtagagcgacactgattttgagtggttcactttcttccctgtgaacttgtgatctgtgtatggcaggatgcgccagttagtgtgtgggtgccatctttctaacccggtgacccatccagtctctggactggggcatctccggtctccagcagcattctgcccgaggctgttctgctgttattggtgactgccgtgagccactgagtcaggccttccagatgaggagagaggtgttggttcaaccaaaccactggccatttgaccaggggcttcgtagcagcaagggccaggagacaggcgtgttctgtgtggtttctgtgtttcatttgtggtggtgtttttggccggggttgggaggttttttgtttgttctgtttgatgacaccatatgtgtgtaacatctccacacagagccacacagtatgtacgtgaagccagctctgcatccttccttagagcagttatgttgttaccttcaaaatttggacttggttattagaacctgaacacataattggaaaaacagtaattccctaaccttgcagggaagactctaggctttccacctgcagcgttagaaacacacagcagtgttcaggtgtgggcaggtagacacacctggggtggtcccgcagactgcatggagatggcagtggctctccgtggcatcactgctgcatgacagtgtgactttcatgtggatctgtgttgctgcaacggaagtctttggctggtagaatttccacatggattttttttttctcttaaaaagcaagagaacaaactgcgattgagcctctttttagcgctcagtagagcaggcgcacaggaagcagatgagaataagcccgtgctggtgcggaagactgtgctattgaagtggcgcgttgcaaaggcctgtccgtgccctctttcctcgcttagctcctgcctgctgcccgccgcccctcctgcccctcctgcccctcagggaatgctgaagcctctgcccctggcctgtgctctgcttctcactgcctgtgtctcacgaccatgggtggaagcaaaacttgccttgcaagtgaagcctcgcttggccattctcgtttcttcccgctgtggtgcaccttcttgggctgtttctggatctgtctgcatgcacccacggccacccccatgctcgcctccacctcagagtaagtgttgtgttttgccacgtttcagactactccaaaggattcggcgggaagtatggggtgcagaaggatcggatggataaggtaaatattccagccccggagcttagtgtcttctgcctgcaggggctcttggtgggcgtggctcaccgtgctggccaacatccacgcgctggggtggggcacaagtgatttccgtcgtggtggtggtggtggtggcgatgactgactcggacagtcttgtgctcctcatggccgtgctgtgggataggcgctggcaccggcccacctcctaactgggggccccaggccaggcaggctcagcacctggctgcagccataggaacccagatgtttaacatgagaggtgagagcctcgagggctcatgtccatttgaacttaaattcttaatagtaaatcagattggaacctgagtgtgttagactgcgtgtggggctacctcgctggcagccagcgctgcgtgtgcggtgatgagccatgtctcccgtcttcagcctcgtagagacgcgggctccacactgcctgctcctgcgcgtggcctgtggtcaggggtggcagggtcagtccttgcctcgcagctgaccacacagcaccatccaggggcgtaggcaggcagagcaggtgttcgggctgggctccccagggagcagtagcctgggctcatttctgagggcaggcgcgagttcaccaggagagaaggctcaggagaccacggaggggcgaggtgtgggcctgggtgggacaggagtcactgcaggttctcaaggagcctagctgcagagggagggtggggagcggggatagagctggccctggagagccccacacaccacagagcattctgcatgtcgtgacatgcgtcacacaccacagagcatctgacgtgcgtggccccagaaaggttagcaggcaaggaacactgggggggtctactttgagaagcccccggcctcagcgtggagtctgggttgtgggggttggcctgggcagaggggtgtggtcctgaagagagctggaggggctcgggtggtggagaagcagggcatggtgtttggcgccaagggacgcaggggagggagcagtcgggtggatgtggggtggacgtggggccaactgtggagccgaagaactgggaggagcagatgtgggtgtcagcgagagagagctggggtagggccggggccgggtccgggtggcgtgcagagttgggaggggagtaggcacgcggtcagaggacactgggcagggggacccagtgaggtagcaggggccttgcaaggcactgggacagacggagggaaggagctcccctgtgggtgagtcagaggttgtggctttgaggccggaacctgagggactgcctgtgttgtgggtgtctgtggtcagcattgctggtgttggcggtgaccgctgagcttgtgagcacgtgaggaggcagtgtgtgggtgtggcctttcgtgtcattgtctttcatccaggagggggtggagcttctctctctgtttgctgtgtgatggaggagtccaaggcttagagagggtgacgtaactgcccgggtccccgtacattgtggggatagggctacaacccaggctgggaggtgaggttgccccctcagggcaaggtgatgtctgcagagggcccagtgggtgatgggacccagtaaggctgatggccttgcctttgaacaggtgcaagcagctcaagggacatggagtagacgctggagctgactgatgggaggagggagcggggtccgaggaaggacaggcgggccaggaagggggcaagcaggaggtgctgggcagaaaggggccatggtatgggggctctggtggcttctgagcctcaggtgttctcatcagaggtagtgaaacccacagtgaaactgggtacggccactggtctccatgagccctgacagtctccacctgagtcagcacgtaagagtcatcgccgtcgcttggtcagaccatagttgagtcccctgcaatgcagaaaggtgtgattgcaccactgggtagtgtgtaggacgggccactggaggcttgcatgtgtccccagcgtcttcgcttcctgtaaatgctcacacctctgggtggcgacggagggtggggaaggcttgaggctgatcccagaattggtttgtggtagagcagatggagatacatgcatttcttatgctaatgctgacccacgattgcctgcaaatgtggttggtaatagttttcatgcctcagttagtttgcaaaaccgagaggccatgaagagtgctctgggctgtgtggctgggcctttgggaagatctggggaaggcatttgcatctggcaggagctaccacagggcatggagaaaagagaaaagtgctctcctgacgcccatgtcctgtctctgcagaatgcgtcaacctttgaggatgtcacccaggtgtcctctgcctaccagaagacagtacctgtcgaagctggtgagtcccggctgataccaggagcaccgtgtggtttcccaggaaaacactgaggggaaggacagttgtgaaacagccaggagcagatgcaccactagttgaaagcgtggttgttgctgcgtatttttgggaaagaaaatgatcgttctgatgaagatcttgtttctcctgcagaggtttagagtcaccttttagacgcagcatggtgcagccgtttcagtggggcccgtttcaaggggctcttcccagagcaaaggggctaagtgcacattcacaagggcctccacactgccagtgaccagcttctgggagcagctggggcctgcccaactccctgagccgctcggtcgcagctgtgacaggaggaagacatcatgacgtcgatgtgcaggcgacagcagacattctctctgctgctcattcttgggcccttgacagggagctcgaaacctgtccttttctgtgctggacacacgatggagtcagatgtgggccctgccccggagagtcaagtagttggggaaaatgttatacgtacatgagagcaagtgctaaatttaacacaggatgcagagagccaccactgacttccttcaggggctatggacccacagttctcagagattgagggcccaggacgctttgacactttgaccactcgtgagaacccccaagatgttgatgtatttgatgacattgactatattagaaattagaggaatgtggcccggcacagtggctcatgcctgtaatcccagcactttgggaggccaagacgggcggatcacgaggtcaggagatcgagaccatcctggctaacatgatgaaaccccgtctctactaaaaatacaaaaacaaaattagccaggcatggtggcgggcgcctgtagtcccagctactcgggaggctgagacaggagaatggcgtgaacccaggaggcggagcttgcagtgagcggagtttgcgccactgcactccagcctggatgacagagcgagactccgtctcaaaaaaaaaaaaaaaagagagagaggaatggtttatttatttatttattttgagaccgggtcttgctgtgtcatccaggcaggactgctgcctcagtctcctgggctcaagcagtcctcttgcctcagcctctggagtagctggaactataagcgcatgccccctgcccagttaaattttttttttttttgagacagagtatcactttgtcgcccaggctggagtgcagtggcgcgaactcggctcactgcaagctccgcctcccatcccgggttcacggcattctcctgcctcaagcctccggagtagctgggactacaggcgcctgccaccacgcccggctaatttttttgttgttgttgctattttttagtagagacggggtttcaccgtgttagccaggatggtctcgatctcctgacctcatgatctgcccgcctcggcctcccaaagtgctgggattacaggcatgagccactgtgcctggccaaatttttttatctttagtagagatgaggtctcactgtgttgcccaggctggtcttgaattcctgagctcaagtgatccacctgcctcggcctctcaaagtgctgggattacaggcatgagcgacattggaaagttattaattggttcacttaaaaagaataaaccttggccgggtgtggtggctcacacctgtaatcccagcactctgggaggccgaggtgggtggatcacctgaggtcaggagtttgagaccagactggccaacatggtgaaaccctgtctctactaaagatacaaaaagtagctgggcatggtggtccacgcctgtaatcccagctactcagaagactgaggtacgagaattgcttgaacctggaggcagaggtcgcagtgagccttgattgcgccactgcactccagcctgggcgacaacagctaaactccatctcaaaaaataaaaataataatataactggccatgcacagtggctcattcctgtaatcccagcacttagtgagaagaccggcattgttttgttttgtaaatctctttaatagaagatggctgcactctcacatctgcctctgcagtctatcgatgttacgttgtttaggaagaacatgtggaatcacctaggtaggcgattgggaaagggaggagtgtttgtaaccttgagggcagcgtgggtgtccttctgcgacgccgcccaagcgtgccaggcagtggtggtcctgcaggccggctcctctggccctgtgagggactcgctacccactgtgcttgggcagcactgtgtgttggtcatttggaaaatgctgggtctctgagttgtgctcgtcttccagttgtggacacttctcattccacaataccaaaaaaaatcacatttgctgtcatcatctatttcatctgcagaagctgagtggaaatcagcatgttcatgtggcagcctcacatttttcaaaattctgtcttttacttgaaagctggaatttcccgttggcaacaaatactccagtaatttttcttgacacaataggcttagttcattcacttgggagaaaactgttgccagatacccagctttgcataaccatagttggactgtcagttgttatttcagggaaaaacgctattccatgagaaaacagtcaacttgcagctctcatggttctattcatgttccttgagacacagacagccaccgccacagtcccggacacagcagggaaggctcatgtgtcttcccacttttttttttttttttttttttttttttgagacagagtctcgctctgtcgcgagactggagtgcagtggcacaatctcggctcactgcaacccctgcctcccaggttcaagcgattctcctgcctcagcctcccgagtagctgggattacaggtgcctgccaccaagcccagctaatttttctatttttagtagagacagggtttcaccatgttggccaggatggtctcgatctcttgacctcgtgatccgcccaccttagcctcccaaagtgctggaattacaggtgtgagccaccatgcccggccatattccccacttttgacttaaatttttatcattttttcttaatagacacggggtctcactgtctttcccaggctggtctcaaactcctgatctcagtcttcccgtctcaacctcccaaagtgctgggattacaggcatgagccactgccctcttttccatttttgtcacactcaatataaaaaaatgtatactctggttgagatttactatactcagtatacgtcaattacagtatacatcagtaccttaactgctttatcaaggacagatgtctttaagggaaattggctgtttttatttaaaaaggtgagtgtggtggtgaagataccgtcttatttagagatcttcctgatgccaggcttagggggattgccagtcccaccaggtacgcagtgccctgcggcaggcgcccagtgctggctccggaccagtgggctgagctggcagctgcaagaacctttccctccttgggggttagggggatgaccggttcctggggaggtcactctgtgtgggtggggctgttgtccaggagcagtgtttttgctgtgctcaaggcacacgtcctccctccttcctctatagtgaccagcaaaacaagtaacatcagagctaactttgaaaacctcgctaaggagaaagagcaggaggacaggcggaaggcggaggcggagagagcccagcggatggccaaggagcggcaggagcaggaagaggccaggaggaagctggaggtgagtggcaaggagtgggccgcagcgcaccctccctgggacctgtgccgaggggattgggagctctggggcctgggcggggcttattctctcctggccaggttttccttggaaggcatgtttaactctcctttaaggctctccattaaggcacttgtcacttgcagccacacataggctgagtctctggccacaatgctccaggcagccggctagcgctatcctggtcttgccggtccagccccaggacccgccaacctgccctgccctctgtccctgagctgacggcagagtcttgtcggcttccacctccacggggctcctgtccaaatgtcagcctgagtctcttctcacccctccccctctggaagcccctcctttcccaggtggaagtttggggtttggaggggactctgtgcagaggcagcctgctctcggggcctgcttctgcttggccagcccagccccgctcacccaagcccctgggctgtggagaagcgctcccagagaaagatgccagaagtgcacccctggcaggggcggagcacgagcctgtgtctgggtgctttgggtctagcctgggtggcttgccactgacaaggactttccttgtgctcagctggatcaggggggttgtgaatagagcctgaggcctacagatgtgtgtgtttgaagttaccaggtgtggttctgcagctggggcgtctgcagtgtggggcaggcccagcctgtctggaagcctgggcttctcctatccaccccaggccctgcccctgcagggccacacaggacgtgctactttgggtgtttaacctgtggatggcatcaggtgacgtgggaatttacaactgtcctgttggggtcaagcaaggccatagatgccaggaaccgtgctagggtctcaagaccaggggtggttttctcttcaaaaagacaagacagcactaccattgttcccgttagagcaaggccgcctgcagagggagaggagggaaggggtttggagtggacacctggggttcagatcctgcctgggcagctccctggccagtgtgcctgtgcctgccacctcaccctcagactcagcgtctggcactggagcccacaggctgtcagtgagtttcctgttgctgccgtagtcaccaccacacacagcacacagctagtgtcttatagctctggaggtcagaggtcctcaaagatgtcagcagggctgtgttcctcctggtggcccctcggccctgtcttcccatctccagcctctgcttttgttgtcaccgcccctcctctgtccctcacactcctgcctccctcctctcaggaccttgtgattacattgggtccacccaggcagtccagagttctccccatcacagaacccttagtttcatcacatacacagtccctccccatggggtctggggactgcgatgtcagcagctttgaggggctattatcctaccccagttcatcctttggcccctaaagcttcatgtccatcccatgtgcaaacatactcagcccattttaaggtccccaaagtctcaacccattgcagcatcaactcagactcccaaatctgtgcctaaactatctaatcgtgtgtgggcgagaatgagctgtggcccagtcccggcactgtcgcgtcactggctgtgtgatctcggcctcagagcacacctgcccctcgaatcctgcctaggtgctggtgggatctgaggcttggtgctccagctcccctgcccctcggatcctgcctgggtgctggtgggatctgagacttggtgctccagctcccctgcccctcggatcctgctggggtgctggtgggatctgaggcttggtgctccagctcagccttggtgtgggtgttttccactcctttccccagagcatgcctagagcttgcacacgatcttctgaaacacggcaggcgtgacagtgcagagtgtcatggcagcattctgattgctgtttgttttcaatcacaggagcaagccagagccaaaacgcaaacgccccctgtgtcgcccgcacctcagccaaccgaggagaggctgccctcgagccccgtctatgaggttggtgtctttggtgtttgaatgagcgtgagtgacttactgccagagcccaggtcctgtttgtggagtggaggaagcccttgcaggcaggcagtgtggcgtgggtgtagagggcatcgctgcattccacgcccggagaggggtgggccaggagcgcctggggggtgggtgacatgcctcagtgctgtcaggggatagctgtgcaggcccacatgccatcctttcacccctctgcagcctgttgtagtctggccactgtcactgcagtccctgctgtagtcacctgctatcagataatcagatgggggtgtcttcaccccacttgctctctgctgtgcaggacgccgcctcactccctaatggcagtgctgccctccttcatcactgagcccctgcctcccgcacaatctgccttctatgcctgccccattcagctctccagccttgcagcccctcatgagtgtggctgcagacatgctgtccggcccctccctgtgctgctgaccccctctcatgggtcccccagtgttccgtccccatccctgagttacggagccgcctgccgatgccccgcccatccctgactttgggtctcctgctccccgccagcacctctgcgctaaccacaccctctacctgtaaccacctctcctgtccccagcccttgccctttctccatcccatggccatcatcctggtctatgcattcatctctgctcgcccaggagccagtcagttacaggggtcacactccagaccatggtagttcaggagcccagagagagccctggagacaggccgggtctgggatctgggcctcgatggccgtggccttgtcaccacccgactgtccctccttggctacgctgctgcctgctgcctttaccctgccgggccctggggggatgtggtaagtgaggccttgcagcttgtcaggaaccggcctccccctctagtccctccccttggcagccagcgccatctgcctcaaggaagctccgaccctgcccctcccgtgcagttcagcccttgtgtggttcctggtgcctgtgctgagggtggagtcgctctccctgcccgattactctccctggctagcagctctccaccactggctcttcccagatctcctgggtgcgcggttctgagctgtcggcattgctttctttcagtcactgccggtgacatcaggtggaccctggtcagaaacattgattaaagccactgtcggtgataagcagttcagaattttcgctggggctccgaagcggcagtggttatagatagagaggtgttccactgcgcacctttgtagttgaatgtgattctttggcgatgccctgtctgtaggctcagctgctctggagccacgtggtgaagccgctgtcaggcagggccagggtcgagcagcacaggcaggagggtggccgctcggcccaagggtgccttgtgtggccttggagatgaggcctaaaaaaagaaatcagccaaattaacccaacaaagacaggctctacccccaggcaacagtgtgggcaggggcgtgtctcctaacctgctgtgtgggcgtggttattgcaggactggagtgtcggtggtgaggaccgggtcatcatcgggaaggaaatggcatgtgggcactcaccaccatggtgaacacaccggcaggcctggccttcctataccgcgtctgttttctcagtcctggctgggactgagaattagatggcccgtgggtttctgctggctgtggcagtacagctactgctctctagaagccagcatgggccccgtgccatgtgcgtgtgacccttccccaggatgcggcttccttcaaggcagagctgagctacagaggccctgtgagtgggacggagccggagcccgtgtacagcatggaggccgctgactaccgagaggccagcagccagcagggcctggcctatgccacagaggctgtctatgaaagcgcagaggccccgggccactatcccgcaggtactggggccccacgctgcagcgccctgcccagggcagggagctcccgggacatcctgctcgacctgtggcgtcgttgcgaggagcgtgggtttcccactgacacgcctttgcttgcttgctatagggtcttcttgtccctggagggaagtgctctaggcacatttggggcatcggctcctgtgctgggtcccaggaagatccccattgtgctggggacgggtgggcaggggcagaggaagggagggtttcagctgccgccctgtgtccttgagacaaaatcttaggaagtgtcgtctaaaacttgagaaggctgggaacctgtgtgggactttgtattgttcagctctgtcatggctttcttttagaggacagcacctacgatgagtacgagaacgatctggggatcacagccgtcgccctgtacgactaccaggctggtgagcggcctgcaaaagcacttggaggggagacccaggcagctgcgcctgcctctgcttgttttctgagaattcacttctctagcgttgttagttttagaagtaatttatgttgagataatttcagagatatagaaaagttgcaggtatagtgccaagaactcccatgttccctacccggattccatctctgcacacacacgcacacaccccctgttatctttagtattctctgaacccttggagagctgcaggggtgctgccacccccaaagactccagcgtgtttcccaagatcaaagatgctgtcccacagggacaccccaccctccaggtgagggggccgcacagatgccttgatgttcgagaccccattcctgcctgtcctgccccggccgccagctcccgtgtggcctgcacgttcccctgagttccctttgcggccttggcagtttaagagcagcctcagctcctgggctggcctcagccgcccgccccgtgttccctcagggccaggctgagctgtgctctggtggcggtgaggtcttggagaccatgcggtggggctctctgctgggccgcatagcatccacttgtccctctgtctcagccaccacctggtccacaaagtcaccaccacccctctgcagttgacaagcatcttacgggtggcgtgggattcccctcagaacccccggcctctgcctcttgagtgcttcccgtttgcaactttccatcagcgcggaagctggcattctaccataagtgagcgctggccgtccccgttcagtgccttttgcacacgtgcatttgcgtacacgcatgtgggtgtgtctgtggcatctcggcacctgtgcgtggattctcatcaccgtggcttttgccctccattcccatttcagccatcacctcgggcttctctccgggtgtcccctggctgtccttccactgcggcctccgcagcccatctccccaccccctccacagacacgtctgccagagagaggggaagggagggcaggcggggctggagttatgtggtggaaacctgctcctgaggaaggggtccgggtggggttggtgcttgtgtgaagggctctgtgcctctttgtactctctcggttcatgttcacaaatcctggccttggtggcatgggagaggcagcaggagcctccgcggtggtccgcatctctgcagggggcatctctgagcaggctgcctgggagcttgctctgggttgtgctttttttctggcagaccaggaaacgcttgcacttcagcatctttctctgtgttctcttccccagcgggcgatgatgagatctcatttgaccctgatgacatcatcaccaacatcgagatgattgacgacggctggtggcgcggggtgtgcaagggccggtacgggctcttcccagccaactatgtggagctgcggcagtagggcccccagcccccccccggagctgcgccctggatcctcacactacagatcaggccttctttggttcttgggtggttttgggttttttctgtttttttttttttttttttttttttgaaggtggggaggggaatatacacattgcttttatatttaatacttttgctgatgcttttgaaaatgtttatgccacagaatttgctaatatattgtaatcacattccttaggaggactttggtaattggttttatgcattgatggttttttttttcttttttgccaaattgactgtcacgcggcagcttcagggagctcgcattctcttgtgttcgtgttgccctcgtgcccatcaagtgcagtcgggacctcccaggacaagcacgaggcctcaggtcggccctgtggcgggtaggcaggaaggactgtcccagacgaggggcttcctctagagtctcactgctggggaggagaggactgggcctgatggaagttaacccggagctaagtcacccagagcacaggagctgccatgtcagatgggaaatctgcctatgtcataccgtgacagcccgcaggatcaggtgacttctagcagagaccctggtttttttcctgtgcccactccggcttgtcctcatctctacccatcccctgatgcccaggtcaccgggagggctgctgggagcctctcctgtccccgccgggcagtgtcactgagtccttgaaatcctcccctgccccgcgggtctctggattgggacgcacagtgcagttgaggtctgcgtcgggcttggcttttcacaaaggctgatgtcttaactgtcacccatatggtccctgggccaccgggcagcctggggcggtgtgtgtgccatgtcacagcatggcctctcggccttgggaaggaaggcagtgcctgctctgctgtgagccgccaggaaccctcctcctgtcaatgggggtgtagtatttttgccaaaatatcatgttcaatttcagtagtttgatcagttgaaggctagaagtgtgaagtgcagatgagtgtgtgttcttccccaaggtccccccacagctccaggacaccgctgtcctggcatttgtggccactcactttgtaggaaactcatctccttcctgaggagccgggaggctggaccagtcccgtcgtgcagtcaggtgggcggtgtgtctttccagaaggtcacgtggaaatgtctcgggacttgggtcccggagtgcccgtgaagcgtgtttttgctcctgaggtgcattttctcatcatccttgctttaccacaatgagcaatgaggtcgggttttatatgcaacttattgtatctgaattcctgtagcacacctcataggtatgatttttttaaattaaagaattcagaataaacattttttgatccacttgcgtgatttgctttggtctgtggtcactccgttctgcagtggaagctgcgtcgacactctcggtgtcctgcgccccggacctgggctggcggaggctctcctgccgtccagtgcgtctcacgcgttctgttctggctttagaccgaaccgtacgttcccttttcagtgttcattcaaaggtgctgcttatgaactgaaatgtgtttactctggcagatgattcagttttttgcacaaaggttccaagcaaaataaaacactctgtgcttaaaagtcatttctgaattacagttctcaaatcctgagggggattttgctcccagtctcgttttgctcaggggcaccagaagttaaaggctaaggggaacagtggcactgagggagcaggtgtgccctcgggagccgggggtgctggggagctgggttccggctcacacttctgcccatcaggtgctcccagggagctttcggctacacgacatgcctgctaccccttccaacctgggtggtgacggagcactgcctcctcccttgggctgtcccacacctgcctctggcttaggaaacagtgcctctgccagctctgcccacaggagcctaacgtgagagtgcccccgccctcctgccacaaccgggctgctgtgggttctgcatcacagggttgcagccctaacggcgagccccgtcccacacagggcctgtctgctgggtgagattgcagccacctgcagggagctgagtcctgaagaaatgaggcatgaaggcttgtgaaccaggggaaagtcccagccagggtgggcagacggggtcagggggctggaggacagggtgtgacgaagggagggggccatgcagaaagggcccccgctggcccactgtctgcccagtcggtgtgtgcctgtgccgcctgcggatgagcatggcaggggctcaacaggagcgggacacatcttgagtgggtctgtgtttctgttctattggtggccaagcgtggcctggcagcttgagtgcctggagccaaccacctgaatcttagccgctttctttttagatgtggttggggttttttgtttctgtttcttgagaaagggtcttgctctttcgcccaggctggagtacagtggcgtgatccgaattc

SEQ ID NO: 22 - DNA, 3243 bp, Homo sapiens
agtagagcctggtgcctgggagcggctggcgcggcggaatccagggccgacccgggccggaccgaccccaggcggcgacggaatcagtccccaatgcctggaaattcctcattggattactgtgttttaaacagaatttcgtgaacagccttttatctccaagcggaaagaaagatgtggaaagcttcagcaggccacgctgtgtccatcgcccaggatgacgcgggggccgatgactgggagaccgaccctgattttgtgaatgatgtgagtgagaaggagcaaagatggggtgccaagacggtgcagggctccgggcaccaggagcatatcaacatacacaagctgagggagaatgtctttcaagagcatcagacccttaaggagaaggaacttgaaacaggaccaaaagcttcccatggctatggagggaaatttggtgtggaacaagaccgaatggataagtcagctgtcggccacgaatatcagtcgaaactttccaagcactgctcgcaggtggactcggtccgtggcttcggaggcaagtttggtgtccagatggacagagttgatcagtctgctgtaggctttgaataccaggggaagactgagaagcatgcctcccagaaagactactccagtggttttggcggcaagtatggcgtgcaggccgaccgagtagacaagagcgcggtgggcttcgactaccagggcaagacggagaagcacgagtcacagagagattactccaaaggtttcggcggcaaatacggtatcgacaaggacaaagtggataagagcgccgttggctttgagtatcaaggcaaaacggagaagcacgagtcccagaaagactatgtgaaagggtttggaggaaaatttggtgtgcagacagacagacaagacaaatgtgcccttggctgggatcaccaggagaaattgcagctgcatgaatcccaaaaagattataagactggttttggaggcaaattcggtgttcagtcggagaggcaggactccgctgctgtggggtttgattacaaggagaagctggccaagcacgagtcccagcaagactactccaaaggattcggcgggaagtatggggtgcagaaggatcggatggataagaatgcgtcaacctttgaggatgtcacccaggtgtcctctgcctaccagaagacagtacctgtcgaagctgtgaccagcaaaacaagtaacatcagagctaactttgaaaacctcgctaaggagaaagagcaggaggacaggcggaaggcggaggcggagagagcccagcggatggccaaggagcggcaggagcaggaagaggccaggaggaagctggaggagcaagccagagccaaaacgcaaacgccccctgtgtcgcccgcacctcagccaaccgaggagaggctgccctcgagccccgtctatgaggatgcggcttccttcaaggcagagctgagctacagaggccctgtgagtgggacggagccggagcccgtgtacagcatggaggccgctgactaccgagaggccagcagccagcagggcctggcctatgccacagaggctgtctatgaaagcgcagaggccccgggccactatcccgcagaggacagcacctacgatgagtacgagaacgatctggggatcacagccgtcgccctgtacgactaccaggctgcgggcgatgatgagatctcatttgaccctgatgacatcatcaccaacatcgagatgattgacgacggctggtggcgcggggtgtgcaagggccggtacgggctcttcccagccaactatgtggagctgcggcagtagggcccccagcccccccccggagctgcgccctggatcctcacactacagatcaggccttctttggttcttgggtggttttgggttttttctgttttttttttttttttttttttttgaaggtggggaggggaatatacacattgcttttatatttaatacttttgctgatgcttttgaaaatgtttatgccacagaatttgctaatatattgtaatcacattccttaggaggactttggtaattggttttatgcattgatggttttttttttcttttttgccaaattgactgtcacgcggcagcttcagggagctcgcattctcttgtgttcgtgttgccctcgtgcccatcaagtgcagtcgggacctcccaggacaagcacgaggcctcaggtcggccctgtggcgggtaggcaggaaggactgtcccagacgaggggcttcctctagagtctcactgctggggaggagaggactgggcctgatggaagttaacccggagctaagtcacccagagcacaggagctgccatgtcagatgggaaatctgcctatgtcataccgtgacagcccgcaggatcaggtgacttctagcagagaccctggtttttttcctgtgcccactccggcttgtcctcatctctacccatcccctgatgcccaggtcaccgggagggctgctgggagcctctcctgtccccgccgggcagtgtcactgagtccttgaaatcctcccctgccccgcgggtctctggattgggacgcacagtgcagttgaggtctgcgtcgggcttggcttttcacaaaggctgatgtcttaactgtcacccatatggtccctgggccaccgggcagcctggggcggtgtgtgtgccatgtcacagcatggcctctcggccttgggaaggaaggcagtgcctgctctgctgtgagccgccaggaaccctcctcctgtcaatgggggtgtagtatttttgccaaaatatcatgttcaatttcagtagtttgatcagttgaaggctagaagtgtgaagtgcagatgagtgtgtgttcttccccaaggtccccccacagctccaggacaccgctgtcctggcatttgtggccactcactttgtaggaaactcatctccttcctgaggagccgggaggctggaccagtcccgtcgtgcagtcaggtgggcggtgtgtctttccagaaggtcacgtggaaatgtctcgggacttgggtcccggagtgcccgtgaagcgtgtttttgctcctgaggtgcattttctcatcatccttgctttaccacaatgagcaatgaggtcgggttttatatgcaacttattgtatctgaattcctgtagcacacctcataggtatgatttttttaaattaaagaattcagaataaacattttttgatccaaaaa

SEQ ID NO: 23 - DNA, 3176 bp, Homo sapiens
agtagagcctggtgcctgggagcggctggcgcggcggaatccagggccgacccgggccggaccgaccccaggcggcgacggaatcagtccccaatgcctggaaattcctcattggattactgtgttttaaacagaatttcgtgaacagccttttatctccaagcggaaagaaagatgtggaaagcttcagcaggccacgctgtgtccatcgcccaggatgacgcgggggccgatgactgggagaccgaccctgattttgtgaatgatgtgagtgagaaggagcaaagatggggtgccaagacggtgcagggctccgggcaccaggagcatatcaacatacacaagctgagggagaatgtctttcaagagcatcagacccttaaggagaaggaacttgaaacaggaccaaaagcttcccatggctatggagggaaatttggtgtggaacaagaccgaatggataagtcagctgtcggccacgaatatcagtcgaaactttccaagcactgctcgcaggtggactcggtccgtggcttcggaggcaagtttggtgtccagatggacagagttgatcagtctgctgtaggctttgaataccaggggaagactgagaagcatgcctcccagaaagactactccagtggttttggcggcaagtatggcgtgcaggccgaccgagtagacaagagcgcggtgggcttcgactaccagggcaagacggagaagcacgagtcacagagagattactccaaaggtttcggcggcaaatacggtatcgacaaggacaaagtggataagagcgccgttggctttgagtatcaaggcaaaacggagaagcacgagtcccagaaagactatgtgaaagggtttggaggaaaatttggtgtgcagacagacagacaagacaaatgtgcccttggctgggatcaccaggagaaattgcagctgcatgaatcccaaaaagactactccaaaggattcggcgggaagtatggggtgcagaaggatcggatggataagaatgcgtcaacctttgaggatgtcacccaggtgtcctctgcctaccagaagacagtacctgtcgaagctgtgaccagcaaaacaagtaacatcagagctaactttgaaaacctcgctaaggagaaagagcaggaggacaggcggaaggcggaggcggagagagcccagcggatggccaaggagcggcaggagcaggaagaggccaggaggaagctggaggagcaagccagagccaaaacgcaaacgccccctgtgtcgcccgcacctcagccaaccgaggagaggctgccctcgagccccgtctatgaggatgcggcttccttcaaggcagagctgagctacagaggccctgtgagtgggacggagccggagcccgtgtacagcatggaggccgctgactaccgagaggccagcagccagcagggcctggcctatgccacagaggctgtctatgaaagcgcagaggccccgggccactatcccgcagaggacagcacctacgatgagtacgagaacgatctggggatcacagccgtcgccctgtacgactaccaggctgcgggcgatgatgagatctcatttgaccctgatgacatcatcaccaacatcgagatgattgacgacggctggtggcgcggggtgtgcaagggccggtacgggctcttcccagccaactatgtggagctgcggcagtagggcccccagcccccccccggagctgcgccctggatcctcacactacagatcaggccttctttggttcttgggtggttttgggttttttctgtttttttttttttttttttttttttgaaggtggggaggggaatatacacattgcttttatatttaatacttttgctgatgcttttgaaaatgtttatgccacagaatttgctaatatattgtaatcacattccttaggaggactttggtaattggttttatgcattgatggttttttttttcttttttgccaaattgactgtcacgcggcagcttcagggagctcgcattctcttgtgttcgtgttgccctcgtgcccatcaagtgcagtcgggacctcccaggacaagcacgaggcctcaggtcggccctgtggcgggtaggcaggaaggactgtcccagacgaggggcttcctctagagtctcactgctggggaggagaggactgggcctgatggaagttaacccggagctaagtcacccagagcacaggagctgccatgtcagatgggaaatctgcctatgtcataccgtgacagcccgcaggatcaggtgacttctagcagagaccctggtttttttcctgtgcccactccggcttgtcctcatctctacccatcccctgatgcccaggtcaccgggagggctgctgggagcctctcctgtccccgccgggcagtgtcactgagtccttgaaatcctcccctgccccgcgggtctctggattgggacgcacagtgcagttgaggtctgcgtcgggcttggcttttcacaaaggctgatgtcttaactgtcacccatatggtccctgggccaccgggcagcctggggcggtgtgtgtgccatgtcacagcatggcctctcggccttgggaaggaaggcagtgcctgctctgctgtgagccgccaggaaccctcctcctgtcaatgggggtgtagtatttttgccaaaatatcatgttcaatttcagtagtttgatcagttgaaggctagaagtgtgaagtgcagatgagtgtgtgttcttccccaaggtccccccacagctccaggacaccgctgtcctggcatttgtggccactcactttgtaggaaactcatctccttcctgaggagccgggaggctggaccagtcccgtcgtgcagtcaggtgggcggtgtgtctttccagaaggtcacgtggaaatgtctcgggacttgggtcccggagtgcccgtgaagcgtgtttttgctcctgaggtgcattttctcatcatccttgctttaccacaatgagcaatgaggtcgggttttatatgcaacttattgtatctgaattcctgtagcacacctcataggtatgatttttttaaattaaagaattcagaataaacattttttgatccacttgcgtgaaaaaaaaaaaaaaaaaaaaaaaaaaaaaaaaaaaaaaa

SEQ ID NO: 24 - protein, 550 aa, Homo sapiens
MWKASAGHAVSIAQDDAGADDWETDPDFVNDVSEKEQRWGAKTVQGSGHQEHINIHKLRENVFQEHQTLKEKELETGPKASHGYGGKFGVEQDRMDKSAVGHEYQSKLSKHCSQVDSVRGFGGKFGVQMDRVDQSAVGFEYQGKTEKHASQKDYSSGFGGKYGVQADRVDKSAVGFDYQGKTEKHESQRDYSKGFGGKYGIDKDKVDKSAVGFEYQGKTEKHESQKDYVKGFGGKFGVQTDRQDKCALGWDHQEKLQLHESQKDYKTGFGGKFGVQSERQDSAAVGFDYKEKLAKHESQQDYSKGFGGKYGVQKDRMDKNASTFEDVTQVSSAYQKTVPVEAVTSKTSNIRANFENLAKEKEQEDRRKAEAERAQRMAKERQEQEEARRKLEEQARAKTQTPPVSPAPQPTEERLPSSPVYEDAASFKAELSYRGPVSGTEPEPVYSMEAADYREASSQQGLAYATEAVYESAEAPGHYPAEDSTYDEYENDLGITAVALYDYQAAGDDEISFDPDDIITNIEMIDDGWWRGVCKGRYGLFPANYVELRQ

SEQ ID NO: 25 - protein, 513 aa, Homo sapiens
MWKASAGHAVSIAQDDAGADDWETDPDFVNDVSEKEQRWGAKTVQGSGHQEHINIHKLRENVFQEHQTLKEKELETGPKASHGYGGKFGVEQDRMDKSAVGHEYQSKLSKHCSQVDSVRGFGGKFGVQMDRVDQSAVGFEYQGKTEKHASQKDYSSGFGGKYGVQADRVDKSAVGFDYQGKTEKHESQRDYSKGFGGKYGIDKDKVDKSAVGFEYQGKTEKHESQKDYVKGFGGKFGVQTDRQDKCALGWDHQEKLQLHESQKDYSKGFGGKYGVQKDRMDKNASTFEDVTQVSSAYQKTVPVEAVTSKTSNIRANFENLAKEKEQEDRRKAEAERAQRMAKERQEQEEARRKLEEQARAKTQTPPVSPAPQPTEERLPSSPVYEDAASFKAELSYRGPVSGTEPEPVYSMEAADYREASSQQGLAYATEAVYESAEAPGHYPAEDSTYDEYENDLGITAVALYDYQAAGDDEISFDPDDIITNIEMIDDGWWRGVCKGRYGLFPANYVELRQ

SEQ ID NO: 26 - DNA, 3065 bp, Homo sapiens
agtagagcctggtgcctgggagcggctggcgcggcggaatccagggccgacccgggccggaccgaccccaggcggcgacggaatcagtccccaatgcctggaaattcctcattggattactgtgttttaaacagaatttcgtgaacagccttttatctccaagcggaaagaaagatgtggaaagcttcagcaggccacgctgtgtccatcgcccaggatgacgcgggggccgatgactgggagaccgaccctgattttgtgaatgatgtgagtgagaaggagcaaagatggggtgccaagacggtgcagggctccgggcaccaggagcatatcaacatacacaagctgagggagaatgtctttcaagagcatcagacccttaaggagaaggaacttgaaacaggaccaaaagcttcccatggctatggagggaaatttggtgtggaacaagaccgaatggataagtcagctgtcggccacgaatatcagtcgaaactttccaagcactgctcgcaggtggactcggtccgtggcttcggaggcaagtttggtgtccagatggacagagttgatcagtctgctgtaggctttgaataccaggggaagactgagaagcatgcctcccagaaagactactccagtggttttggcggcaagtatggcgtgcaggccgaccgagtagacaagagcgcggtgggcttcgactaccagggcaagacggagaagcacgagtcacagagagattactccaaaggtttcggcggcaaatacggtatcgacaaggacaaagtggataagagcgccgttggctttgagtatcaaggcaaaacggagaagcacgagtcccagaaagactatgtgaaaggattcggcgggaagtatggggtgcagaaggatcggatggataagaatgcgtcaacctttgaggatgtcacccaggtgtcctctgcctaccagaagacagtacctgtcgaagctgtgaccagcaaaacaagtaacatcagagctaactttgaaaacctcgctaaggagaaagagcaggaggacaggcggaaggcggaggcggagagagcccagcggatggccaaggagcggcaggagcaggaagaggccaggaggaagctggaggagcaagccagagccaaaacgcaaacgccccctgtgtcgcccgcacctcagccaaccgaggagaggctgccctcgagccccgtctatgaggatgcggcttccttcaaggcagagctgagctacagaggccctgtgagtgggacggagccggagcccgtgtacagcatggaggccgctgactaccgagaggccagcagccagcagggcctggcctatgccacagaggctgtctatgaaagcgcagaggccccgggccactatcccgcagaggacagcacctacgatgagtacgagaacgatctggggatcacagccgtcgccctgtacgactaccaggctgcgggcgatgatgagatctcatttgaccctgatgacatcatcaccaacatcgagatgattgacgacggctggtggcgcggggtgtgcaagggccggtacgggctcttcccagccaactatgtggagctgcggcagtagggcccccagcccccccccggagctgcgccctggatcctcacactacagatcaggccttctttggttcttgggtggttttgggttttttctgtttttttttttttttttttttttttgaaggtggggaggggaatatacacattgcttttatatttaatacttttgctgatgcttttgaaaatgtttatgccacagaatttgctaatatattgtaatcacattccttaggaggactttggtaattggttttatgcattgatggttttttttttcttttttgccaaattgactgtcacgcggcagcttcagggagctcgcattctcttgtgttcgtgttgccctcgtgcccatcaagtgcagtcgggacctcccaggacaagcacgaggcctcaggtcggccctgtggcgggtaggcaggaaggactgtcccagacgaggggcttcctctagagtctcactgctggggaggagaggactgggcctgatggaagttaacccggagctaagtcacccagagcacaggagctgccatgtcagatgggaaatctgcctatgtcataccgtgacagcccgcaggatcaggtgacttctagcagagaccctggtttttttcctgtgcccactccggcttgtcctcatctctacccatcccctgatgcccaggtcaccgggagggctgctgggagcctctcctgtccccgccgggcagtgtcactgagtccttgaaatcctcccctgccccgcgggtctctggattgggacgcacagtgcagttgaggtctgcgtcgggcttggcttttcacaaaggctgatgtcttaactgtcacccatatggtccctgggccaccgggcagcctggggcggtgtgtgtgccatgtcacagcatggcctctcggccttgggaaggaaggcagtgcctgctctgctgtgagccgccaggaaccctcctcctgtcaatgggggtgtagtatttttgccaaaatatcatgttcaatttcagtagtttgatcagttgaaggctagaagtgtgaagtgcagatgagtgtgtgttcttccccaaggtccccccacagctccaggacaccgctgtcctggcatttgtggccactcactttgtaggaaactcatctccttcctgaggagccgggaggctggaccagtcccgtcgtgcagtcaggtgggcggtgtgtctttccagaaggtcacgtggaaatgtctcgggacttgggtcccggagtgcccgtgaagcgtgtttttgctcctgaggtgcattttctcatcatccttgctttaccacaatgagcaatgaggtcgggttttatatgcaacttattgtatctgaattcctgtagcacacctcataggtatgatttttttaaattaaagaattcagaataaacattttttgatccacttgcgtgaaaaaaaaaaaaaaaaaaaaaaaaaaaaaaaaaaaaaaa

SEQ ID NO: 27 - protein, 476 aa, Homo sapiens
MWKASAGHAVSIAQDDAGADDWETDPDFVNDVSEKEQRWGAKTVQGSGHQEHINIHKLRENVFQEHQTLKEKELETGPKASHGYGGKFGVEQDRMDKSAVGHEYQSKLSKHCSQVDSVRGFGGKFGVQMDRVDQSAVGFEYQGKTEKHASQKDYSSGFGGKYGVQADRVDKSAVGFDYQGKTEKHESQRDYSKGFGGKYGIDKDKVDKSAVGFEYQGKTEKHESQKDYVKGFGGKYGVQKDRMDKNASTFEDVTQVSSAYQKTVPVEAVTSKTSNIRANFENLAKEKEQEDRRKAEAERAQRMAKERQEQEEARRKLEEQARAKTQTPPVSPAPQPTEERLPSSPVYEDAASFKAELSYRGPVSGTEPEPVYSMEAADYREASSQQGLAYATEAVYESAEAPGHYPAEDSTYDEYENDLGITAVALYDYQAAGDDEISFDPDDIITNIEMIDDGWWRGVCKGRYGLFPANYVELRQ

SEQ ID NO: 28 - DNA, 1641 bp, Mus sp.
atgtggaaagcctctgcaggccatgctgtgtccatcacgcaggatgatggaggagctgatgactgggagactgatcctgattttgtgaatgatgtgagtgaaaaggagcagagatggggtgctaaaaccgtgcagggatcggggcaccaggaacacatcaacattcacaagcttcgagagaatgtcttccaagaacaccagacgctcaaggagaaggagctggaaacgggacccaaggcttcccacggctatggcgggaagttcggtgtggagcaggataggatggacagatcagccgtgggccatgagtaccagtcgaagctttccaagcactgctcacaagtggactcggtccggggcttcggaggcaagttcggtgtccagatggacagggtggatcagtctgctgtaggctttgaataccaggggaagactgagaagcatgcctcccagaaagactactctagtggcttcggtggcaaatacggtgtgcaagctgaccgtgtagacaagagtgccgtgggctttgactaccagggcaagacggagaagcatgagtctcagaaagattactccaaaggttttggtggcaaatatgggattgacaaggacaaggtggataaaagtgctgtgggctttgagtatcaaggcaagacagagaagcacgaatcccagaaagactatgtaaaaggctttggaggaaagtttggtgtgcagacagacagacaggacaagtgtgcccttggctgggaccatcaggagaagctgcagctgcatgaatcccaaaaagactataagactggtttcggaggcaaatttggtgttcagtccgagaggcaggactcctccgctgtggggtttgattacaaggagagattggccaagcacgagccccagcaagactatgccaaaggattcggcgggaagtatggggtgcagaaggatcggatggacaagaatgcatccacctttgaagaagtggtccaggtgccatctgcctatcagaagactgtccccattgaggccgtaaccagcaaaaccagtaatatccgtgctaactttgaaaacctggcaaaggagagagagcaggaggacaggcggaaggcagaagccgagagagctcagcggatggccaaagaaagacaggagcaggaggaggcgcgcaggaagctggaagagcaagccagagccaagaagcagacgccccctgcatcccctagtcctcaaccaattgaagacagaccaccctccagccccatctatgaggatgcagctccgttcaaggccgagccgagctaccgaggtagcgaacctgagcctgagtacagcatcgaggccgcaggcattcctgaggctggcagccagcaaggcctgacctatacatcagagcccgtgtacgagactacagaggctcctggccactatcaagcagaggatgacacctacgatgggtatgagagtgacctgggcatcacagccatcgccctgtatgactaccaggctgctggcgatgatgagatctcctttgaccctgatgacatcatcaccaacatagaaatgattgacgatggctggtggcgtggggtgtgcaagggcagatacgggctcttcccagccaactatgtggagctgcggcagtag

SEQ ID NO: 29 - protein, 546 aa, Mus sp.
MWKASAGHAVSITQDDGGADDWETDPDFVNDVSEKEQRWGAKTVQGSGHQEHINIHKLRENVFQEHQTLKEKELETGPKASHGYGGKFGVEQDRMDRSAVGHEYQSKLSKHCSQVDSVRGFGGKFGVQMDRVDQSAVGFEYQGKTEKHASQKDYSSGFGGKYGVQADRVDKSAVGFDYQGKTEKHESQKDYSKGFGGKYGIDKDKVDKSAVGFEYQGKTEKHESQKDYVKGFGGKFGVQTDRQDKCALGWDHQEKLQLHESQKDYKTGFGGKFGVQSERQDSSAVGFDYKERLAKHEPQQDYAKGFGGKYGVQKDRMDKNASTFEEVVQVPSAYQKTVPIEAVTSKTSNIRANFENLAKEREQEDRRKAEAERAQRMAKERQEQEEARRKLEEQARAKKQTPPASPSPQPIEDRPPSSPIYEDAAPFKAEPSYRGSEPEPEYSIEAAGIPEAGSQQGLTYTSEPVYETTEAPGHYQAEDDTYDGYESDLGITAIALYDYQAAGDDEISFDPDDIITNIEMIDDGWWRGVCKGRYGLFPANYVELRQ